(19) United States
(12) Patent Application Publication
(10) Pub. No.: US 2022/0069226 A1
(43) Pub. Date: Mar. 3, 2022

(54) COMPOSITION FOR ORGANIC OPTOELECTRONIC ELEMENT, ORGANIC OPTOELECTRONIC ELEMENT, AND DISPLAY DEVICE

(71) Applicants: SAMSUNG SDI CO., LTD., Yongin-si, Gyeonggi-do (KR); SAMSUNG ELECTRONICS CO., LTD., Suwon-si, Gyeonggi-do (KR)

(72) Inventors: Byoungkwan LEE, Yongin-si, Gyeonggi-do (KR); Dong Min KANG, Yongin-si, Gyeonggi-do (KR); Dongyeong KIM, Yongin-si, Gyeonggi-do (KR); Jun Seok KIM, Yongin-si, Gyeonggi-do (KR); Jinhyun LUI, Yongin-si, Gyeonggi-do (KR); Sangshin LEE, Yongin-si, Gyeonggi-do (KR); Byungku KIM, Yongin-si, Gyeonggi-do (KR); Jaehoon KIM, Yongin-si, Gyeonggi-do (KR); Eun Sun YU, Yongin-si, Gyeonggi-do (KR); Ho Kuk JUNG, Yongin-si, Gyeonggi-do (KR)

(21) Appl. No.: 17/272,396

(22) PCT Filed: Sep. 6, 2019
(86) PCT No.: PCT/KR2019/011571

§ 371 Date: Mar. 1, 2021

(30) Foreign Application Priority Data
Sep. 27, 2018 (KR) 10-2018-0115287

(51) Int. Cl. H01L 51/00; C07D 209/88; C07D 405/12; C07D 495/04

(52) U.S. Cl. CPC H01L 51/0061; C07D 209/88; C07D 405/12; H01L 51/5016; H01L 51/0065; H01L 51/0072; H01L 51/0073; C07D 495/04

(57) ABSTRACT

The present invention relates to a composition for an organic optoelectronic element, an organic optoelectronic element and a display device, the composition including a first compound for an organic optoelectronic element, represented by a combination of Chemical Formula 1 and Chemical Formula 2; and a second compound for an organic optoelectronic element, represented by a combination of Chemical Formula 3 and Chemical Formula 4.

In Chemical Formula 1 to Chemical Formula 4, each substituent is defined as that in the specification.

### TECHNICAL FIELD

A composition for an organic optoelectronic element, an organic optoelectronic element, and a display device are disclosed.

### BACKGROUND ART

An organic optoelectronic element (organic optoelectronic diode) is a device that converts electrical energy into photoenergy, and vice versa.

An organic optoelectronic element may be classified as follows in accordance with its driving principles. One is a photoelectric element that generates electrical energy by separating excitons formed by light energy into electrons and holes, and transferring the electrons and holes to different electrodes, respectively and the other is a light emitting element that generates light energy from electrical energy by supplying voltage or current to the electrodes.

Examples of the organic optoelectronic element include an organic photoelectric element, an organic light emitting diode, an organic solar cell, and an organic photo conductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic element capable of implementing a high efficiency and long life-span organic optoelectronic element.

Another embodiment provides an organic optoelectronic element including the composition.

Another embodiment provides a display device including the organic optoelectronic element.

### Technical Solution

According to an embodiment, a composition for an organic optoelectronic element includes a first compound for an organic optoelectronic element, represented by a combination of Chemical Formula 1 and Chemical Formula 2, and a second compound for an organic optoelectronic element, represented by a combination of Chemical Formula 3 and Chemical Formula 4.

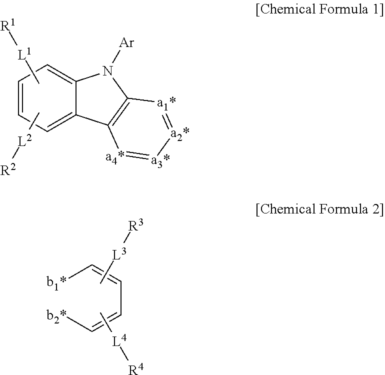

In Chemical Formula 1 and Chemical Formula 2,

Ar is a substituted or unsubstituted C6 to C30 aryl group, a substituted or unsubstituted C2 to C30 heterocyclic group, or a combination thereof,

adjacent two of a₁* to a₄* are linked with b₁* and b₂*, respectively,

the rest of a₁* to a₄*, not linked with b₁* and b₂* are independently C-L^{a}-R^{a},

L^{a} and L¹ to L⁴ are independently a single bond, a substituted or unsubstituted C6 to C20 arylene group, a substituted or unsubstituted C2 to C20 heterocyclic group, or a combination thereof,

R^{a} and R¹ to R⁴ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and

at least one of R^{a} and R¹ to R⁴ is a group represented by Chemical Formula a,

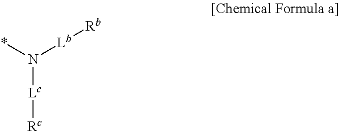

wherein, in Chemical Formula a,

L^{b} and L^{c} are independently a single bond, a substituted or unsubstituted C6 to C20 arylene group, a substituted or unsubstituted C2 to C20 heterocyclic group, or a combination thereof,

R^{b} and R^{c} are independently a substituted or unsubstituted C6 to C30 aryl group, a substituted or unsubstituted C2 to C30 heterocyclic group, or a combination thereof, and

* is a linking point with L^{a} and L¹ to L⁴;

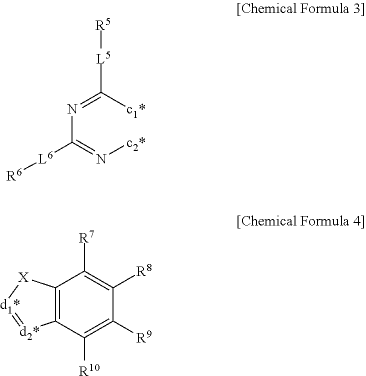

wherein, in Chemical Formula 3 and Chemical Formula 4,

X is O or S,

c₁* and c₂* are linked with d₁* and d₂* or d₂* and d₁*, respectively,

L⁵ and L⁶ are independently a single bond, a substituted or unsubstituted C6 to C20 arylene group, a substituted or unsubstituted C2 to C20 heterocyclic group, or a combination thereof,

R⁵ to R¹⁰ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and

at least one of R⁵ and R⁶ is a substituted or unsubstituted C6 to C30 aryl group, a substituted or unsubstituted C2 to C30 heterocyclic group, or a combination thereof.

According to another embodiment, an organic optoelectronic element includes an anode and a cathode facing each other, and at least one organic layer between the anode and the cathode, and the organic layer includes the composition for an organic optoelectronic element.

According to another embodiment, a display device including the organic optoelectronic element is provided.

### Advantageous Effects

High efficiency and long life-span organic optoelectronic elements may be implemented.

### DESCRIPTION OF THE DRAWINGS

FIGS. 1 and 2 are cross-sectional views each illustrating an organic light emitting diode according to embodiments.

### DESCRIPTION OF SYMBOLS

100, 200: organic light emitting diode

105: organic layer

110: cathode

120: anode

130: light emitting layer

140: hole auxiliary layer

### BEST MODE

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, or a C2 to C30 heteroaryl group. In addition, in specific examples of the present invention, “substituted” refers to replacement of at least one hydrogen of a substituent or a compound by deuterium, a C1 to C20 alkyl group, or a C6 to C30 aryl group. In addition, in specific examples of the present invention, “substituted” refers to replacement of at least one hydrogen of a substituent or a compound by deuterium, a C1 to C5 alkyl group, a C6 to C18 aryl group, a pyridinyl group, a quinolinyl group, an isoquinolinyl group, a dibenzofuranyl group, a dibenzothiophenyl group, or a carbazolyl group. In addition, in specific examples of the present invention, “substituted” refers to replacement of at least one hydrogen of a substituent or a compound by deuterium, a C1 to C5 alkyl group, a C6 to C18 aryl group, a dibenzofuranyl group, or a dibenzothiophenyl group. In addition, in specific examples of the present invention, “substituted” refers to replacement of at least one hydrogen of a substituent or a compound by deuterium, a methyl group, an ethyl group, a propanyl group, a butyl group, a phenyl group, a biphenyl group, a terphenyl group, a naphthyl group, a triphenyl group, a dibenzofuranyl group, or a dibenzothiophenyl group.

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a composition for an organic optoelectronic element according to an embodiment is described.

The composition for an organic optoelectronic element according to an embodiment includes a first compound for an organic optoelectronic element having hole characteristics and a second compound for an organic optoelectronic element having electron characteristics.

The first compound for an organic optoelectronic element is represented by a combination of Chemical Formula 1 and Chemical Formula 2.

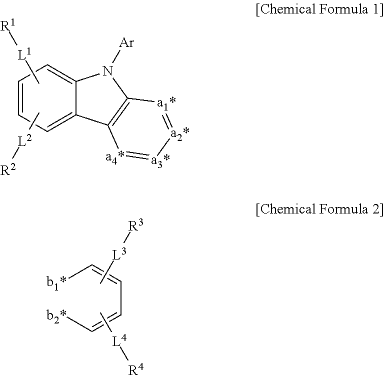

In Chemical Formula 1 and Chemical Formula 2,

Ar is a substituted or unsubstituted C6 to C30 aryl group, a substituted or unsubstituted C2 to C30 heterocyclic group, or a combination thereof,

adjacent two of a₁* to a₄* are linked with b₁* and b₂*, respectively,

the rest of a₁* to a₄*, not linked with b₁* and b₂* are independently C-L^{a}-R^{a},

L^{a} and L¹ to L⁴ are independently a single bond, a substituted or unsubstituted C6 to C20 arylene group, a substituted or unsubstituted C2 to C20 heterocyclic group, or a combination thereof,

R^{a} and R¹ to R⁴ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and

at least one of R^{a} and R¹ to R⁴ is a group represented by Chemical Formula a,

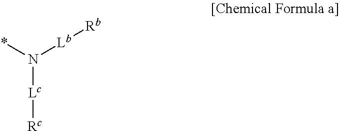

wherein, in Chemical Formula a,

L^{b} and L^{c} are independently a single bond, a substituted or unsubstituted C6 to C20 arylene group, a substituted or unsubstituted C2 to C20 heterocyclic group, or a combination thereof,

R^{b} and R^{c} are independently a substituted or unsubstituted C6 to C30 aryl group, a substituted or unsubstituted C2 to C30 heterocyclic group, or a combination thereof, and

* is a linking point with L^{a} and L¹ to L⁴.

The first compound for an organic optoelectronic element has a structure in which benzocarbazole is substituted with an amine, so that the HOMO electron cloud expands from amine to benzocarbazole, so that it has high HOMO energy, and has excellent hole injection and transfer characteristics.

In addition, since benzocarbazole has a relatively high HOMO energy compared with bicarbazole and indolocarbazole, a device having a low driving voltage may be implemented by applying a structure in which benzocarbazole substituted with an amine.

In addition, bicarbazole and indolocarbazole have a high T1 energy and are not suitable as a red host, whereas a structure in which benzocarbazole substituted with an amine has a T1 energy suitable as a red host. Accordingly, the device to which the compsition according to the present invention is applied may realize high efficiency/long life-span characteristics.

Meanwhile, since it is included with the second compound for an organic optoelectronic element, good interfacial characteristics and a hole transport capability and electron transport capability are exhibited, thereby reducing a driving voltage of a device including it.

For example, R^{b} and R^{c} may independently be a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, or a substituted or unsubstituted dibenzothiophenyl group.

For example, R^{b} and R^{c} may independently be a substituted or unsubstituted phenyl group, a substituted or unsubstituted p-biphenyl group, a substituted or unsubstituted fluorenyl group, a substituted or unsubstituted dibenzofuranyl group, or a substituted or unsubstituted dibenzothiophenyl group.

For example, L^{b} and L^{c} may independently be a single bond, a phenylene group, a biphenylene group, a naphthylene group, an anthracenylene group, or a phenanthrenylene group.

For example, L^{b} and L^{c} may independently be a single bond or a phenylene group.

For example, Ar may independently be a substituted or unsubstituted C6 to C20 aryl group or a substituted or unsubstituted C2 to C20 heterocyclic group.

For example, Ar may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted dibenzofuranyl group, a substituted or unsubstituted dibenzothiphenyl group, a substituted or unsubstituted carbazolyl group, or a combination thereof.

For example, Ar may be a substituted or unsubstituted phenyl group, a substituted or unsubstituted biphenyl group, a substituted or unsubstituted naphthyl group, a substituted or unsubstituted dibenzofuranyl group, a substituted or unsubstituted dibenzothiophenyl group, or a substituted or unsubstituted carbazolyl group, but is not limited thereto.

For example, L^{a} and L¹ to L⁴ may independently be a single bond or a substituted or unsubstituted C6 to C20 arylene group.

For example, L^{a} and L¹ to L⁴ may independently be a single bond, a substituted or unsubstituted phenylene group, a substituted or unsubstituted biphenylene group, a substituted or unsubstituted terphenylene group, or a substituted or unsubstituted naphthylene group.

For example, L^{a} and L¹ to L⁴ may independently be a single bond, a substituted or unsubstituted m-phenylene group, a substituted or unsubstituted p-phenylene group, a substituted or unsubstituted o-phenylene group, a substituted or unsubstituted m-biphenylene group, a substituted or unsubstituted p-biphenylene group, a substituted or unsubstituted o-biphenylene group, a substituted or unsubstituted m-terphenylene group, a substituted or unsubstituted p-terphenylene group, or a substituted or unsubstituted o-terphenylene group. Herein, “substituted” may for example refer to replacement of at least one hydrogen by deuterium, a C1 to C20 alkyl group, a C6 to C20 aryl group, halogen, a cyano group, or a combination thereof.

For example, R^{a} and R¹ to R⁴ are independently hydrogen, deuterium, a cyano group, a substituted or unsubstituted C1 to C10 alkyl group, a substituted or unsubstituted C6 to C20 aryl group, a substituted or unsubstituted C2 to C20 heterocyclic group, or the group represented by Chemical Formula a.

For example, R^{a} and R¹ to R⁴ may independently be hydrogen or a group represented by Chemical Formula a, but are not limited thereto.

As an example, the first compound for an organic optoelectronic element may be, for example, represented by one of Chemical Formula 1A to Chemical Formula 1C depending on the fusion position of Chemical Formula 1 and Chemical Formula 2.

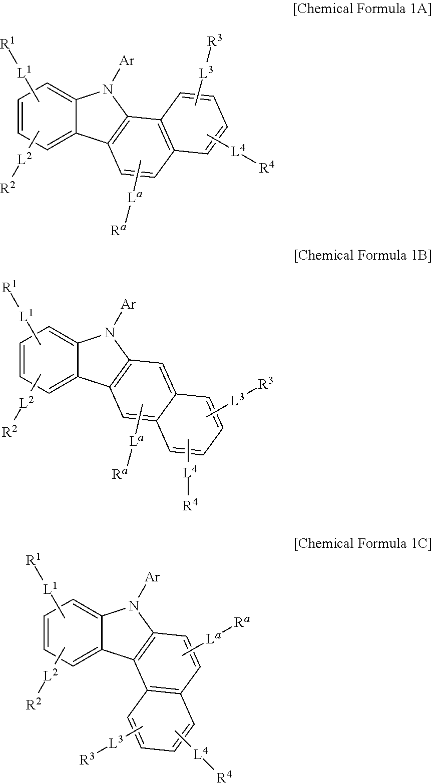

In Chemical Formula 1A to Chemical Formula 1C, Ar, L^{a}, and L¹ to L⁴, and R^{a} and R¹ to R⁴ are the same as described above.

For example, Chemical Formula 1A may be represented by one of Chemical Formula 1A-1 to Chemical Formula 1A-3, depending on the position of substitution of the group represented by Chemical Formula a.

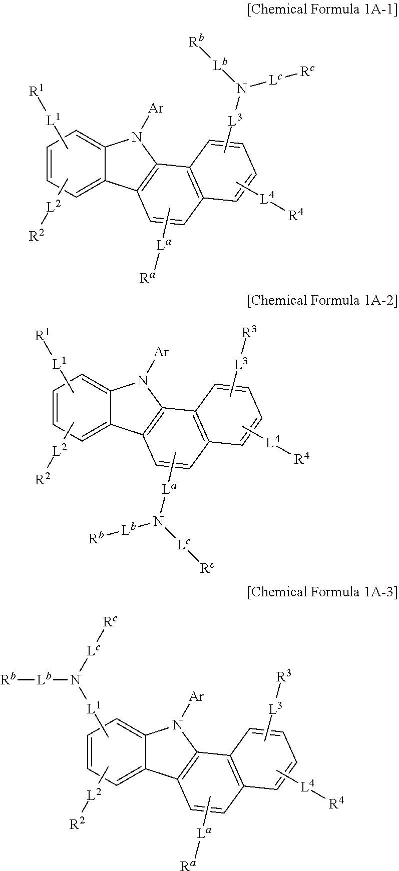

In Chemical Formula 1A-1 to Chemical Formula 1A-3, Ar, L^{a}, L^{b}, L^{c}, L¹ to L⁴, R^{a}, R¹ to R⁴, R^{b}, and R^{c} are the same as described above.

For example, Chemical Formula 1A-1 may be represented by one of Chemical Formula 1A-1-a to Chemical Formula 1A-1-d, depending on the specific substitution position of the group represented by Chemical Formula a.

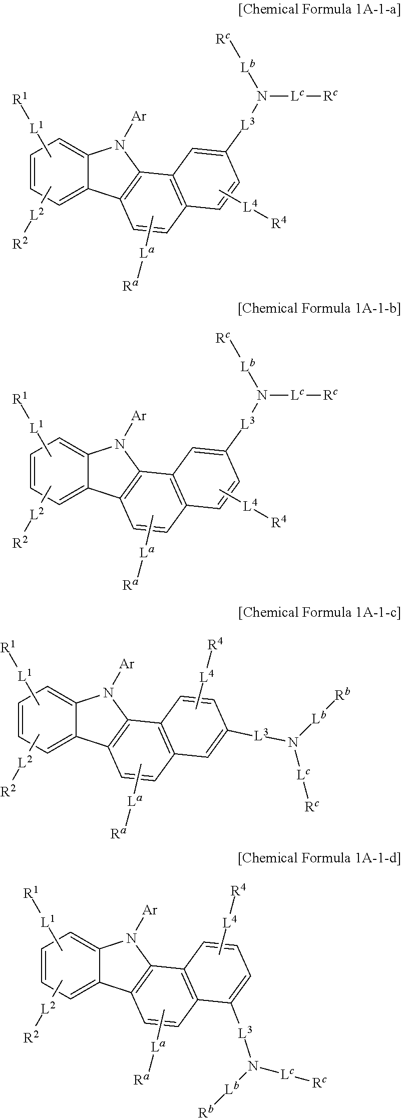

In Chemical Formula 1A-1-a to Chemical Formula 1A-1-d, Ar, L^{a}, L^{b}, L^{c}, L¹ to L⁴, R^{a}, R¹ to R⁴, R^{b}, and R^{c} are the same as described above.

In an embodiment, Chemical Formula 1A-1 may be represented by Chemical Formula 1A-1-b or Chemical Formula 1A-1-c.

For example, Chemical Formula 1A-2 may be represented by Chemical Formula 1A-2-a or Chemical Formula 1A-2-b, depending on the specific substitution position of the group represented by Chemical Formula a.

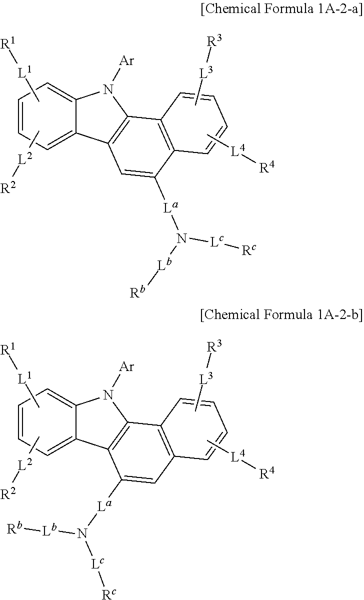

In Chemical Formula 1A-2-a and Chemical Formula 1A-2-b, Ar, L^{a}, L^{b}, L^{c}, L¹ to L⁴, R¹ to R⁴, R^{b}, and R^{c} are the same as described above.

In an embodiment, Chemical Formula 1A-2 may be represented by Chemical Formula 1A-2-a.

For example, Chemical Formula 1A-3 may be represented by one of Chemical Formula 1A-3-a to Chemical Formula 1A-3-d, depending on the specific substitution position of the group represented by Chemical Formula a.

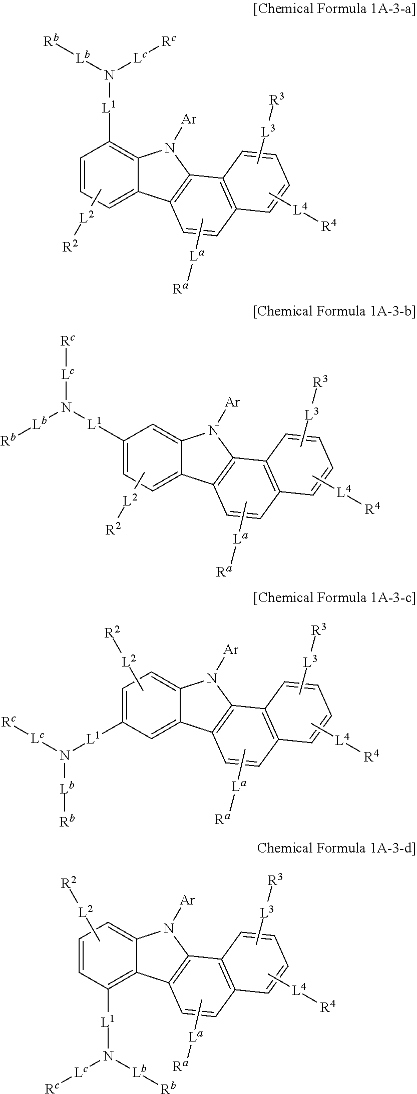

In Chemical Formula 1A-3-a to Chemical Formula 1A-3-d, Ar, L^{a}, L^{b}, L^{c}, L¹ to L⁴, R^{a}, R¹ to R⁴, R^{b}, and R^{c} are the same as described above.

In an embodiment, Chemical Formula 1A-3 may be represented by Chemical Formula 1A-3-b or Chemical Formula 1A-3-c.

For example, Chemical Formula 1B may be represented by one of Chemical Formula 1B-1 to Chemical Formula 1B-3, depending on the position of substitution of the group represented by Chemical Formula a.

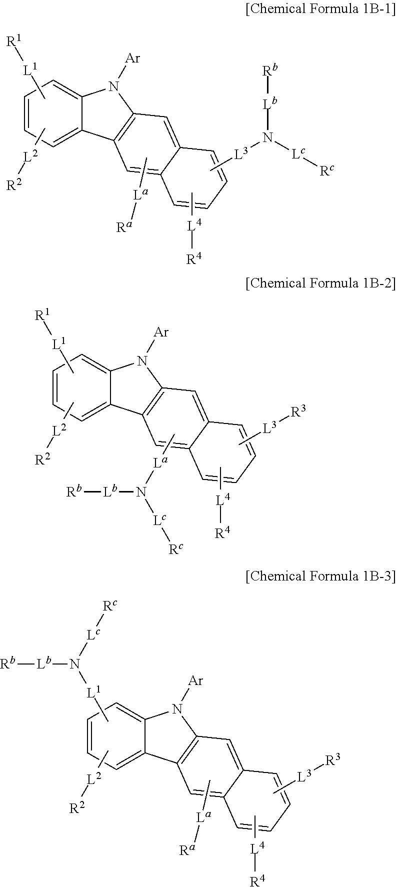

In Chemical Formula 1B-1 to Chemical Formula 1B-3, Ar, L^{a}, L^{b}, L^{c}, L¹ to L⁴, R^{a}, R¹ to R⁴, R^{b}, and R^{c} are the same as described above.

For example, Chemical Formula 1B-1 may be represented by one of Chemical Formula 1B-1-a to Chemical Formula 1B-1-d, depending on the specific substitution position of the group represented by Chemical Formula a.

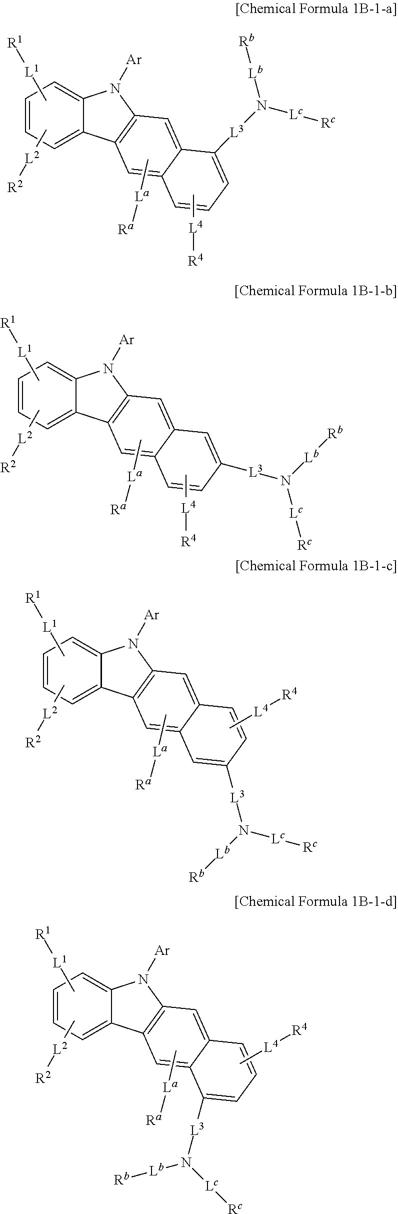

In Chemical Formula 1B-1-a to Chemical Formula 1B-1-d, Ar, L^{a}, L^{b}, L^{c}, L¹ to L⁴, R^{a}, R¹ to R⁴, R^{b}, and R^{c} are the same as described above.

For example, Chemical Formula 1B-2 may be represented by Chemical Formula 1B-2-a or Chemical Formula 1B-2-b, depending on the specific substitution position of the group represented by Chemical Formula a.

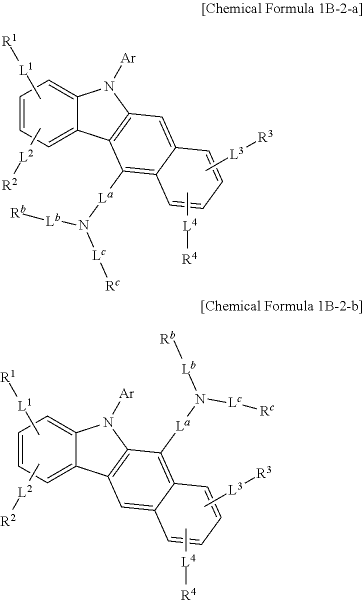

In Chemical Formula 1B-2-a and Chemical Formula 1B-2-b, Ar, L^{a}, L^{b}, L^{c}, L¹ to L⁴, R¹ to R⁴, R^{b}, and R^{c} are the same as described above.

For example, Chemical Formula 1B-3 may be represented by one of Chemical Formula 1B-3-a to Chemical Formula 1B-3-d, depending on the specific substitution position of the group represented by Chemical Formula a.

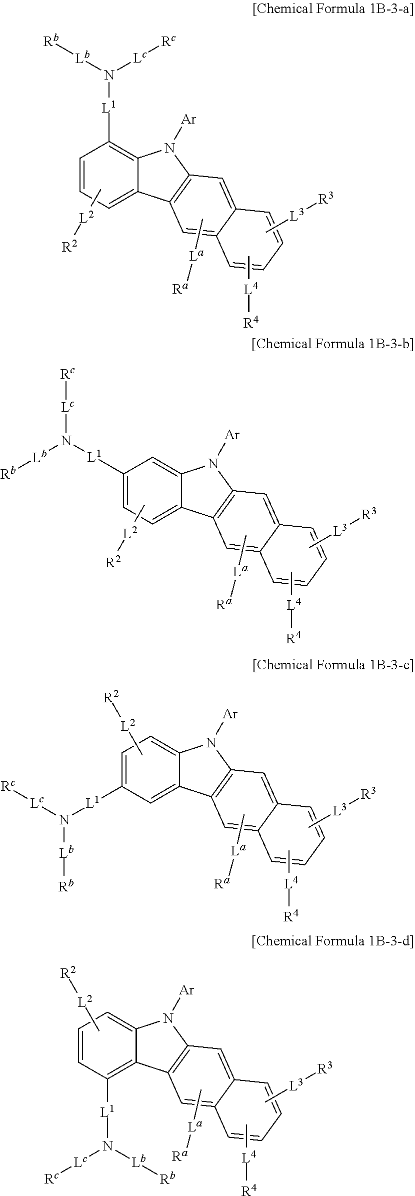

In Chemical Formula 1B-3-a to Chemical Formula 1B-3-d, Ar, L^{a}, L^{b}, L^{c}, L¹ to L⁴, R^{a}, R¹ to R⁴, R^{b}, and R^{c} are the same as described above.

In one embodiment, Chemical Formula 1B-3 may be represented by Chemical Formula 1B-3-b.

For example, Chemical Formula 1C may be represented by one of Chemical Formula 1C-1 to Chemical Formula 1C-3, depending on the substitution position of the group represented by Chemical Formula a.

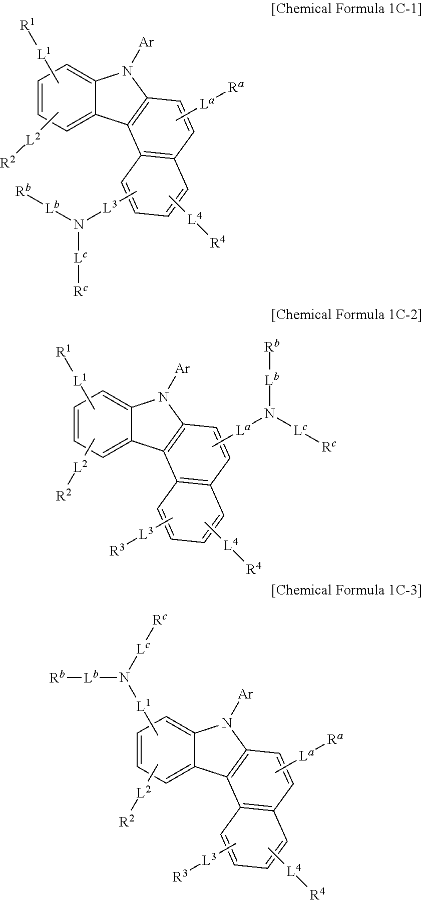

In Chemical Formula 1C-1 to Chemical Formula 1C-3, Ar, L^{a}, L^{b}, L^{c}, L¹ to L⁴, R^{a}, R¹ to R⁴, R^{b}, and R^{c} are the same as described above.

For example, Chemical Formula 1C-1 may be represented by one of Chemical Formula 1C-1-a to Chemical Formula 1C-1-d depending on the specific substitution position of the group represented by Chemical Formula a.

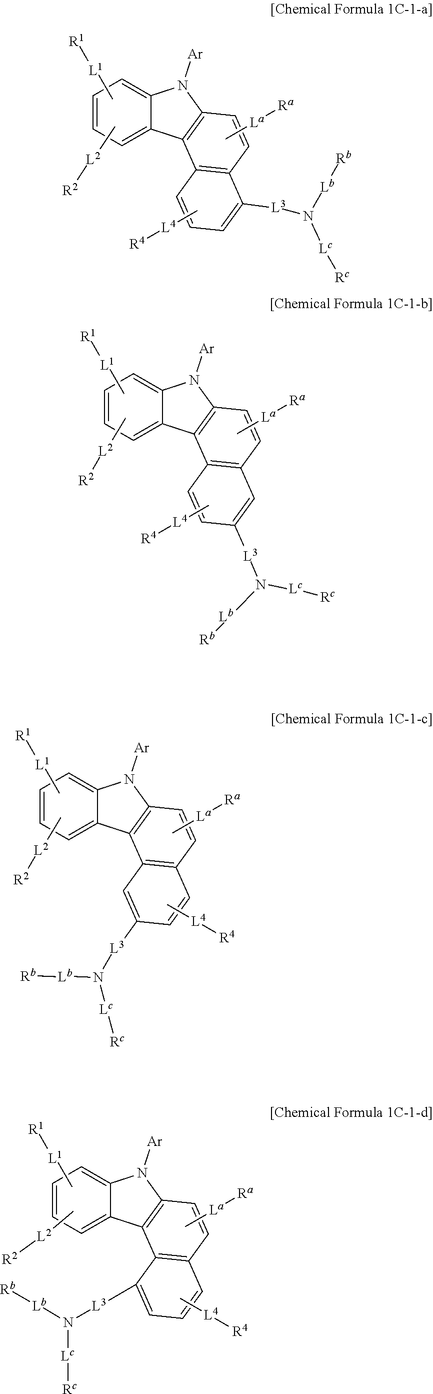

In Chemical Formula 1C-1-a to Chemical Formula 1C-1-d, Ar, L^{a}, L^{b}, L^{c}, L¹ to L⁴, R^{a}, R¹ to R⁴, R^{b}, and R^{c} are the same as described above.

In one embodiment, Chemical Formula 1C-1 may be represented by Chemical Formula 1C-1-b.

For example, Chemical Formula 1C-2 may be represented by Chemical Formula 1C-2-a or Chemical Formula 1C-2-b according to the specific substitution position of the group represented by Chemical Formula a.

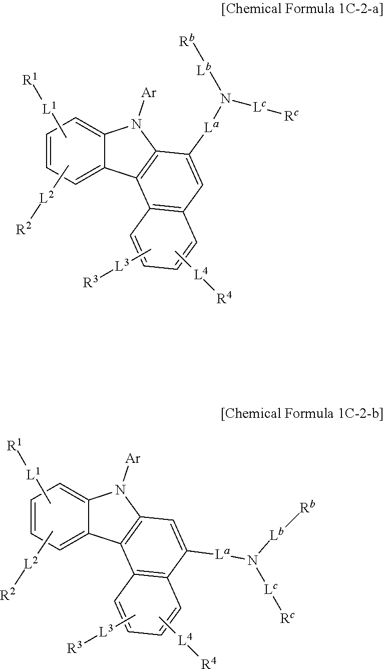

In Chemical Formula 1C-2-a and Chemical Formula 1C-2-b, Ar, L^{a}, L^{b}, L^{c}, L¹ to L⁴, R¹ to R⁴, R^{b}, and R^{c} are the same as described above.

For example, Chemical Formula 1C-3 may be represented by one of Chemical Formula 1C-3-a to Chemical Formula 1C-3-d, depending on the specific substitution position of the group represented by Chemical Formula a.

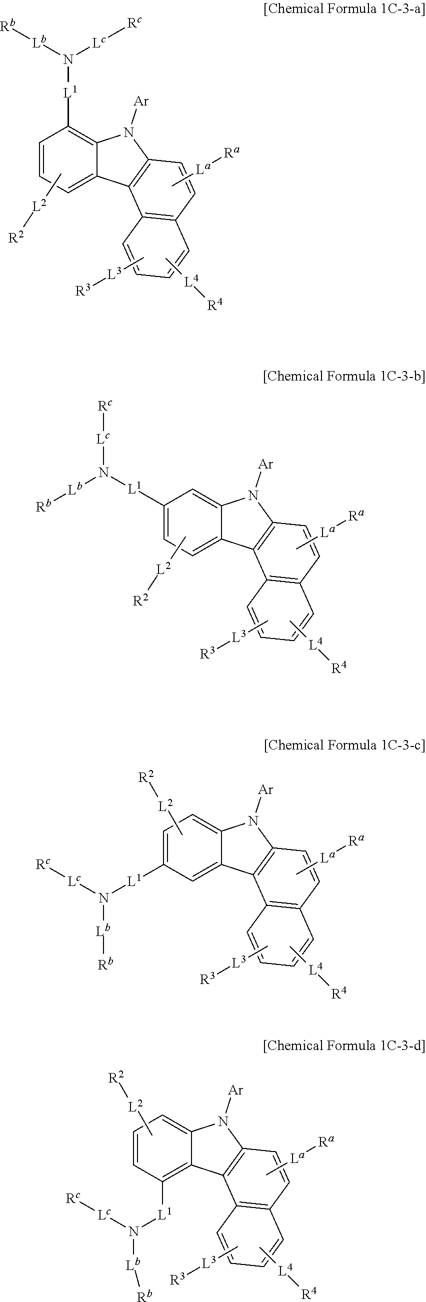

In Chemical Formula 1C-3-a to Chemical Formula 1C-3-d, Ar, L^{a}, L^{b}, L^{c}, L¹ to L⁴, R^{a}, R¹ to R⁴, R^{b}, and R^{c} are the same as described above.

In an embodiment, Chemical Formula 1C-3 may be represented by Chemical Formula 1C-3-b.

In a specific embodiment of the present invention, the first compound for an organic optoelectronic element may be represented by Chemical Formula 1A, specifically, Chemical Formula 1A-1, for example, Chemical Formula 1A-1-b.

The first compound for an organic optoelectronic element may be, for example, one selected from compounds of Group 1, but is not limited thereto.

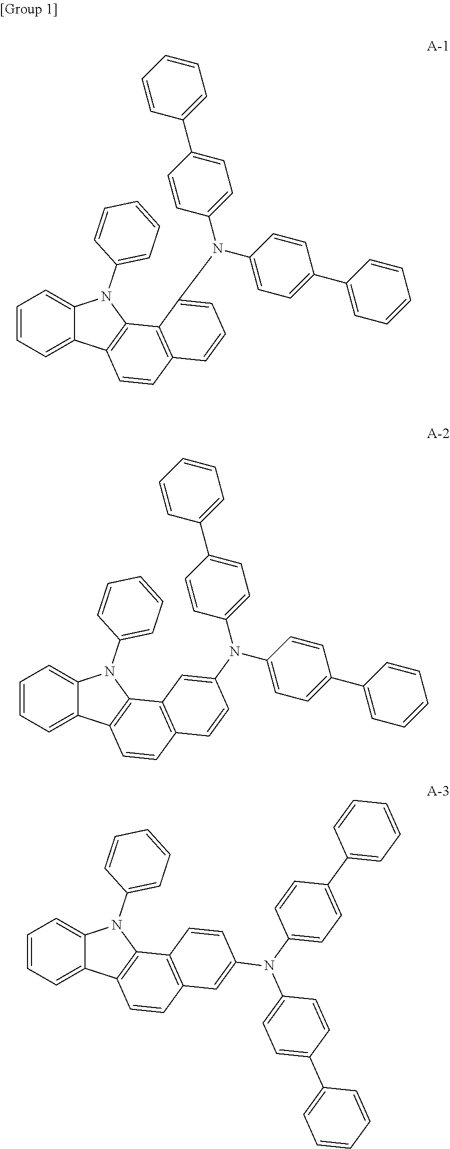 [SMILES: c1ccc(-c2ccc(N(c3ccc(-c4ccccc4)cc3)c3cccc4ccc5c6ccccc6n(-c6ccccc6)c5c34)cc2)cc1 ; c1ccc(-c2ccc(N(c3ccc(-c4ccccc4)cc3)c3ccc4ccc5c6ccccc6n(-c6ccccc6)c5c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1]

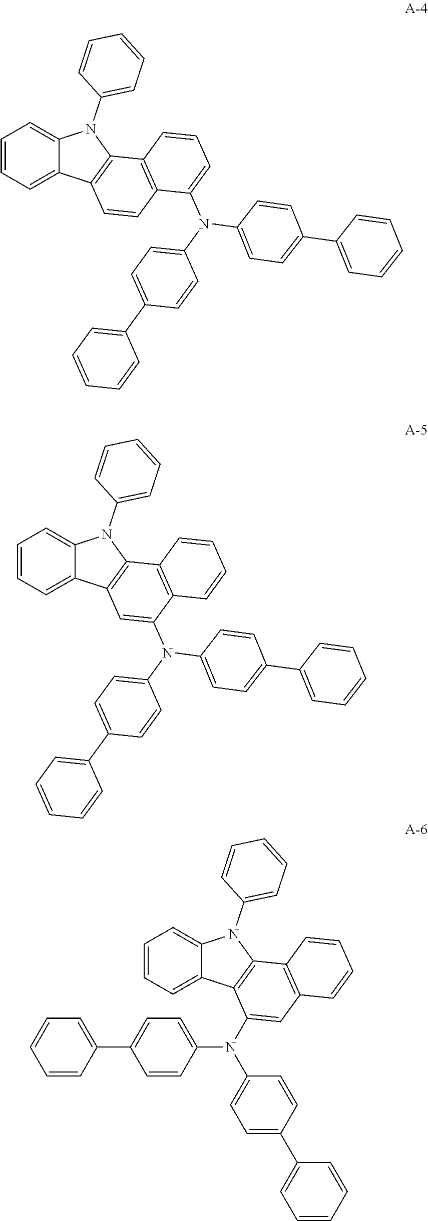 [SMILES: c1ccc(-c2ccc(N(c3ccc(-c4ccccc4)cc3)c3cccc4c3ccc3c5ccccc5n(-c5ccccc5)c43)cc2)cc1 ; c1ccc(-c2ccc(N(c3ccc(-c4ccccc4)cc3)c3cc4c5ccccc5n(-c5ccccc5)c4c4ccccc34)cc2)cc1 ; c1ccc(-c2ccc(N(c3ccc(-c4ccccc4)cc3)c3cc4ccccc4c4c3c3ccccc3n4-c3ccccc3)cc2)cc1]

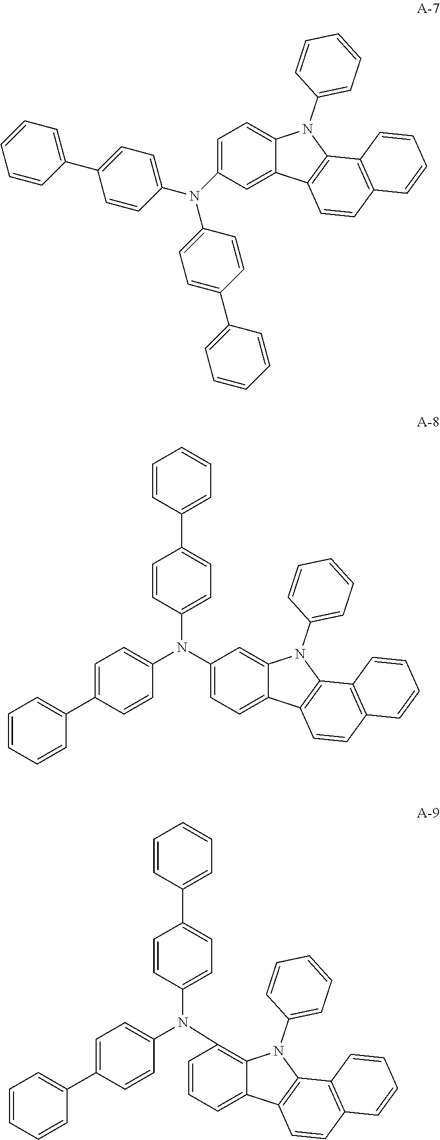 [SMILES: c1ccc(-c2ccc(N(c3ccc(-c4ccccc4)cc3)c3ccc4c(c3)c3ccc5ccccc5c3n4-c3ccccc3)cc2)cc1 ; c1ccc(-c2ccc(N(c3ccc(-c4ccccc4)cc3)c3ccc4c5ccc6ccccc6c5n(-c5ccccc5)c4c3)cc2)cc1 ; c1ccc(-c2ccc(N(c3ccc(-c4ccccc4)cc3)c3cccc4c5ccc6ccccc6c5n(-c5ccccc5)c34)cc2)cc1]

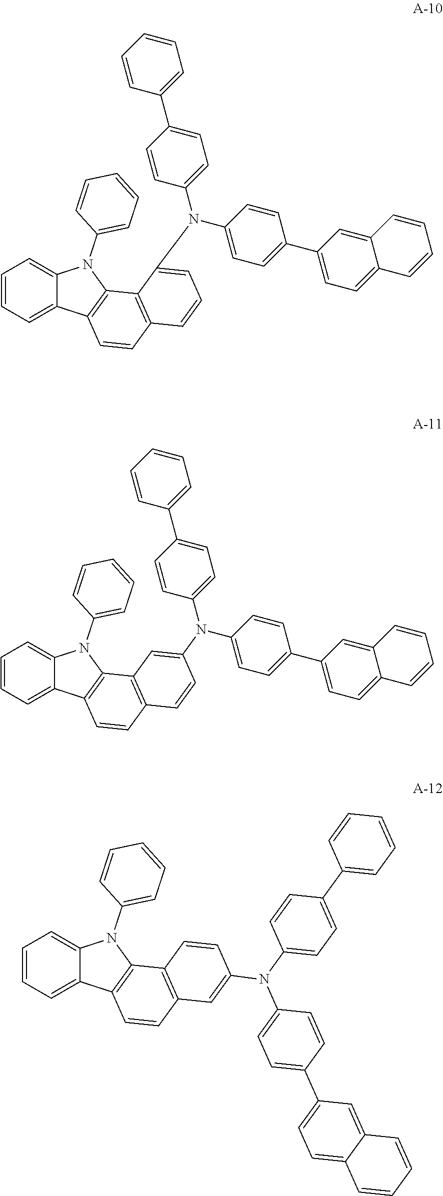 [SMILES: c1ccc(-c2ccc(N(c3ccc(-c4ccc5ccccc5c4)cc3)c3cccc4ccc5c6ccccc6n(-c6ccccc6)c5c34)cc2)cc1 ; c1ccc(-c2ccc(N(c3ccc(-c4ccc5ccccc5c4)cc3)c3ccc4ccc5c6ccccc6n(-c6ccccc6)c5c4c3)cc2)cc1 ; c1ccc(-c2ccc(N(c3ccc(-c4ccc5ccccc5c4)cc3)c3ccc4c(ccc5c6ccccc6n(-c6ccccc6)c45)c3)cc2)cc1]

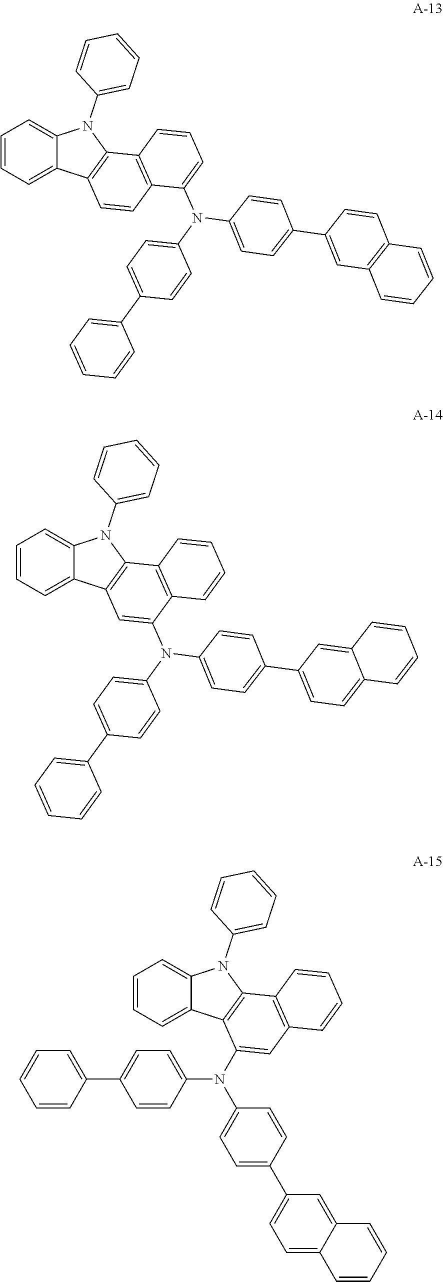 [SMILES: c1ccc(-c2ccc(N(c3ccc(-c4ccc5ccccc5c4)cc3)c3cccc4c3ccc3c5ccccc5n(-c5ccccc5)c43)cc2)cc1 ; c1ccc(-c2ccc(N(c3ccc(-c4ccc5ccccc5c4)cc3)c3cc4c5ccccc5n(-c5ccccc5)c4c4ccccc34)cc2)cc1 ; c1ccc(-c2ccc(N(c3ccc(-c4ccc5ccccc5c4)cc3)c3cc4ccccc4c4c3c3ccccc3n4-c3ccccc3)cc2)cc1]

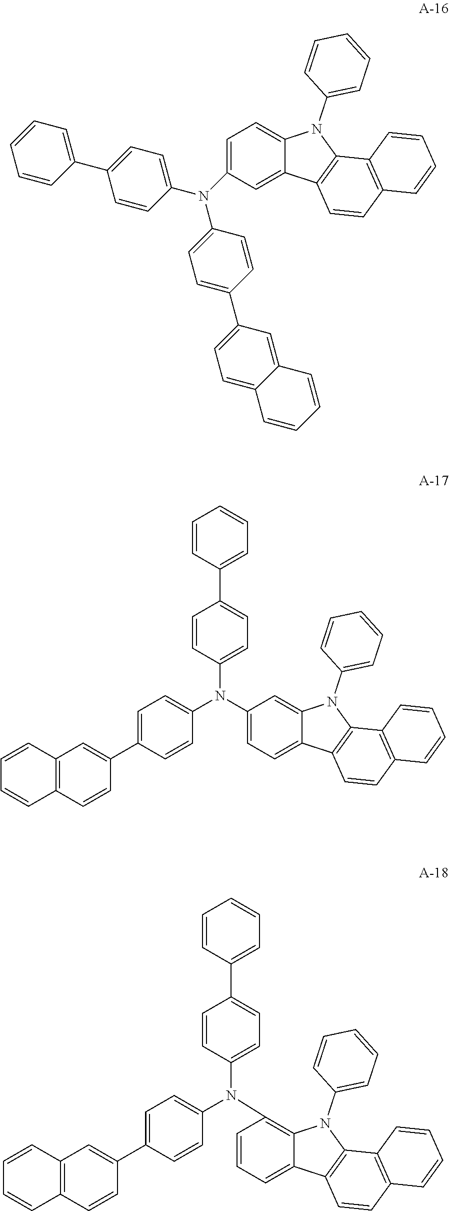 [SMILES: c1ccc(-c2ccc(N(c3ccc(-c4ccc5ccccc5c4)cc3)c3ccc4c(c3)c3ccc5ccccc5c3n4-c3ccccc3)cc2)cc1 ; c1ccc(-c2ccc(N(c3ccc(-c4ccc5ccccc5c4)cc3)c3ccc4c5ccc6ccccc6c5n(-c5ccccc5)c4c3)cc2)cc1 ; c1ccc(-c2ccc(N(c3ccc(-c4ccc5ccccc5c4)cc3)c3cccc4c5ccc6ccccc6c5n(-c5ccccc5)c34)cc2)cc1]

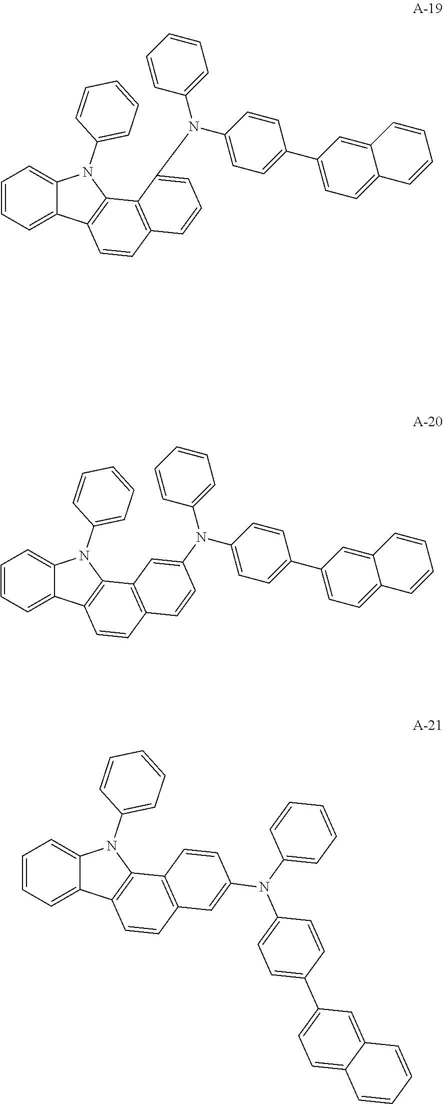 [SMILES: c1ccc(N(c2ccc(-c3ccc4ccccc4c3)cc2)c2cccc3ccc4c5ccccc5n(-c5ccccc5)c4c23)cc1 ; c1ccc(N(c2ccc(-c3ccc4ccccc4c3)cc2)c2ccc3ccc4c5ccccc5n(-c5ccccc5)c4c3c2)cc1 ; c1ccc(N(c2ccc(-c3ccc4ccccc4c3)cc2)c2ccc3c(ccc4c5ccccc5n(-c5ccccc5)c34)c2)cc1]

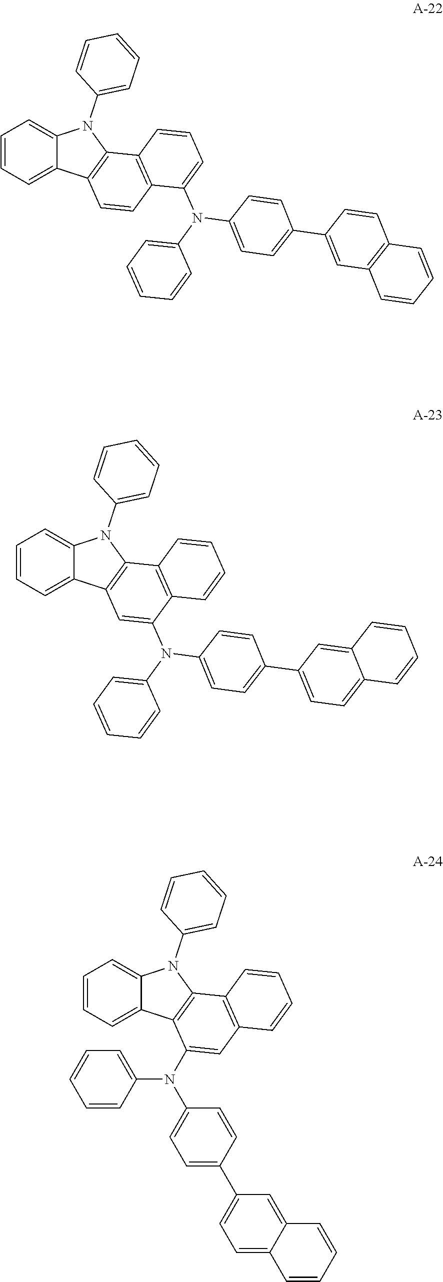 [SMILES: c1ccc(N(c2ccc(-c3ccc4ccccc4c3)cc2)c2cccc3c2ccc2c4ccccc4n(-c4ccccc4)c32)cc1 ; c1ccc(N(c2ccc(-c3ccc4ccccc4c3)cc2)c2cc3c4ccccc4n(-c4ccccc4)c3c3ccccc23)cc1 ; c1ccc(N(c2ccc(-c3ccc4ccccc4c3)cc2)c2cc3ccccc3c3c2c2ccccc2n3-c2ccccc2)cc1]

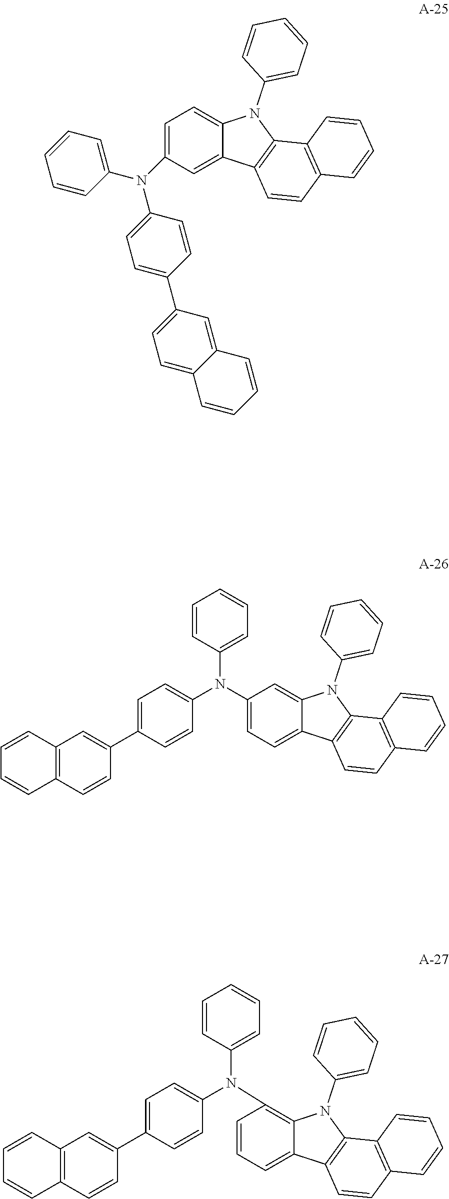 [SMILES: c1ccc(N(c2ccc(-c3ccc4ccccc4c3)cc2)c2ccc3c(c2)c2ccc4ccccc4c2n3-c2ccccc2)cc1 ; c1ccc(N(c2ccc(-c3ccc4ccccc4c3)cc2)c2ccc3c4ccc5ccccc5c4n(-c4ccccc4)c3c2)cc1 ; c1ccc(N(c2ccc(-c3ccc4ccccc4c3)cc2)c2cccc3c4ccc5ccccc5c4n(-c4ccccc4)c23)cc1]

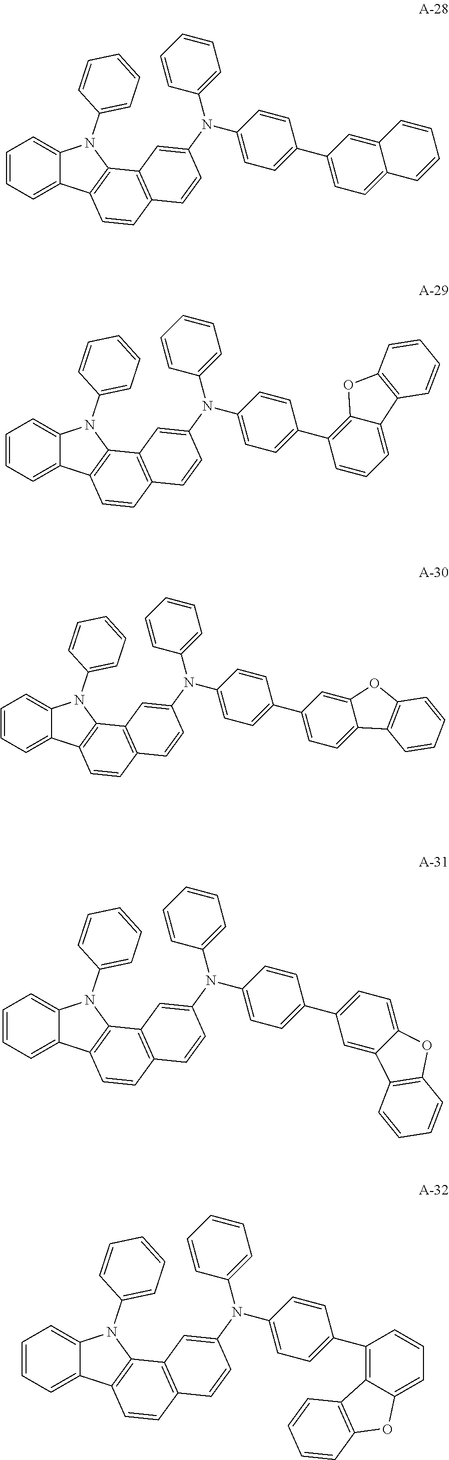 [SMILES: c1ccc(N(c2ccc(-c3ccc4ccccc4c3)cc2)c2ccc3ccc4c5ccccc5n(-c5ccccc5)c4c3c2)cc1 ; c1ccc(N(c2ccc(-c3cccc4c3oc3ccccc34)cc2)c2ccc3ccc4c5ccccc5n(-c5ccccc5)c4c3c2)cc1 ; c1ccc(N(c2ccc(-c3ccc4c(c3)oc3ccccc34)cc2)c2ccc3ccc4c5ccccc5n(-c5ccccc5)c4c3c2)cc1 ; c1ccc(N(c2ccc(-c3ccc4oc5ccccc5c4c3)cc2)c2ccc3ccc4c5ccccc5n(-c5ccccc5)c4c3c2)cc1 ; c1ccc(N(c2ccc(-c3cccc4oc5ccccc5c34)cc2)c2ccc3ccc4c5ccccc5n(-c5ccccc5)c4c3c2)cc1]

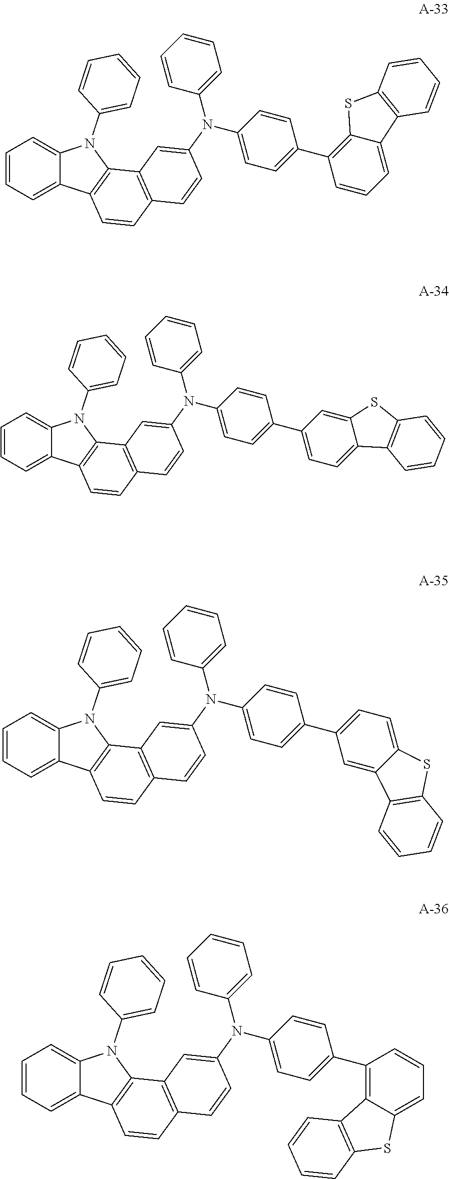 [SMILES: c1ccc(N(c2ccc(-c3cccc4c3sc3ccccc34)cc2)c2ccc3ccc4c5ccccc5n(-c5ccccc5)c4c3c2)cc1 ; c1ccc(N(c2ccc(-c3ccc4c(c3)sc3ccccc34)cc2)c2ccc3ccc4c5ccccc5n(-c5ccccc5)c4c3c2)cc1 ; c1ccc(N(c2ccc(-c3ccc4sc5ccccc5c4c3)cc2)c2ccc3ccc4c5ccccc5n(-c5ccccc5)c4c3c2)cc1 ; c1ccc(N(c2ccc(-c3cccc4sc5ccccc5c34)cc2)c2ccc3ccc4c5ccccc5n(-c5ccccc5)c4c3c2)cc1]

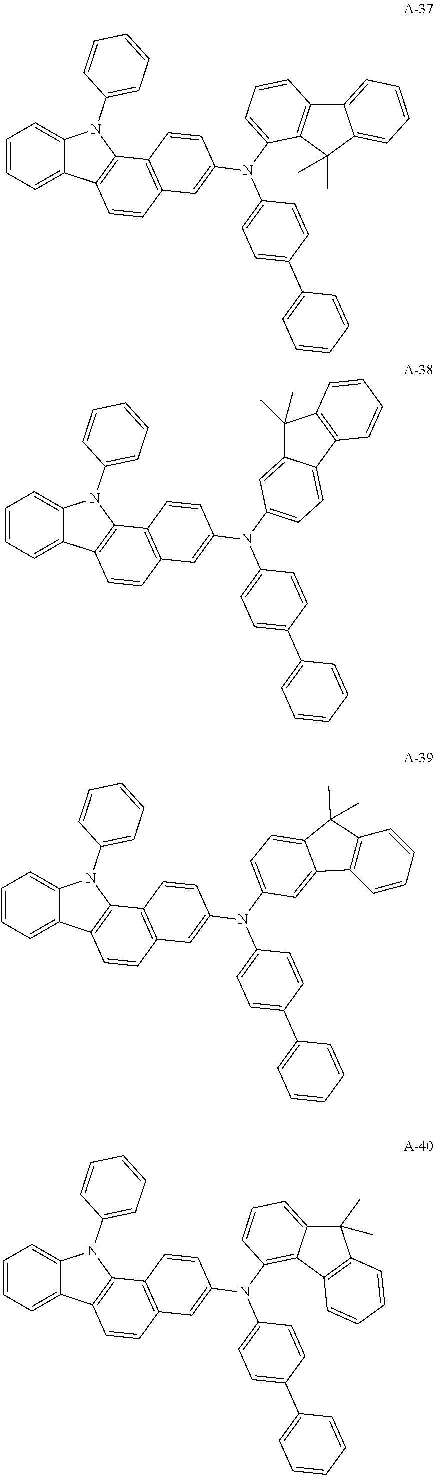

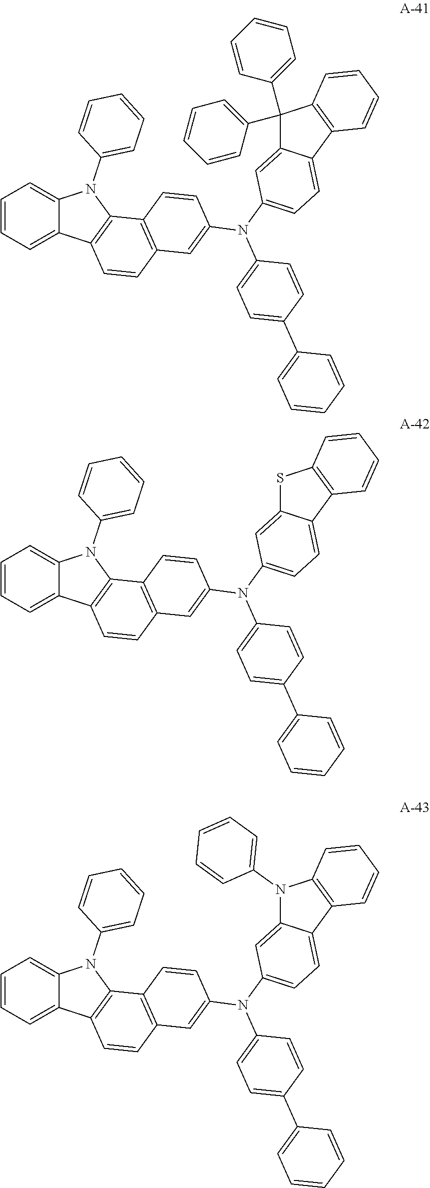

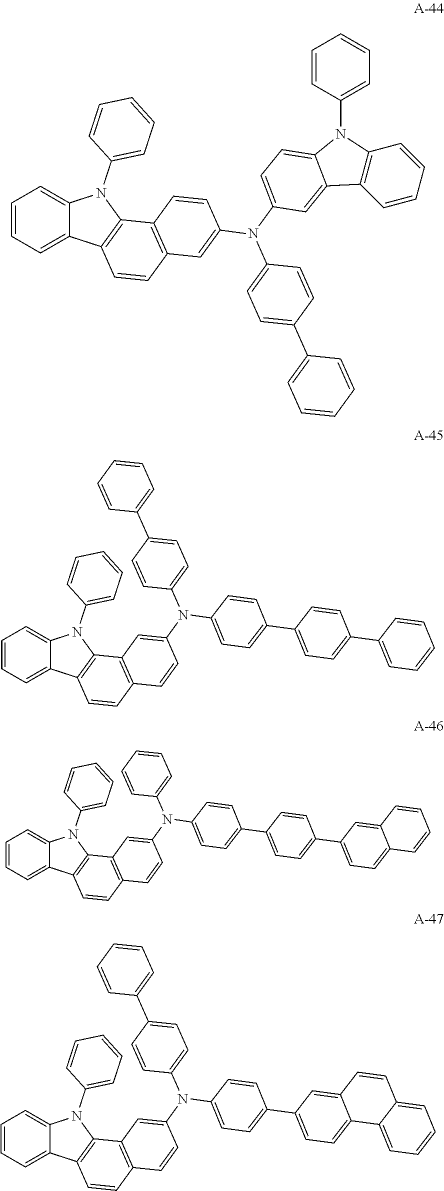 [SMILES: c1ccc(-c2ccc(N(c3ccc4c(ccc5c6ccccc6n(-c6ccccc6)c45)c3)c3ccc4c(c3)c3ccccc3n4-c3ccccc3)cc2)cc1 ; c1ccc(-c2ccc(-c3ccc(N(c4ccc(-c5ccccc5)cc4)c4ccc5ccc6c7ccccc7n(-c7ccccc7)c6c5c4)cc3)cc2)cc1 ; c1ccc(N(c2ccc(-c3ccc(-c4ccc5ccccc5c4)cc3)cc2)c2ccc3ccc4c5ccccc5n(-c5ccccc5)c4c3c2)cc1 ; c1ccc(-c2ccc(N(c3ccc(-c4ccc5c(ccc6ccccc65)c4)cc3)c3ccc4ccc5c6ccccc6n(-c6ccccc6)c5c4c3)cc2)cc1]

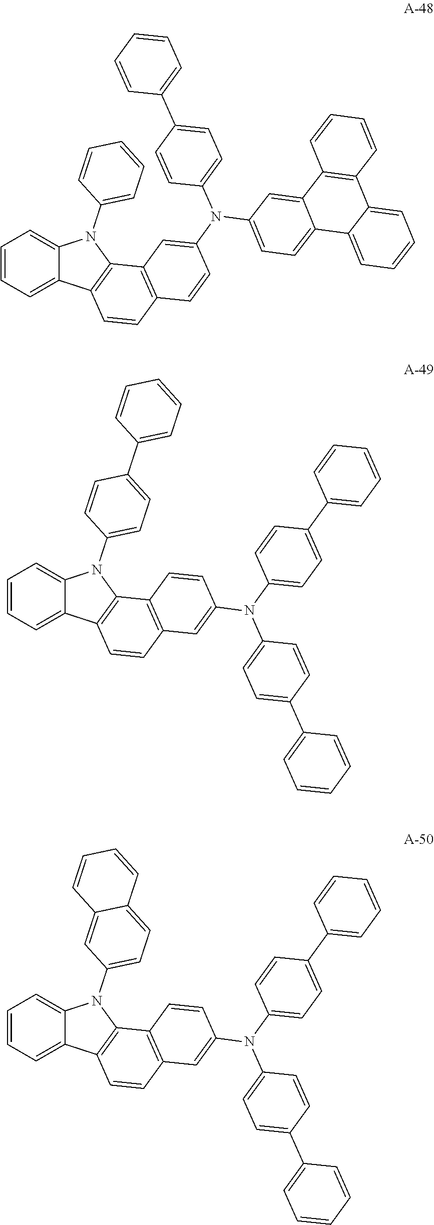 [SMILES: c1ccc(-c2ccc(N(c3ccc4c5ccccc5c5ccccc5c4c3)c3ccc4ccc5c6ccccc6n(-c6ccccc6)c5c4c3)cc2)cc1 ; c1ccc(-c2ccc(N(c3ccc(-c4ccccc4)cc3)c3ccc4c(ccc5c6ccccc6n(-c6ccc(-c7ccccc7)cc6)c45)c3)cc2)cc1 ; c1ccc(-c2ccc(N(c3ccc(-c4ccccc4)cc3)c3ccc4c(ccc5c6ccccc6n(-c6ccc7ccccc7c6)c45)c3)cc2)cc1]

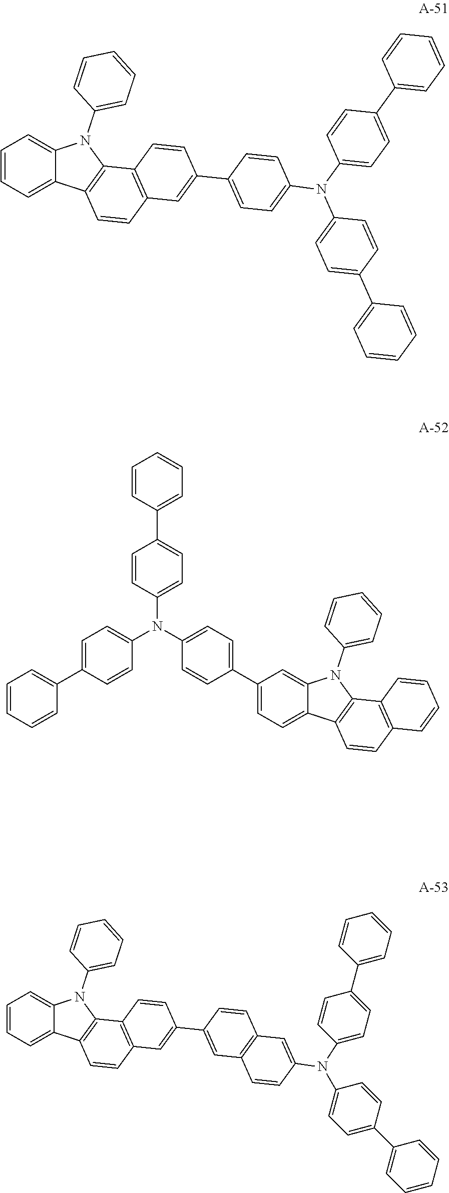 [SMILES: c1ccc(-c2ccc(N(c3ccc(-c4ccccc4)cc3)c3ccc(-c4ccc5c(ccc6c7ccccc7n(-c7ccccc7)c56)c4)cc3)cc2)cc1 ; c1ccc(-c2ccc(N(c3ccc(-c4ccccc4)cc3)c3ccc(-c4ccc5c6ccc7ccccc7c6n(-c6ccccc6)c5c4)cc3)cc2)cc1 ; c1ccc(-c2ccc(N(c3ccc(-c4ccccc4)cc3)c3ccc4cc(-c5ccc6c(ccc7c8ccccc8n(-c8ccccc8)c67)c5)ccc4c3)cc2)cc1]

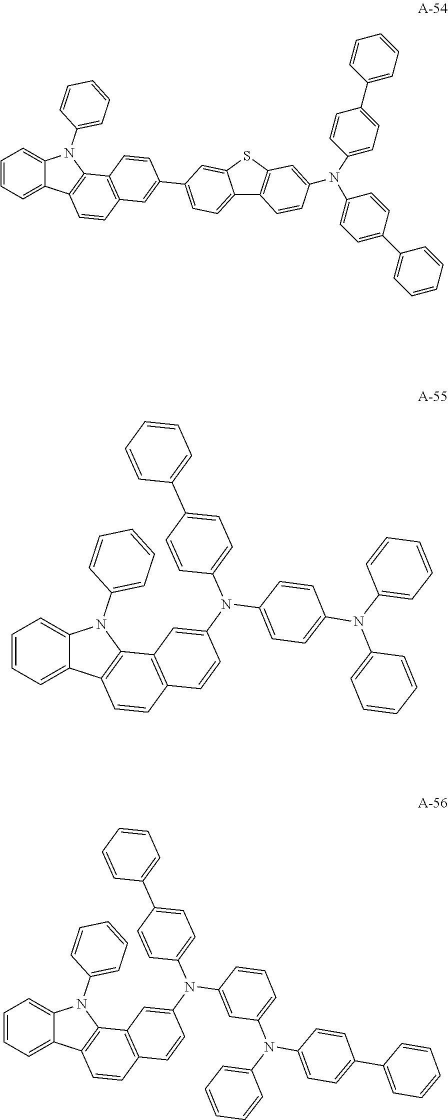 [SMILES: c1ccc(-c2ccc(N(c3ccc(-c4ccccc4)cc3)c3ccc4c(c3)sc3cc(-c5ccc6c(ccc7c8ccccc8n(-c8ccccc8)c67)c5)ccc34)cc2)cc1 ; c1ccc(-c2ccc(N(c3ccc(N(c4ccccc4)c4ccccc4)cc3)c3ccc4ccc5c6ccccc6n(-c6ccccc6)c5c4c3)cc2)cc1 ; c1ccc(-c2ccc(N(c3ccccc3)c3cccc(N(c4ccc(-c5ccccc5)cc4)c4ccc5ccc6c7ccccc7n(-c7ccccc7)c6c5c4)c3)cc2)cc1]

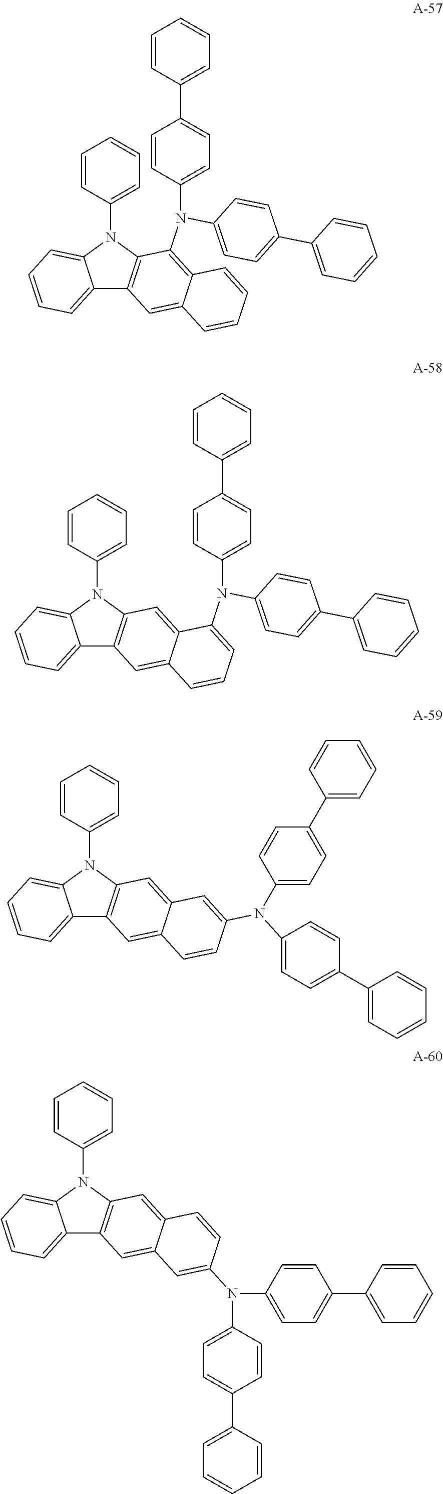 [SMILES: c1ccc(-c2ccc(N(c3ccc(-c4ccccc4)cc3)c3c4ccccc4cc4c5ccccc5n(-c5ccccc5)c34)cc2)cc1 ; c1ccc(-c2ccc(N(c3ccc(-c4ccccc4)cc3)c3cccc4cc5c6ccccc6n(-c6ccccc6)c5cc34)cc2)cc1 ; c1ccc(-c2ccc(N(c3ccc(-c4ccccc4)cc3)c3ccc4cc5c6ccccc6n(-c6ccccc6)c5cc4c3)cc2)cc1 ; c1ccc(-c2ccc(N(c3ccc(-c4ccccc4)cc3)c3ccc4cc5c(cc4c3)c3ccccc3n5-c3ccccc3)cc2)cc1]

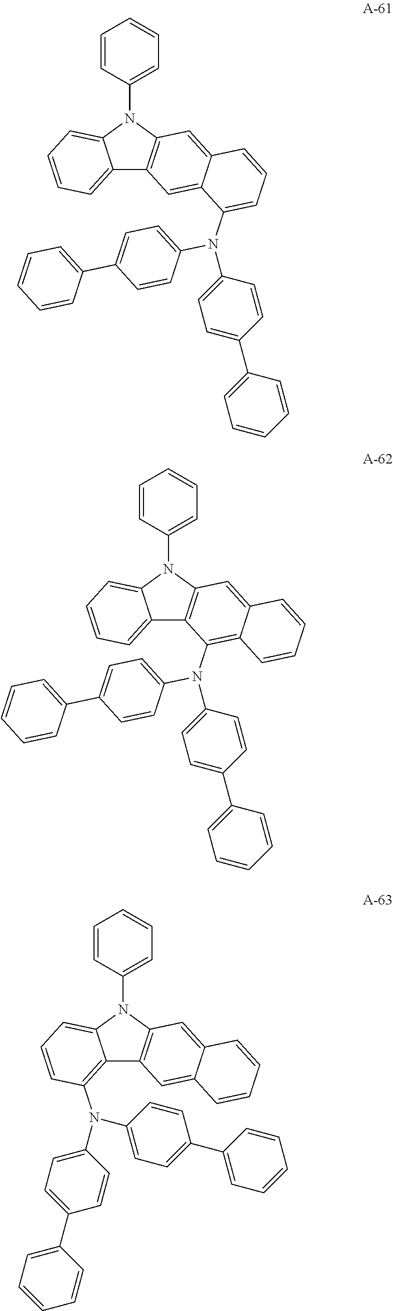 [SMILES: c1ccc(-c2ccc(N(c3ccc(-c4ccccc4)cc3)c3cccc4cc5c(cc34)c3ccccc3n5-c3ccccc3)cc2)cc1 ; c1ccc(-c2ccc(N(c3ccc(-c4ccccc4)cc3)c3c4ccccc4cc4c3c3ccccc3n4-c3ccccc3)cc2)cc1 ; c1ccc(-c2ccc(N(c3ccc(-c4ccccc4)cc3)c3cccc4c3c3cc5ccccc5cc3n4-c3ccccc3)cc2)cc1]

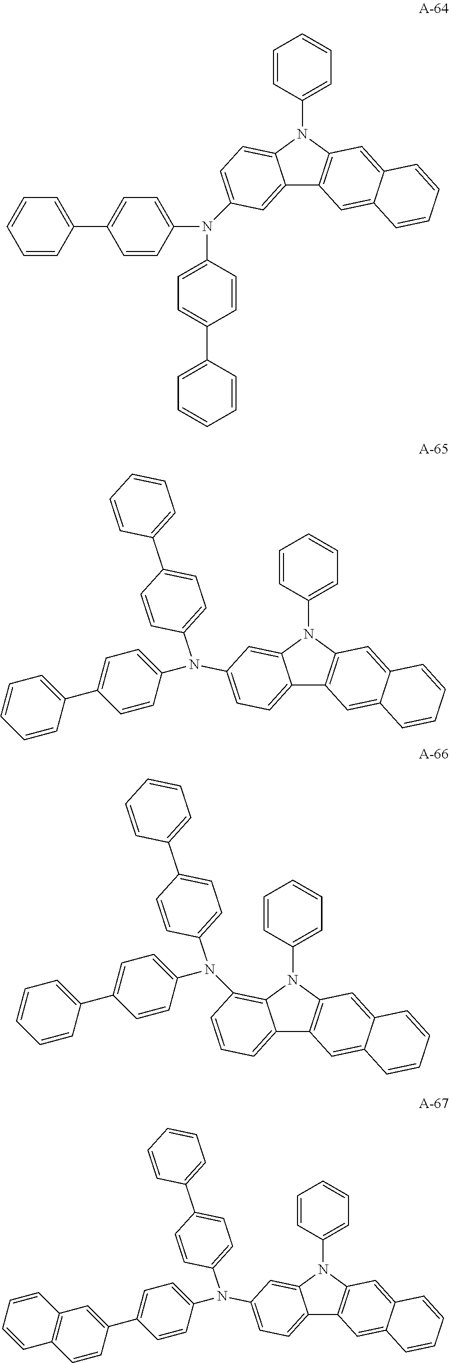 [SMILES: c1ccc(-c2ccc(N(c3ccc(-c4ccccc4)cc3)c3ccc4c(c3)c3cc5ccccc5cc3n4-c3ccccc3)cc2)cc1 ; c1ccc(-c2ccc(N(c3ccc(-c4ccccc4)cc3)c3ccc4c5cc6ccccc6cc5n(-c5ccccc5)c4c3)cc2)cc1 ; c1ccc(-c2ccc(N(c3ccc(-c4ccccc4)cc3)c3cccc4c5cc6ccccc6cc5n(-c5ccccc5)c34)cc2)cc1 ; c1ccc(-c2ccc(N(c3ccc(-c4ccc5ccccc5c4)cc3)c3ccc4c5cc6ccccc6cc5n(-c5ccccc5)c4c3)cc2)cc1]

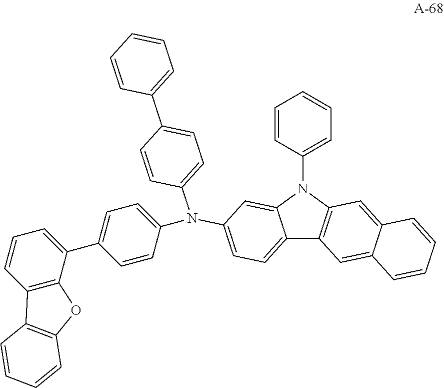 [SMILES: c1ccc(-c2ccc(N(c3ccc(-c4cccc5c4oc4ccccc45)cc3)c3ccc4c5cc6ccccc6cc5n(-c5ccccc5)c4c3)cc2)cc1]

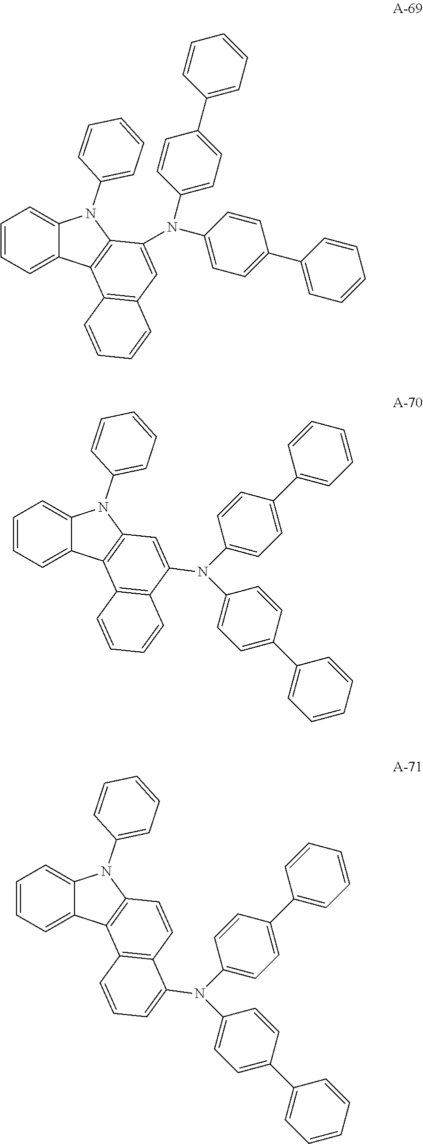 [SMILES: c1ccc(-c2ccc(N(c3ccc(-c4ccccc4)cc3)c3cc4ccccc4c4c5ccccc5n(-c5ccccc5)c34)cc2)cc1 ; c1ccc(-c2ccc(N(c3ccc(-c4ccccc4)cc3)c3cc4c(c5ccccc35)c3ccccc3n4-c3ccccc3)cc2)cc1 ; c1ccc(-c2ccc(N(c3ccc(-c4ccccc4)cc3)c3cccc4c3ccc3c4c4ccccc4n3-c3ccccc3)cc2)cc1]

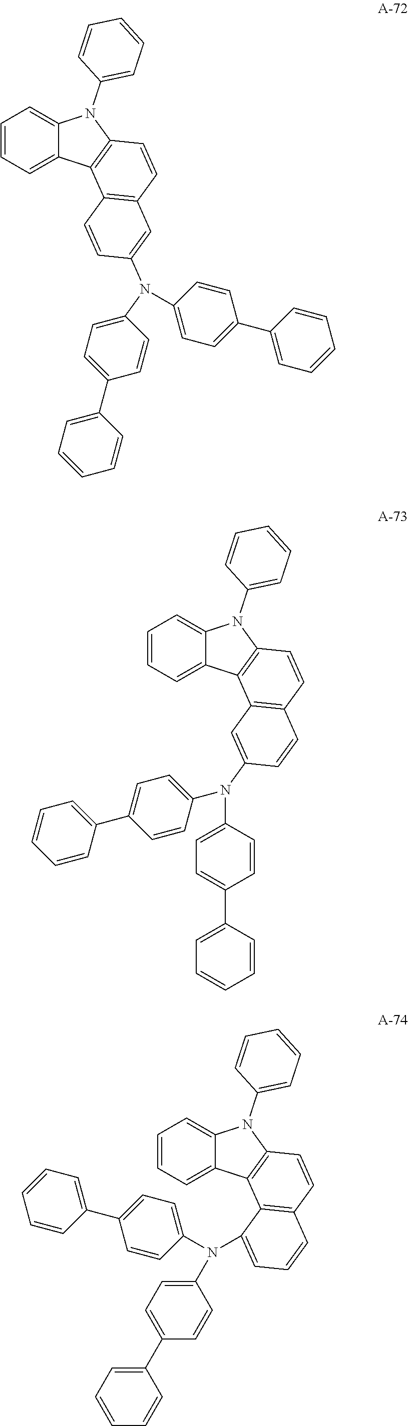 [SMILES: c1ccc(-c2ccc(N(c3ccc(-c4ccccc4)cc3)c3ccc4c(ccc5c4c4ccccc4n5-c4ccccc4)c3)cc2)cc1 ; c1ccc(-c2ccc(N(c3ccc(-c4ccccc4)cc3)c3ccc4ccc5c(c4c3)c3ccccc3n5-c3ccccc3)cc2)cc1 ; c1ccc(-c2ccc(N(c3ccc(-c4ccccc4)cc3)c3cccc4ccc5c(c6ccccc6n5-c5ccccc5)c34)cc2)cc1]

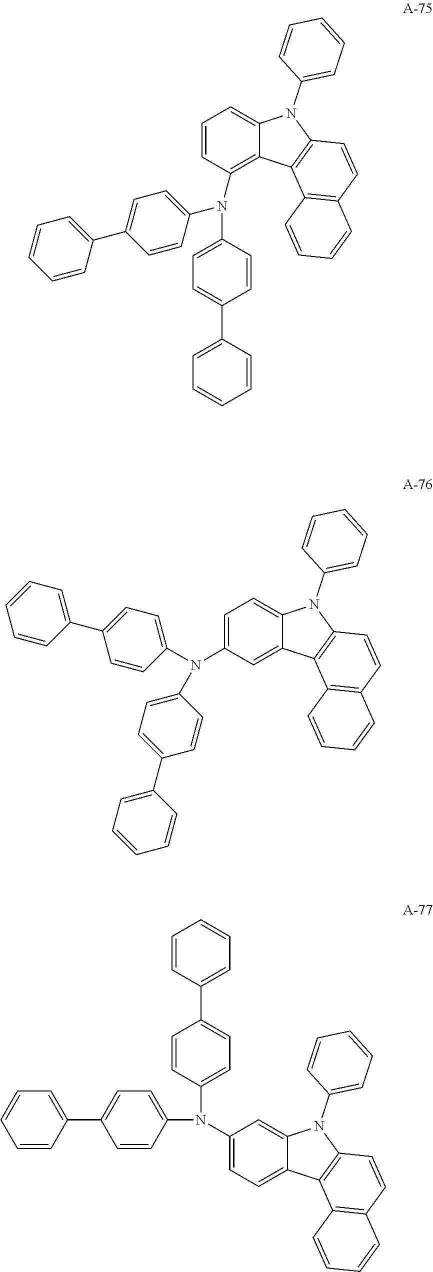 [SMILES: c1ccc(-c2ccc(N(c3ccc(-c4ccccc4)cc3)c3cccc4c3c3c5ccccc5ccc3n4-c3ccccc3)cc2)cc1 ; c1ccc(-c2ccc(N(c3ccc(-c4ccccc4)cc3)c3ccc4c(c3)c3c5ccccc5ccc3n4-c3ccccc3)cc2)cc1 ; c1ccc(-c2ccc(N(c3ccc(-c4ccccc4)cc3)c3ccc4c5c6ccccc6ccc5n(-c5ccccc5)c4c3)cc2)cc1]

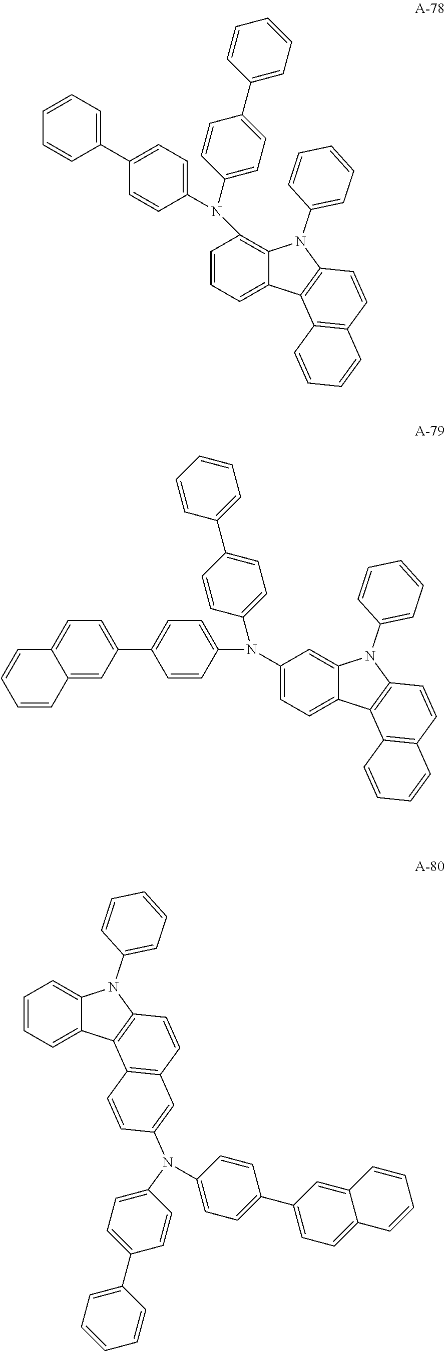 [SMILES: c1ccc(-c2ccc(N(c3ccc(-c4ccccc4)cc3)c3cccc4c5c6ccccc6ccc5n(-c5ccccc5)c34)cc2)cc1 ; c1ccc(-c2ccc(N(c3ccc(-c4ccc5ccccc5c4)cc3)c3ccc4c5c6ccccc6ccc5n(-c5ccccc5)c4c3)cc2)cc1 ; c1ccc(-c2ccc(N(c3ccc(-c4ccc5ccccc5c4)cc3)c3ccc4c(ccc5c4c4ccccc4n5-c4ccccc4)c3)cc2)cc1]

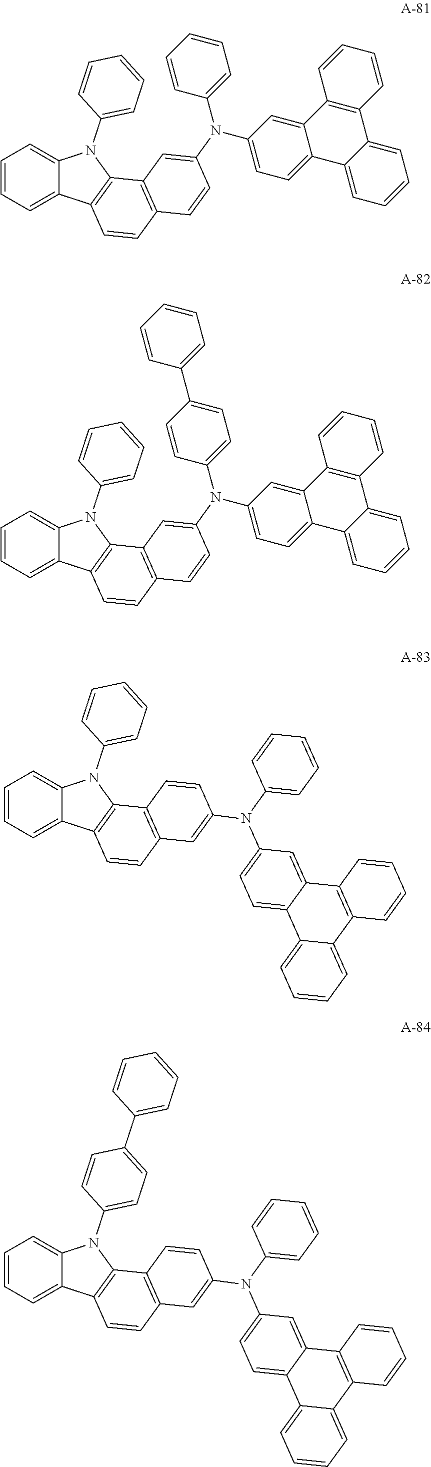 [SMILES: c1ccc(N(c2ccc3c4ccccc4c4ccccc4c3c2)c2ccc3ccc4c5ccccc5n(-c5ccccc5)c4c3c2)cc1 ; c1ccc(-c2ccc(N(c3ccc4c5ccccc5c5ccccc5c4c3)c3ccc4ccc5c6ccccc6n(-c6ccccc6)c5c4c3)cc2)cc1 ; c1ccc(N(c2ccc3c(ccc4c5ccccc5n(-c5ccccc5)c34)c2)c2ccc3c4ccccc4c4ccccc4c3c2)cc1 ; c1ccc(-c2ccc(-n3c4ccccc4c4ccc5cc(N(c6ccccc6)c6ccc7c8ccccc8c8ccccc8c7c6)ccc5c43)cc2)cc1]

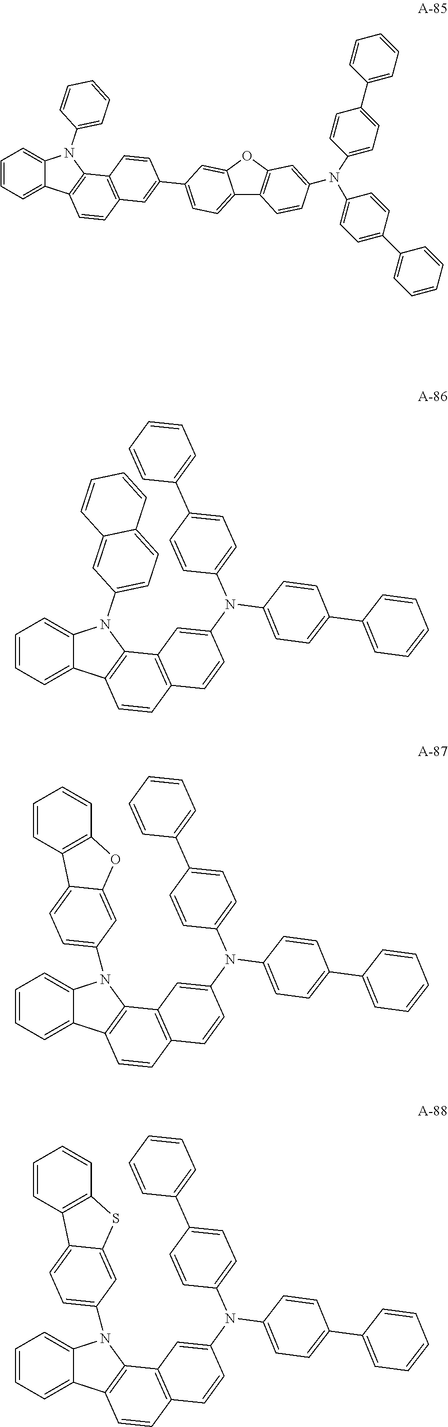 [SMILES: c1ccc(-c2ccc(N(c3ccc(-c4ccccc4)cc3)c3ccc4c(c3)oc3cc(-c5ccc6c(ccc7c8ccccc8n(-c8ccccc8)c67)c5)ccc34)cc2)cc1 ; c1ccc(-c2ccc(N(c3ccc(-c4ccccc4)cc3)c3ccc4ccc5c6ccccc6n(-c6ccc7ccccc7c6)c5c4c3)cc2)cc1 ; c1ccc(-c2ccc(N(c3ccc(-c4ccccc4)cc3)c3ccc4ccc5c6ccccc6n(-c6ccc7c(c6)oc6ccccc67)c5c4c3)cc2)cc1 ; c1ccc(-c2ccc(N(c3ccc(-c4ccccc4)cc3)c3ccc4ccc5c6ccccc6n(-c6ccc7c(c6)sc6ccccc67)c5c4c3)cc2)cc1]

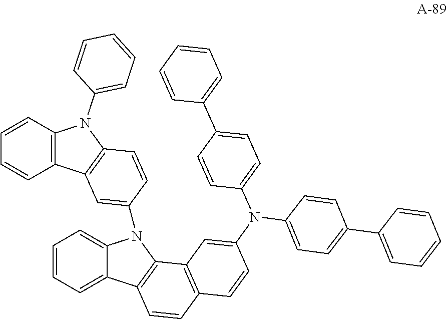 [SMILES: c1ccc(-c2ccc(N(c3ccc(-c4ccccc4)cc3)c3ccc4ccc5c6ccccc6n(-c6ccc7c(c6)c6ccccc6n7-c6ccccc6)c5c4c3)cc2)cc1]

The second compound for an organic optoelectronic element is represented by a combination of Chemical Formula 3 and Chemical Formula 4.

The second compound for an organic optoelectronic element is a compound having electron characteristics, and may be included together with the aforementioned first compound for an organic optoelectronic element to exhibit bipolar characteristics.

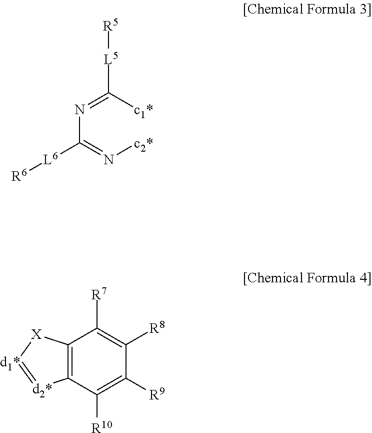

In Chemical Formula 3 and Chemical Formula 4,

X is O or S,

c₁* and c₂* are linked with d₁* and d₂* or d₂* and d₁*, respectively,

L⁵ and L⁶ are independently a single bond, a substituted or unsubstituted C6 to C20 arylene group, a substituted or unsubstituted C2 to C20 heterocyclic group, or a combination thereof,

R⁵ to R¹⁰ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and

at least one of R⁵ and R⁶ is a substituted or unsubstituted C6 to C30 aryl group, a substituted or unsubstituted C2 to C30 heterocyclic group, or a combination thereof.

The second compound for an organic optoelectronic element is a compound capable of receiving electrons when an electric field is applied, that is, electron characteristics. Specifically, it has a core in which a pyrimidine ring and a benzene ring are condensed on both sides of a pentagonal ring. For example, when the compound represented by the combination of Chemical Formula 3 and Chemical Formula 4 is used as a host in the light emitting layer of an organic light emitting diode, a balance between holes and electrons is achieved with the compound for the first compound for an organic optoelectronic element, resulting in high efficiency and long life-span emission.

For example, the second compound for an organic optoelectronic element may be represented by Chemical Formula 2-I or Chemical Formula 2-II.

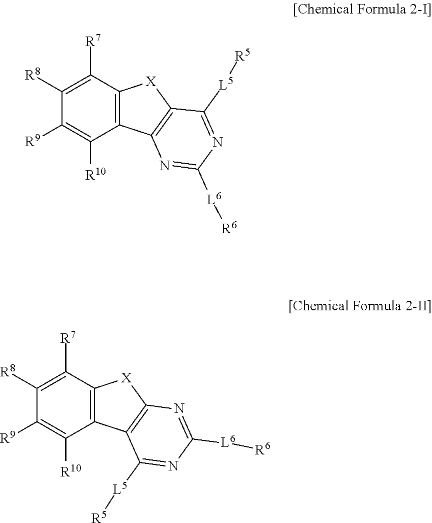

In Chemical Formulas 2-I and 2-II, X, L⁵, L⁶, and R⁵ to R¹⁰ are as described above.

As a specific example, R⁵ and R⁶ may independently be a substituted or unsubstituted C6 to C30 aryl group, a substituted or unsubstituted C2 to C30 heterocyclic group, or a combination thereof.

For example, R⁵ and R⁶ may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted dibenzofuranyl group, a substituted or unsubstituted dibenzothiophenyl group, a substituted or unsubstituted carbazolyl group, a substituted or unsubstituted indolocarbazolyl group, or a combination thereof.

For example, R⁵ and R⁶ may independently be a substituted or unsubstituted phenyl group, a substituted or unsubstituted biphenyl group, a substituted or unsubstituted naphthyl group, a substituted or unsubstituted dibenzofuranyl group, a substituted or unsubstituted dibenzothiophenyl group, a substituted or unsubstituted carbazolyl group, or a combination thereof, but are not limited thereto.

For example, R⁷ to R¹⁰ may independently be hydrogen, deuterium, a cyano group, a substituted or unsubstituted C1 to C10 alkyl group, a substituted or unsubstituted C6 to C20 aryl group, or a substituted or unsubstituted C2 to C20 heterocyclic group.

As a specific example, R⁷ to R¹⁰ may independently be hydrogen, deuterium, a cyano group, a substituted or unsubstituted C1 to C10 alkyl group, a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group.

More specifically, R⁷ to R¹⁰ may independently be hydrogen, deuterium, a substituted or unsubstituted C1 to C10 alkyl group, a substituted or unsubstituted C6 to C12 aryl group, or a substituted or unsubstituted C6 to C12 heterocyclic group.

In an embodiment, R⁷ to R¹⁰ may independently be hydrogen, but is not limited thereto.

For example, at least one of R⁷ to R¹⁰ may be a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group and the rest may be hydrogen, but are not limited thereto.

In an embodiment, one of R⁷ to R¹⁰ may be a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group and the rest may be hydrogen, but is not limited thereto.

In an embodiment, R⁷ and R⁹ may independently be a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group, and R⁸ and R¹⁰ may independently be hydrogen, but are not limited thereto.

In an embodiment, R⁸ and R¹⁰ may independently be a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group, and R⁷ and R⁹ may independently be hydrogen, but is not limited thereto.

The second compound for an organic optoelectronic element may be, for example, one selected from compounds of Group 2, but is not limited thereto.

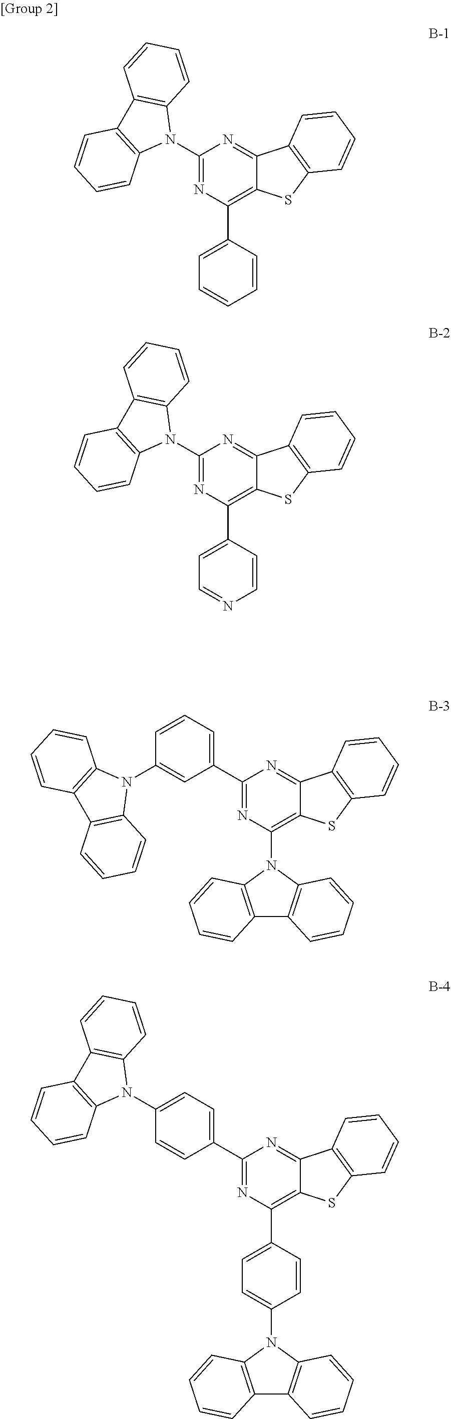 [SMILES: c1ccc(-c2nc(-n3c4ccccc4c4ccccc43)nc3c2sc2ccccc23)cc1 ; c1ccc2c(c1)sc1c(-c3ccncc3)nc(-n3c4ccccc4c4ccccc43)nc12 ; c1cc(-c2nc(-n3c4ccccc4c4ccccc43)c3sc4ccccc4c3n2)cc(-n2c3ccccc3c3ccccc32)c1 ; c1ccc2c(c1)sc1c(-c3ccc(-n4c5ccccc5c5ccccc54)cc3)nc(-c3ccc(-n4c5ccccc5c5ccccc54)cc3)nc12]

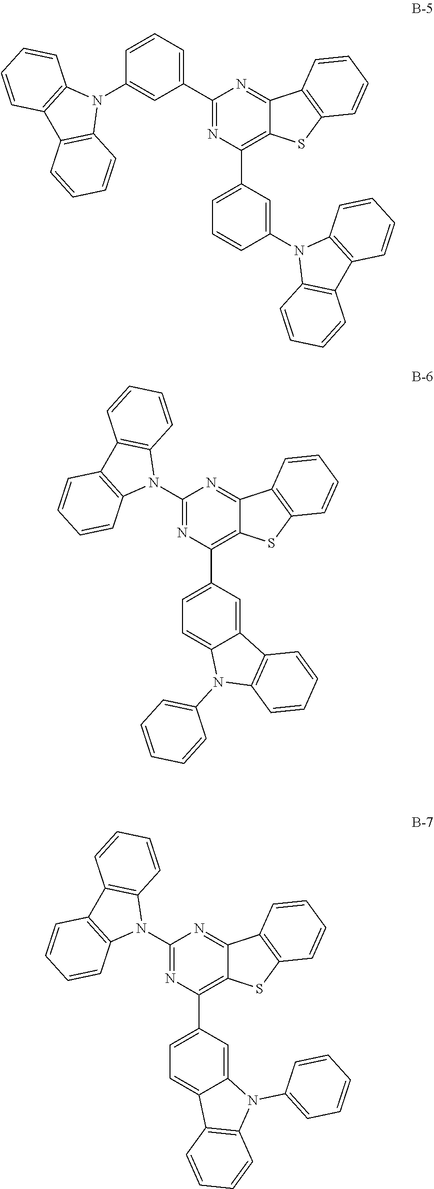 [SMILES: c1cc(-c2nc(-c3cccc(-n4c5ccccc5c5ccccc54)c3)c3sc4ccccc4c3n2)cc(-n2c3ccccc3c3ccccc32)c1 ; c1ccc(-n2c3ccccc3c3cc(-c4nc(-n5c6ccccc6c6ccccc65)nc5c4sc4ccccc45)ccc32)cc1 ; c1ccc(-n2c3ccccc3c3ccc(-c4nc(-n5c6ccccc6c6ccccc65)nc5c4sc4ccccc45)cc32)cc1]

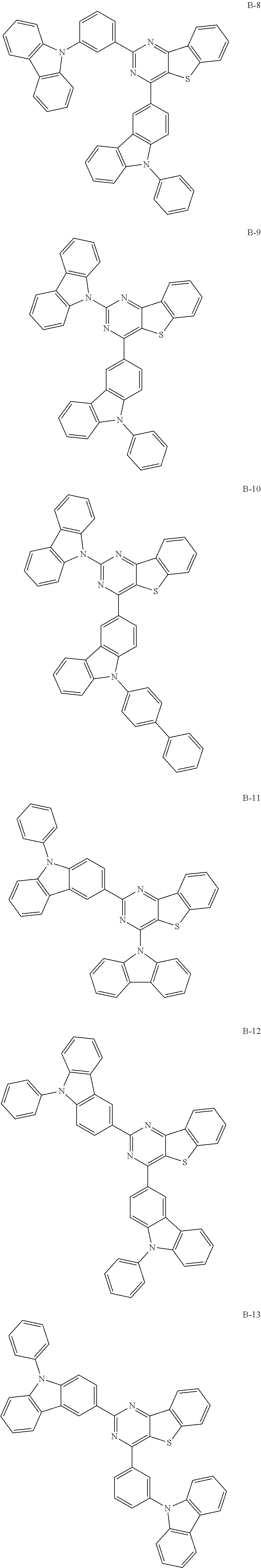

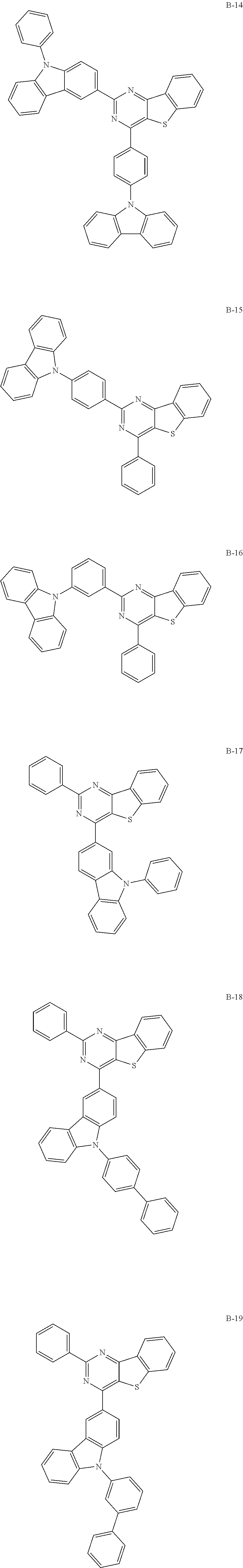 [SMILES: c1ccc(-n2c3ccccc3c3cc(-c4nc(-c5ccc(-n6c7ccccc7c7ccccc76)cc5)c5sc6ccccc6c5n4)ccc32)cc1 ; c1ccc(-c2nc(-c3ccc(-n4c5ccccc5c5ccccc54)cc3)nc3c2sc2ccccc23)cc1 ; c1ccc(-c2nc(-c3cccc(-n4c5ccccc5c5ccccc54)c3)nc3c2sc2ccccc23)cc1 ; c1ccc(-c2nc(-c3ccc4c5ccccc5n(-c5ccccc5)c4c3)c3sc4ccccc4c3n2)cc1 ; c1ccc(-c2ccc(-n3c4ccccc4c4cc(-c5nc(-c6ccccc6)nc6c5sc5ccccc56)ccc43)cc2)cc1 ; c1ccc(-c2cccc(-n3c4ccccc4c4cc(-c5nc(-c6ccccc6)nc6c5sc5ccccc56)ccc43)c2)cc1]

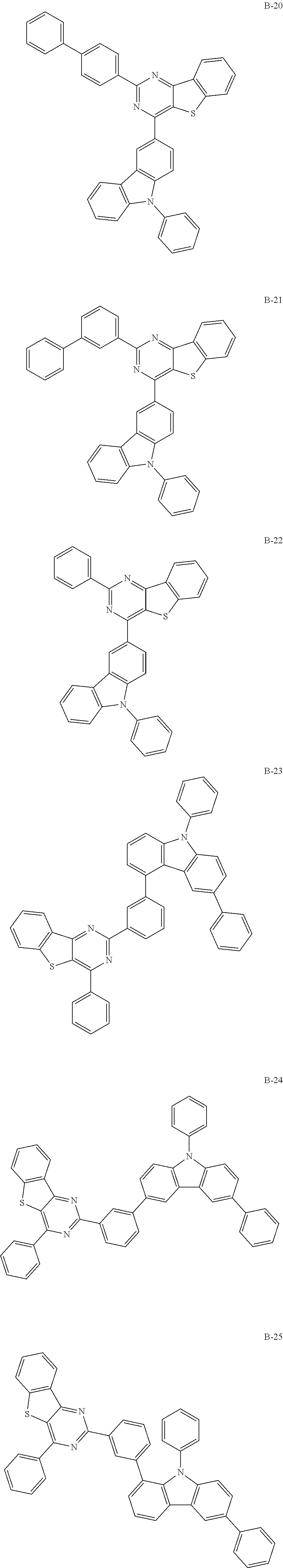 [SMILES: c1ccc(-c2ccc(-c3nc(-c4ccc5c(c4)c4ccccc4n5-c4ccccc4)c4sc5ccccc5c4n3)cc2)cc1 ; c1ccc(-c2cccc(-c3nc(-c4ccc5c(c4)c4ccccc4n5-c4ccccc4)c4sc5ccccc5c4n3)c2)cc1 ; c1ccc(-c2nc(-c3ccc4c(c3)c3ccccc3n4-c3ccccc3)c3sc4ccccc4c3n2)cc1 ; c1ccc(-c2ccc3c(c2)c2c(-c4cccc(-c5nc(-c6ccccc6)c6sc7ccccc7c6n5)c4)cccc2n3-c2ccccc2)cc1 ; c1ccc(-c2ccc3c(c2)c2cc(-c4cccc(-c5nc(-c6ccccc6)c6sc7ccccc7c6n5)c4)ccc2n3-c2ccccc2)cc1 ; c1ccc(-c2ccc3c(c2)c2cccc(-c4cccc(-c5nc(-c6ccccc6)c6sc7ccccc7c6n5)c4)c2n3-c2ccccc2)cc1]

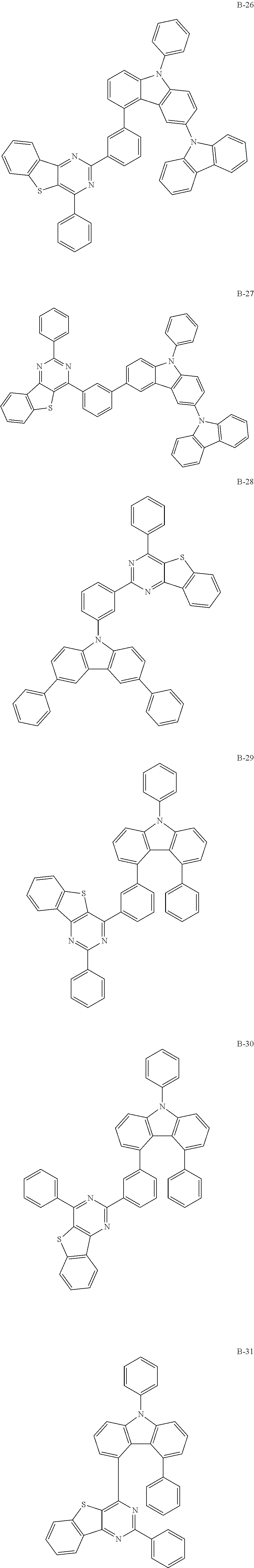

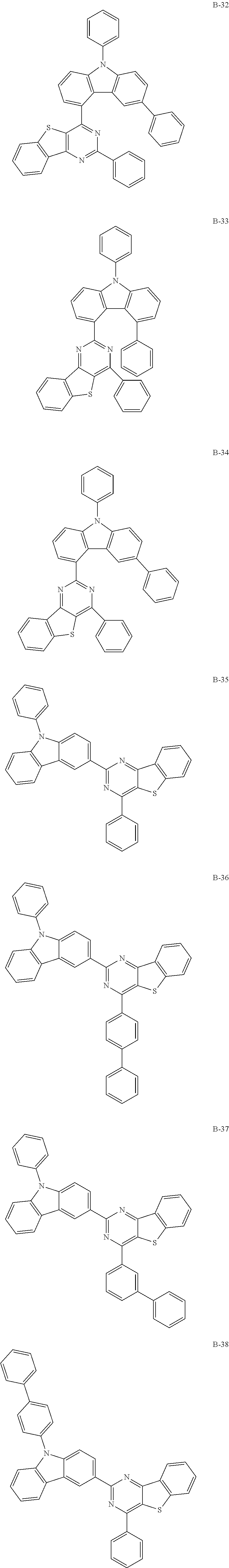

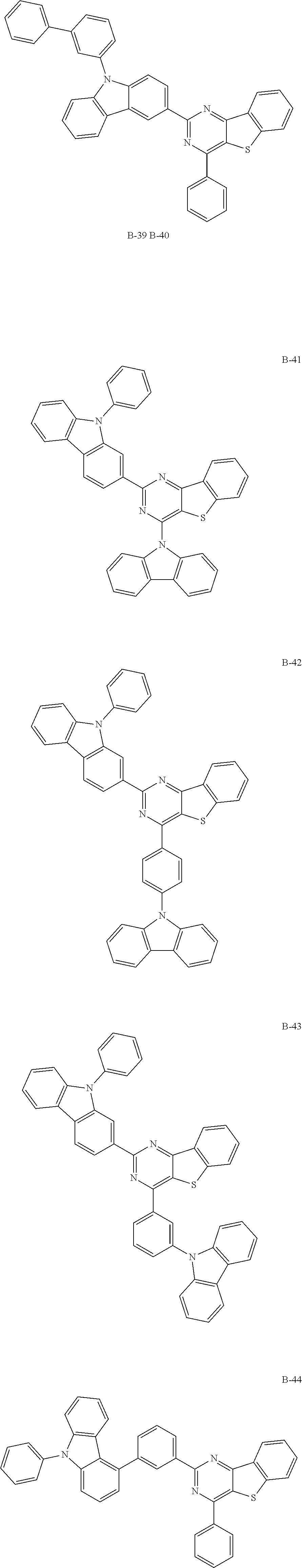 [SMILES: c1ccc(-c2cccc(-n3c4ccccc4c4cc(-c5nc(-c6ccccc6)c6sc7ccccc7c6n5)ccc43)c2)cc1 ; c1ccc(-n2c3ccccc3c3ccc(-c4nc(-n5c6ccccc6c6ccccc65)c5sc6ccccc6c5n4)cc32)cc1 ; c1ccc(-n2c3ccccc3c3ccc(-c4nc(-c5ccc(-n6c7ccccc7c7ccccc76)cc5)c5sc6ccccc6c5n4)cc32)cc1 ; c1ccc(-n2c3ccccc3c3ccc(-c4nc(-c5cccc(-n6c7ccccc7c7ccccc76)c5)c5sc6ccccc6c5n4)cc32)cc1 ; c1ccc(-c2nc(-c3cccc(-c4cccc5c4c4ccccc4n5-c4ccccc4)c3)nc3c2sc2ccccc23)cc1]

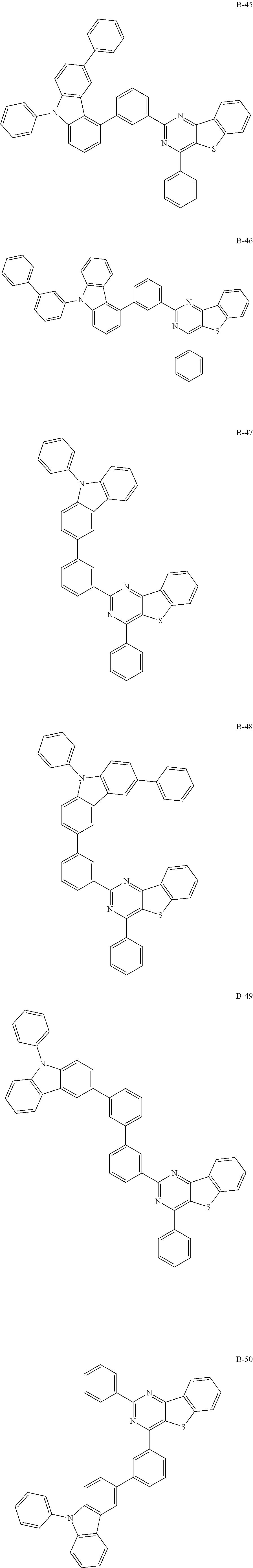

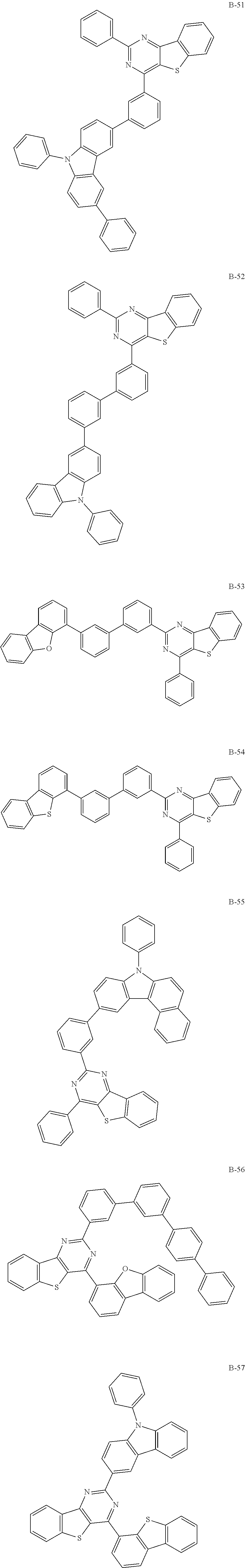

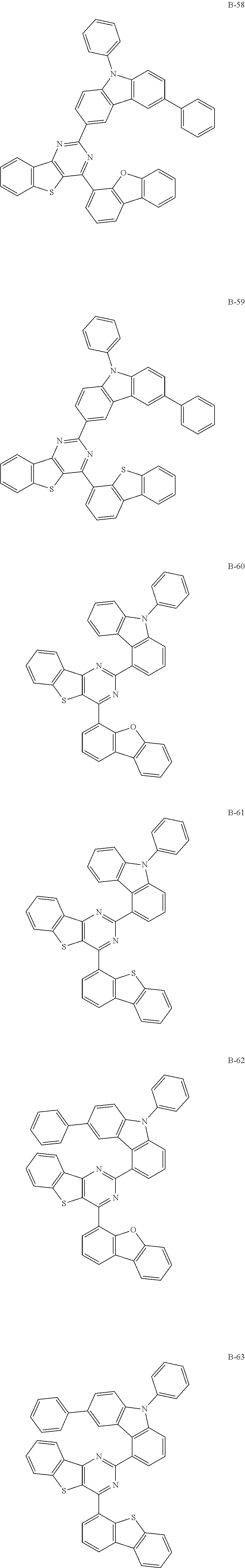

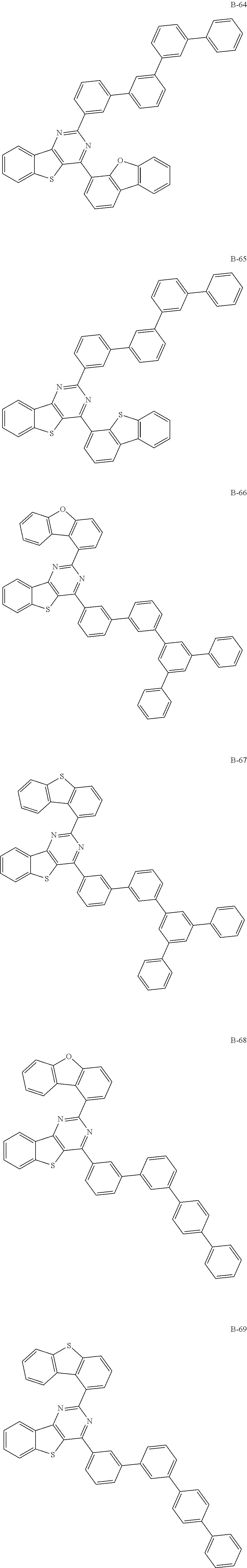

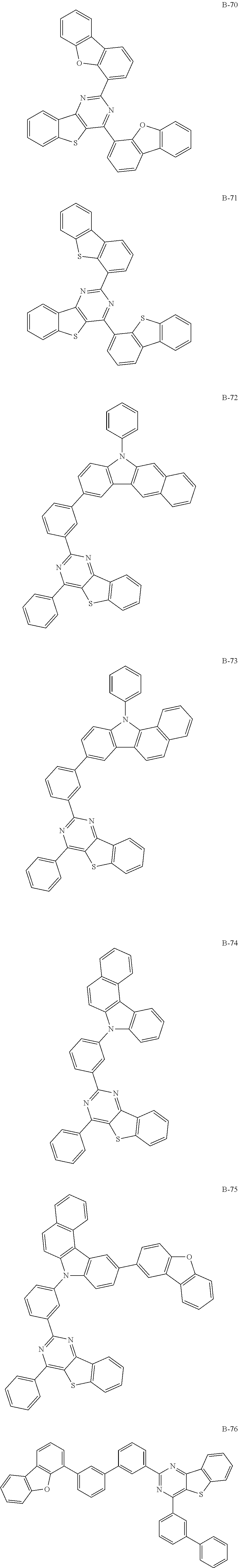

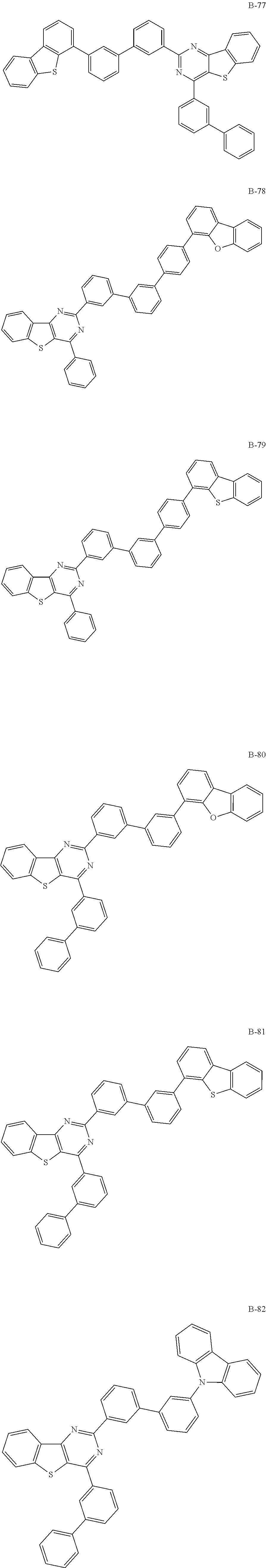

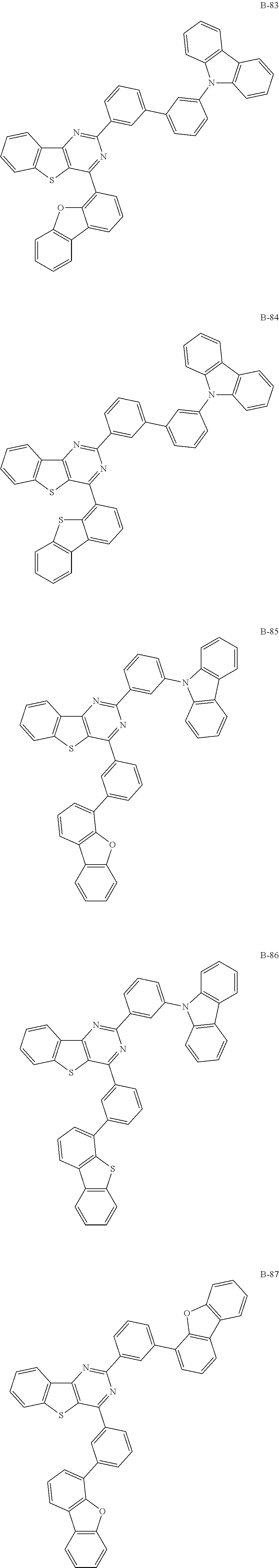 [SMILES: c1cc(-c2cccc(-n3c4ccccc4c4ccccc43)c2)cc(-c2nc(-c3cccc4c3oc3ccccc34)c3sc4ccccc4c3n2)c1 ; c1cc(-c2cccc(-n3c4ccccc4c4ccccc43)c2)cc(-c2nc(-c3cccc4c3sc3ccccc34)c3sc4ccccc4c3n2)c1 ; c1cc(-c2cccc3c2oc2ccccc23)cc(-c2nc(-c3cccc(-n4c5ccccc5c5ccccc54)c3)nc3c2sc2ccccc23)c1 ; c1cc(-c2cccc3c2sc2ccccc23)cc(-c2nc(-c3cccc(-n4c5ccccc5c5ccccc54)c3)nc3c2sc2ccccc23)c1 ; c1cc(-c2nc(-c3cccc(-c4cccc5c4oc4ccccc45)c3)c3sc4ccccc4c3n2)cc(-c2cccc3c2oc2ccccc23)c1]

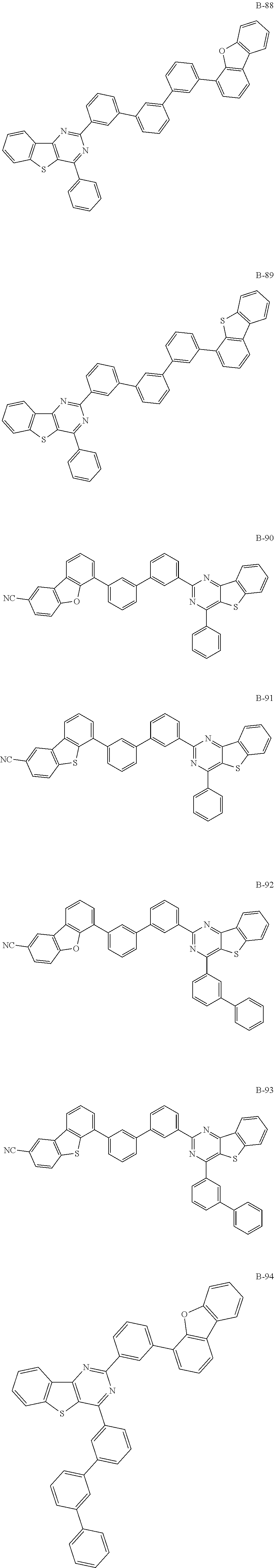

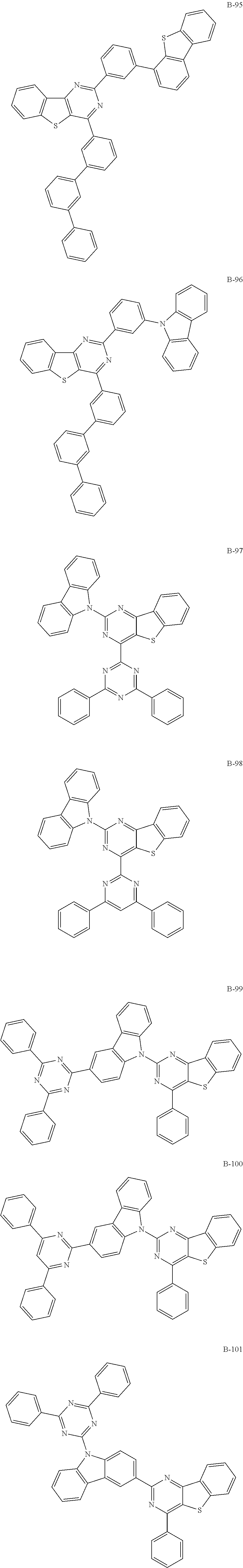

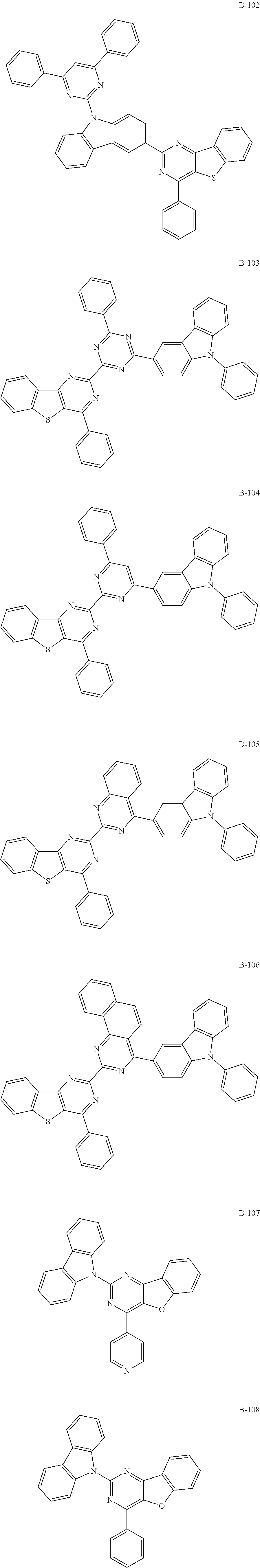

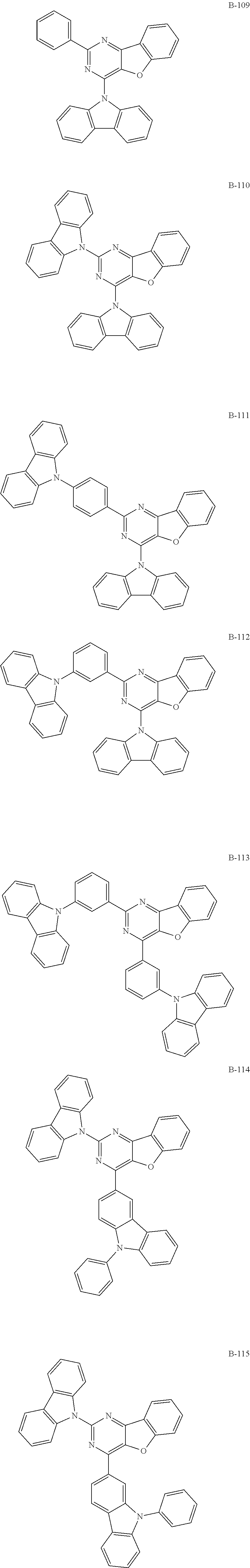

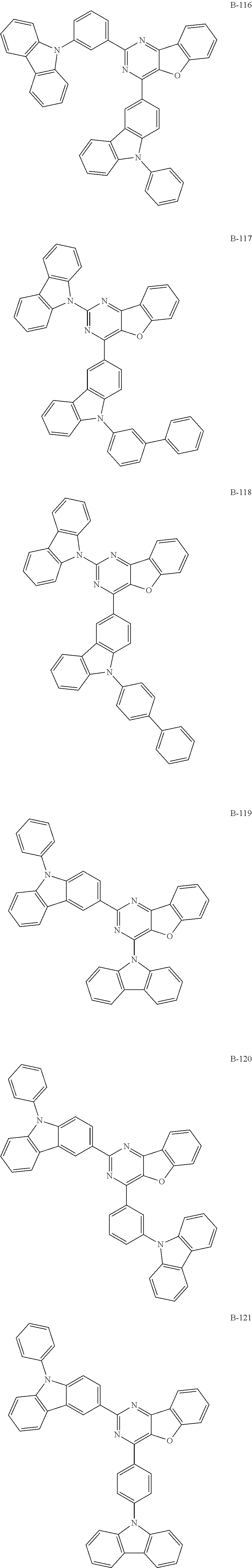

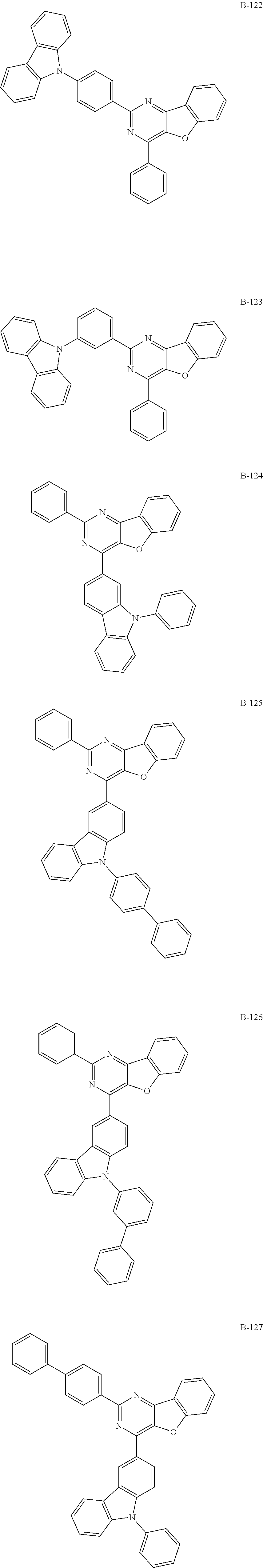 [SMILES: c1ccc(-c2nc(-c3ccc(-n4c5ccccc5c5ccccc54)cc3)nc3c2oc2ccccc23)cc1 ; c1ccc(-c2nc(-c3cccc(-n4c5ccccc5c5ccccc54)c3)nc3c2oc2ccccc23)cc1 ; c1ccc(-c2nc(-c3ccc4c5ccccc5n(-c5ccccc5)c4c3)c3oc4ccccc4c3n2)cc1 ; c1ccc(-c2ccc(-n3c4ccccc4c4cc(-c5nc(-c6ccccc6)nc6c5oc5ccccc56)ccc43)cc2)cc1 ; c1ccc(-c2cccc(-n3c4ccccc4c4cc(-c5nc(-c6ccccc6)nc6c5oc5ccccc56)ccc43)c2)cc1 ; c1ccc(-c2ccc(-c3nc(-c4ccc5c(c4)c4ccccc4n5-c4ccccc4)c4oc5ccccc5c4n3)cc2)cc1]

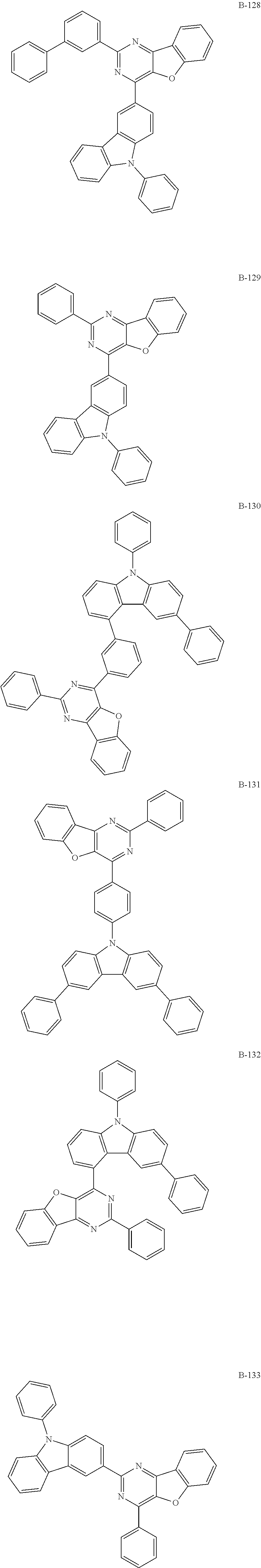

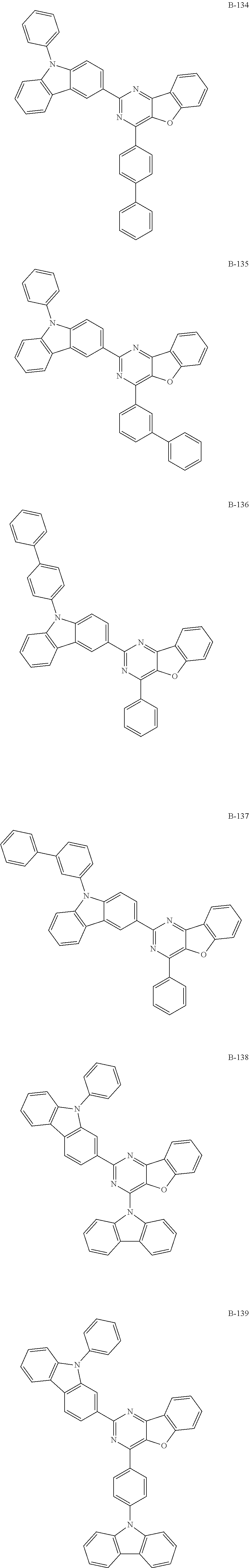 [SMILES: c1ccc(-c2ccc(-c3nc(-c4ccc5c(c4)c4ccccc4n5-c4ccccc4)nc4c3oc3ccccc34)cc2)cc1 ; c1ccc(-c2cccc(-c3nc(-c4ccc5c(c4)c4ccccc4n5-c4ccccc4)nc4c3oc3ccccc34)c2)cc1 ; c1ccc(-c2ccc(-n3c4ccccc4c4cc(-c5nc(-c6ccccc6)c6oc7ccccc7c6n5)ccc43)cc2)cc1 ; c1ccc(-c2cccc(-n3c4ccccc4c4cc(-c5nc(-c6ccccc6)c6oc7ccccc7c6n5)ccc43)c2)cc1 ; c1ccc(-n2c3ccccc3c3ccc(-c4nc(-n5c6ccccc6c6ccccc65)c5oc6ccccc6c5n4)cc32)cc1 ; c1ccc(-n2c3ccccc3c3ccc(-c4nc(-c5ccc(-n6c7ccccc7c7ccccc76)cc5)c5oc6ccccc6c5n4)cc32)cc1]

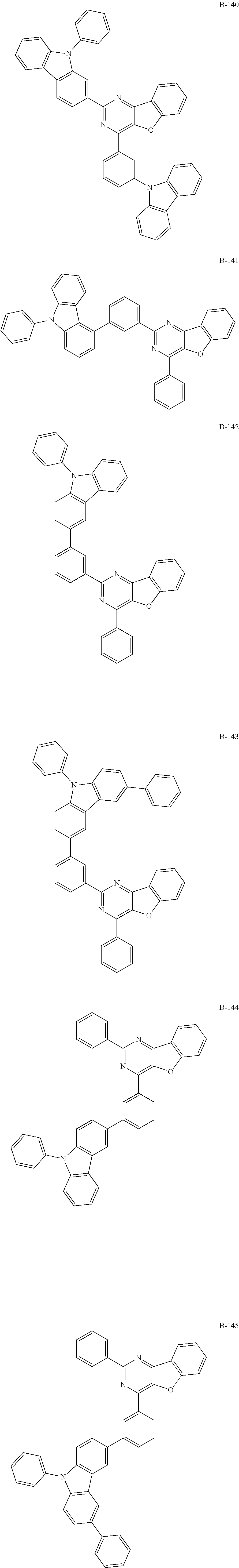 [SMILES: c1ccc(-n2c3ccccc3c3ccc(-c4nc(-c5cccc(-n6c7ccccc7c7ccccc76)c5)c5oc6ccccc6c5n4)cc32)cc1 ; c1ccc(-c2nc(-c3cccc(-c4cccc5c4c4ccccc4n5-c4ccccc4)c3)nc3c2oc2ccccc23)cc1 ; c1ccc(-c2nc(-c3cccc(-c4ccc5c(c4)c4ccccc4n5-c4ccccc4)c3)nc3c2oc2ccccc23)cc1 ; c1ccc(-c2ccc3c(c2)c2cc(-c4cccc(-c5nc(-c6ccccc6)c6oc7ccccc7c6n5)c4)ccc2n3-c2ccccc2)cc1 ; c1ccc(-c2nc(-c3cccc(-c4ccc5c(c4)c4ccccc4n5-c4ccccc4)c3)c3oc4ccccc4c3n2)cc1 ; c1ccc(-c2ccc3c(c2)c2cc(-c4cccc(-c5nc(-c6ccccc6)nc6c5oc5ccccc56)c4)ccc2n3-c2ccccc2)cc1]

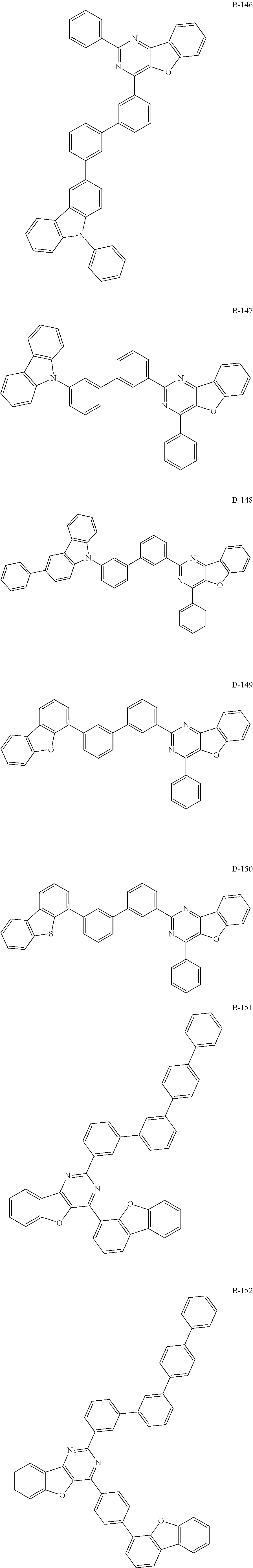

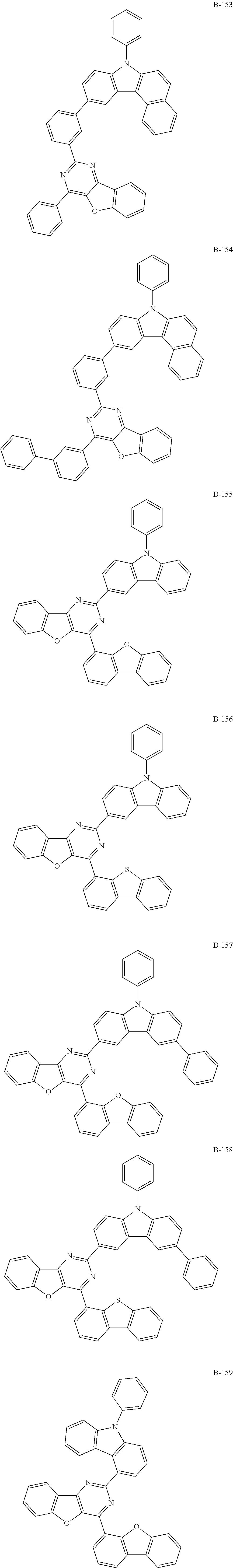

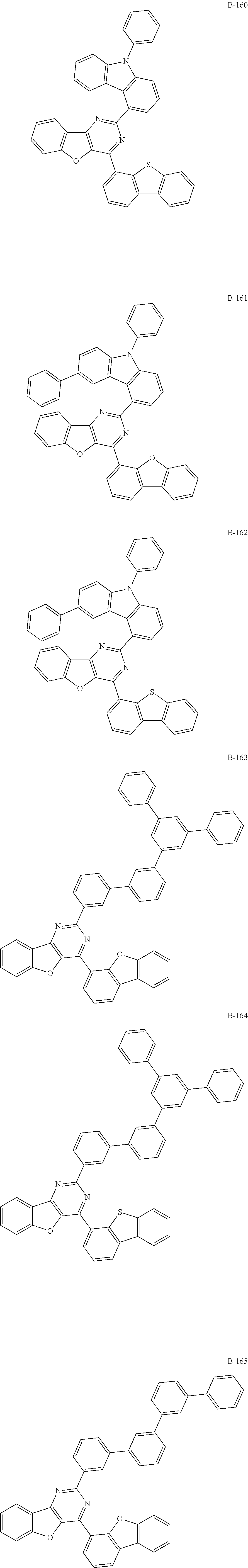

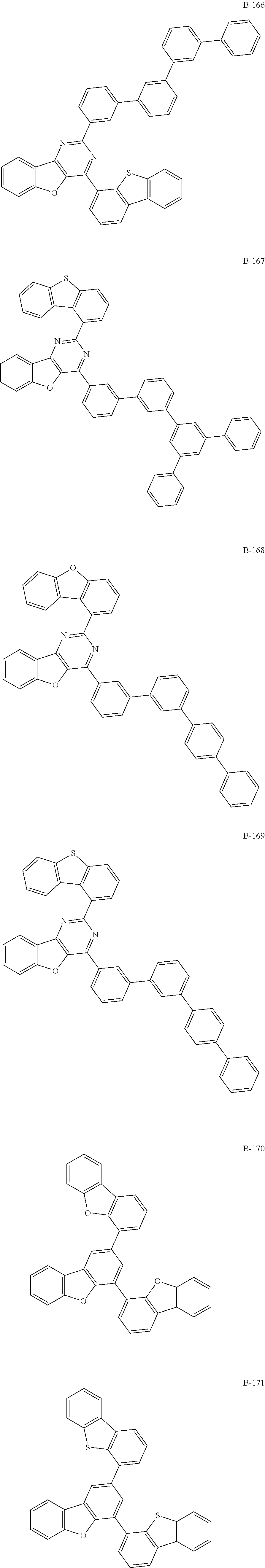 [SMILES: c1ccc(-c2cccc(-c3cccc(-c4cccc(-c5nc(-c6cccc7c6sc6ccccc67)c6oc7ccccc7c6n5)c4)c3)c2)cc1 ; c1ccc(-c2cc(-c3ccccc3)cc(-c3cccc(-c4cccc(-c5nc(-c6cccc7sc8ccccc8c67)nc6c5oc5ccccc56)c4)c3)c2)cc1 ; c1ccc(-c2ccc(-c3cccc(-c4cccc(-c5nc(-c6cccc7oc8ccccc8c67)nc6c5oc5ccccc56)c4)c3)cc2)cc1 ; c1ccc(-c2ccc(-c3cccc(-c4cccc(-c5nc(-c6cccc7sc8ccccc8c67)nc6c5oc5ccccc56)c4)c3)cc2)cc1 ; c1ccc2c(c1)oc1c(-c3cc(-c4cccc5c4oc4ccccc45)c4oc5ccccc5c4c3)cccc12 ; c1ccc2c(c1)oc1c(-c3cccc4c3sc3ccccc34)cc(-c3cccc4c3sc3ccccc34)cc12]

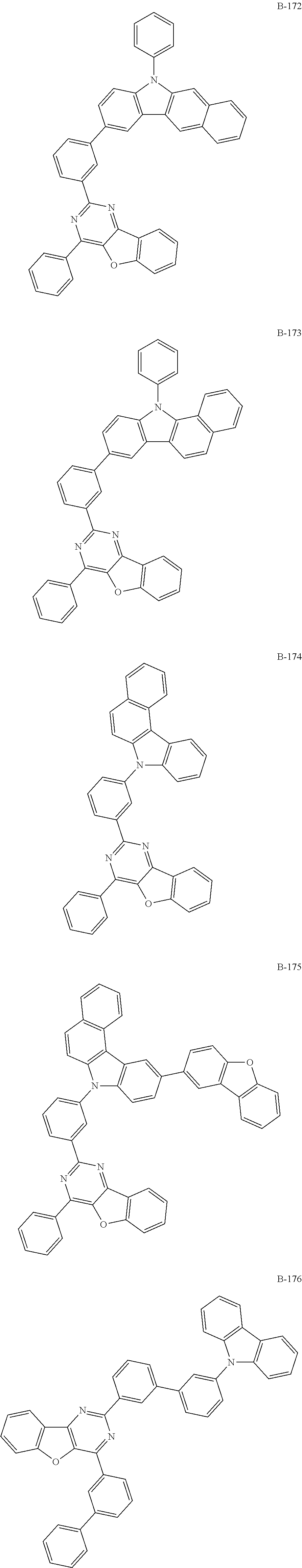 [SMILES: c1ccc(-c2nc(-c3cccc(-c4ccc5c(c4)c4cc6ccccc6cc4n5-c4ccccc4)c3)nc3c2oc2ccccc23)cc1 ; c1ccc(-c2nc(-c3cccc(-c4ccc5c(c4)c4ccc6ccccc6c4n5-c4ccccc4)c3)nc3c2oc2ccccc23)cc1 ; c1ccc(-c2nc(-c3cccc(-n4c5ccccc5c5c6ccccc6ccc54)c3)nc3c2oc2ccccc23)cc1 ; c1ccc(-c2nc(-c3cccc(-n4c5ccc(-c6ccc7oc8ccccc8c7c6)cc5c5c6ccccc6ccc54)c3)nc3c2oc2ccccc23)cc1 ; c1ccc(-c2cccc(-c3nc(-c4cccc(-c5cccc(-n6c7ccccc7c7ccccc76)c5)c4)nc4c3oc3ccccc34)c2)cc1]

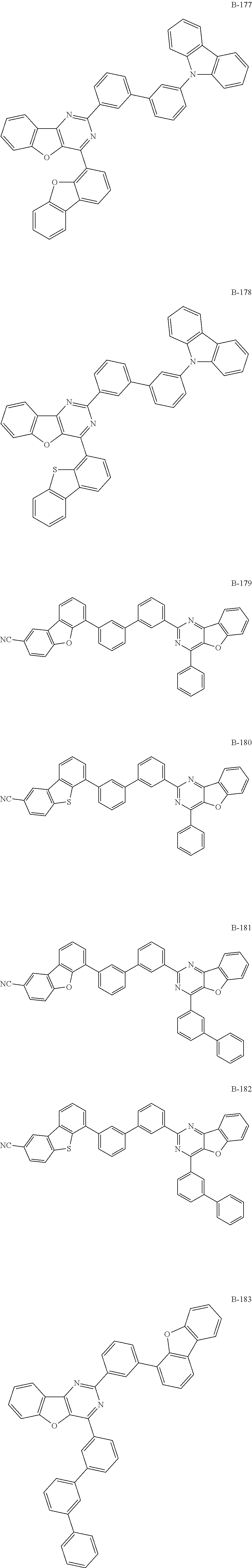

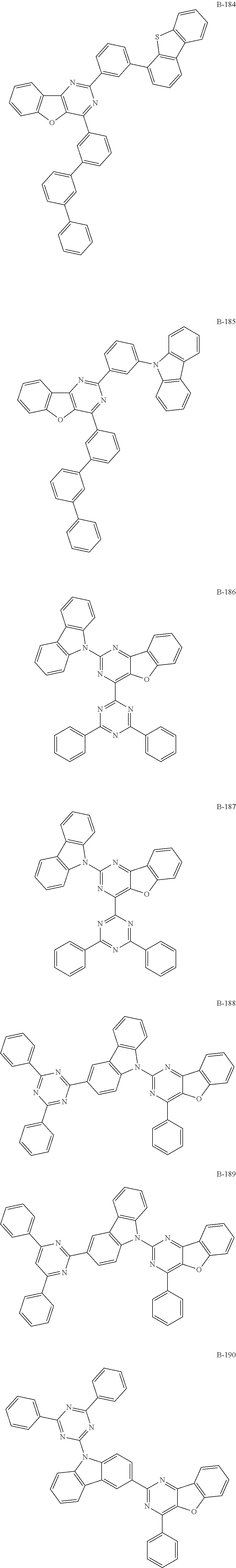

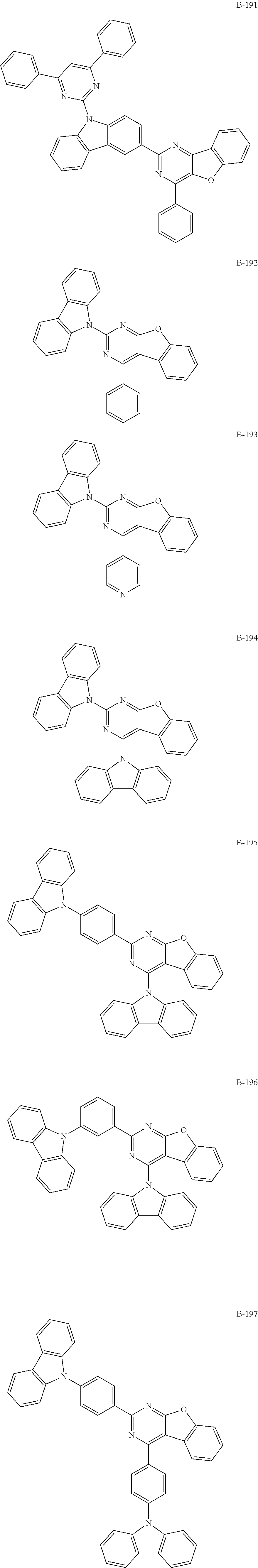

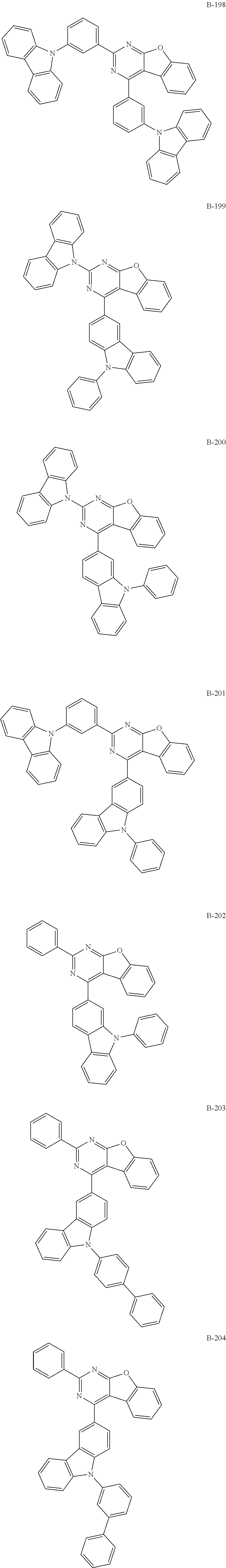

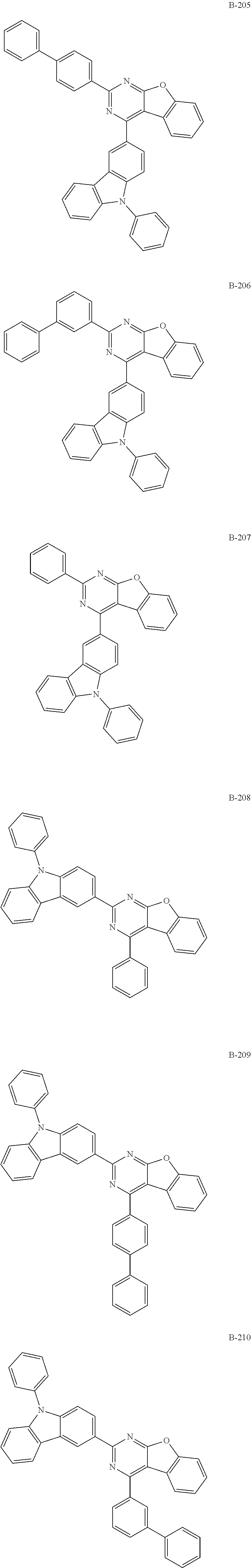 [SMILES: c1ccc(-c2ccc(-c3nc(-c4ccc5c(c4)c4ccccc4n5-c4ccccc4)c4c(n3)oc3ccccc34)cc2)cc1 ; c1ccc(-c2cccc(-c3nc(-c4ccc5c(c4)c4ccccc4n5-c4ccccc4)c4c(n3)oc3ccccc34)c2)cc1 ; c1ccc(-c2nc(-c3ccc4c(c3)c3ccccc3n4-c3ccccc3)c3c(n2)oc2ccccc23)cc1 ; c1ccc(-c2nc(-c3ccc4c(c3)c3ccccc3n4-c3ccccc3)nc3oc4ccccc4c23)cc1 ; c1ccc(-c2ccc(-c3nc(-c4ccc5c(c4)c4ccccc4n5-c4ccccc4)nc4oc5ccccc5c34)cc2)cc1 ; c1ccc(-c2cccc(-c3nc(-c4ccc5c(c4)c4ccccc4n5-c4ccccc4)nc4oc5ccccc5c34)c2)cc1]

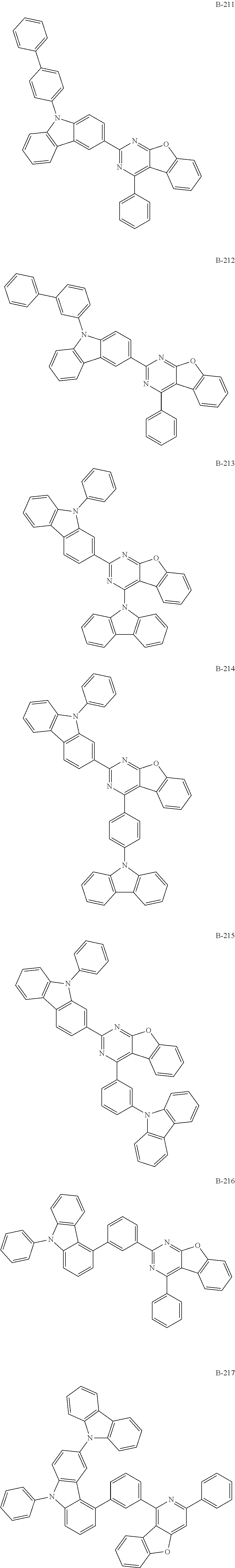

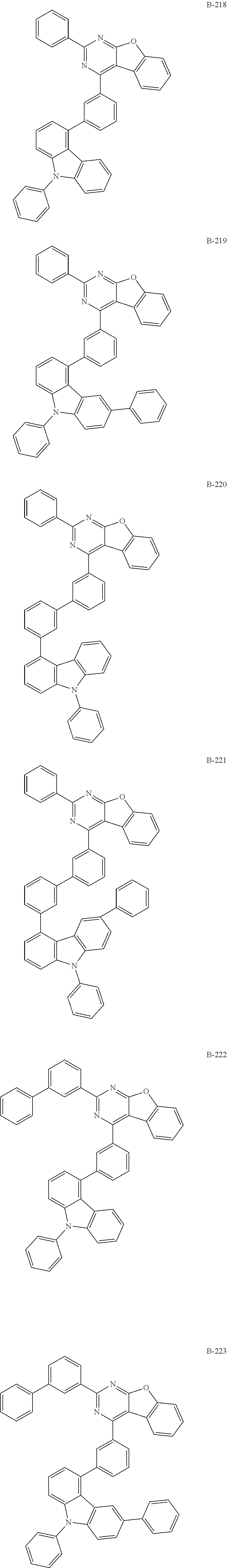

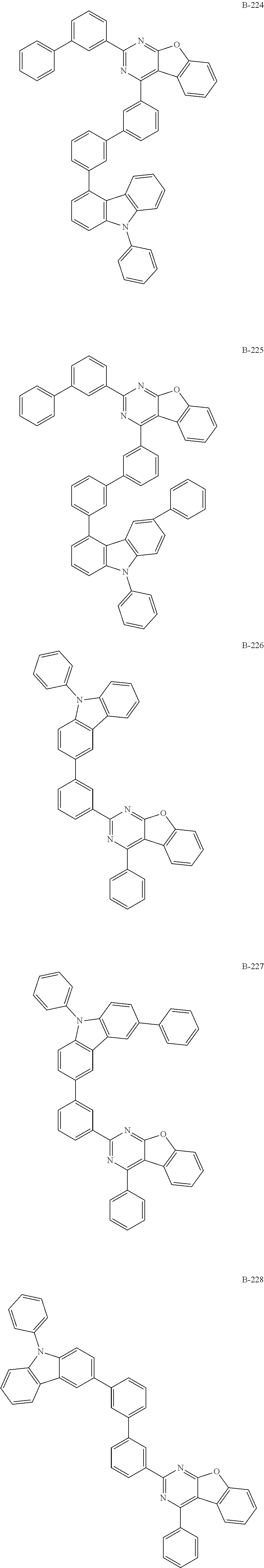 [SMILES: c1ccc(-c2cccc(-c3nc(-c4cccc(-c5cccc(-c6cccc7c6c6ccccc6n7-c6ccccc6)c5)c4)c4c(n3)oc3ccccc34)c2)cc1 ; c1ccc(-c2cccc(-c3nc(-c4cccc(-c5cccc(-c6cccc7c6c6cc(-c8ccccc8)ccc6n7-c6ccccc6)c5)c4)c4c(n3)oc3ccccc34)c2)cc1 ; c1ccc(-c2nc(-c3cccc(-c4ccc5c(c4)c4ccccc4n5-c4ccccc4)c3)nc3oc4ccccc4c23)cc1 ; c1ccc(-c2ccc3c(c2)c2cc(-c4cccc(-c5nc(-c6ccccc6)c6c(n5)oc5ccccc56)c4)ccc2n3-c2ccccc2)cc1 ; c1ccc(-c2nc(-c3cccc(-c4cccc(-c5ccc6c(c5)c5ccccc5n6-c5ccccc5)c4)c3)nc3oc4ccccc4c23)cc1]

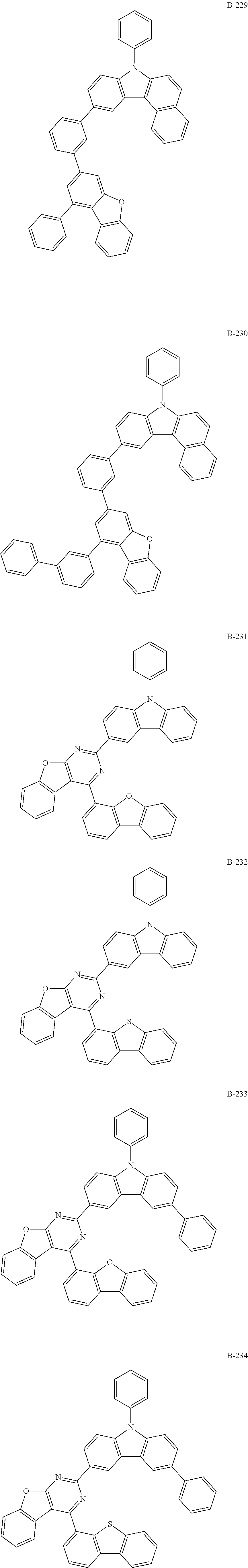

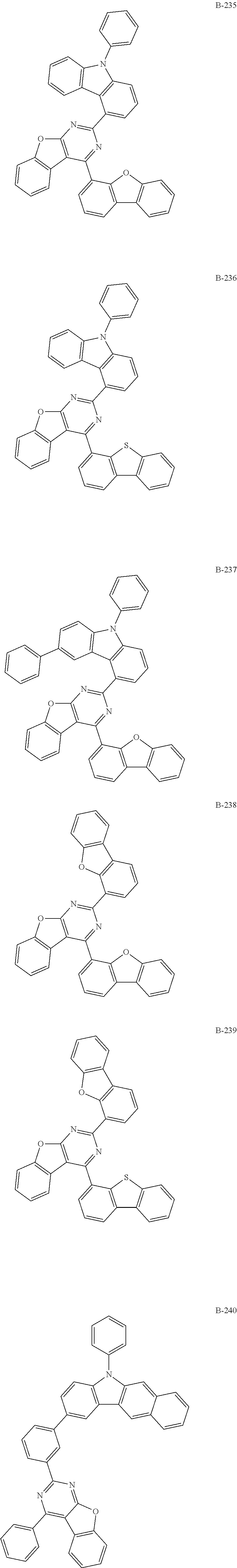 [SMILES: c1ccc(-n2c3ccccc3c3c(-c4nc(-c5cccc6c5oc5ccccc56)c5c(n4)oc4ccccc45)cccc32)cc1 ; c1ccc(-n2c3ccccc3c3c(-c4nc(-c5cccc6c5sc5ccccc56)c5c(n4)oc4ccccc45)cccc32)cc1 ; c1ccc(-c2ccc3c(c2)c2c(-c4nc(-c5cccc6c5oc5ccccc56)c5c(n4)oc4ccccc45)cccc2n3-c2ccccc2)cc1 ; c1ccc2c(c1)oc1c(-c3nc(-c4cccc5c4oc4ccccc45)c4c(n3)oc3ccccc34)cccc12 ; c1ccc2c(c1)oc1c(-c3nc(-c4cccc5c4sc4ccccc45)c4c(n3)oc3ccccc34)cccc12 ; c1ccc(-c2nc(-c3cccc(-c4ccc5c(c4)c4cc6ccccc6cc4n5-c4ccccc4)c3)nc3oc4ccccc4c23)cc1]

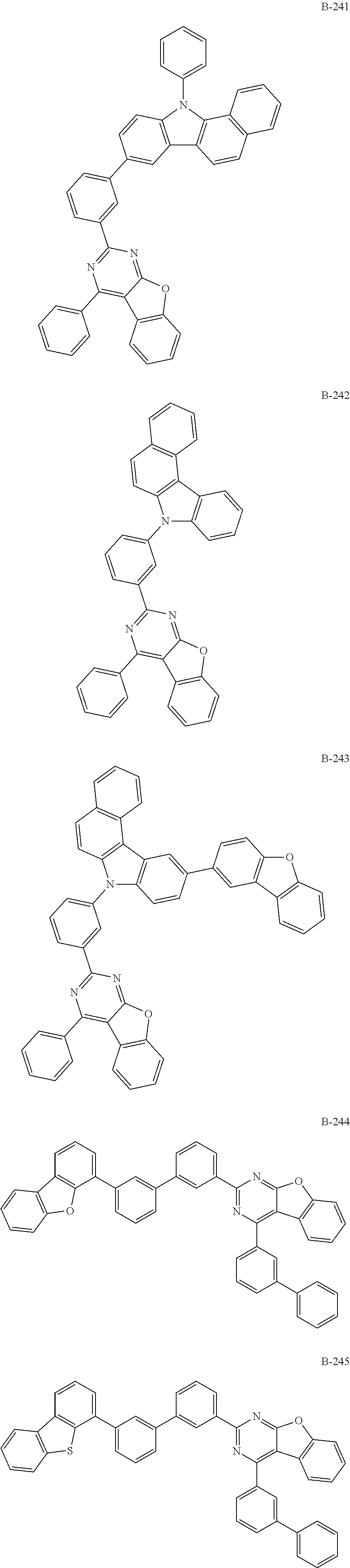 [SMILES: c1ccc(-c2nc(-c3cccc(-c4ccc5c(c4)c4ccc6ccccc6c4n5-c4ccccc4)c3)nc3oc4ccccc4c23)cc1 ; c1ccc(-c2nc(-c3cccc(-n4c5ccccc5c5c6ccccc6ccc54)c3)nc3oc4ccccc4c23)cc1 ; c1ccc(-c2nc(-c3cccc(-n4c5ccc(-c6ccc7oc8ccccc8c7c6)cc5c5c6ccccc6ccc54)c3)nc3oc4ccccc4c23)cc1 ; c1ccc(-c2cccc(-c3nc(-c4cccc(-c5cccc(-c6cccc7c6oc6ccccc67)c5)c4)nc4oc5ccccc5c34)c2)cc1 ; c1ccc(-c2cccc(-c3nc(-c4cccc(-c5cccc(-c6cccc7c6sc6ccccc67)c5)c4)nc4oc5ccccc5c34)c2)cc1]

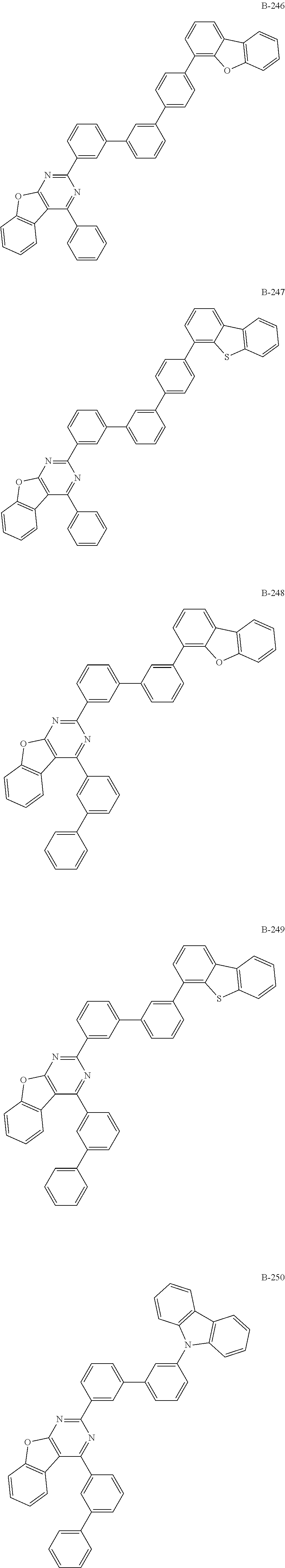 [SMILES: c1ccc(-c2nc(-c3cccc(-c4cccc(-c5ccc(-c6cccc7c6oc6ccccc67)cc5)c4)c3)nc3oc4ccccc4c23)cc1 ; c1ccc(-c2nc(-c3cccc(-c4cccc(-c5ccc(-c6cccc7c6sc6ccccc67)cc5)c4)c3)nc3oc4ccccc4c23)cc1 ; c1ccc(-c2cccc(-c3nc(-c4cccc(-c5cccc(-c6cccc7c6oc6ccccc67)c5)c4)nc4oc5ccccc5c34)c2)cc1 ; c1ccc(-c2cccc(-c3nc(-c4cccc(-c5cccc(-c6cccc7c6sc6ccccc67)c5)c4)nc4oc5ccccc5c34)c2)cc1 ; c1ccc(-c2cccc(-c3nc(-c4cccc(-c5cccc(-n6c7ccccc7c7ccccc76)c5)c4)nc4oc5ccccc5c34)c2)cc1]

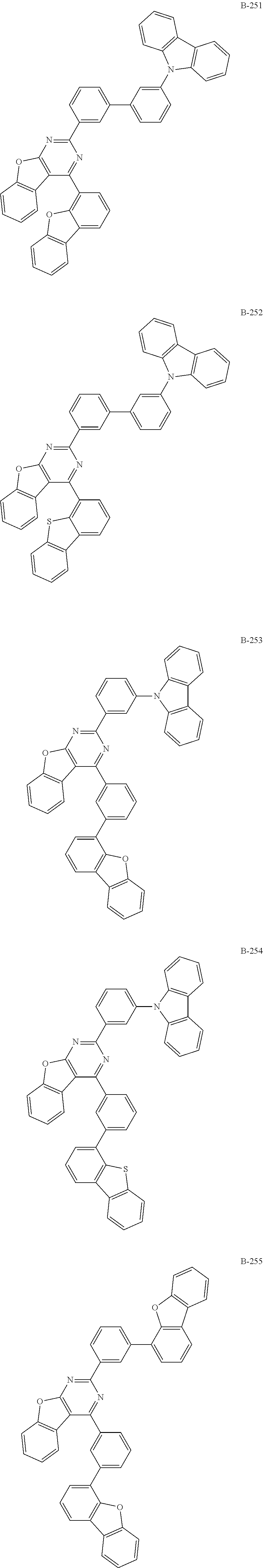 [SMILES: c1cc(-c2cccc(-n3c4ccccc4c4ccccc43)c2)cc(-c2nc(-c3cccc4c3oc3ccccc34)c3c(n2)oc2ccccc23)c1 ; c1cc(-c2cccc(-n3c4ccccc4c4ccccc43)c2)cc(-c2nc(-c3cccc4c3sc3ccccc34)c3c(n2)oc2ccccc23)c1 ; c1cc(-c2cccc3c2oc2ccccc23)cc(-c2nc(-c3cccc(-n4c5ccccc5c5ccccc54)c3)nc3oc4ccccc4c23)c1 ; c1cc(-c2cccc3c2sc2ccccc23)cc(-c2nc(-c3cccc(-n4c5ccccc5c5ccccc54)c3)nc3oc4ccccc4c23)c1 ; c1cc(-c2nc(-c3cccc(-c4cccc5c4oc4ccccc45)c3)c3c(n2)oc2ccccc23)cc(-c2cccc3c2oc2ccccc23)c1]

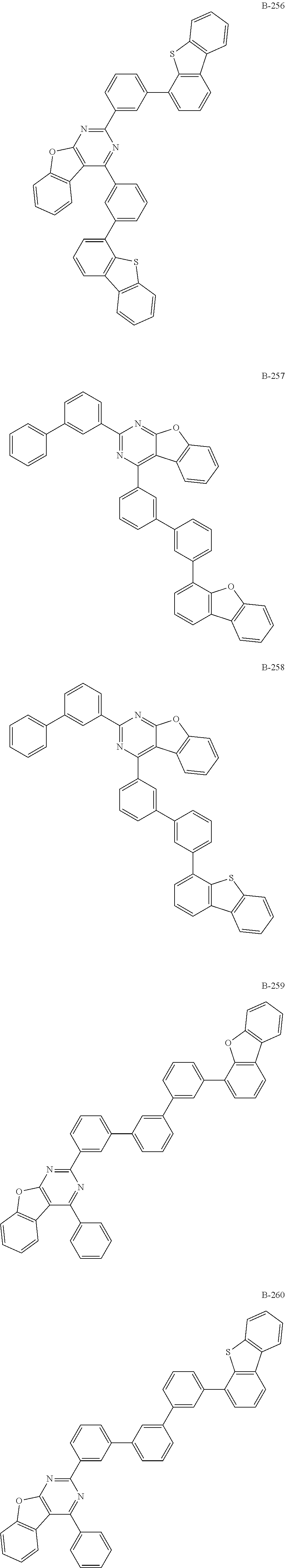 [SMILES: c1cc(-c2nc(-c3cccc(-c4cccc5c4sc4ccccc45)c3)c3c(n2)oc2ccccc23)cc(-c2cccc3c2sc2ccccc23)c1 ; c1ccc(-c2cccc(-c3nc(-c4cccc(-c5cccc(-c6cccc7c6oc6ccccc67)c5)c4)c4c(n3)oc3ccccc34)c2)cc1 ; c1ccc(-c2cccc(-c3nc(-c4cccc(-c5cccc(-c6cccc7c6sc6ccccc67)c5)c4)c4c(n3)oc3ccccc34)c2)cc1 ; c1ccc(-c2nc(-c3cccc(-c4cccc(-c5cccc(-c6cccc7c6oc6ccccc67)c5)c4)c3)nc3oc4ccccc4c23)cc1 ; c1ccc(-c2nc(-c3cccc(-c4cccc(-c5cccc(-c6cccc7c6sc6ccccc67)c5)c4)c3)nc3oc4ccccc4c23)cc1]

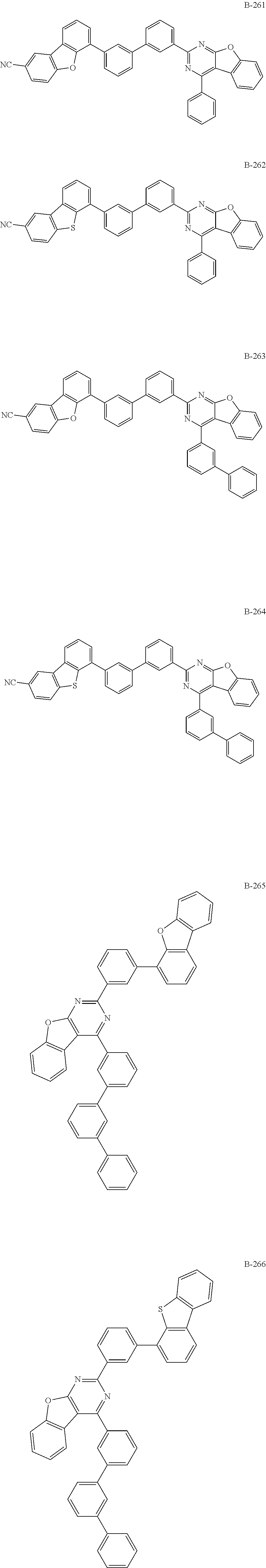

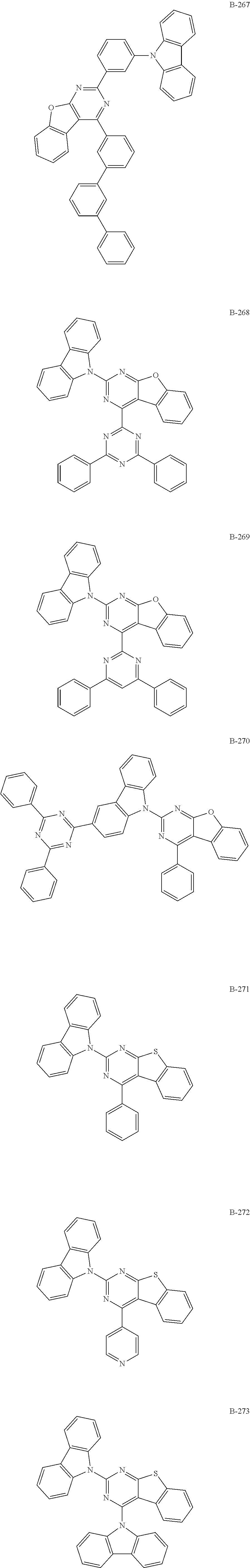

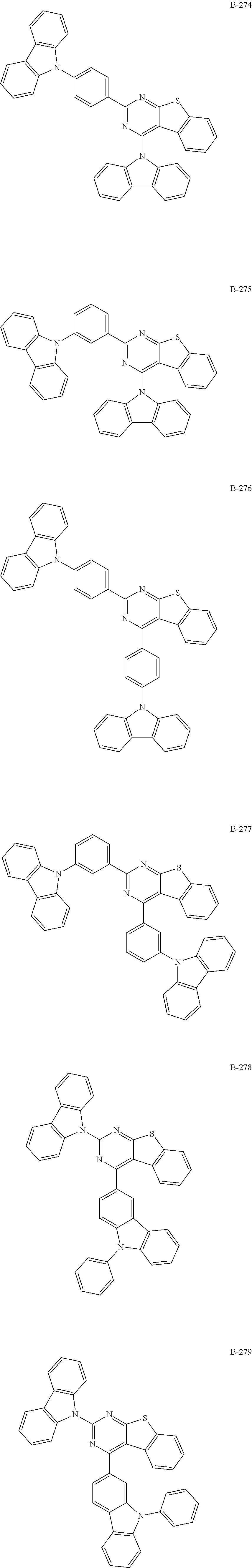 [SMILES: c1ccc2c(c1)sc1nc(-c3ccc(-n4c5ccccc5c5ccccc54)cc3)nc(-n3c4ccccc4c4ccccc43)c12 ; c1cc(-c2nc(-n3c4ccccc4c4ccccc43)c3c(n2)sc2ccccc23)cc(-n2c3ccccc3c3ccccc32)c1 ; c1ccc2c(c1)sc1nc(-c3ccc(-n4c5ccccc5c5ccccc54)cc3)nc(-c3ccc(-n4c5ccccc5c5ccccc54)cc3)c12 ; c1cc(-c2nc(-c3cccc(-n4c5ccccc5c5ccccc54)c3)c3c(n2)sc2ccccc23)cc(-n2c3ccccc3c3ccccc32)c1 ; c1ccc(-n2c3ccccc3c3cc(-c4nc(-n5c6ccccc6c6ccccc65)nc5sc6ccccc6c45)ccc32)cc1 ; c1ccc(-n2c3ccccc3c3ccc(-c4nc(-n5c6ccccc6c6ccccc65)nc5sc6ccccc6c45)cc32)cc1]

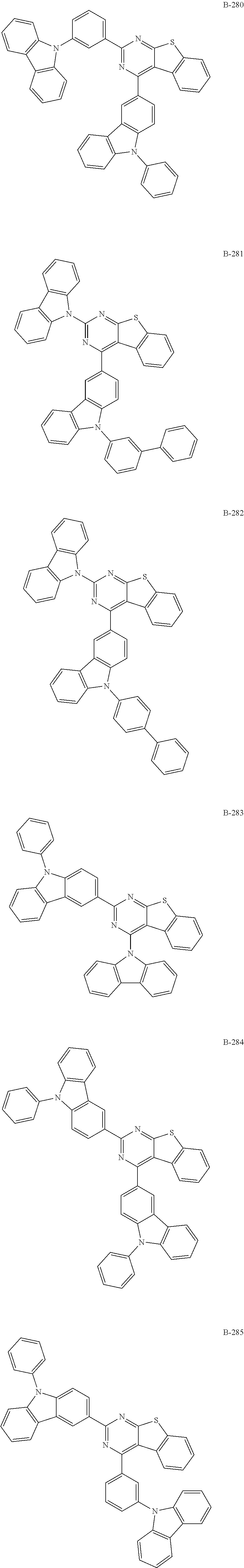

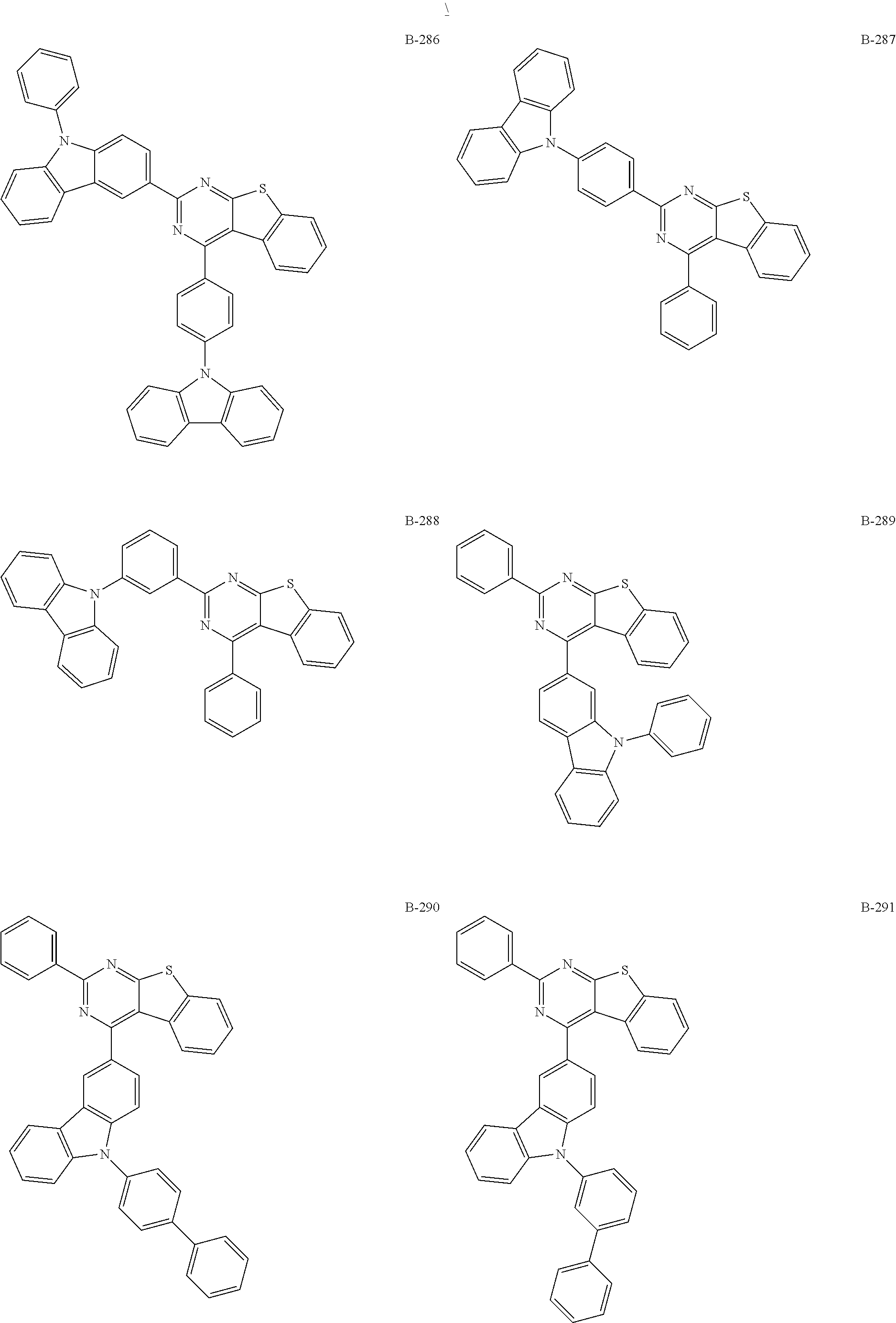 [SMILES: c1ccc(-n2c3ccccc3c3cc(-c4nc(-c5ccc(-n6c7ccccc7c7ccccc76)cc5)c5c(n4)sc4ccccc45)ccc32)cc1 ; c1ccc(-c2nc(-c3ccc(-n4c5ccccc5c5ccccc54)cc3)nc3sc4ccccc4c23)cc1 ; c1ccc(-c2nc(-c3cccc(-n4c5ccccc5c5ccccc54)c3)nc3sc4ccccc4c23)cc1 ; c1ccc(-c2nc(-c3ccc4c5ccccc5n(-c5ccccc5)c4c3)c3c(n2)sc2ccccc23)cc1 ; c1ccc(-c2ccc(-n3c4ccccc4c4cc(-c5nc(-c6ccccc6)nc6sc7ccccc7c56)ccc43)cc2)cc1 ; c1ccc(-c2cccc(-n3c4ccccc4c4cc(-c5nc(-c6ccccc6)nc6sc7ccccc7c56)ccc43)c2)cc1]

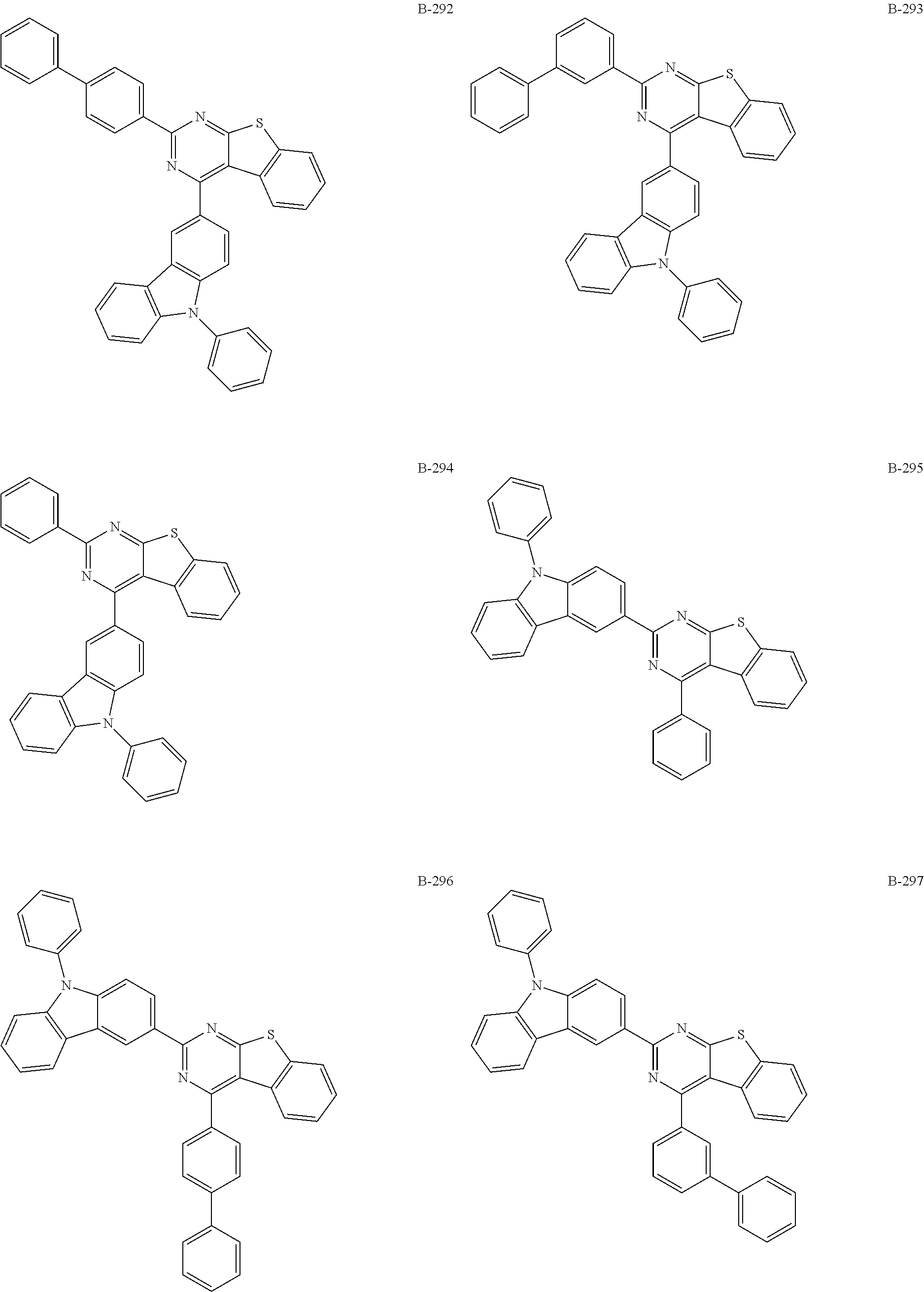 [SMILES: c1ccc(-c2ccc(-c3nc(-c4ccc5c(c4)c4ccccc4n5-c4ccccc4)c4c(n3)sc3ccccc34)cc2)cc1 ; c1ccc(-c2cccc(-c3nc(-c4ccc5c(c4)c4ccccc4n5-c4ccccc4)c4c(n3)sc3ccccc34)c2)cc1 ; c1ccc(-c2nc(-c3ccc4c(c3)c3ccccc3n4-c3ccccc3)c3c(n2)sc2ccccc23)cc1 ; c1ccc(-c2nc(-c3ccc4c(c3)c3ccccc3n4-c3ccccc3)nc3sc4ccccc4c23)cc1 ; c1ccc(-c2ccc(-c3nc(-c4ccc5c(c4)c4ccccc4n5-c4ccccc4)nc4sc5ccccc5c34)cc2)cc1 ; c1ccc(-c2cccc(-c3nc(-c4ccc5c(c4)c4ccccc4n5-c4ccccc4)nc4sc5ccccc5c34)c2)cc1]

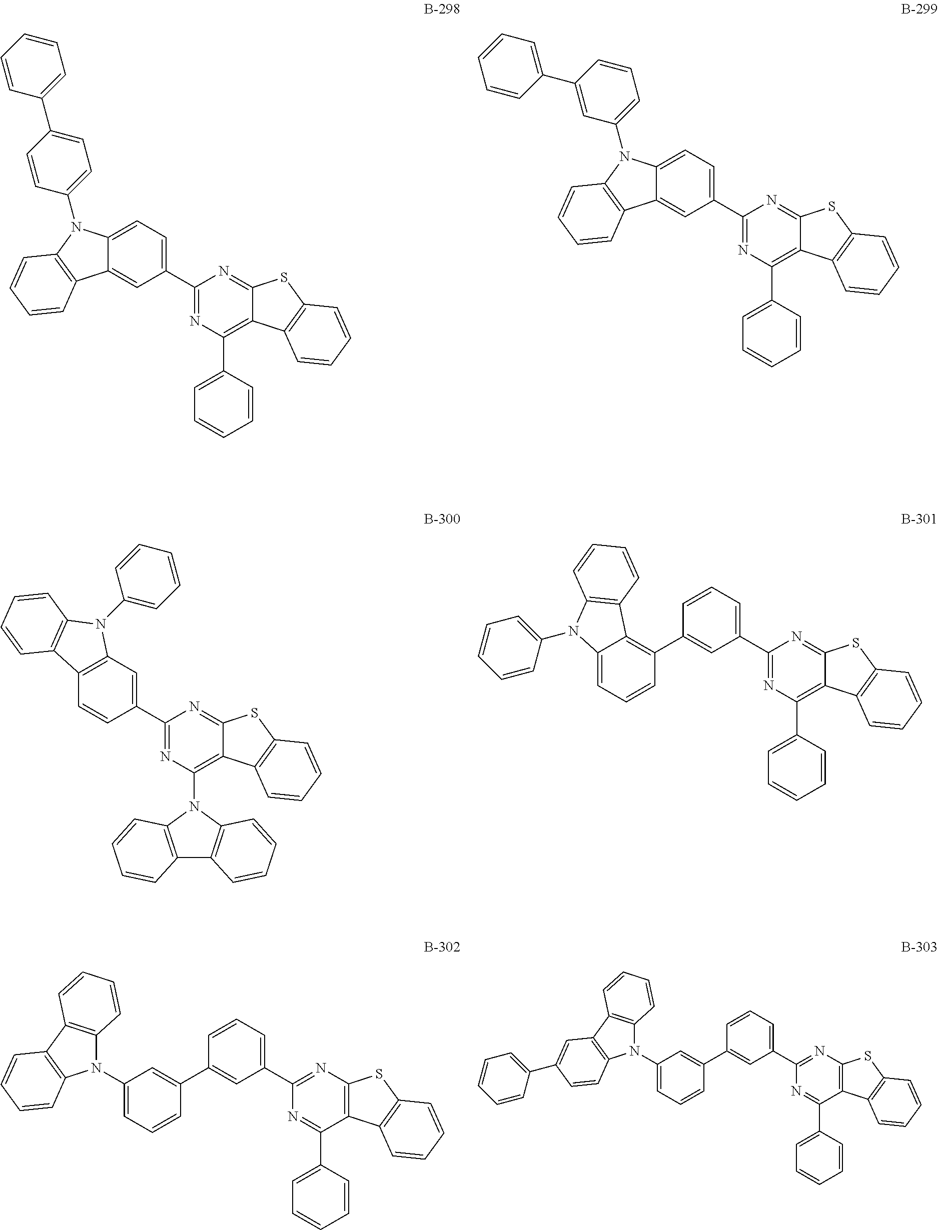 [SMILES: c1ccc(-c2ccc(-n3c4ccccc4c4cc(-c5nc(-c6ccccc6)c6c(n5)sc5ccccc56)ccc43)cc2)cc1 ; c1ccc(-c2cccc(-n3c4ccccc4c4cc(-c5nc(-c6ccccc6)c6c(n5)sc5ccccc56)ccc43)c2)cc1 ; c1ccc(-n2c3ccccc3c3ccc(-c4nc(-n5c6ccccc6c6ccccc65)c5c(n4)sc4ccccc45)cc32)cc1 ; c1ccc(-c2nc(-c3cccc(-c4cccc5c4c4ccccc4n5-c4ccccc4)c3)nc3sc4ccccc4c23)cc1 ; c1ccc(-c2nc(-c3cccc(-c4cccc(-n5c6ccccc6c6ccccc65)c4)c3)nc3sc4ccccc4c23)cc1 ; c1ccc(-c2ccc3c(c2)c2ccccc2n3-c2cccc(-c3cccc(-c4nc(-c5ccccc5)c5c(n4)sc4ccccc45)c3)c2)cc1]

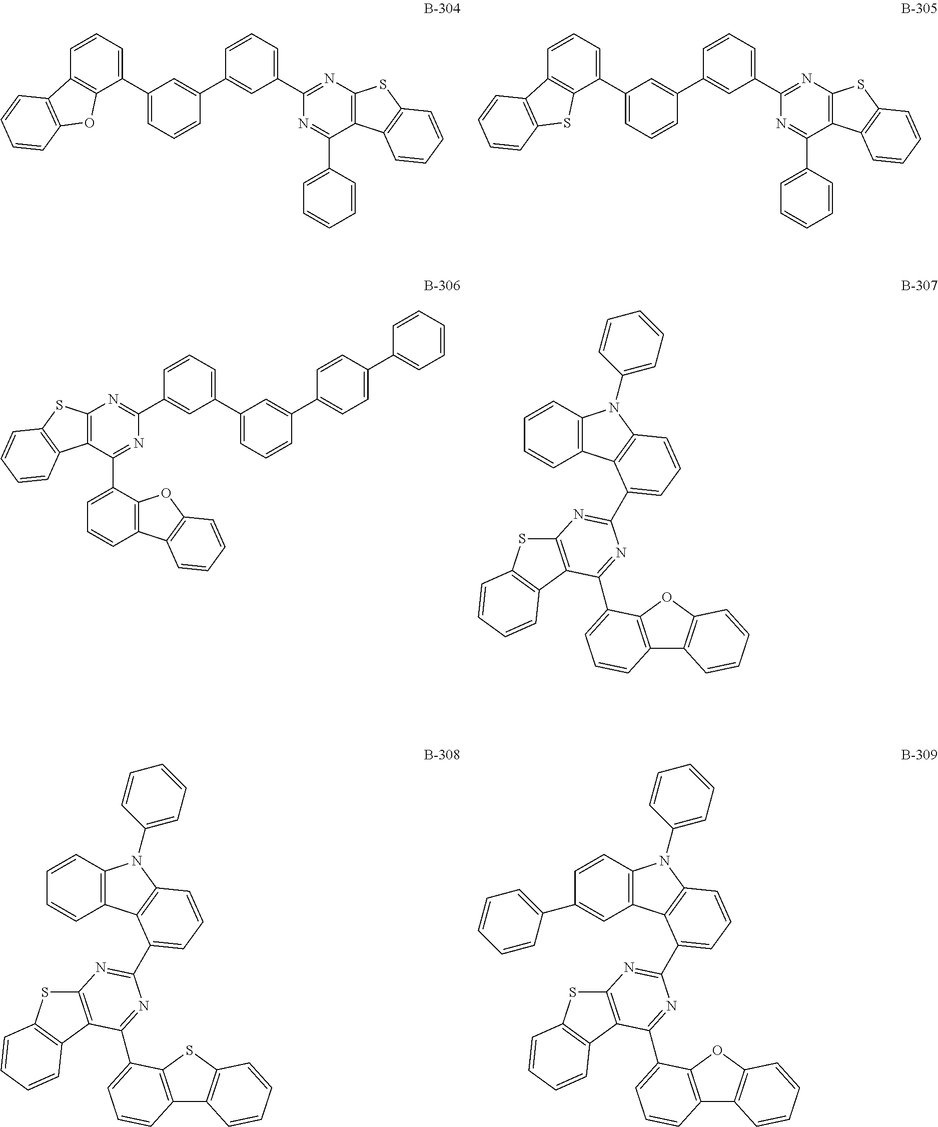

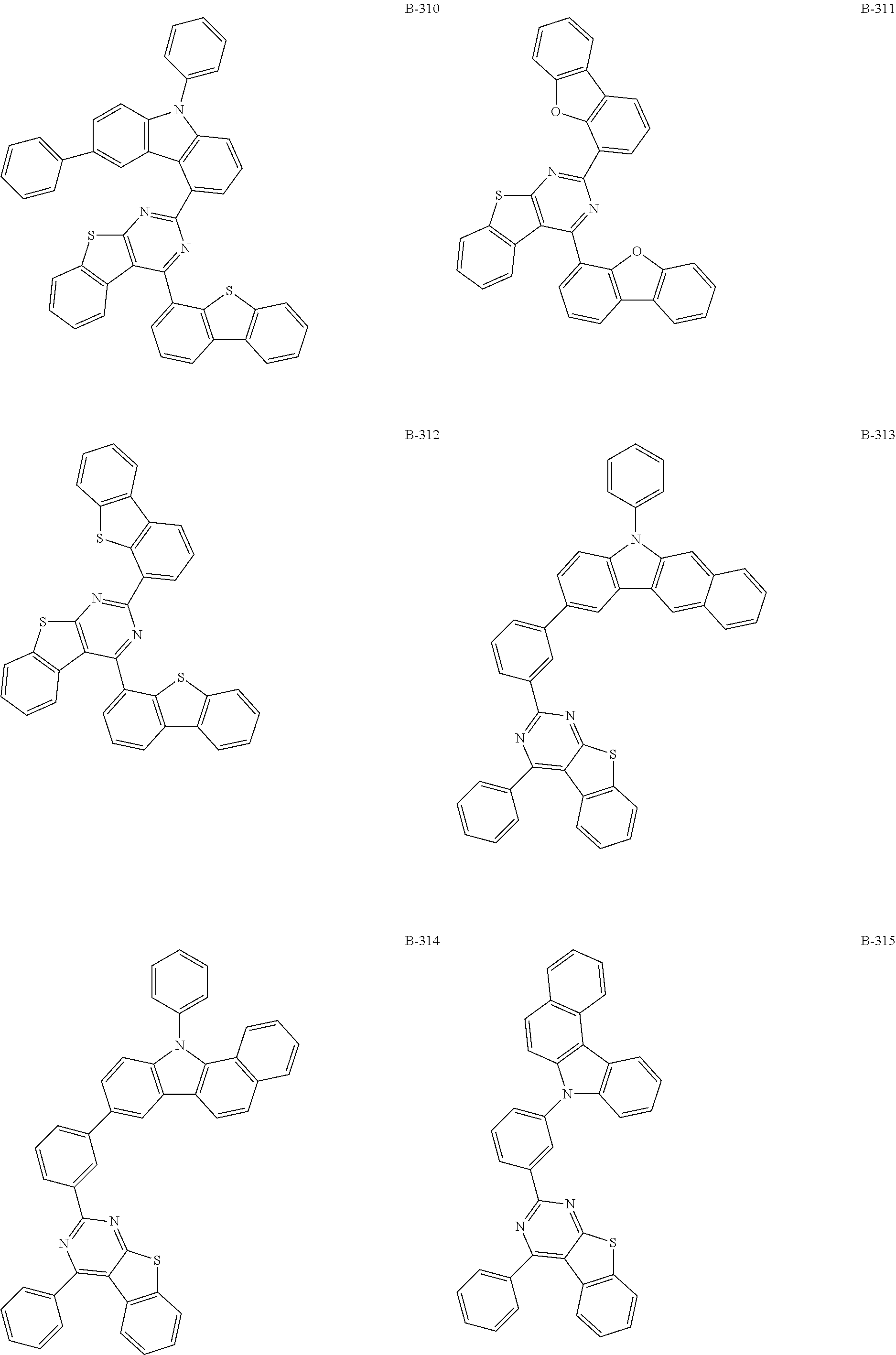 [SMILES: c1ccc(-c2ccc3c(c2)c2c(-c4nc(-c5cccc6c5sc5ccccc56)c5c(n4)sc4ccccc45)cccc2n3-c2ccccc2)cc1 ; c1ccc2c(c1)oc1c(-c3nc(-c4cccc5c4oc4ccccc45)c4c(n3)sc3ccccc34)cccc12 ; c1ccc2c(c1)sc1c(-c3nc(-c4cccc5c4sc4ccccc45)c4c(n3)sc3ccccc34)cccc12 ; c1ccc(-c2nc(-c3cccc(-c4ccc5c(c4)c4cc6ccccc6cc4n5-c4ccccc4)c3)nc3sc4ccccc4c23)cc1 ; c1ccc(-c2nc(-c3cccc(-c4ccc5c(c4)c4ccc6ccccc6c4n5-c4ccccc4)c3)nc3sc4ccccc4c23)cc1 ; c1ccc(-c2nc(-c3cccc(-n4c5ccccc5c5c6ccccc6ccc54)c3)nc3sc4ccccc4c23)cc1]

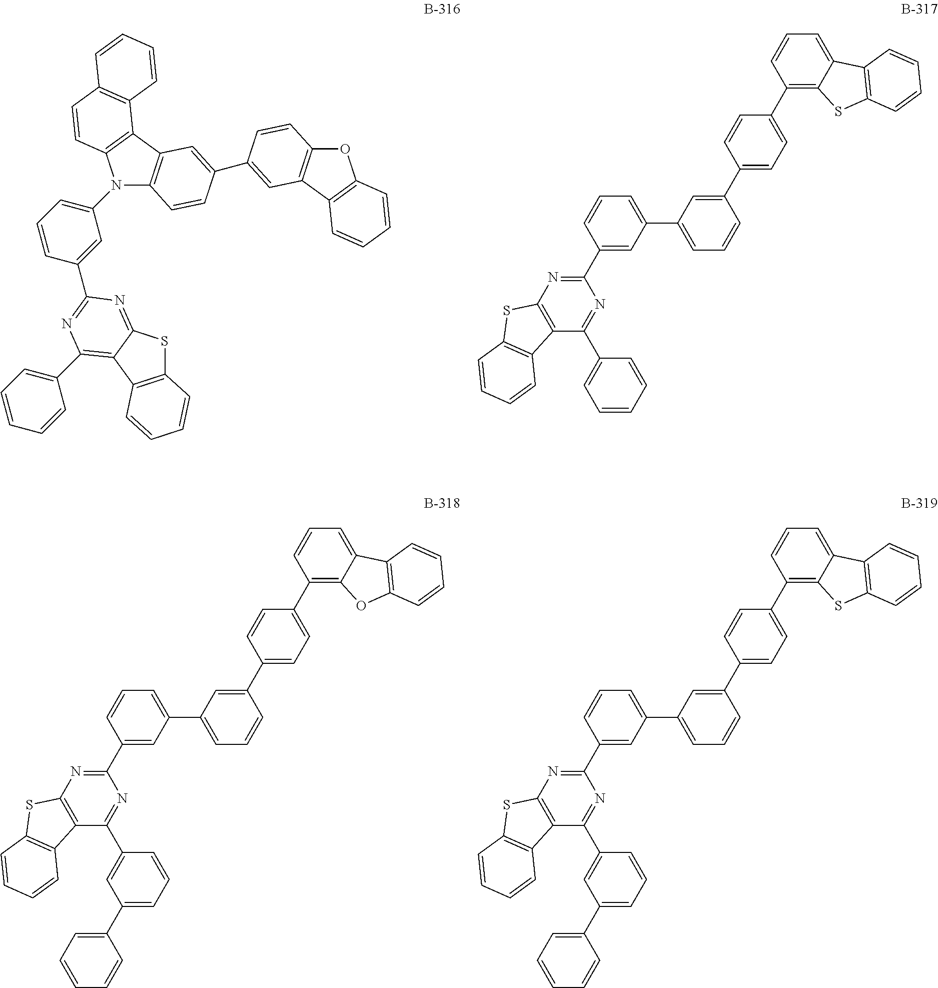 [SMILES: c1ccc(-c2nc(-c3cccc(-n4c5ccc(-c6ccc7oc8ccccc8c7c6)cc5c5c6ccccc6ccc54)c3)nc3sc4ccccc4c23)cc1 ; c1ccc(-c2nc(-c3cccc(-c4cccc(-c5ccc(-c6cccc7c6sc6ccccc67)cc5)c4)c3)nc3sc4ccccc4c23)cc1 ; c1ccc(-c2cccc(-c3nc(-c4cccc(-c5cccc(-c6ccc(-c7cccc8c7oc7ccccc78)cc6)c5)c4)nc4sc5ccccc5c34)c2)cc1 ; c1ccc(-c2cccc(-c3nc(-c4cccc(-c5cccc(-c6ccc(-c7cccc8c7sc7ccccc78)cc6)c5)c4)nc4sc5ccccc5c34)c2)cc1]

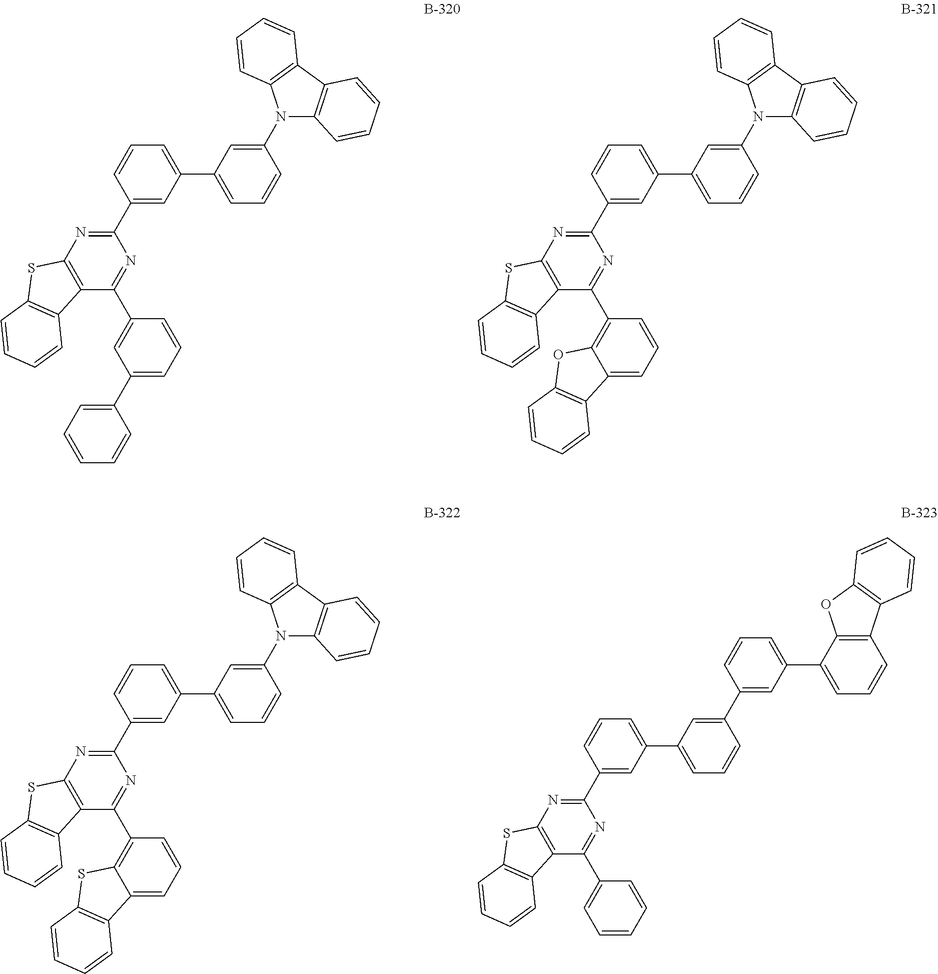 [SMILES: c1ccc(-c2cccc(-c3nc(-c4cccc(-c5cccc(-n6c7ccccc7c7ccccc76)c5)c4)nc4sc5ccccc5c34)c2)cc1 ; c1cc(-c2cccc(-n3c4ccccc4c4ccccc43)c2)cc(-c2nc(-c3cccc4c3oc3ccccc34)c3c(n2)sc2ccccc23)c1 ; c1cc(-c2cccc(-n3c4ccccc4c4ccccc43)c2)cc(-c2nc(-c3cccc4c3sc3ccccc34)c3c(n2)sc2ccccc23)c1 ; c1ccc(-c2nc(-c3cccc(-c4cccc(-c5cccc(-c6cccc7c6oc6ccccc67)c5)c4)c3)nc3sc4ccccc4c23)cc1]

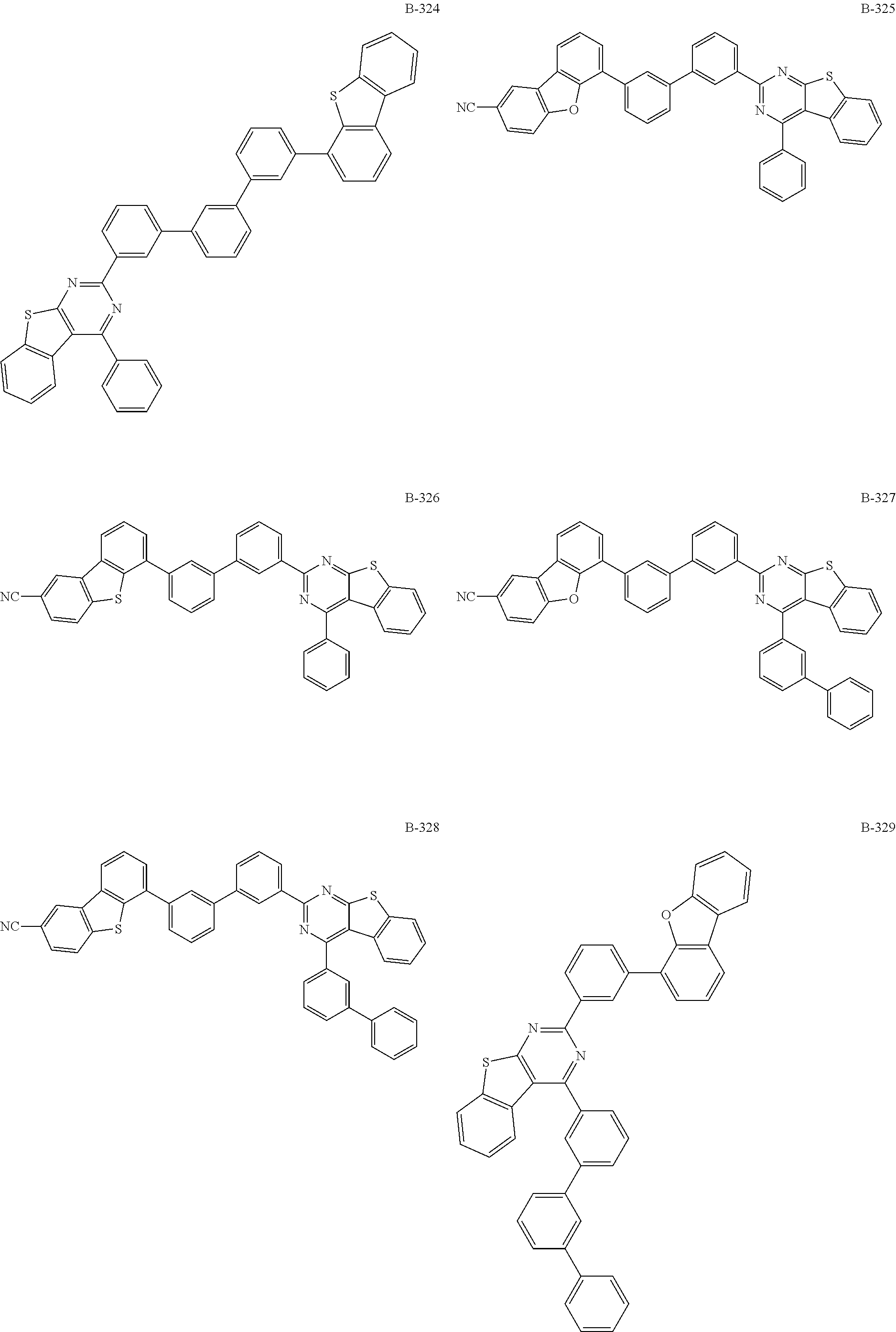

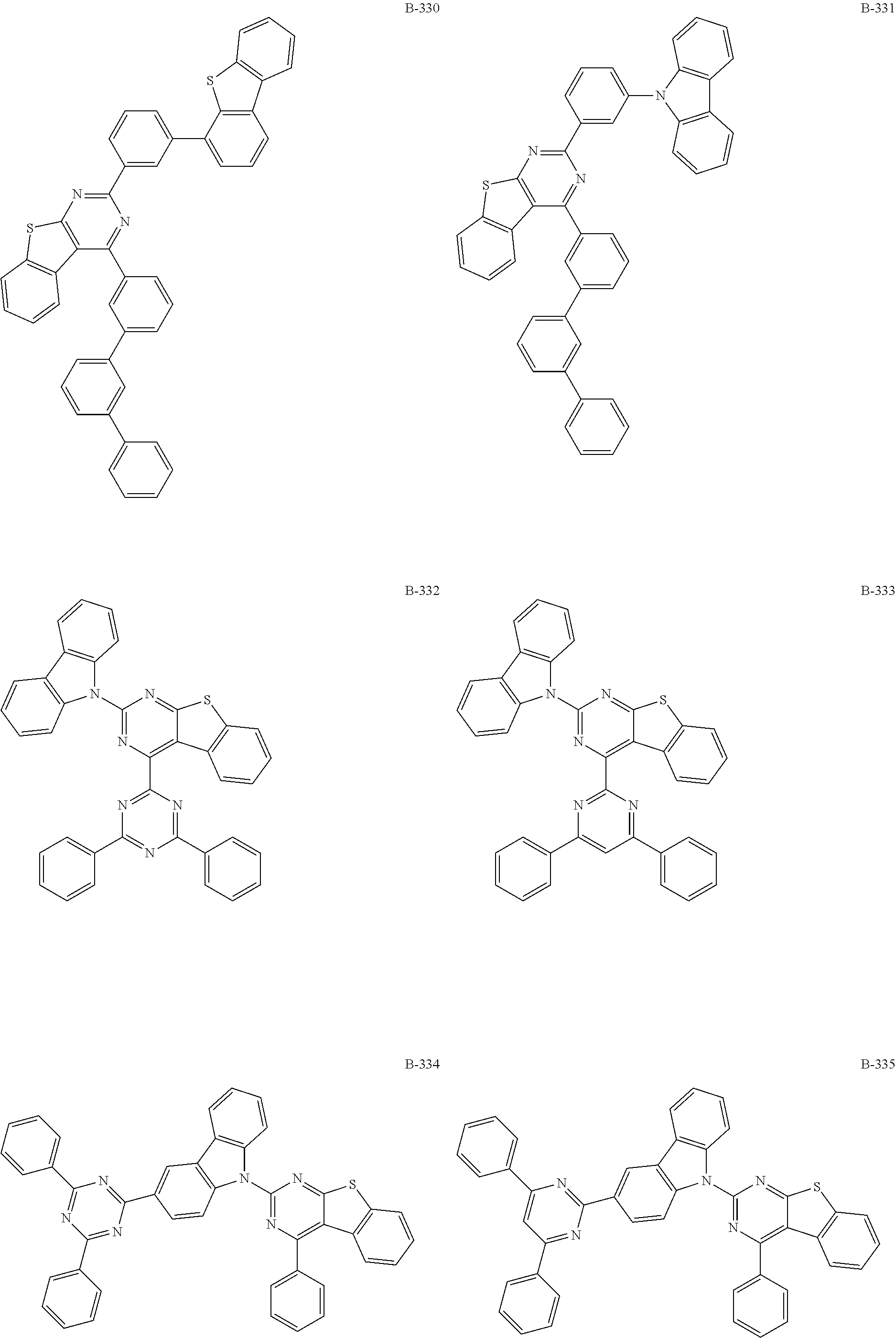

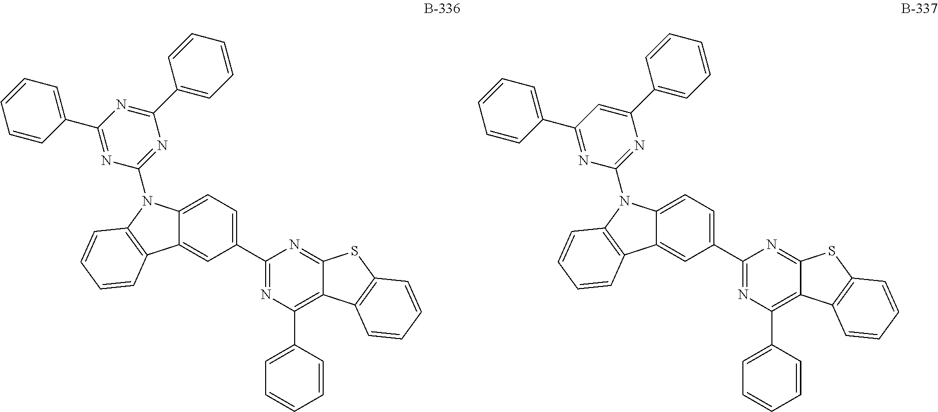 [SMILES: c1ccc(-c2nc(-c3ccccc3)nc(-n3c4ccccc4c4cc(-c5nc(-c6ccccc6)c6c(n5)sc5ccccc56)ccc43)n2)cc1 ; c1ccc(-c2cc(-c3ccccc3)nc(-n3c4ccccc4c4cc(-c5nc(-c6ccccc6)c6c(n5)sc5ccccc56)ccc43)n2)cc1]

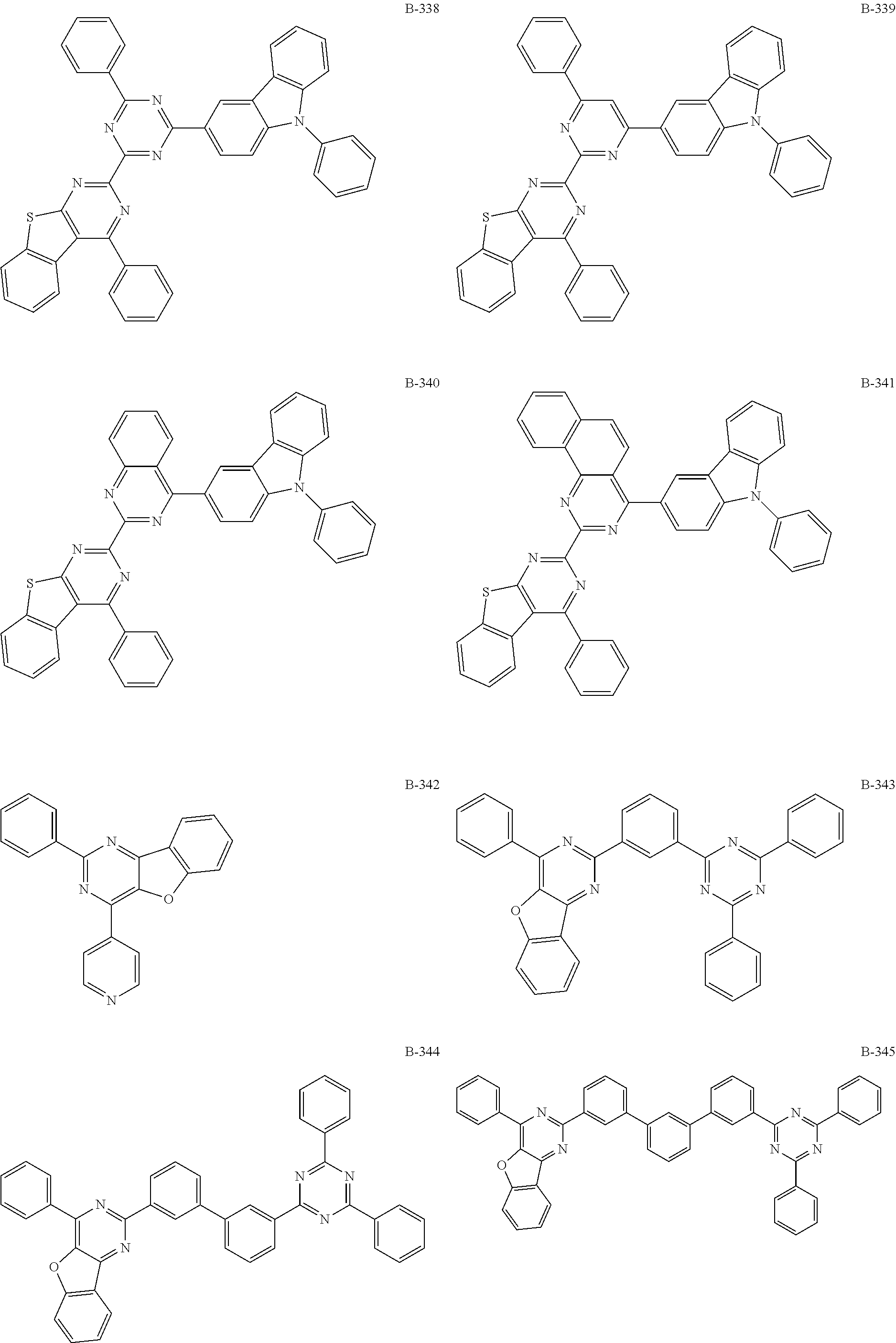

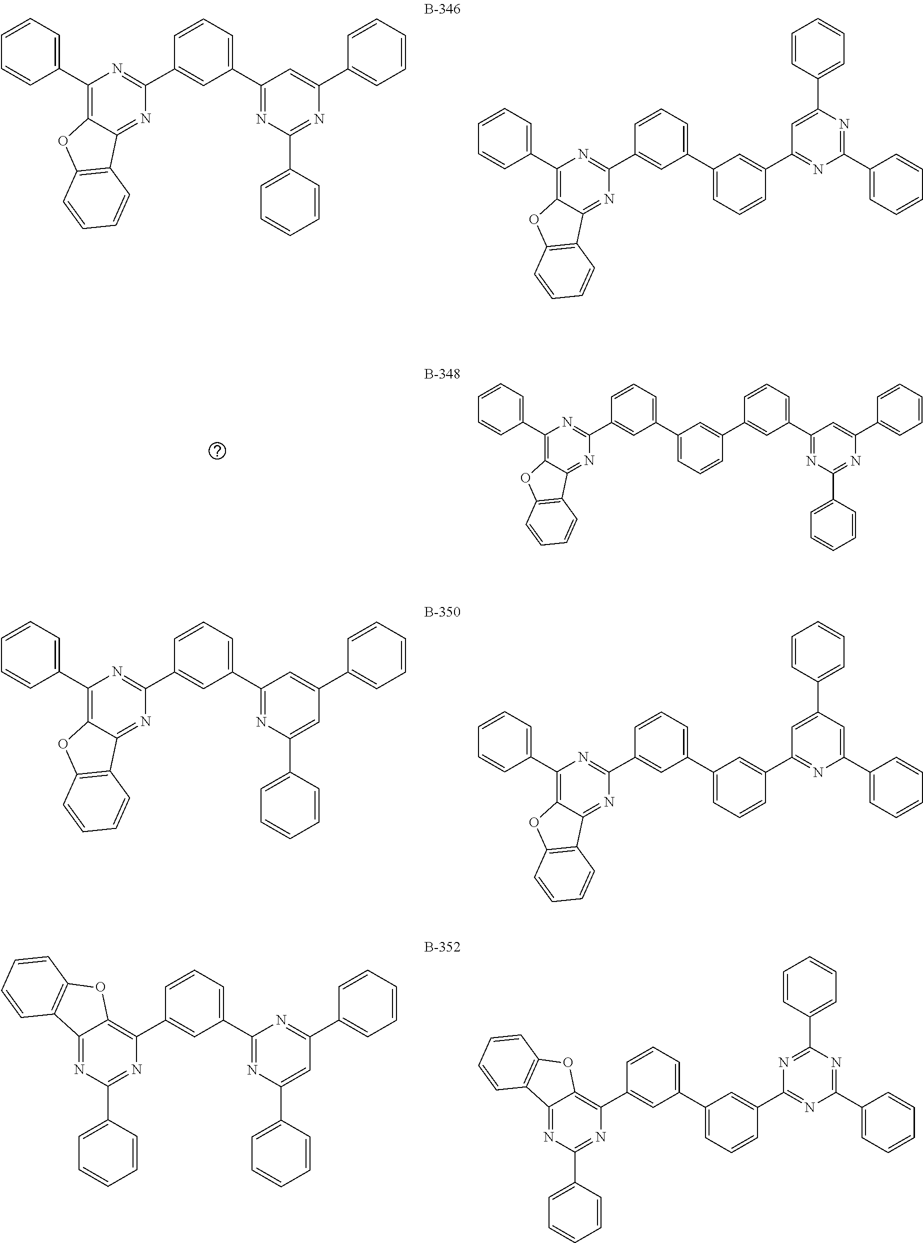

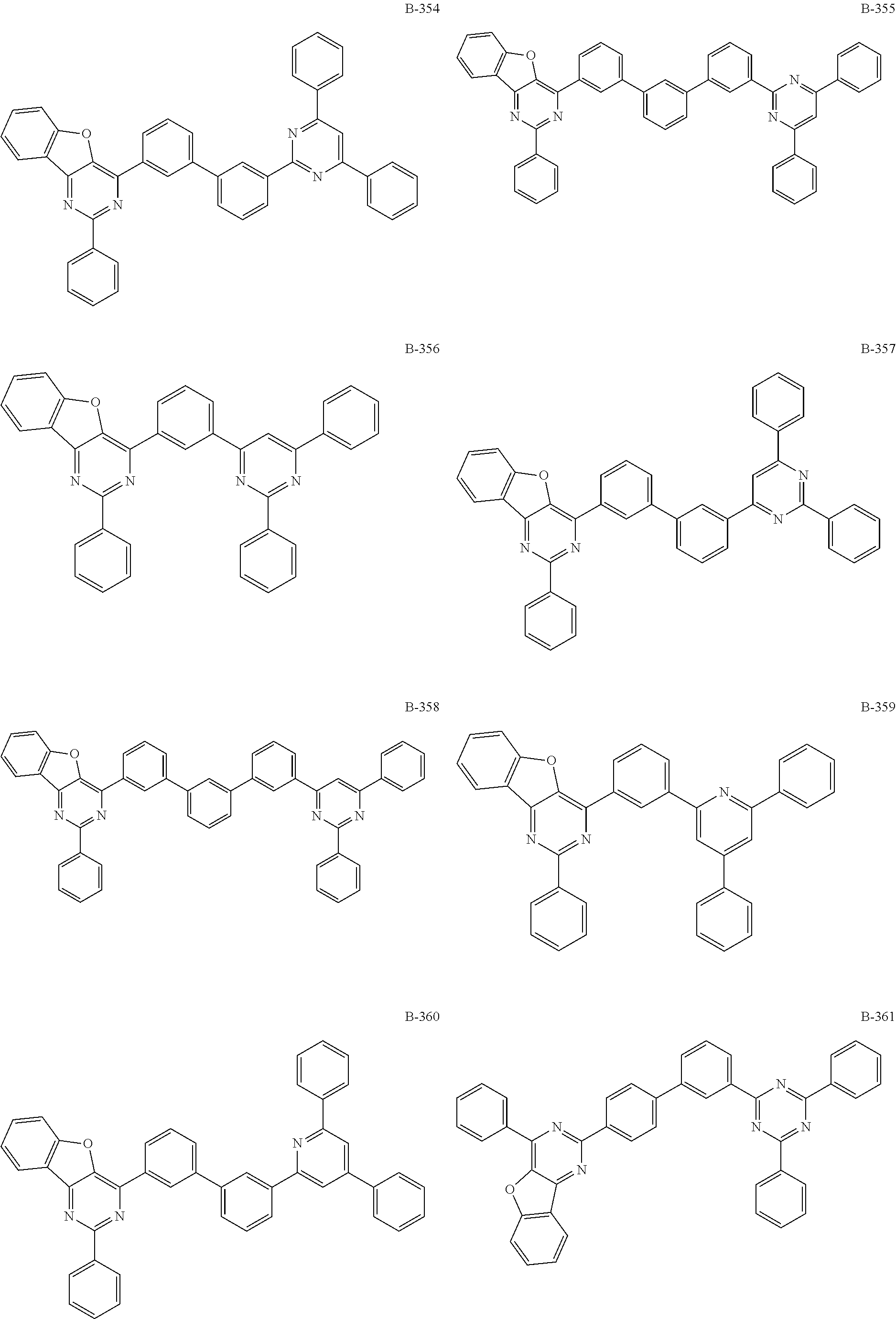

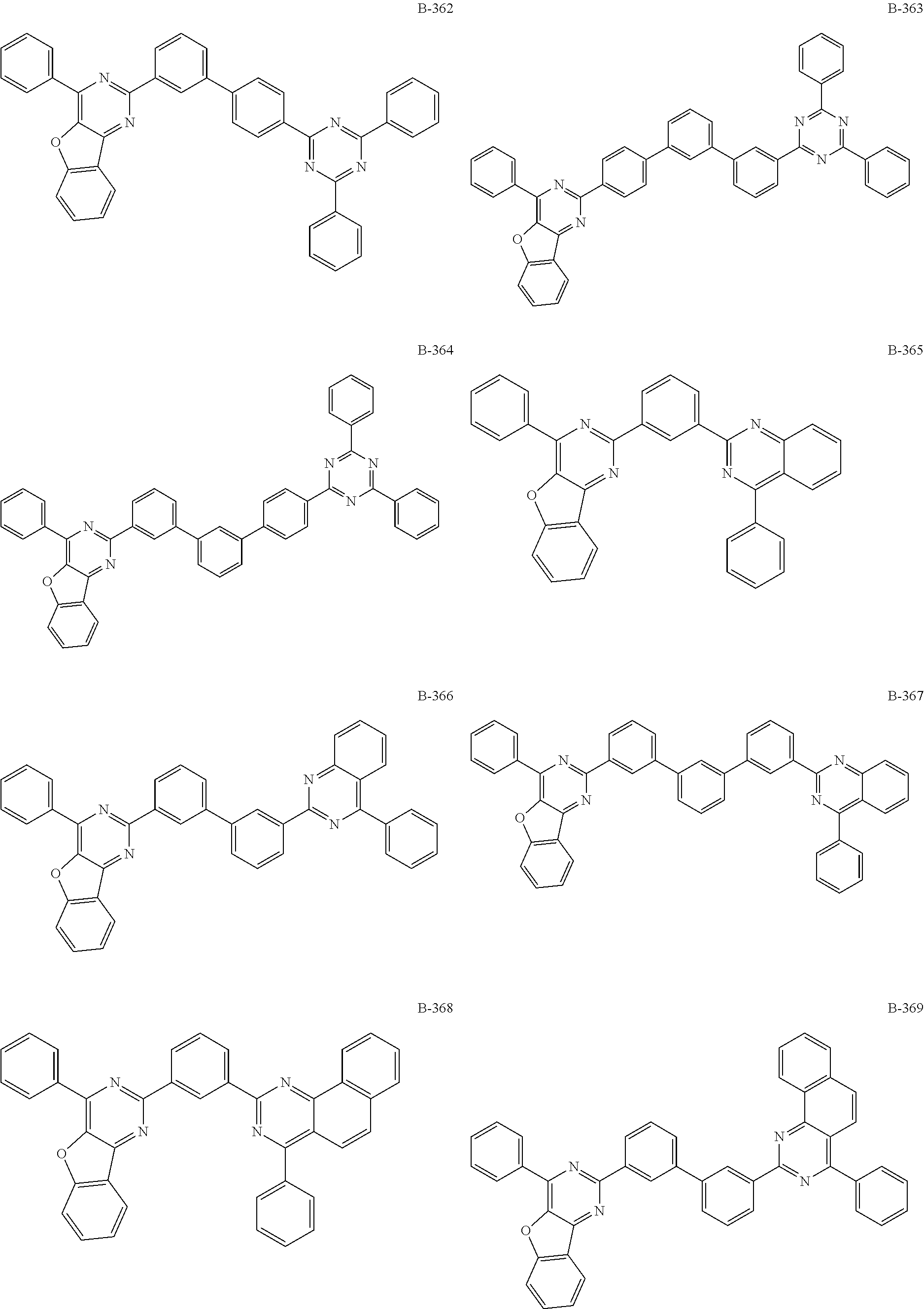

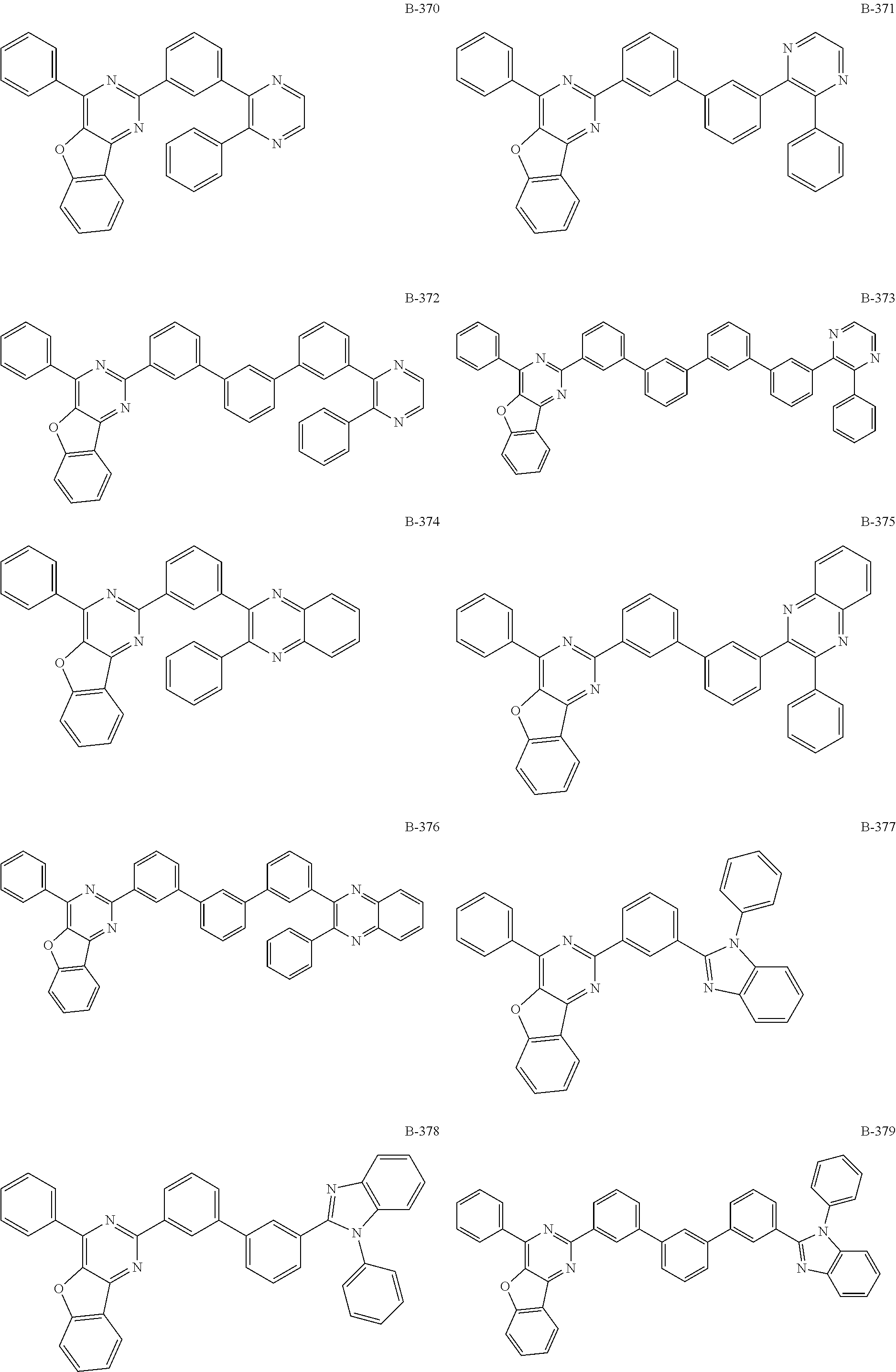

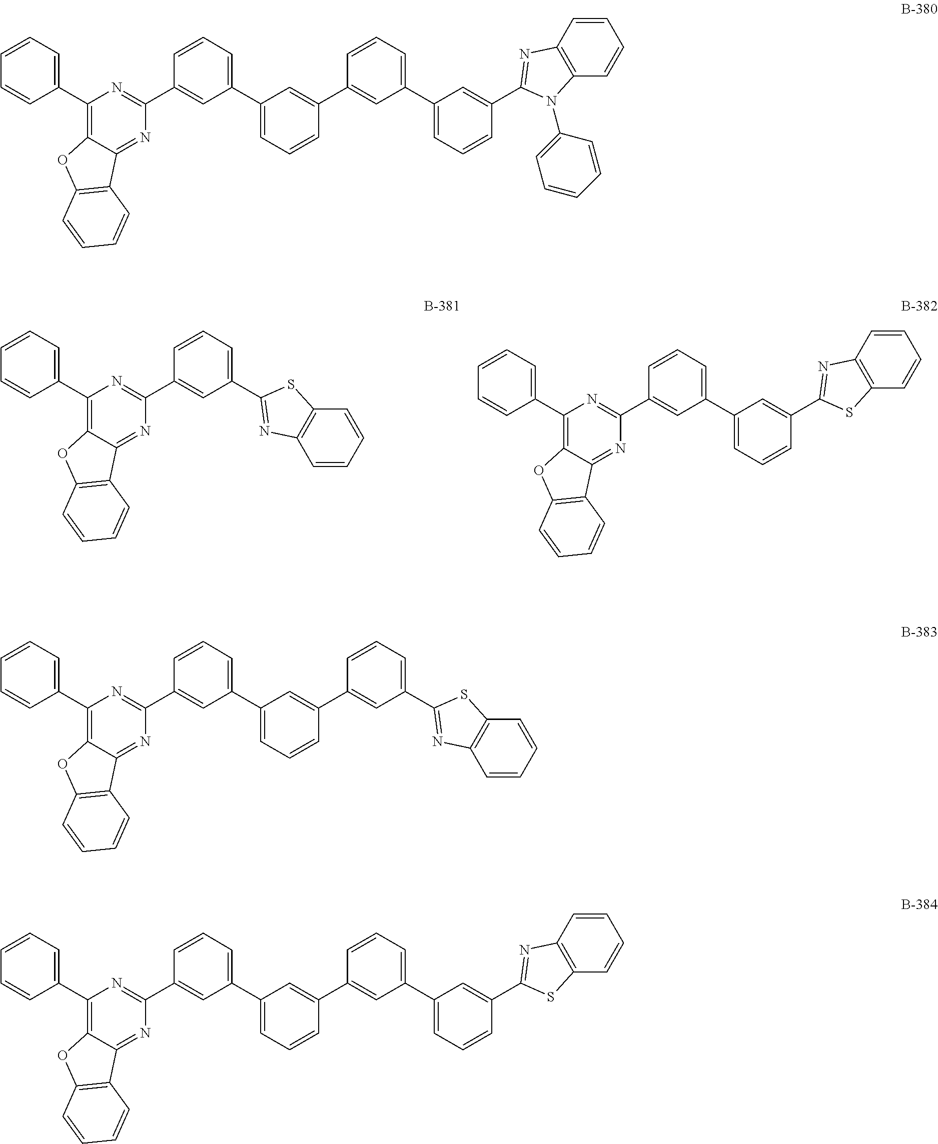 [SMILES: c1ccc(-c2nc(-c3cccc(-c4cccc(-c5cccc(-c6cccc(-c7nc8ccccc8n7-c7ccccc7)c6)c5)c4)c3)nc3c2oc2ccccc23)cc1 ; c1ccc(-c2nc(-c3cccc(-c4nc5ccccc5s4)c3)nc3c2oc2ccccc23)cc1 ; c1ccc(-c2nc(-c3cccc(-c4cccc(-c5nc6ccccc6s5)c4)c3)nc3c2oc2ccccc23)cc1 ; c1ccc(-c2nc(-c3cccc(-c4cccc(-c5cccc(-c6nc7ccccc7s6)c5)c4)c3)nc3c2oc2ccccc23)cc1 ; c1ccc(-c2nc(-c3cccc(-c4cccc(-c5cccc(-c6cccc(-c7nc8ccccc8s7)c6)c5)c4)c3)nc3c2oc2ccccc23)cc1]

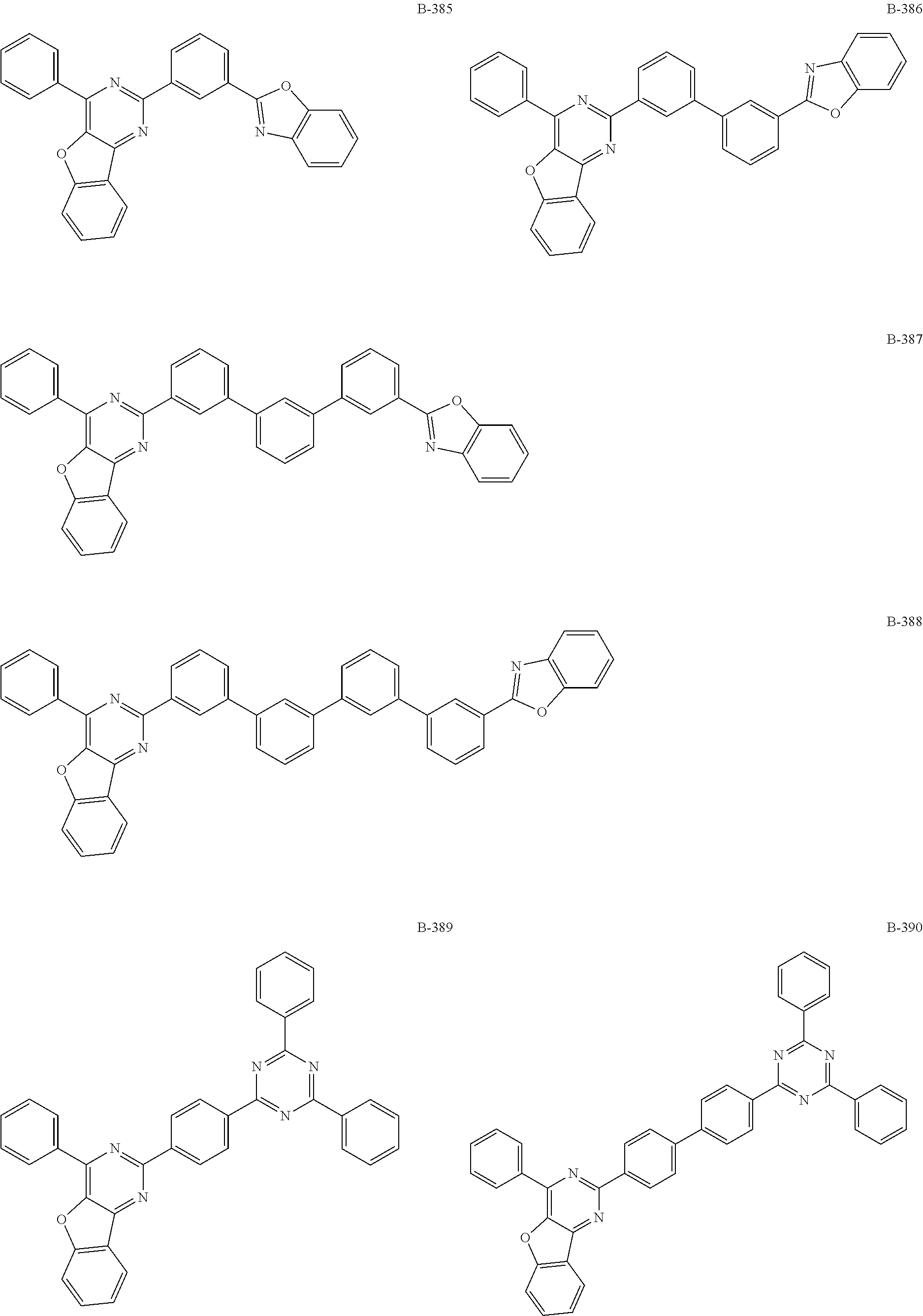 [SMILES: c1ccc(-c2nc(-c3cccc(-c4nc5ccccc5o4)c3)nc3c2oc2ccccc23)cc1 ; c1ccc(-c2nc(-c3cccc(-c4cccc(-c5nc6ccccc6o5)c4)c3)nc3c2oc2ccccc23)cc1 ; c1ccc(-c2nc(-c3cccc(-c4cccc(-c5cccc(-c6nc7ccccc7o6)c5)c4)c3)nc3c2oc2ccccc23)cc1 ; c1ccc(-c2nc(-c3cccc(-c4cccc(-c5cccc(-c6cccc(-c7nc8ccccc8o7)c6)c5)c4)c3)nc3c2oc2ccccc23)cc1 ; c1ccc(-c2nc(-c3ccccc3)nc(-c3ccc(-c4nc(-c5ccccc5)c5oc6ccccc6c5n4)cc3)n2)cc1 ; c1ccc(-c2nc(-c3ccccc3)nc(-c3ccc(-c4ccc(-c5nc(-c6ccccc6)c6oc7ccccc7c6n5)cc4)cc3)n2)cc1]

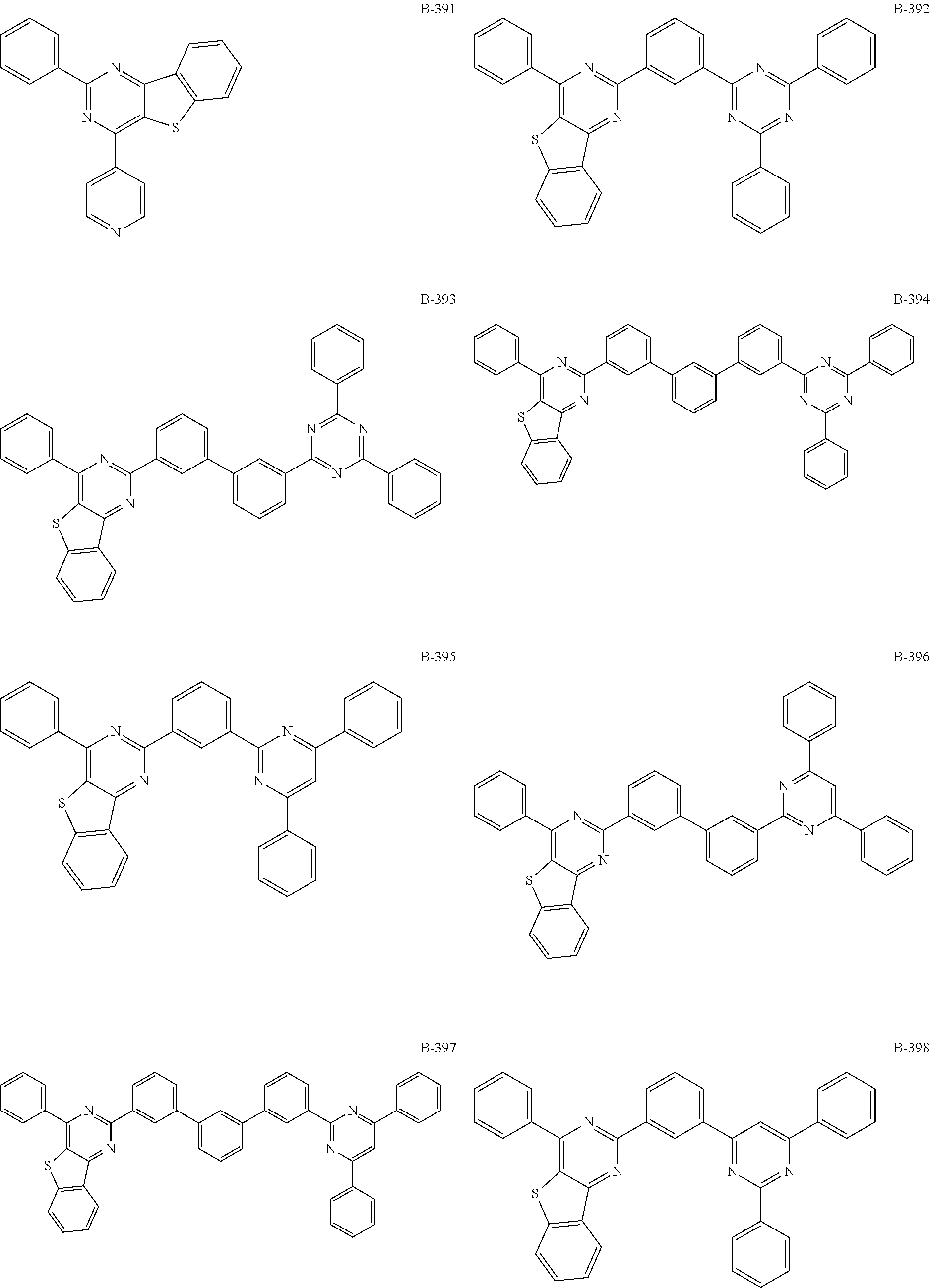

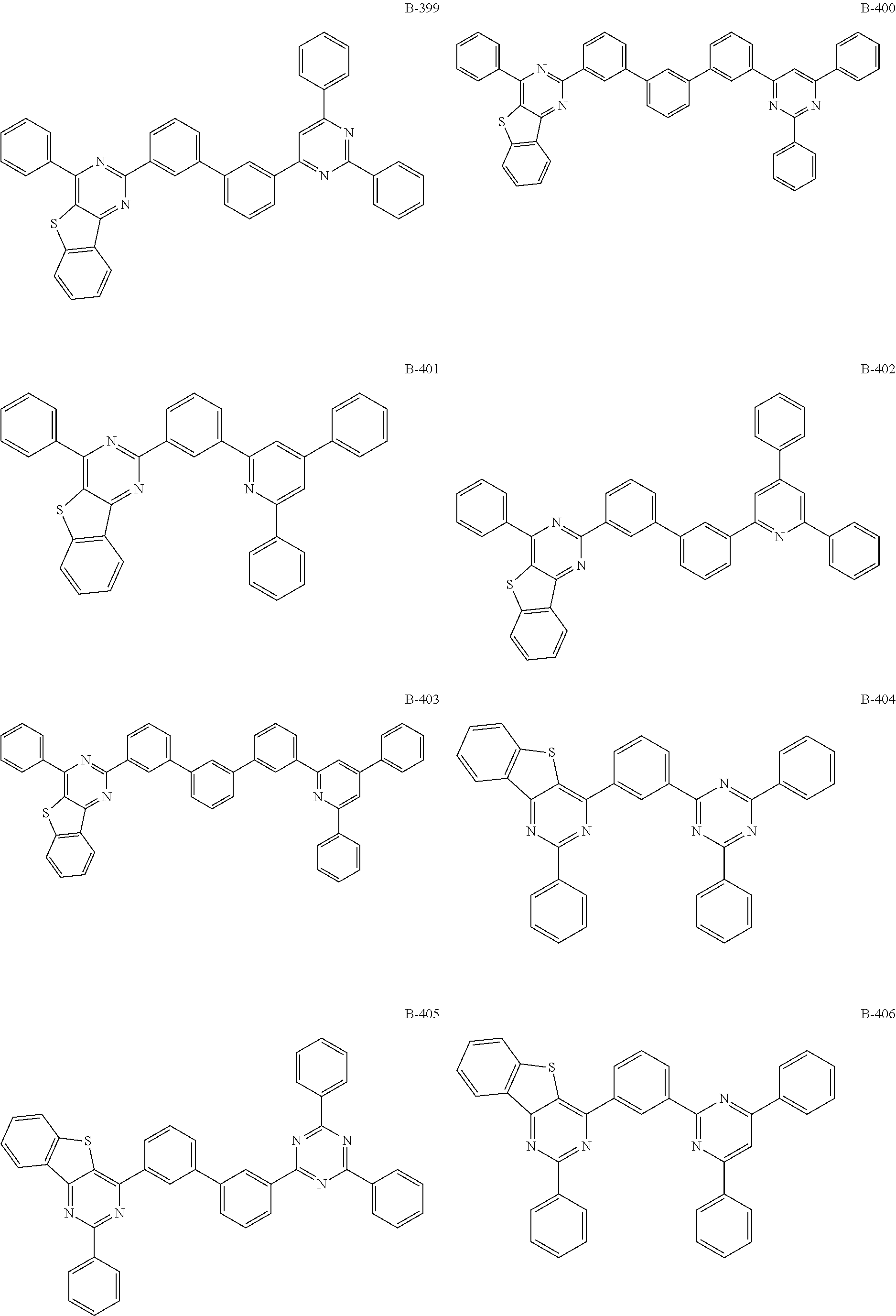

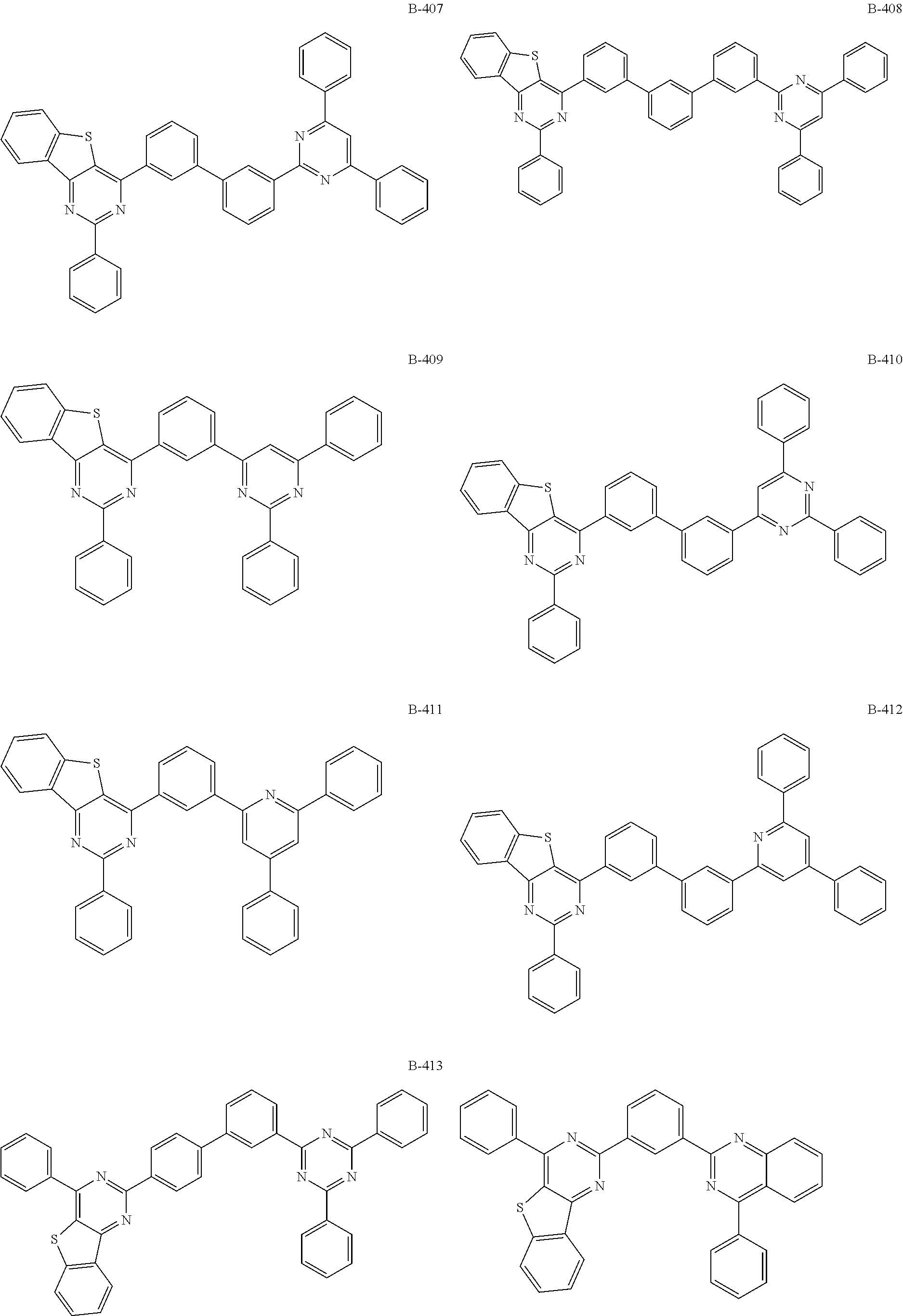

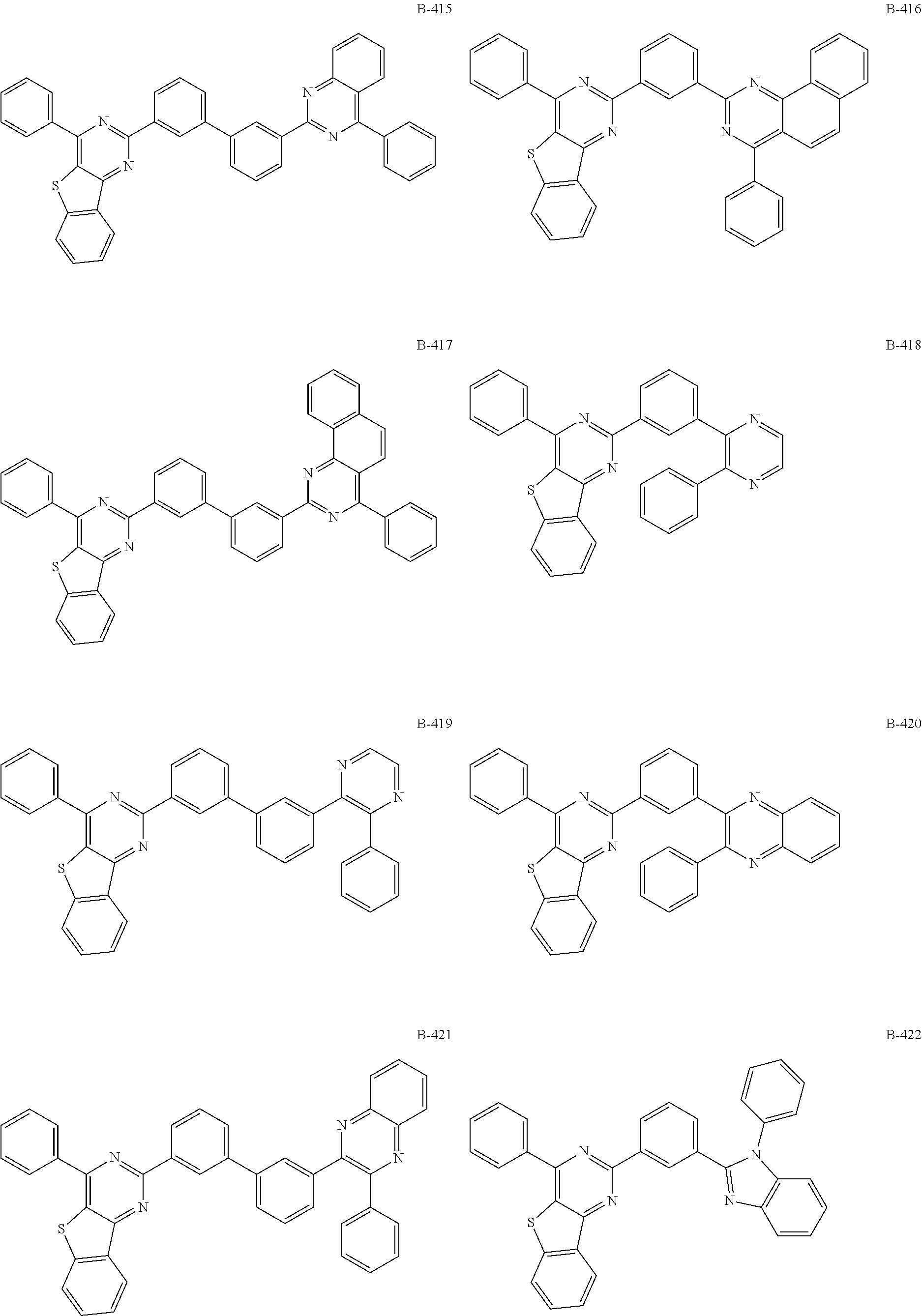

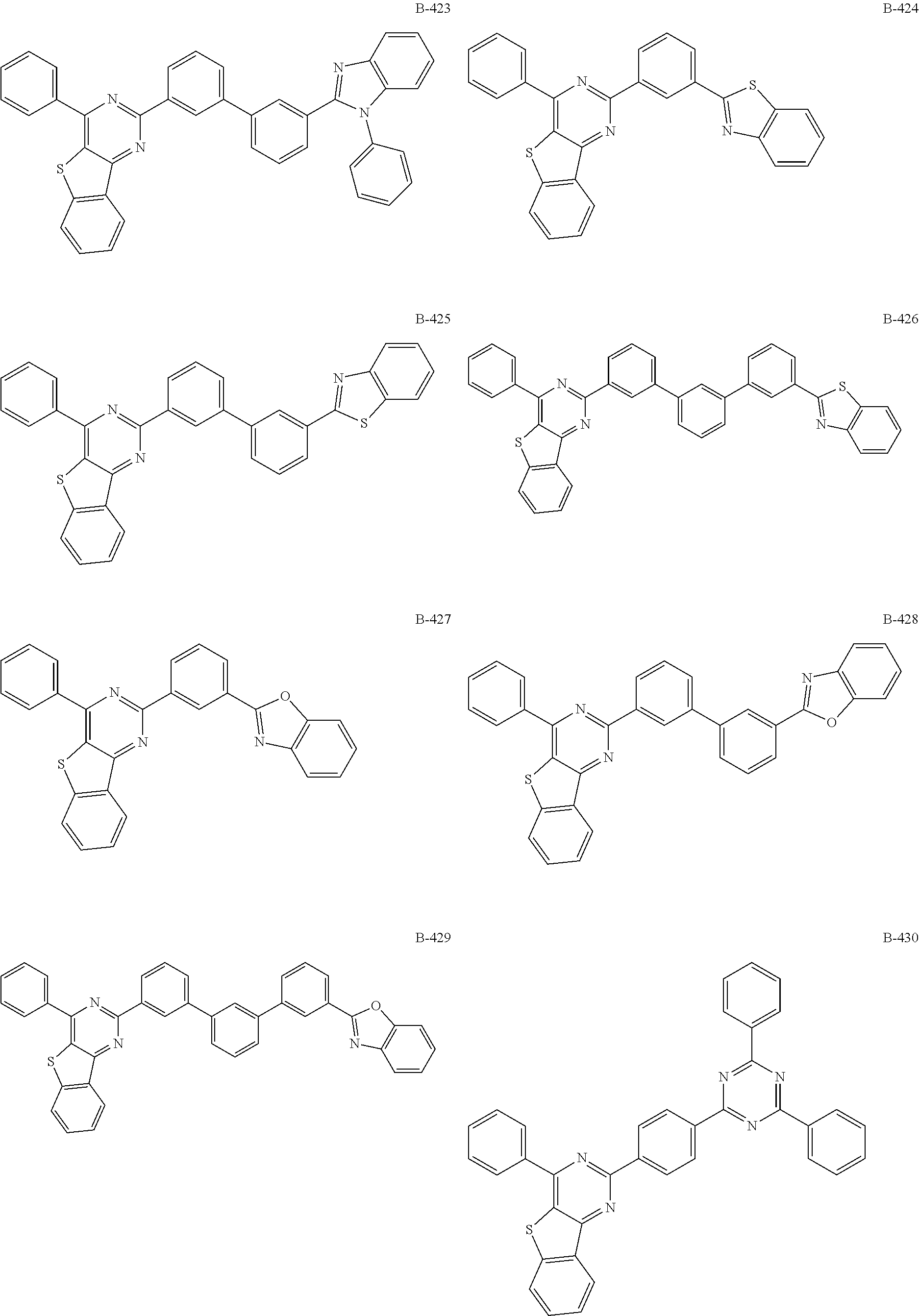

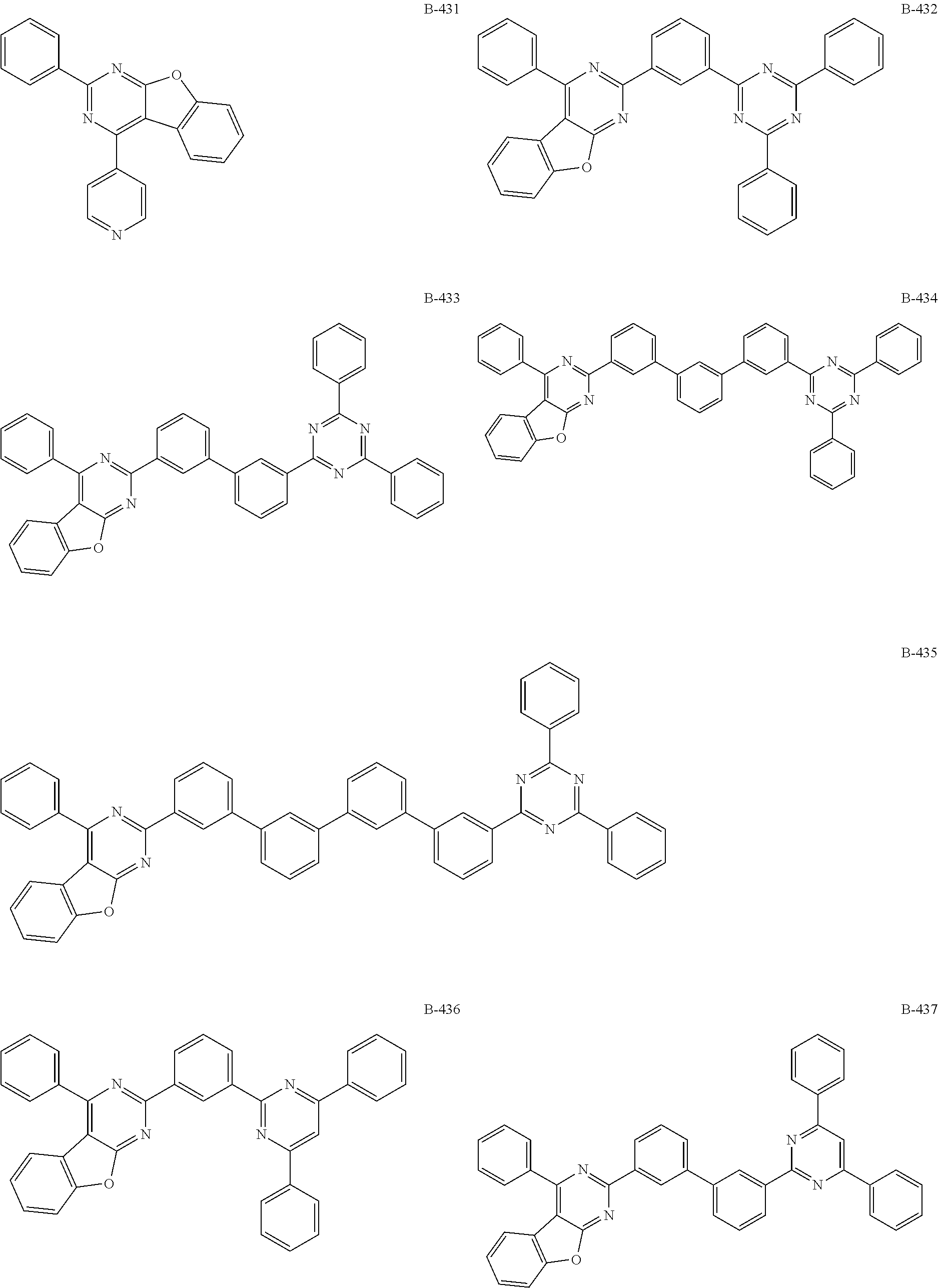

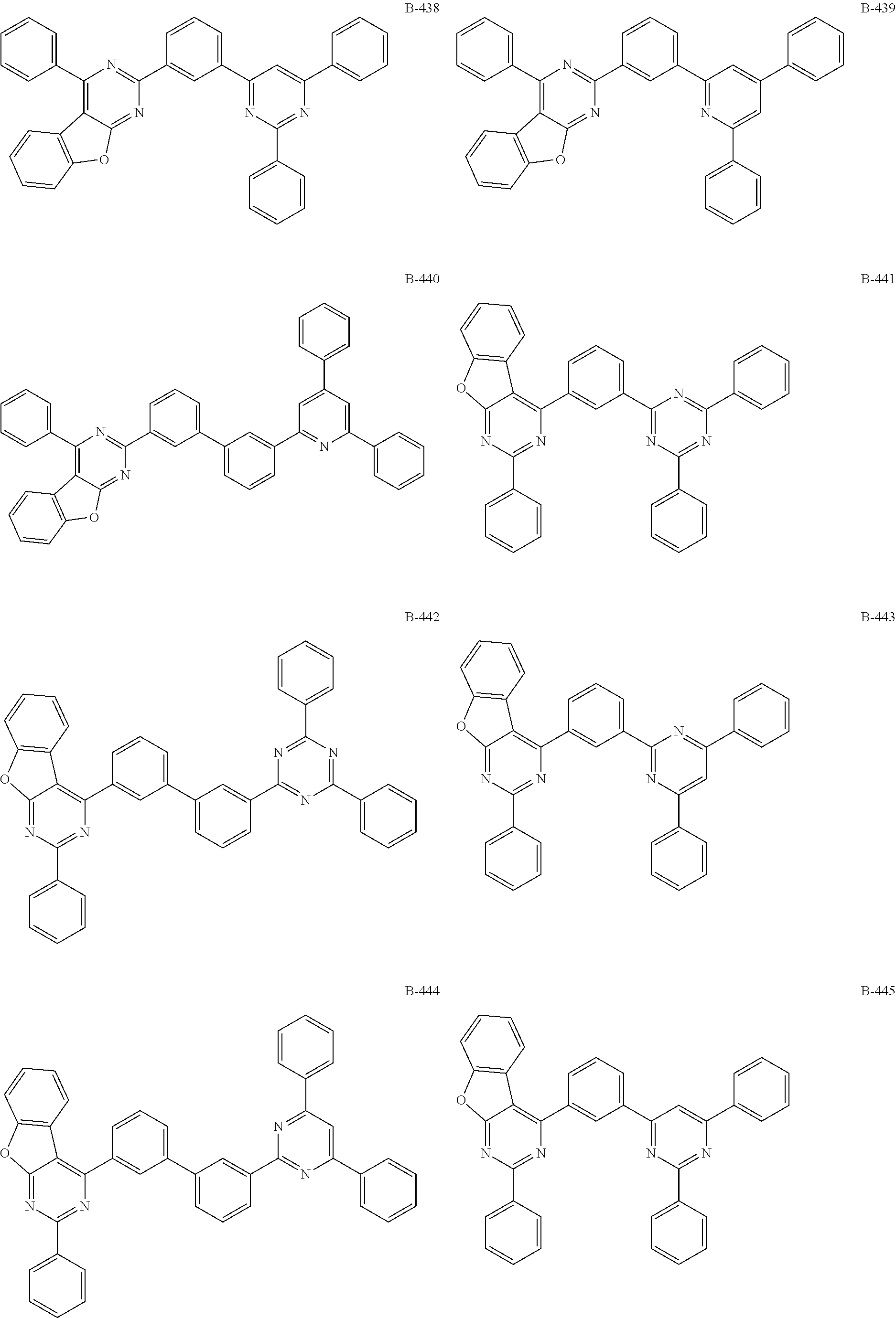

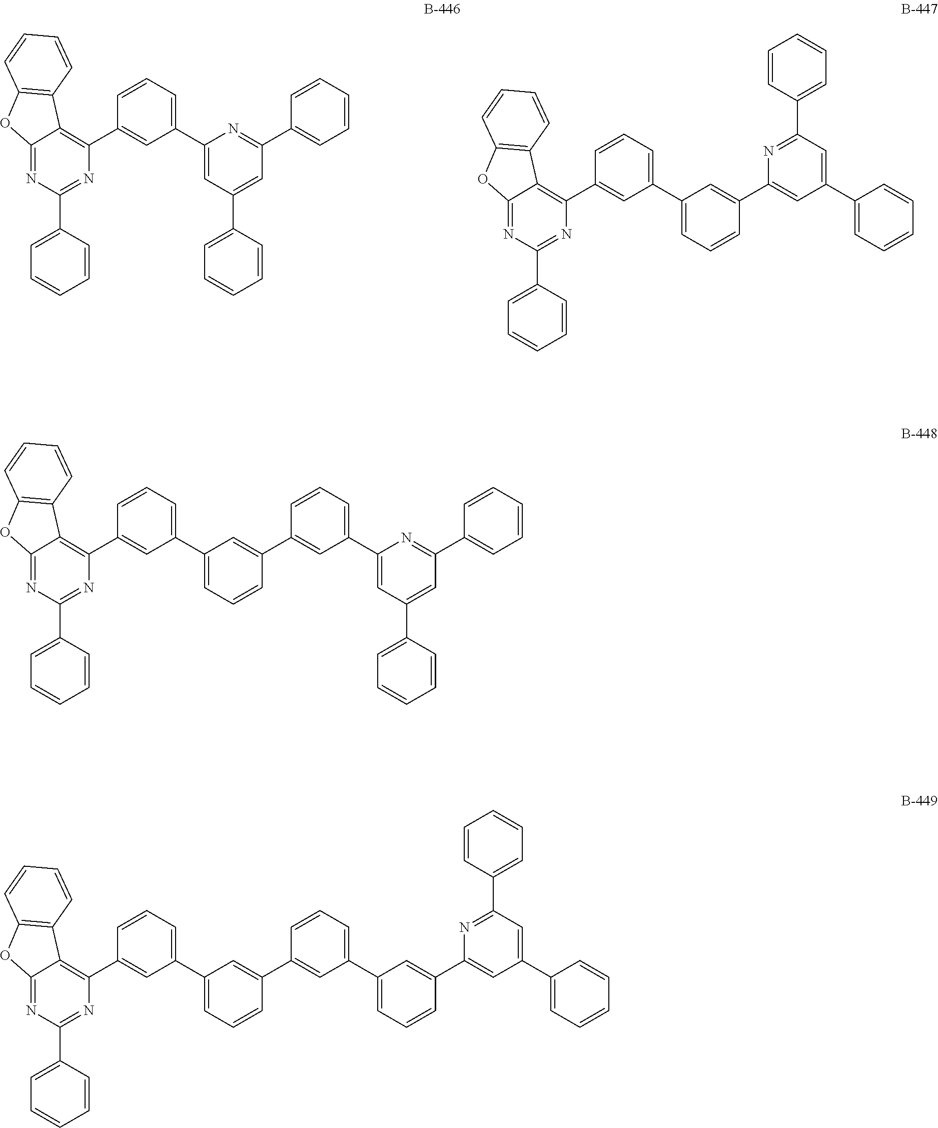 [SMILES: c1ccc(-c2cc(-c3ccccc3)nc(-c3cccc(-c4nc(-c5ccccc5)nc5oc6ccccc6c45)c3)c2)cc1 ; c1ccc(-c2cc(-c3ccccc3)nc(-c3cccc(-c4cccc(-c5nc(-c6ccccc6)nc6oc7ccccc7c56)c4)c3)c2)cc1 ; c1ccc(-c2cc(-c3ccccc3)nc(-c3cccc(-c4cccc(-c5cccc(-c6nc(-c7ccccc7)nc7oc8ccccc8c67)c5)c4)c3)c2)cc1 ; c1ccc(-c2cc(-c3ccccc3)nc(-c3cccc(-c4cccc(-c5cccc(-c6cccc(-c7nc(-c8ccccc8)nc8oc9ccccc9c78)c6)c5)c4)c3)c2)cc1]

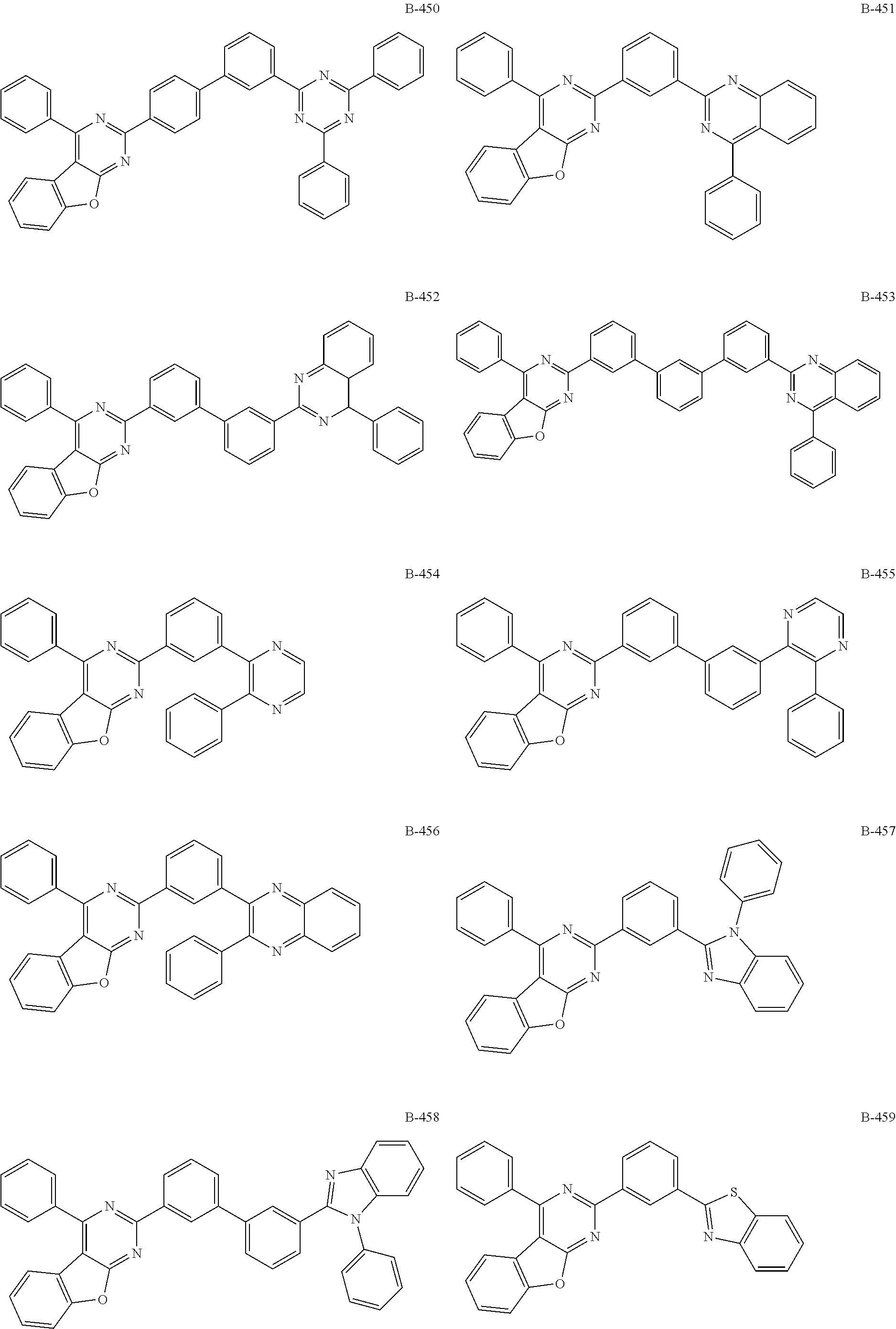

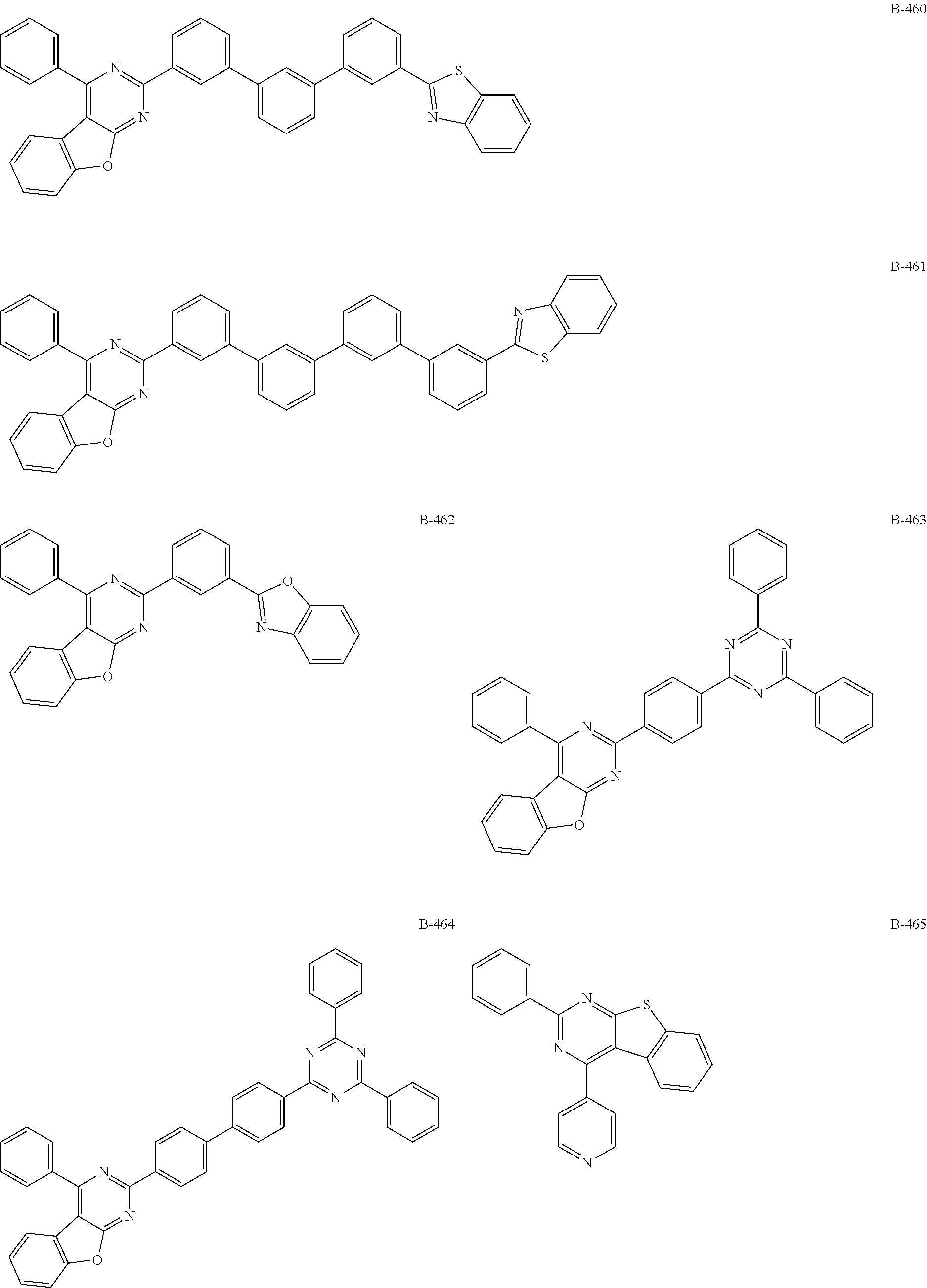 [SMILES: c1ccc(-c2nc(-c3cccc(-c4cccc(-c5cccc(-c6nc7ccccc7s6)c5)c4)c3)nc3oc4ccccc4c23)cc1 ; c1ccc(-c2nc(-c3cccc(-c4cccc(-c5cccc(-c6cccc(-c7nc8ccccc8s7)c6)c5)c4)c3)nc3oc4ccccc4c23)cc1 ; c1ccc(-c2nc(-c3cccc(-c4nc5ccccc5o4)c3)nc3oc4ccccc4c23)cc1 ; c1ccc(-c2nc(-c3ccccc3)nc(-c3ccc(-c4nc(-c5ccccc5)c5c(n4)oc4ccccc45)cc3)n2)cc1 ; c1ccc(-c2nc(-c3ccccc3)nc(-c3ccc(-c4ccc(-c5nc(-c6ccccc6)c6c(n5)oc5ccccc56)cc4)cc3)n2)cc1 ; c1ccc(-c2nc(-c3ccncc3)c3c(n2)sc2ccccc23)cc1]

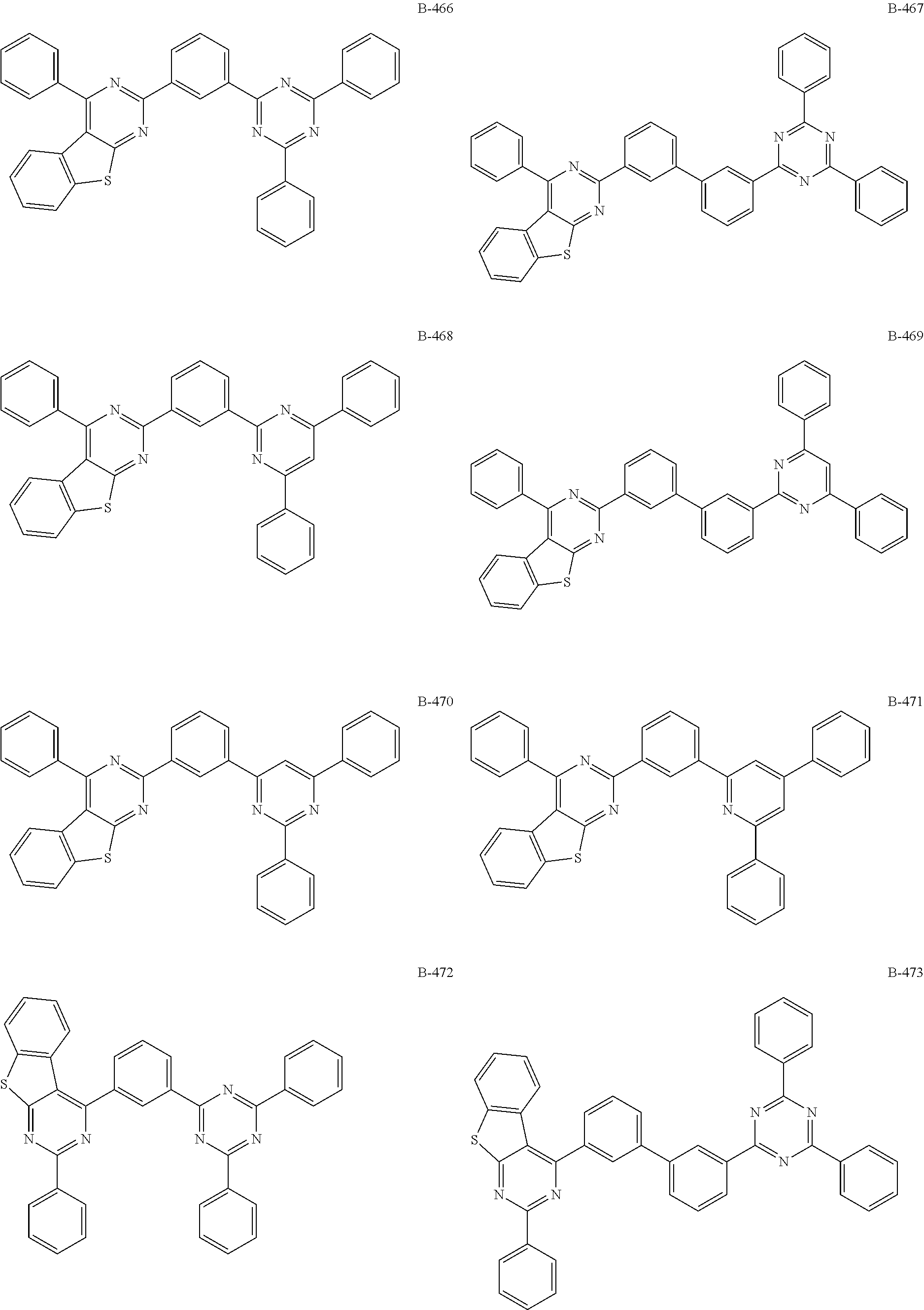

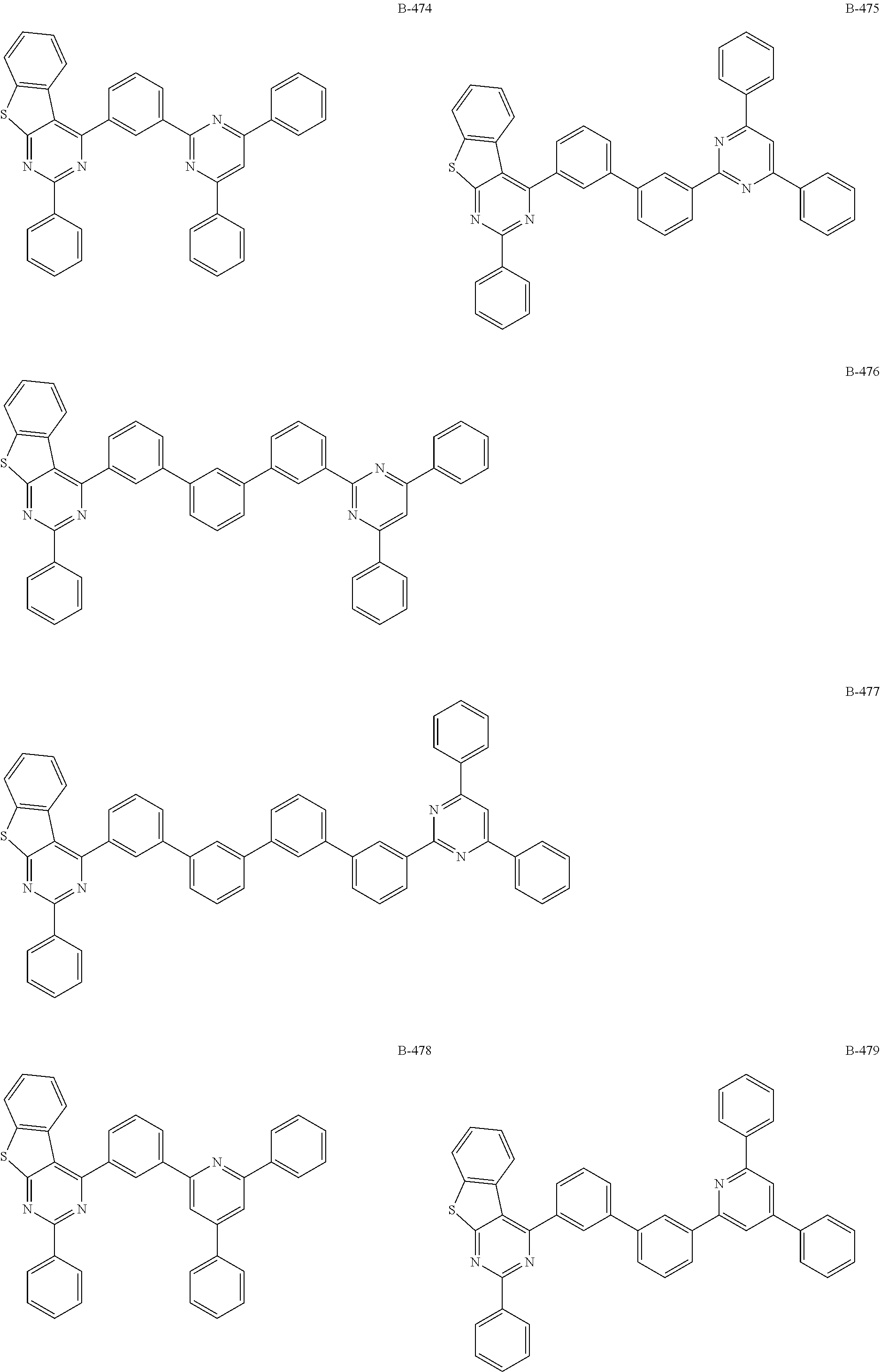

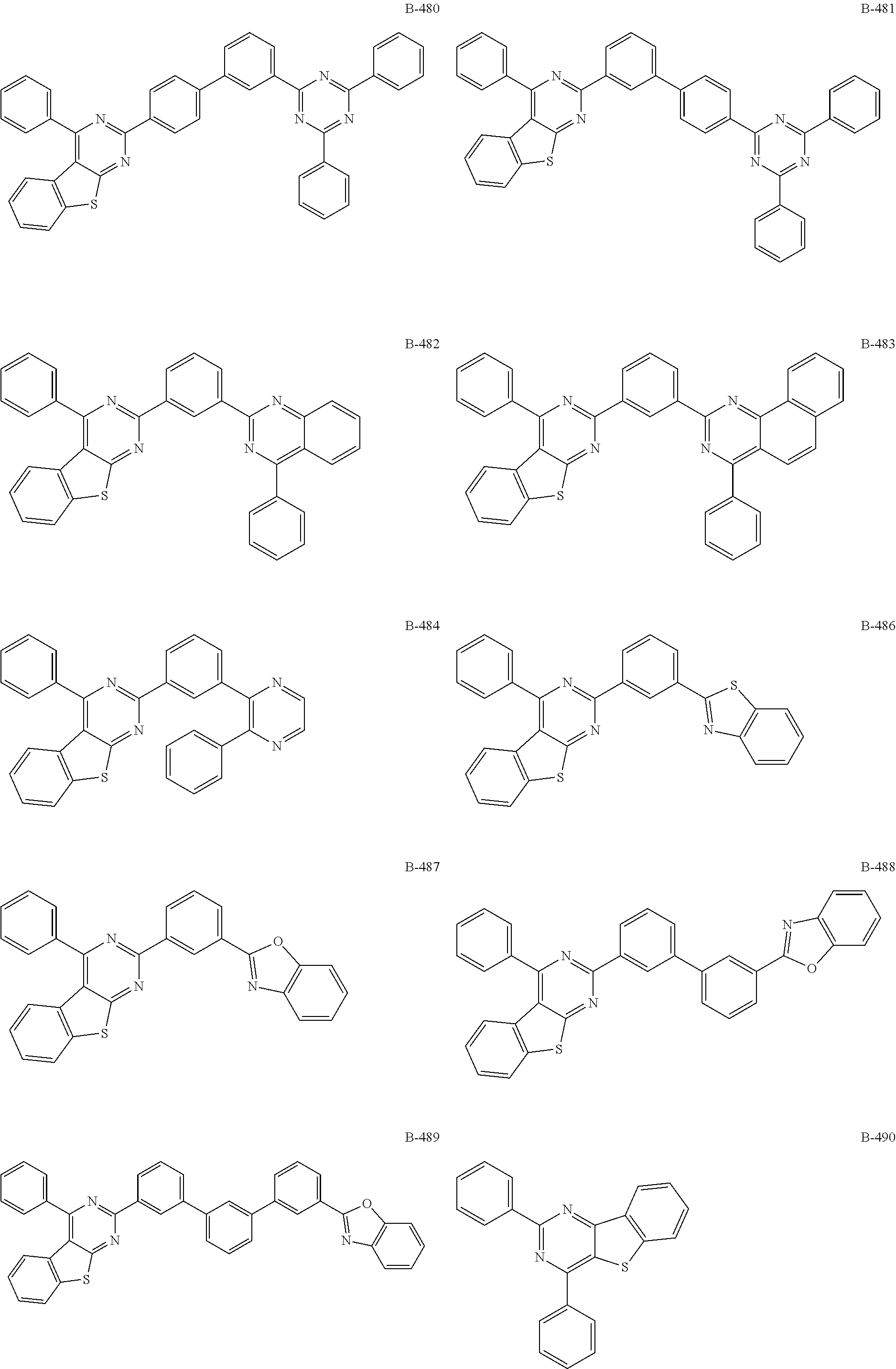

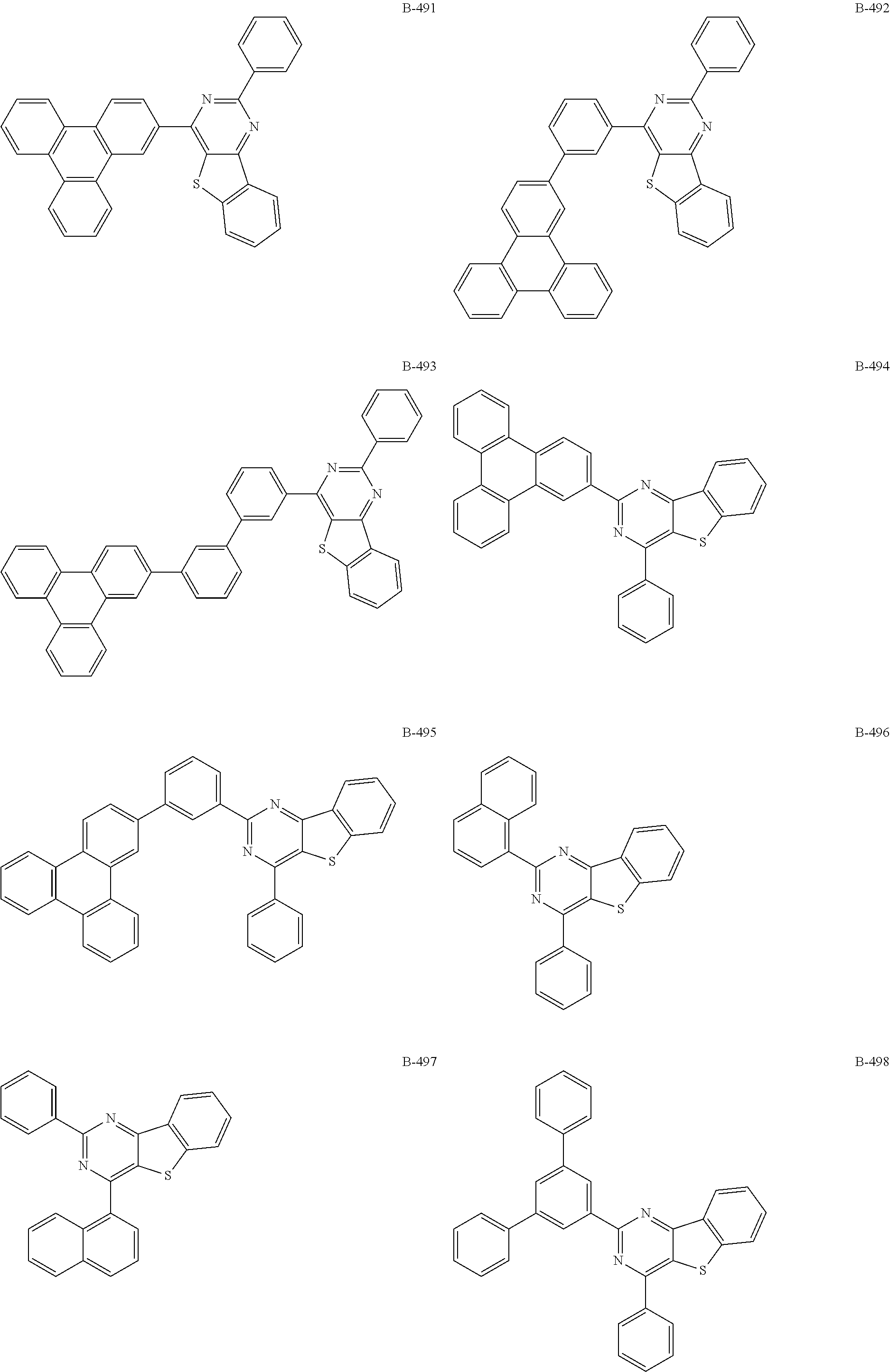

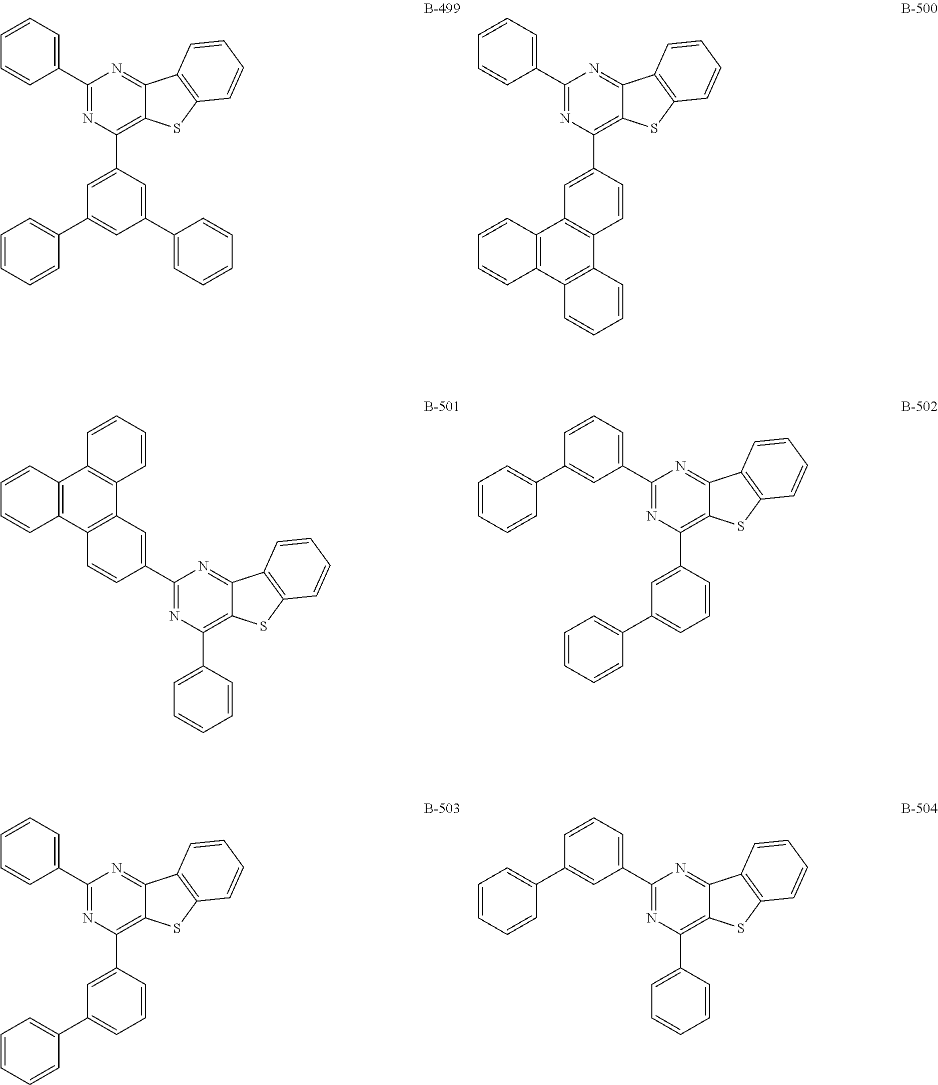 [SMILES: c1ccc(-c2cc(-c3ccccc3)cc(-c3nc(-c4ccccc4)nc4c3sc3ccccc34)c2)cc1 ; c1ccc(-c2nc(-c3ccc4c5ccccc5c5ccccc5c4c3)c3sc4ccccc4c3n2)cc1 ; c1ccc(-c2nc(-c3ccc4c5ccccc5c5ccccc5c4c3)nc3c2sc2ccccc23)cc1 ; c1ccc(-c2cccc(-c3nc(-c4cccc(-c5ccccc5)c4)c4sc5ccccc5c4n3)c2)cc1 ; c1ccc(-c2cccc(-c3nc(-c4ccccc4)nc4c3sc3ccccc34)c2)cc1 ; c1ccc(-c2cccc(-c3nc(-c4ccccc4)c4sc5ccccc5c4n3)c2)cc1]

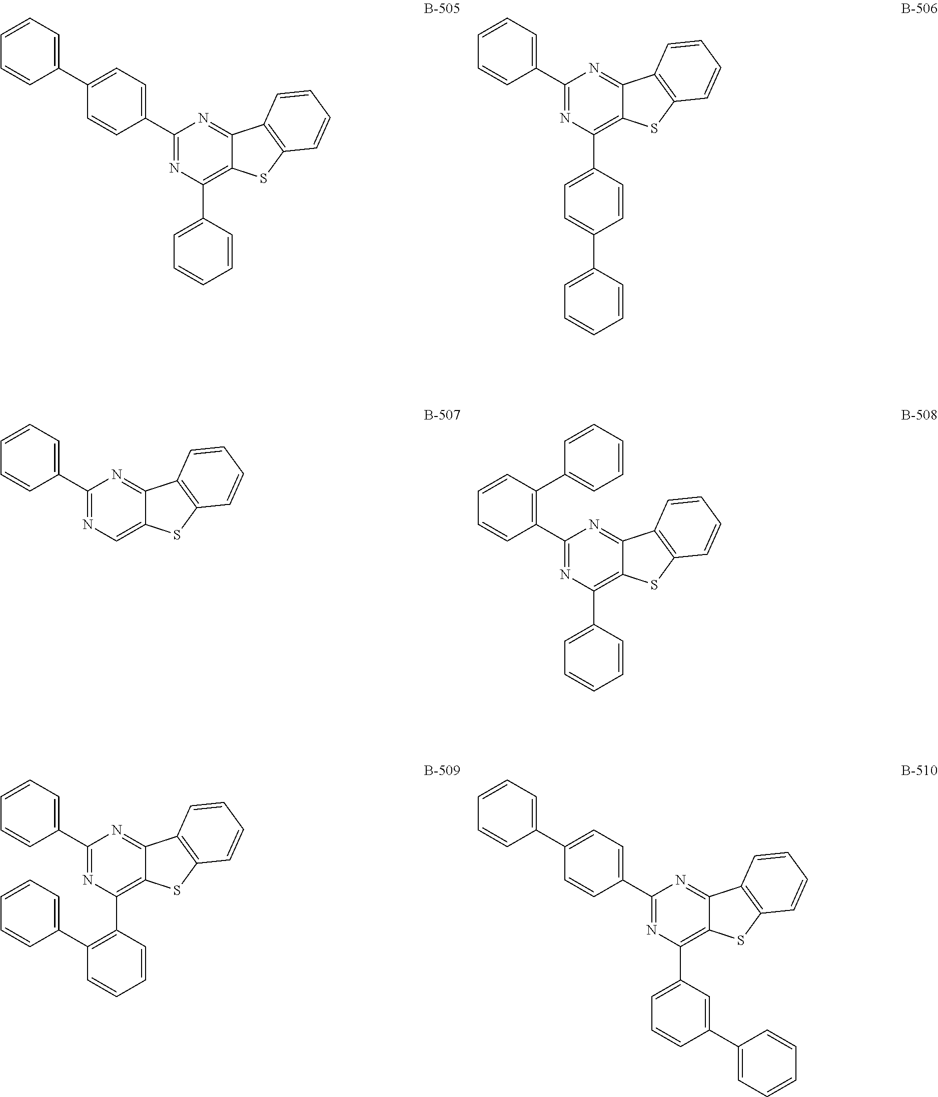 [SMILES: c1ccc(-c2ccc(-c3nc(-c4ccccc4)c4sc5ccccc5c4n3)cc2)cc1 ; c1ccc(-c2ccc(-c3nc(-c4ccccc4)nc4c3sc3ccccc34)cc2)cc1 ; c1ccc(-c2ncc3sc4ccccc4c3n2)cc1 ; c1ccc(-c2ccccc2-c2nc(-c3ccccc3)c3sc4ccccc4c3n2)cc1 ; c1ccc(-c2nc(-c3ccccc3-c3ccccc3)c3sc4ccccc4c3n2)cc1 ; c1ccc(-c2ccc(-c3nc(-c4cccc(-c5ccccc5)c4)c4sc5ccccc5c4n3)cc2)cc1]

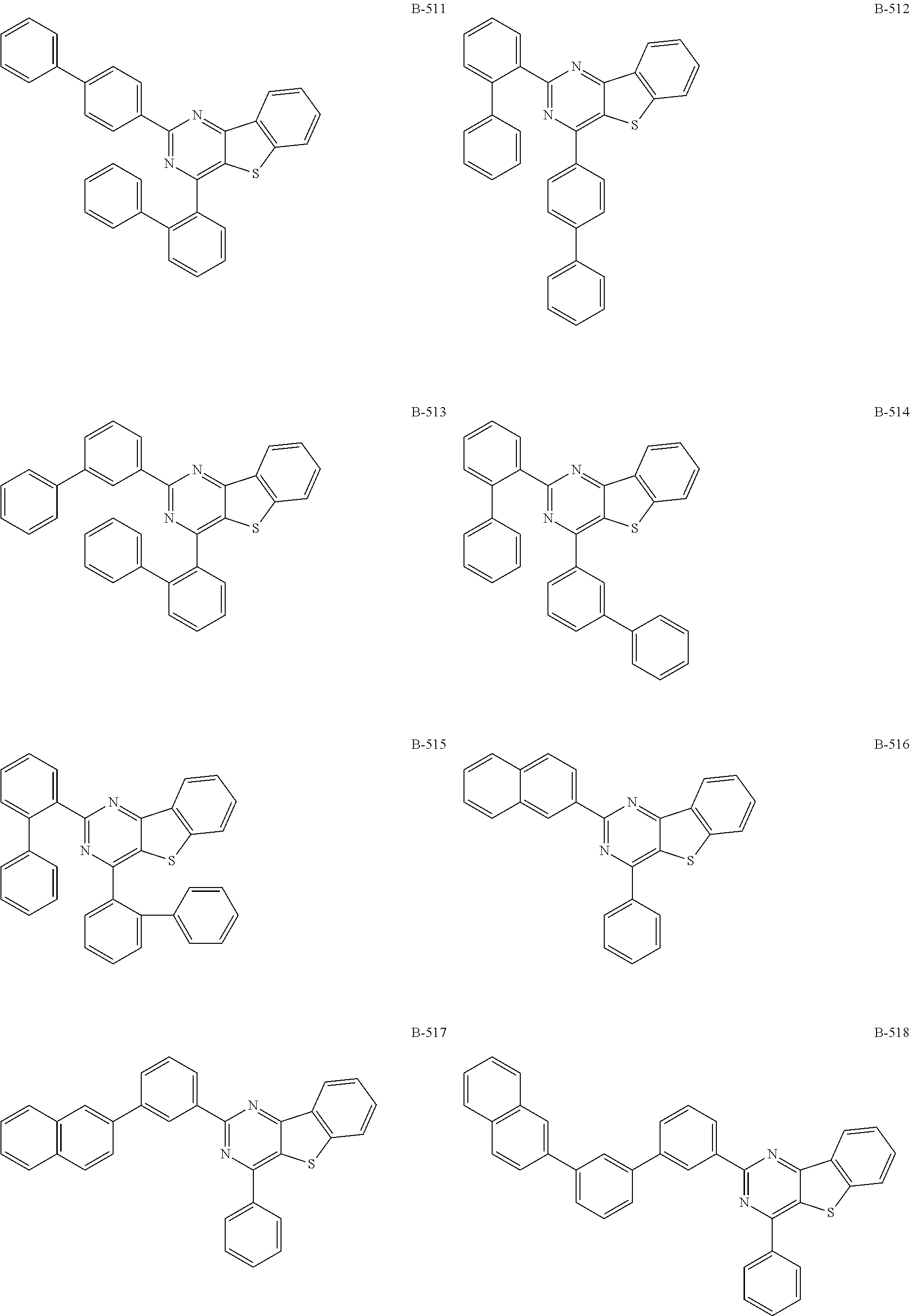

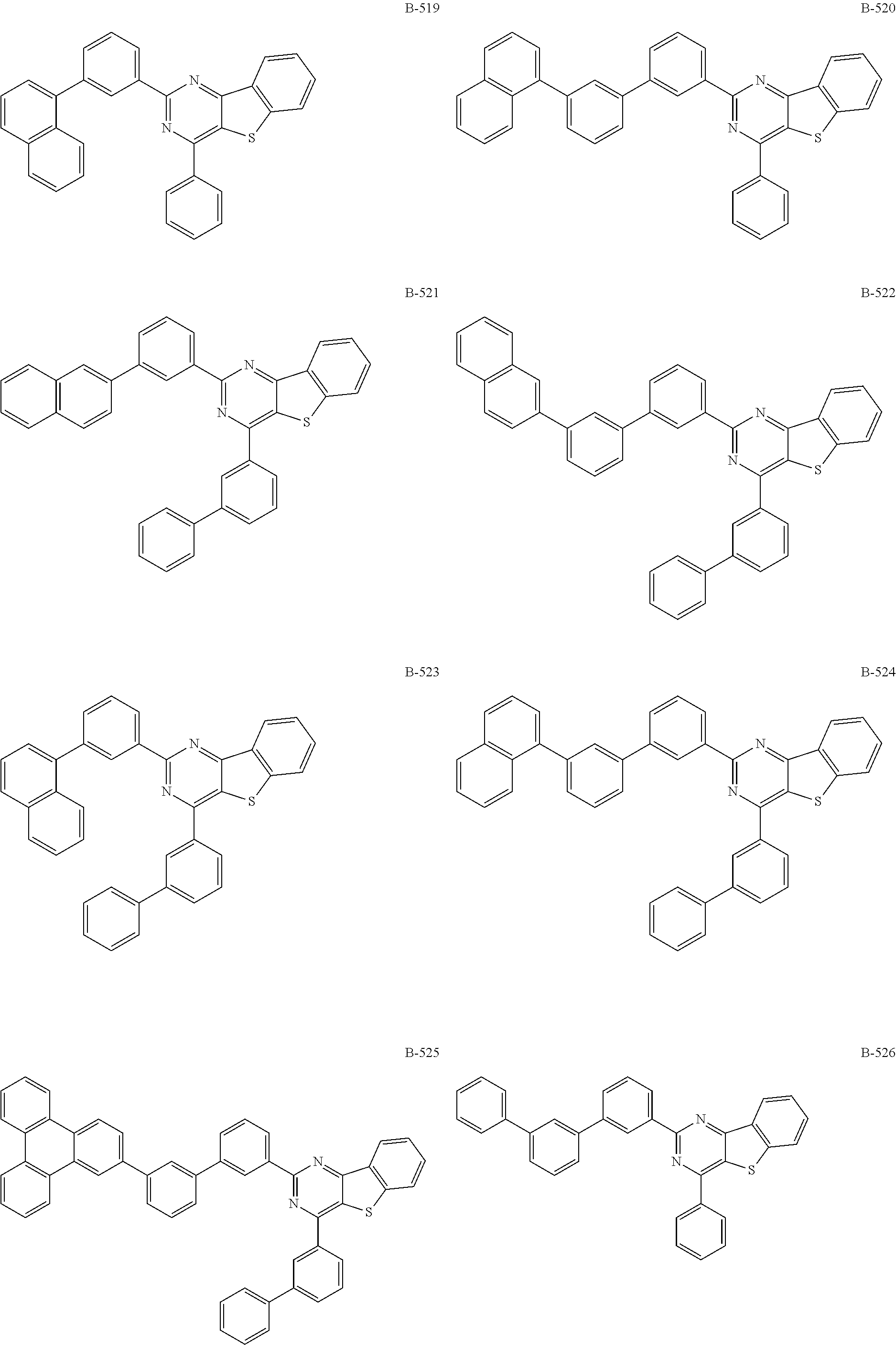

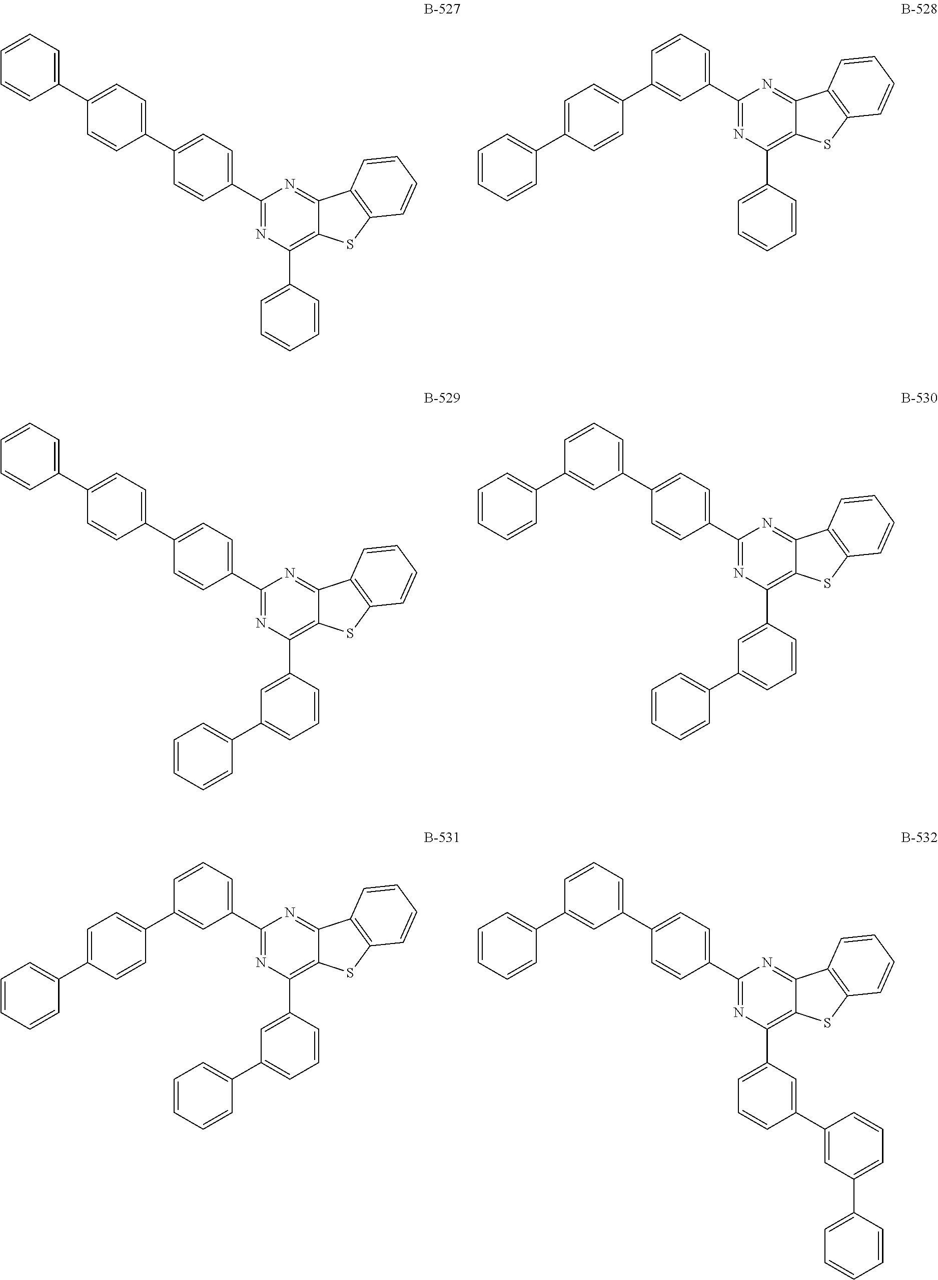 [SMILES: c1ccc(-c2ccc(-c3ccc(-c4nc(-c5ccccc5)c5sc6ccccc6c5n4)cc3)cc2)cc1 ; c1ccc(-c2ccc(-c3cccc(-c4nc(-c5ccccc5)c5sc6ccccc6c5n4)c3)cc2)cc1 ; c1ccc(-c2ccc(-c3ccc(-c4nc(-c5cccc(-c6ccccc6)c5)c5sc6ccccc6c5n4)cc3)cc2)cc1 ; c1ccc(-c2cccc(-c3ccc(-c4nc(-c5cccc(-c6ccccc6)c5)c5sc6ccccc6c5n4)cc3)c2)cc1 ; c1ccc(-c2ccc(-c3cccc(-c4nc(-c5cccc(-c6ccccc6)c5)c5sc6ccccc6c5n4)c3)cc2)cc1 ; c1ccc(-c2cccc(-c3ccc(-c4nc(-c5cccc(-c6cccc(-c7ccccc7)c6)c5)c5sc6ccccc6c5n4)cc3)c2)cc1]

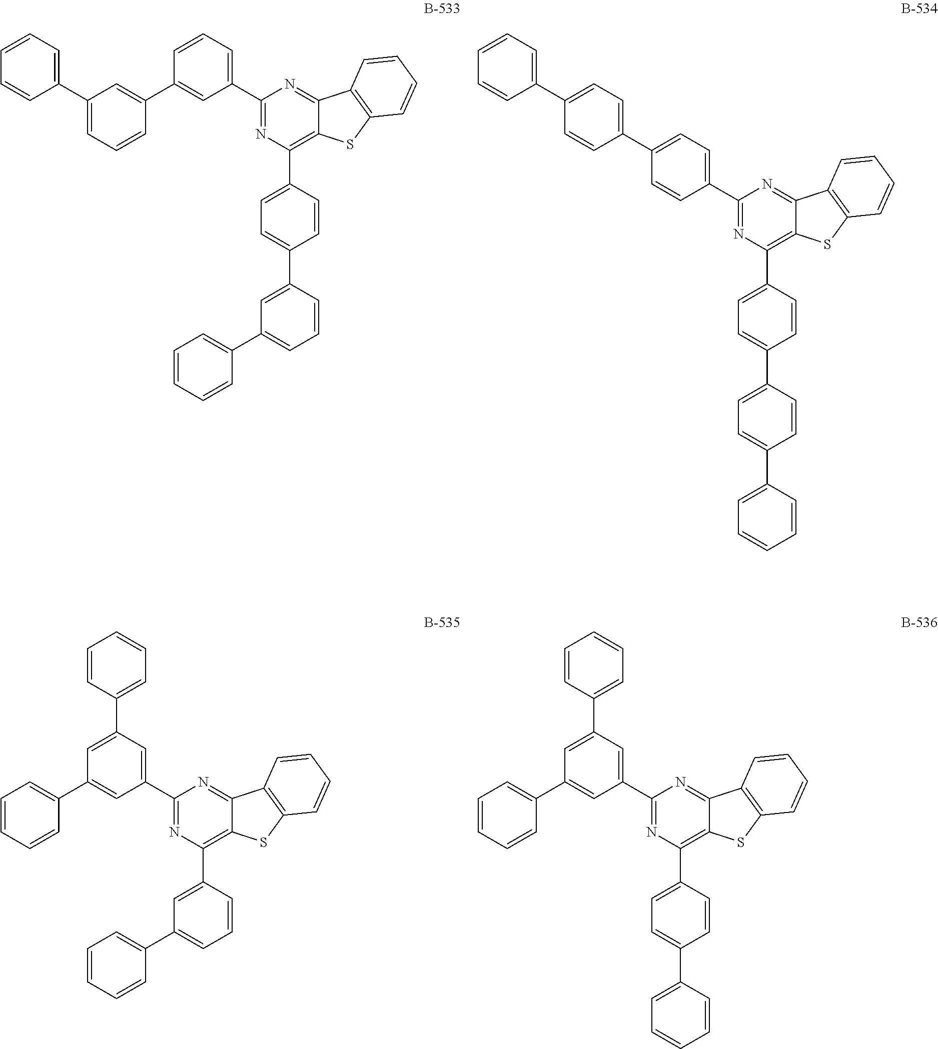 [SMILES: c1ccc(-c2cccc(-c3ccc(-c4nc(-c5cccc(-c6cccc(-c7ccccc7)c6)c5)nc5c4sc4ccccc45)cc3)c2)cc1 ; c1ccc(-c2ccc(-c3ccc(-c4nc(-c5ccc(-c6ccc(-c7ccccc7)cc6)cc5)c5sc6ccccc6c5n4)cc3)cc2)cc1 ; c1ccc(-c2cc(-c3ccccc3)cc(-c3nc(-c4cccc(-c5ccccc5)c4)c4sc5ccccc5c4n3)c2)cc1 ; c1ccc(-c2ccc(-c3nc(-c4cc(-c5ccccc5)cc(-c5ccccc5)c4)nc4c3sc3ccccc34)cc2)cc1]

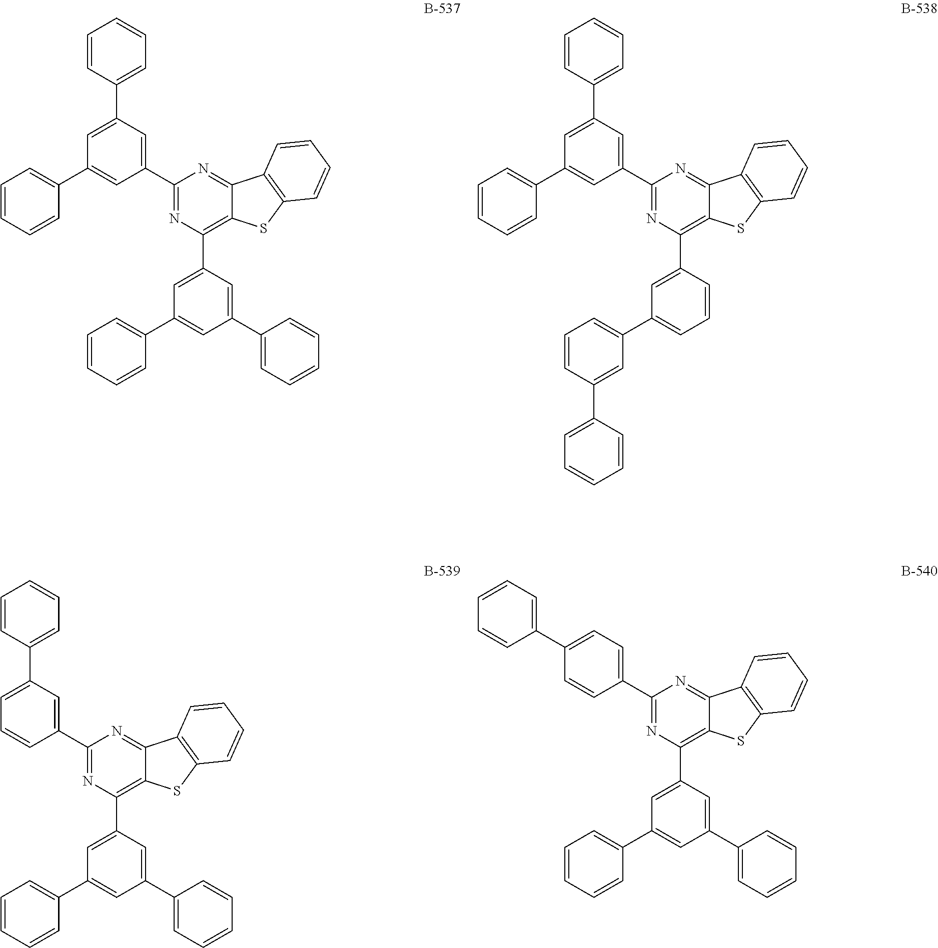 [SMILES: c1ccc(-c2cc(-c3ccccc3)cc(-c3nc(-c4cc(-c5ccccc5)cc(-c5ccccc5)c4)c4sc5ccccc5c4n3)c2)cc1 ; c1ccc(-c2cccc(-c3cccc(-c4nc(-c5cc(-c6ccccc6)cc(-c6ccccc6)c5)nc5c4sc4ccccc45)c3)c2)cc1 ; c1ccc(-c2cccc(-c3nc(-c4cc(-c5ccccc5)cc(-c5ccccc5)c4)c4sc5ccccc5c4n3)c2)cc1 ; c1ccc(-c2ccc(-c3nc(-c4cc(-c5ccccc5)cc(-c5ccccc5)c4)c4sc5ccccc5c4n3)cc2)cc1]

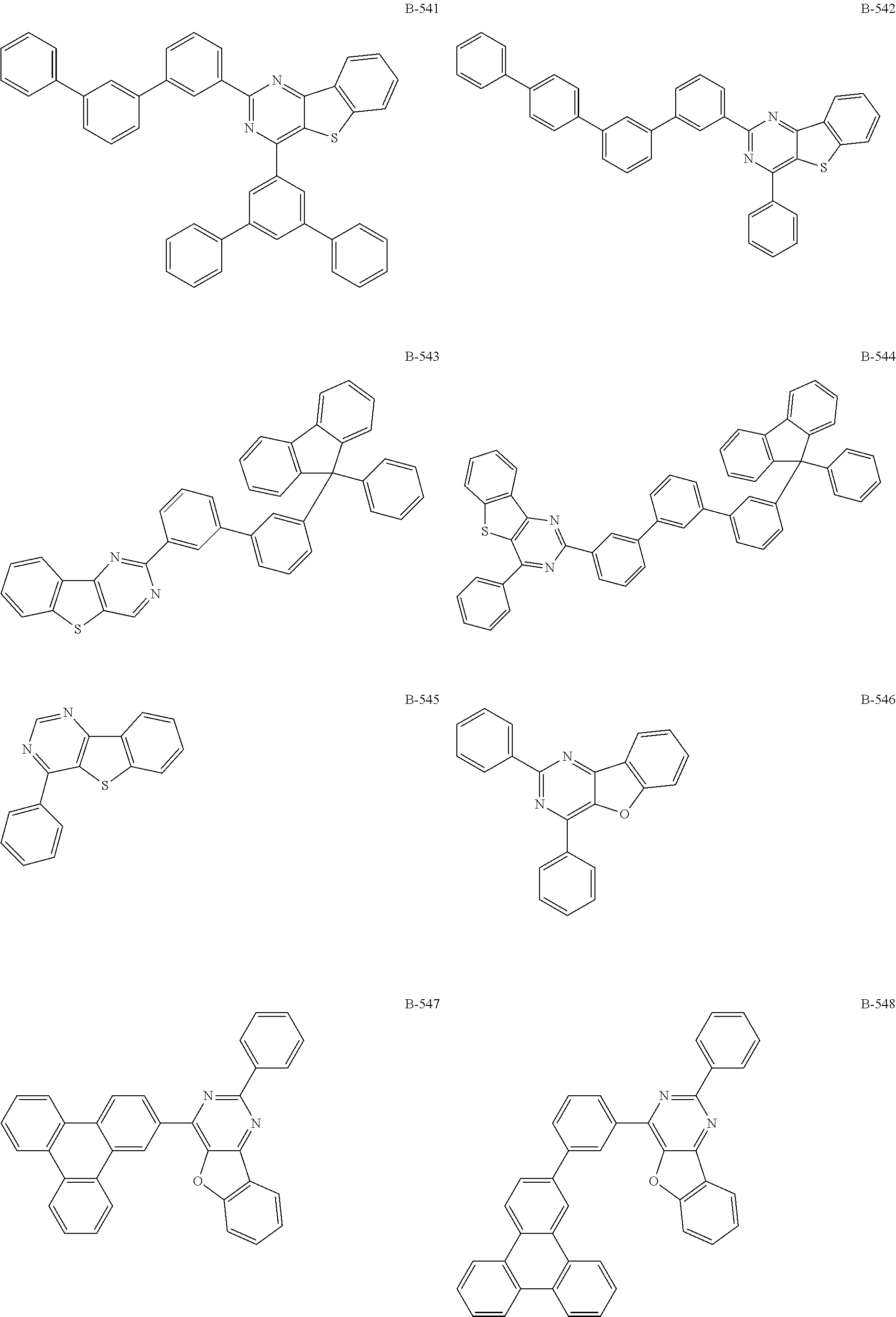

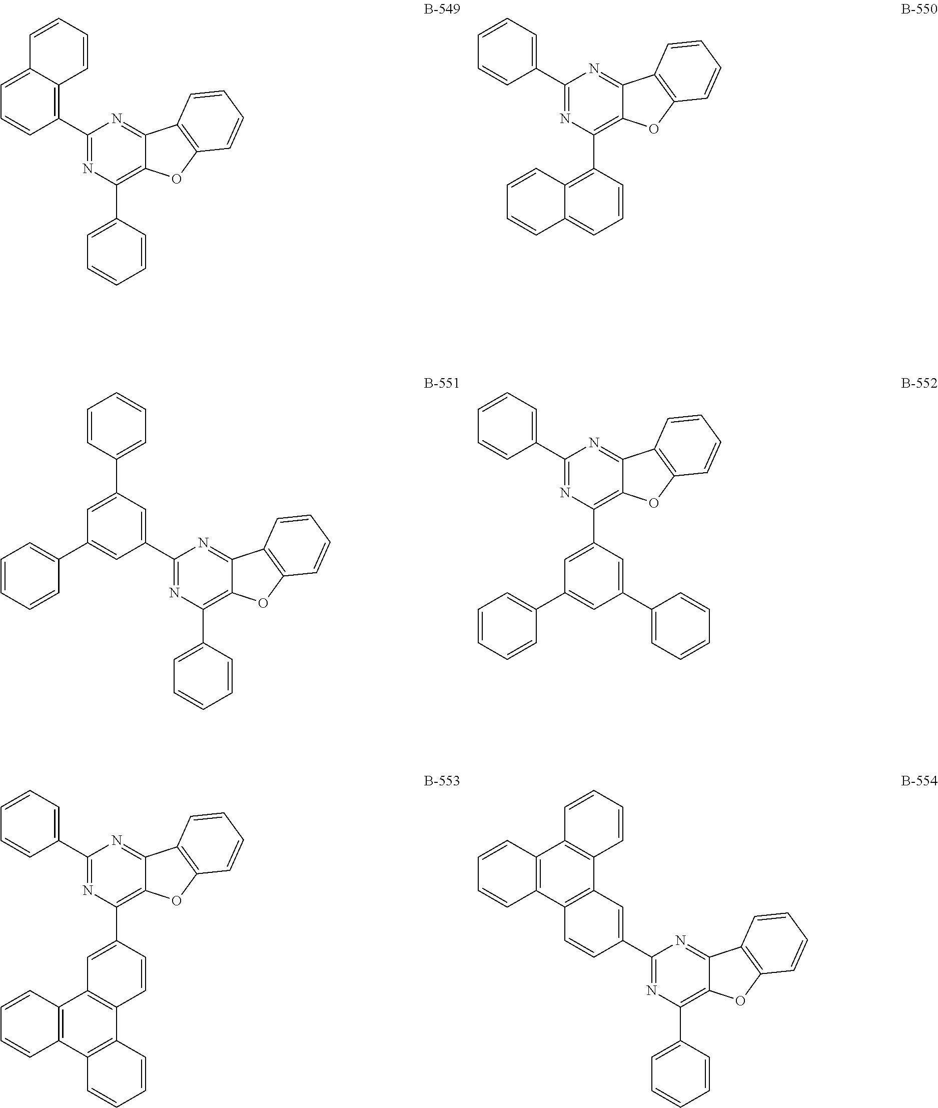 [SMILES: c1ccc(-c2nc(-c3cccc4ccccc34)nc3c2oc2ccccc23)cc1 ; c1ccc(-c2nc(-c3cccc4ccccc34)c3oc4ccccc4c3n2)cc1 ; c1ccc(-c2cc(-c3ccccc3)cc(-c3nc(-c4ccccc4)c4oc5ccccc5c4n3)c2)cc1 ; c1ccc(-c2cc(-c3ccccc3)cc(-c3nc(-c4ccccc4)nc4c3oc3ccccc34)c2)cc1 ; c1ccc(-c2nc(-c3ccc4c5ccccc5c5ccccc5c4c3)c3oc4ccccc4c3n2)cc1 ; c1ccc(-c2nc(-c3ccc4c5ccccc5c5ccccc5c4c3)nc3c2oc2ccccc23)cc1]

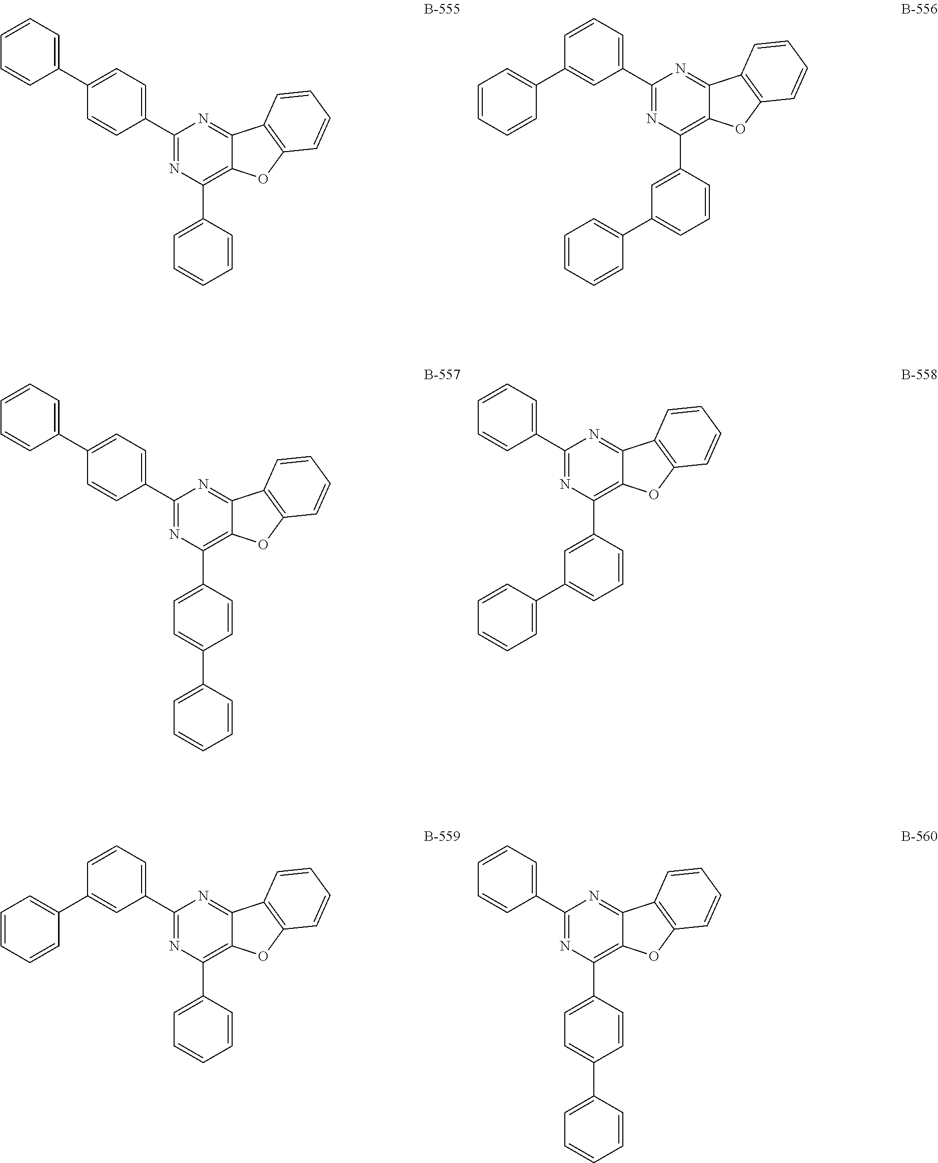 [SMILES: c1ccc(-c2ccc(-c3nc(-c4ccccc4)c4oc5ccccc5c4n3)cc2)cc1 ; c1ccc(-c2cccc(-c3nc(-c4cccc(-c5ccccc5)c4)c4oc5ccccc5c4n3)c2)cc1 ; c1ccc(-c2ccc(-c3nc(-c4ccc(-c5ccccc5)cc4)c4oc5ccccc5c4n3)cc2)cc1 ; c1ccc(-c2cccc(-c3nc(-c4ccccc4)nc4c3oc3ccccc34)c2)cc1 ; c1ccc(-c2cccc(-c3nc(-c4ccccc4)c4oc5ccccc5c4n3)c2)cc1 ; c1ccc(-c2ccc(-c3nc(-c4ccccc4)nc4c3oc3ccccc34)cc2)cc1]

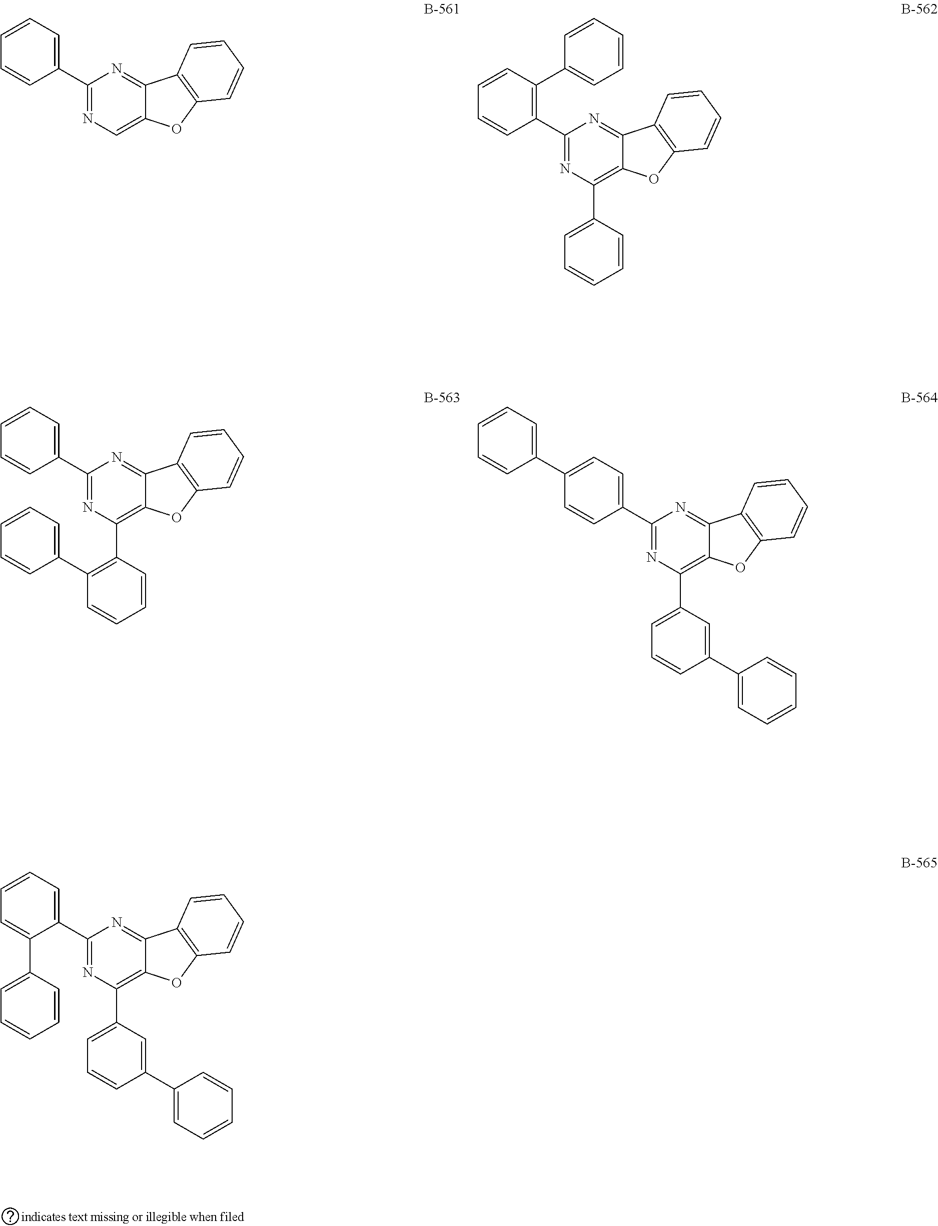 [SMILES: c1ccc(-c2ncc3oc4ccccc4c3n2)cc1 ; c1ccc(-c2ccccc2-c2nc(-c3ccccc3)c3oc4ccccc4c3n2)cc1 ; c1ccc(-c2nc(-c3ccccc3-c3ccccc3)c3oc4ccccc4c3n2)cc1 ; c1ccc(-c2ccc(-c3nc(-c4cccc(-c5ccccc5)c4)c4oc5ccccc5c4n3)cc2)cc1 ; c1ccc(-c2cccc(-c3nc(-c4ccccc4-c4ccccc4)nc4c3oc3ccccc34)c2)cc1]

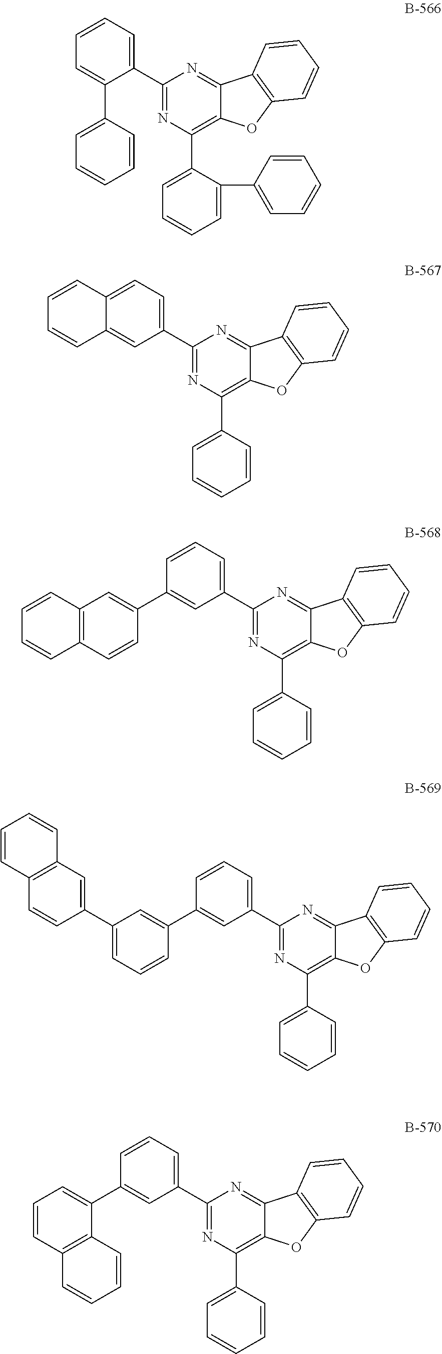 [SMILES: c1ccc(-c2ccccc2-c2nc(-c3ccccc3-c3ccccc3)c3oc4ccccc4c3n2)cc1 ; c1ccc(-c2nc(-c3ccc4ccccc4c3)nc3c2oc2ccccc23)cc1 ; c1ccc(-c2nc(-c3cccc(-c4ccc5ccccc5c4)c3)nc3c2oc2ccccc23)cc1 ; c1ccc(-c2nc(-c3cccc(-c4cccc(-c5ccc6ccccc6c5)c4)c3)nc3c2oc2ccccc23)cc1 ; c1ccc(-c2nc(-c3cccc(-c4cccc5ccccc45)c3)nc3c2oc2ccccc23)cc1]

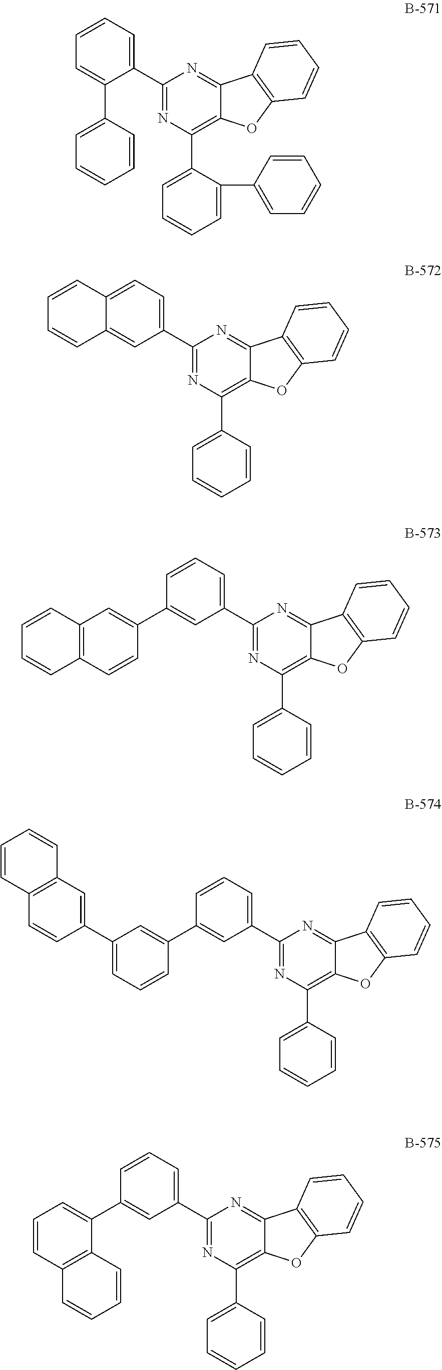 [SMILES: c1ccc(-c2ccccc2-c2nc(-c3ccccc3-c3ccccc3)c3oc4ccccc4c3n2)cc1 ; c1ccc(-c2nc(-c3ccc4ccccc4c3)nc3c2oc2ccccc23)cc1 ; c1ccc(-c2nc(-c3cccc(-c4ccc5ccccc5c4)c3)nc3c2oc2ccccc23)cc1 ; c1ccc(-c2nc(-c3cccc(-c4cccc(-c5ccc6ccccc6c5)c4)c3)nc3c2oc2ccccc23)cc1 ; c1ccc(-c2nc(-c3cccc(-c4cccc5ccccc45)c3)nc3c2oc2ccccc23)cc1]

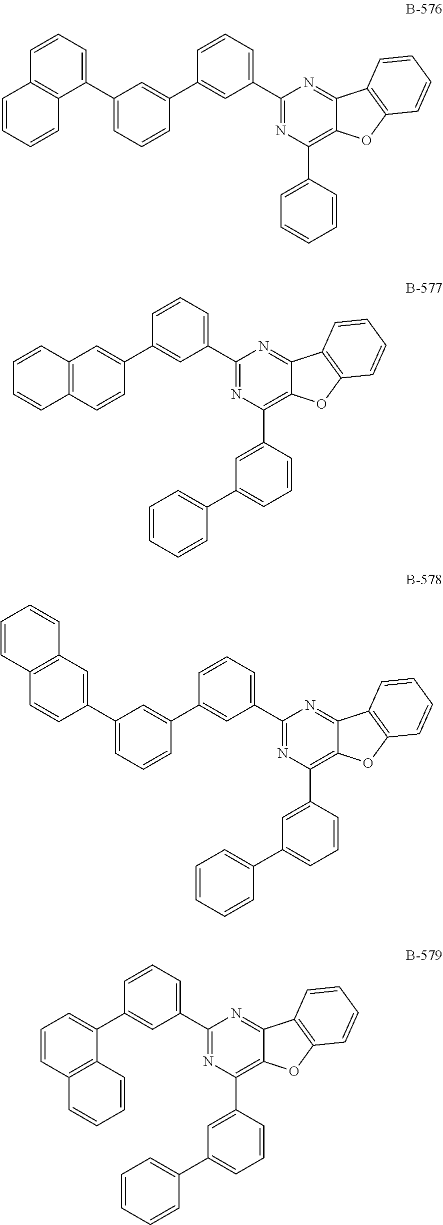 [SMILES: c1ccc(-c2nc(-c3cccc(-c4cccc(-c5cccc6ccccc56)c4)c3)nc3c2oc2ccccc23)cc1 ; c1ccc(-c2cccc(-c3nc(-c4cccc(-c5ccc6ccccc6c5)c4)nc4c3oc3ccccc34)c2)cc1 ; c1ccc(-c2cccc(-c3nc(-c4cccc(-c5cccc(-c6ccc7ccccc7c6)c5)c4)nc4c3oc3ccccc34)c2)cc1 ; c1ccc(-c2cccc(-c3nc(-c4cccc(-c5cccc6ccccc56)c4)nc4c3oc3ccccc34)c2)cc1]

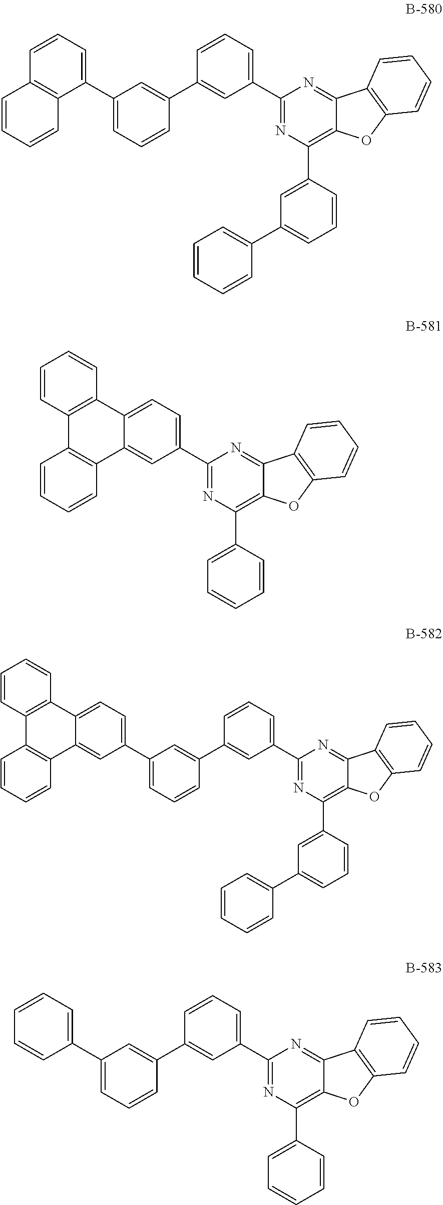 [SMILES: c1ccc(-c2cccc(-c3nc(-c4cccc(-c5cccc(-c6cccc7ccccc67)c5)c4)nc4c3oc3ccccc34)c2)cc1 ; c1ccc(-c2nc(-c3ccc4c5ccccc5c5ccccc5c4c3)nc3c2oc2ccccc23)cc1 ; c1ccc(-c2cccc(-c3nc(-c4cccc(-c5cccc(-c6ccc7c8ccccc8c8ccccc8c7c6)c5)c4)nc4c3oc3ccccc34)c2)cc1 ; c1ccc(-c2cccc(-c3cccc(-c4nc(-c5ccccc5)c5oc6ccccc6c5n4)c3)c2)cc1]

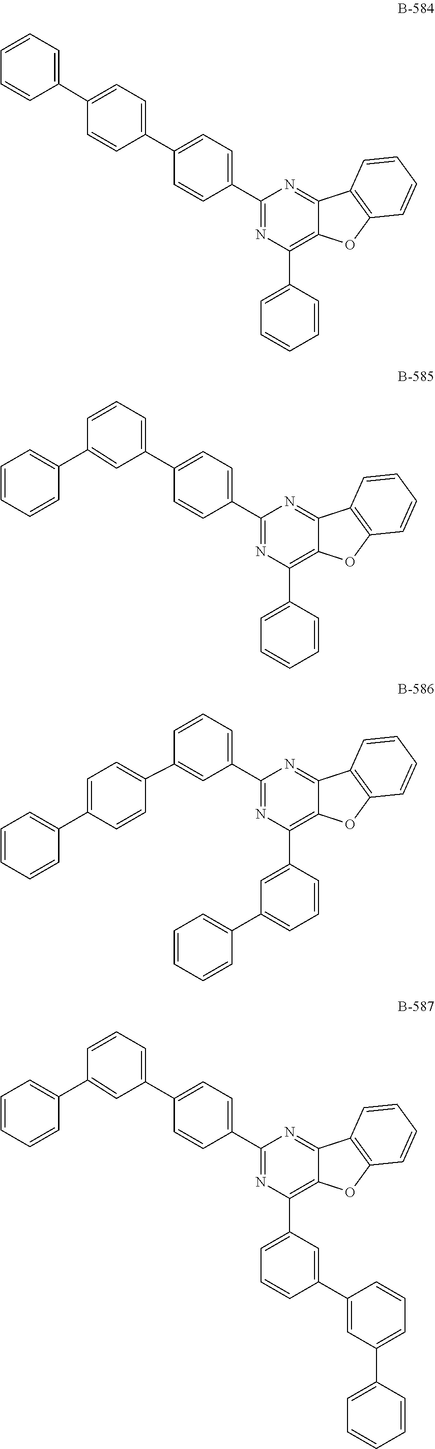 [SMILES: c1ccc(-c2ccc(-c3ccc(-c4nc(-c5ccccc5)c5oc6ccccc6c5n4)cc3)cc2)cc1 ; c1ccc(-c2cccc(-c3ccc(-c4nc(-c5ccccc5)c5oc6ccccc6c5n4)cc3)c2)cc1 ; c1ccc(-c2ccc(-c3cccc(-c4nc(-c5cccc(-c6ccccc6)c5)c5oc6ccccc6c5n4)c3)cc2)cc1 ; c1ccc(-c2cccc(-c3ccc(-c4nc(-c5cccc(-c6cccc(-c7ccccc7)c6)c5)c5oc6ccccc6c5n4)cc3)c2)cc1]

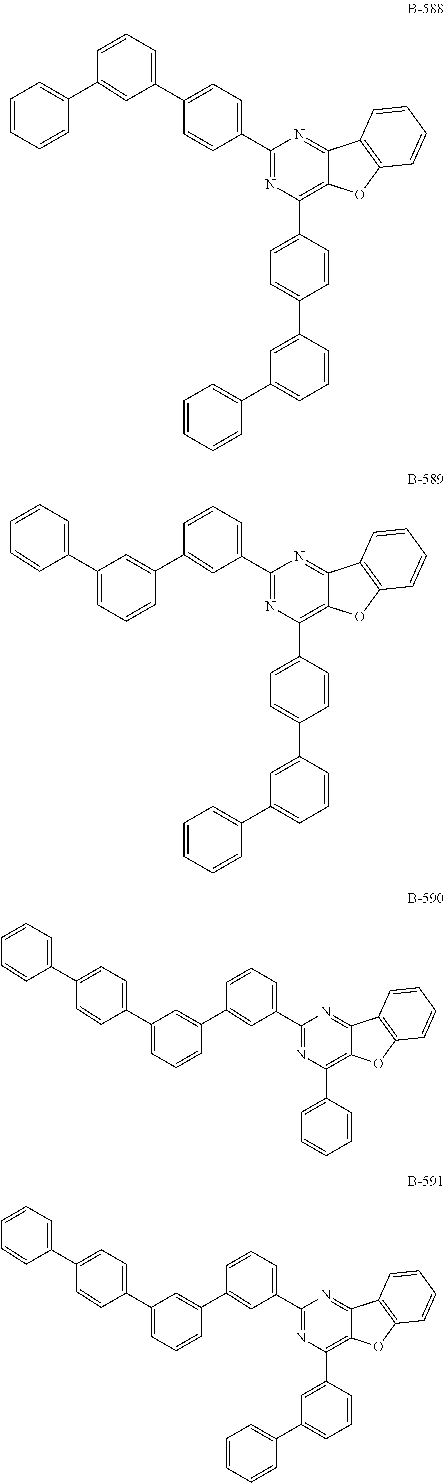 [SMILES: c1ccc(-c2cccc(-c3ccc(-c4nc(-c5ccc(-c6cccc(-c7ccccc7)c6)cc5)c5oc6ccccc6c5n4)cc3)c2)cc1 ; c1ccc(-c2cccc(-c3ccc(-c4nc(-c5cccc(-c6cccc(-c7ccccc7)c6)c5)nc5c4oc4ccccc45)cc3)c2)cc1 ; c1ccc(-c2ccc(-c3cccc(-c4cccc(-c5nc(-c6ccccc6)c6oc7ccccc7c6n5)c4)c3)cc2)cc1 ; c1ccc(-c2ccc(-c3cccc(-c4cccc(-c5nc(-c6cccc(-c7ccccc7)c6)c6oc7ccccc7c6n5)c4)c3)cc2)cc1]

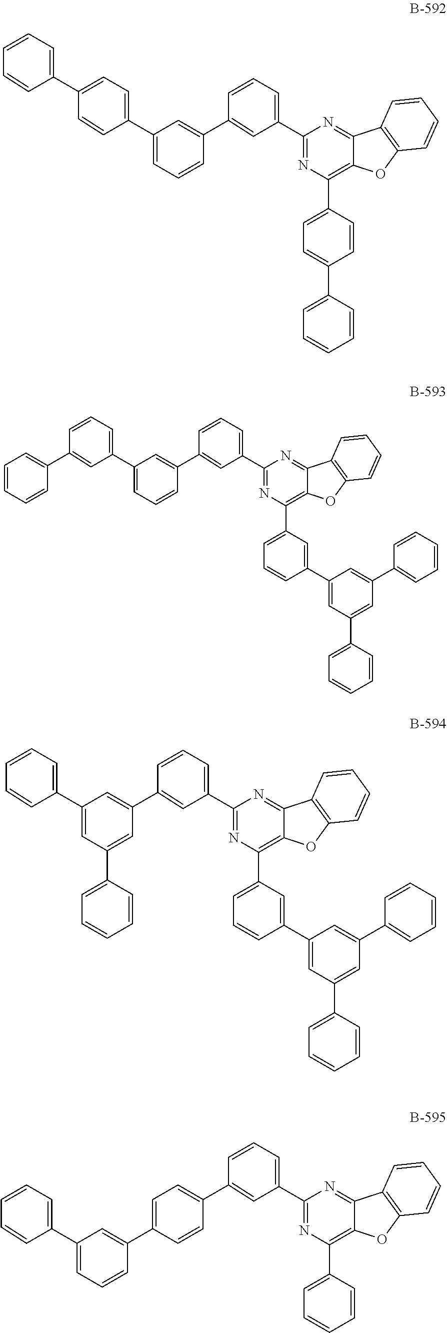 [SMILES: c1ccc(-c2ccc(-c3cccc(-c4cccc(-c5nc(-c6ccc(-c7ccccc7)cc6)c6oc7ccccc7c6n5)c4)c3)cc2)cc1 ; c1ccc(-c2cccc(-c3cccc(-c4cccc(-c5nc(-c6cccc(-c7cc(-c8ccccc8)cc(-c8ccccc8)c7)c6)c6oc7ccccc7c6n5)c4)c3)c2)cc1 ; c1ccc(-c2cc(-c3ccccc3)cc(-c3cccc(-c4nc(-c5cccc(-c6cc(-c7ccccc7)cc(-c7ccccc7)c6)c5)c5oc6ccccc6c5n4)c3)c2)cc1 ; c1ccc(-c2cccc(-c3ccc(-c4cccc(-c5nc(-c6ccccc6)c6oc7ccccc7c6n5)c4)cc3)c2)cc1]

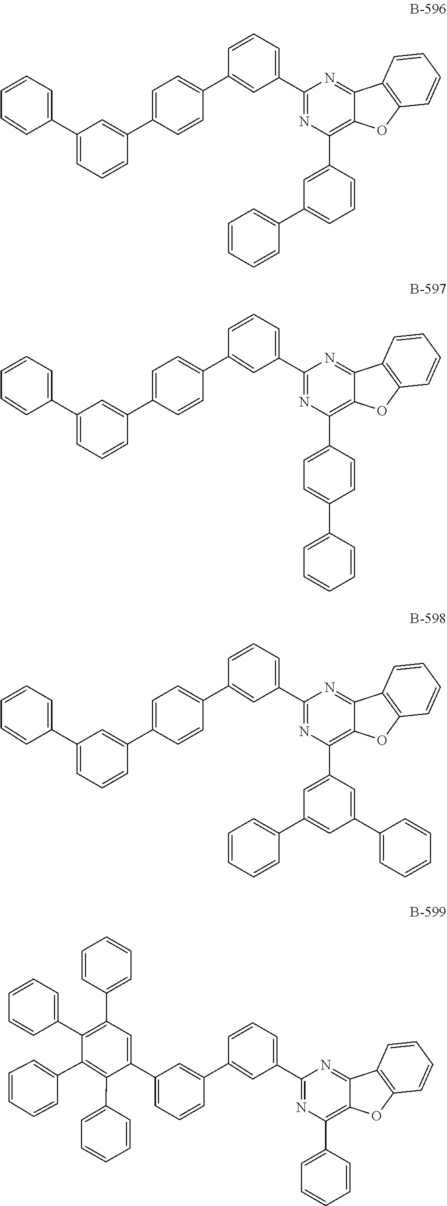 [SMILES: c1ccc(-c2cccc(-c3ccc(-c4cccc(-c5nc(-c6cccc(-c7ccccc7)c6)c6oc7ccccc7c6n5)c4)cc3)c2)cc1 ; c1ccc(-c2ccc(-c3nc(-c4cccc(-c5ccc(-c6cccc(-c7ccccc7)c6)cc5)c4)nc4c3oc3ccccc34)cc2)cc1 ; c1ccc(-c2cccc(-c3ccc(-c4cccc(-c5nc(-c6cc(-c7ccccc7)cc(-c7ccccc7)c6)c6oc7ccccc7c6n5)c4)cc3)c2)cc1 ; c1ccc(-c2cc(-c3cccc(-c4cccc(-c5nc(-c6ccccc6)c6oc7ccccc7c6n5)c4)c3)c(-c3ccccc3)c(-c3ccccc3)c2-c2ccccc2)cc1]

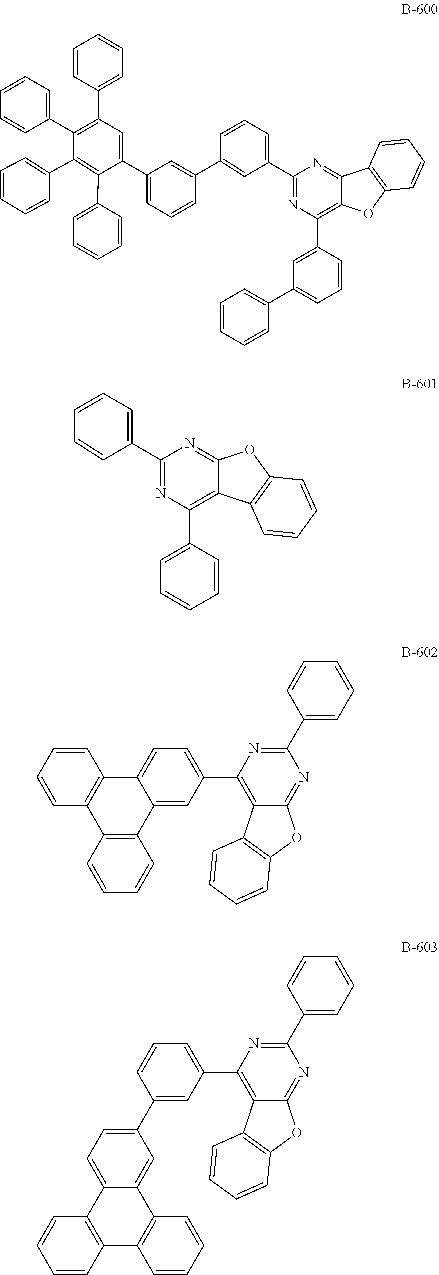 [SMILES: c1ccc(-c2cccc(-c3nc(-c4cccc(-c5cccc(-c6cc(-c7ccccc7)c(-c7ccccc7)c(-c7ccccc7)c6-c6ccccc6)c5)c4)nc4c3oc3ccccc34)c2)cc1 ; c1ccc(-c2nc(-c3ccccc3)c3c(n2)oc2ccccc23)cc1 ; c1ccc(-c2nc(-c3ccc4c5ccccc5c5ccccc5c4c3)c3c(n2)oc2ccccc23)cc1 ; c1ccc(-c2nc(-c3cccc(-c4ccc5c6ccccc6c6ccccc6c5c4)c3)c3c(n2)oc2ccccc23)cc1]

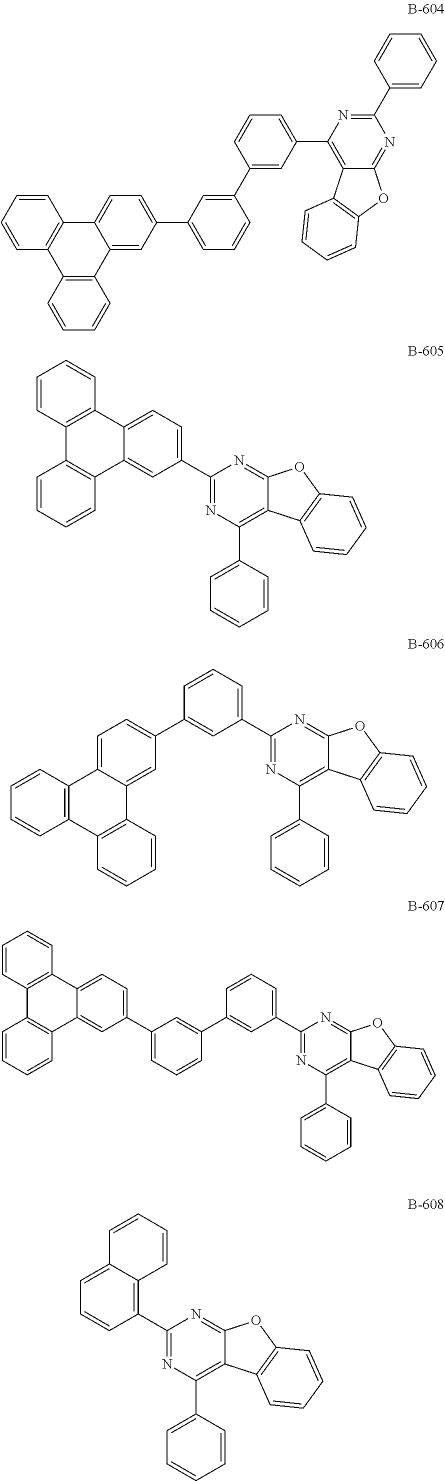

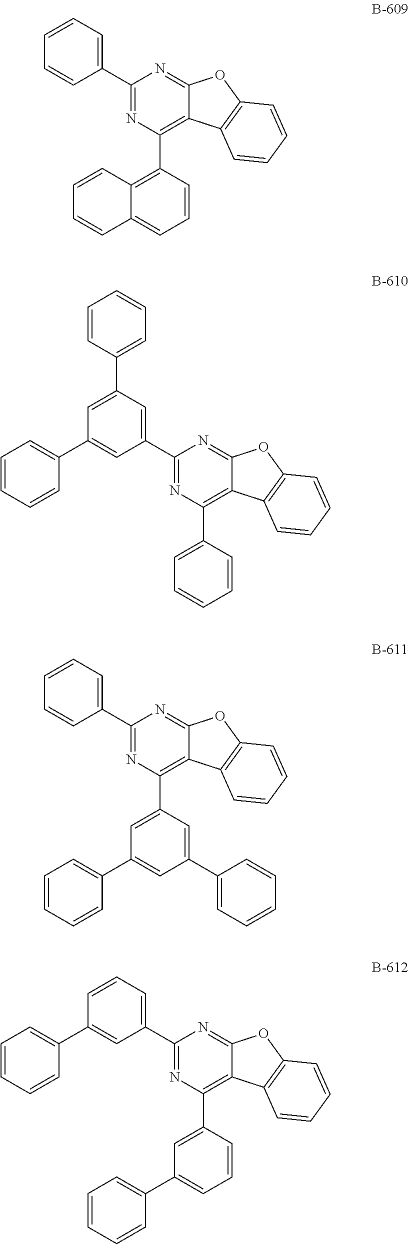 [SMILES: c1ccc(-c2nc(-c3cccc4ccccc34)c3c(n2)oc2ccccc23)cc1 ; c1ccc(-c2cc(-c3ccccc3)cc(-c3nc(-c4ccccc4)c4c(n3)oc3ccccc34)c2)cc1 ; c1ccc(-c2cc(-c3ccccc3)cc(-c3nc(-c4ccccc4)nc4oc5ccccc5c34)c2)cc1 ; c1ccc(-c2cccc(-c3nc(-c4cccc(-c5ccccc5)c4)c4c(n3)oc3ccccc34)c2)cc1]

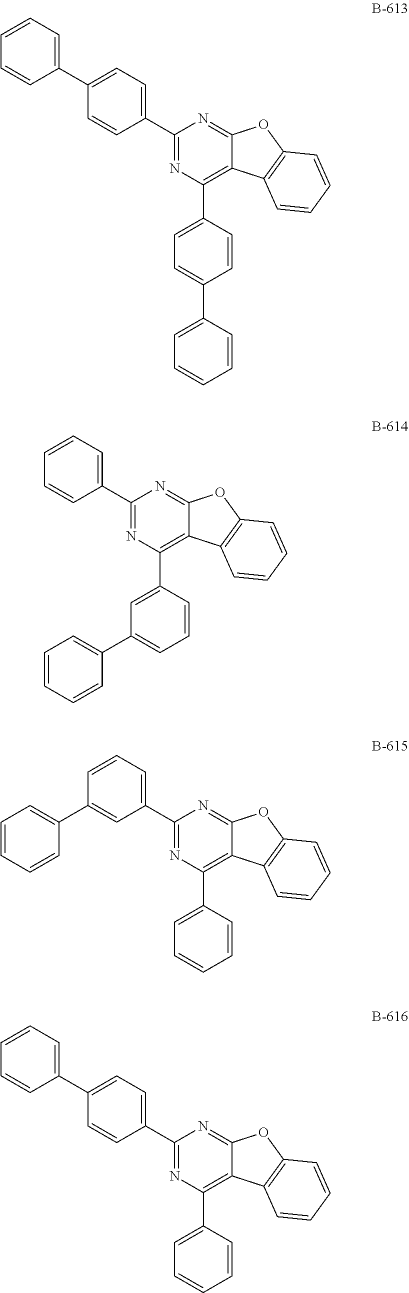 [SMILES: c1ccc(-c2ccc(-c3nc(-c4ccc(-c5ccccc5)cc4)c4c(n3)oc3ccccc34)cc2)cc1 ; c1ccc(-c2cccc(-c3nc(-c4ccccc4)nc4oc5ccccc5c34)c2)cc1 ; c1ccc(-c2cccc(-c3nc(-c4ccccc4)c4c(n3)oc3ccccc34)c2)cc1 ; c1ccc(-c2ccc(-c3nc(-c4ccccc4)c4c(n3)oc3ccccc34)cc2)cc1]

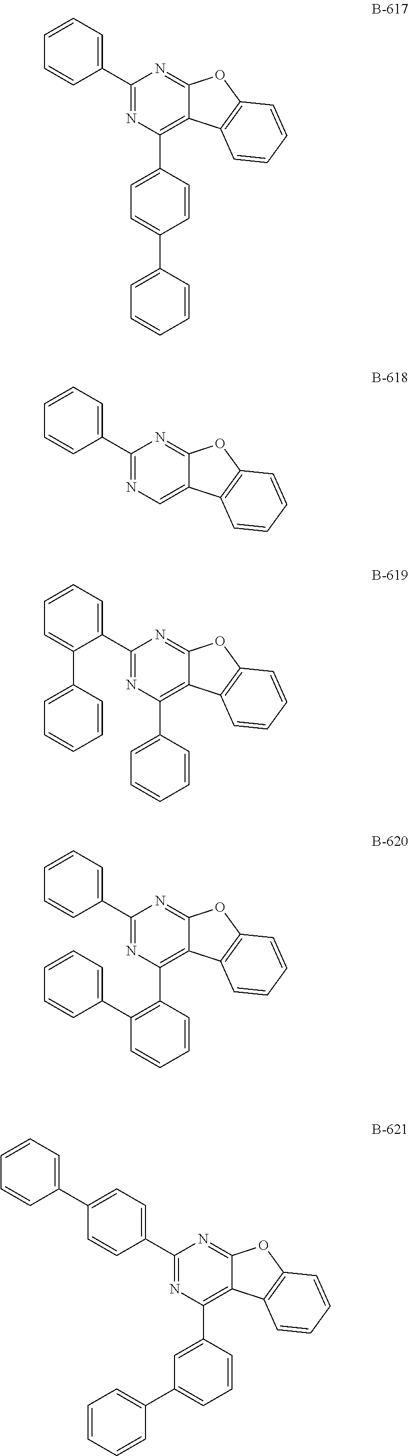 [SMILES: c1ccc(-c2ccc(-c3nc(-c4ccccc4)nc4oc5ccccc5c34)cc2)cc1 ; c1ccc(-c2ncc3c(n2)oc2ccccc23)cc1 ; c1ccc(-c2ccccc2-c2nc(-c3ccccc3)c3c(n2)oc2ccccc23)cc1 ; c1ccc(-c2nc(-c3ccccc3-c3ccccc3)c3c(n2)oc2ccccc23)cc1 ; c1ccc(-c2ccc(-c3nc(-c4cccc(-c5ccccc5)c4)c4c(n3)oc3ccccc34)cc2)cc1]

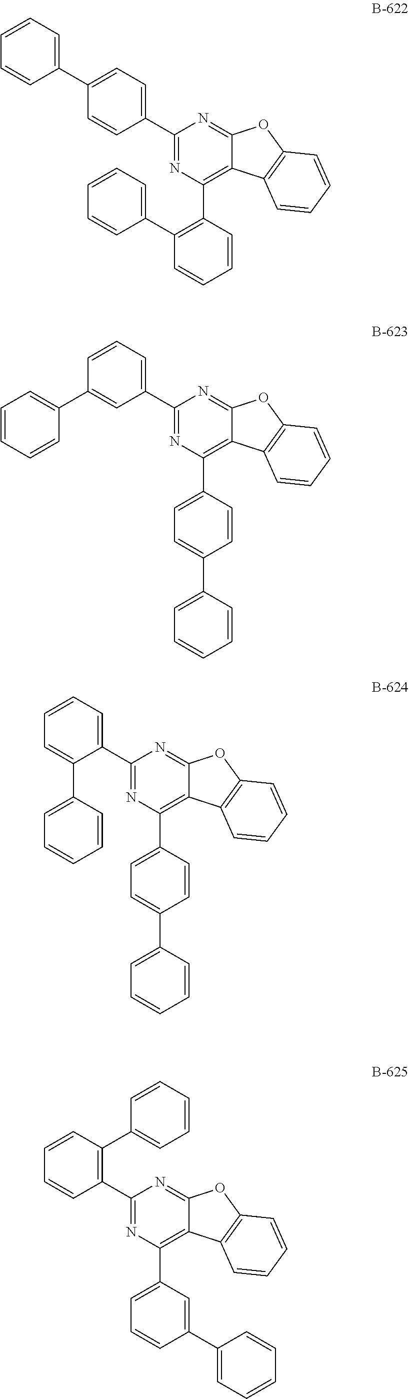 [SMILES: c1ccc(-c2ccc(-c3nc(-c4ccccc4-c4ccccc4)c4c(n3)oc3ccccc34)cc2)cc1 ; c1ccc(-c2ccc(-c3nc(-c4cccc(-c5ccccc5)c4)nc4oc5ccccc5c34)cc2)cc1 ; c1ccc(-c2ccc(-c3nc(-c4ccccc4-c4ccccc4)nc4oc5ccccc5c34)cc2)cc1 ; c1ccc(-c2cccc(-c3nc(-c4ccccc4-c4ccccc4)nc4oc5ccccc5c34)c2)cc1]

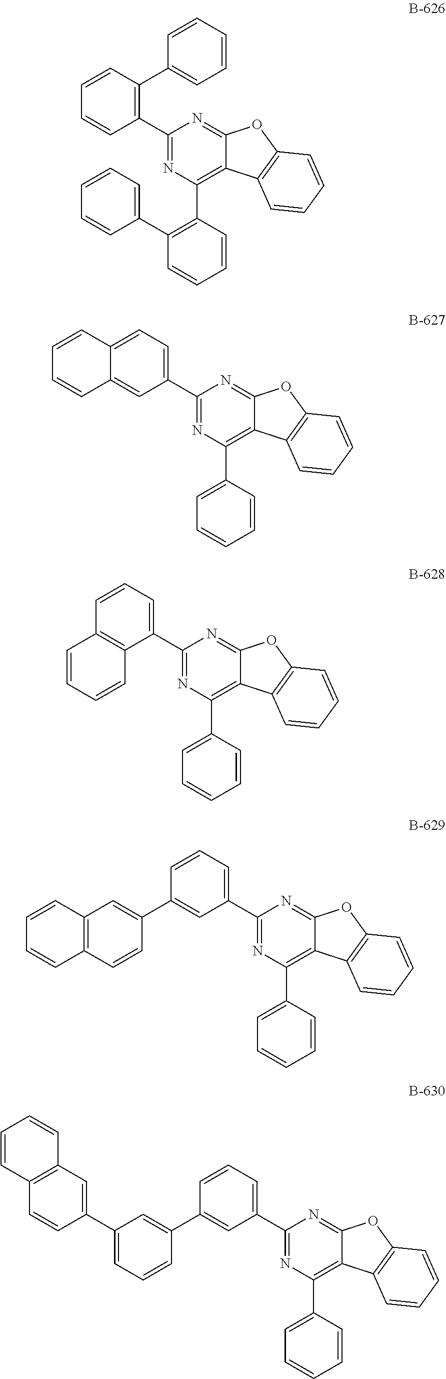 [SMILES: c1ccc(-c2ccccc2-c2nc(-c3ccccc3-c3ccccc3)c3c(n2)oc2ccccc23)cc1 ; c1ccc(-c2nc(-c3ccc4ccccc4c3)nc3oc4ccccc4c23)cc1 ; c1ccc(-c2nc(-c3cccc4ccccc34)nc3oc4ccccc4c23)cc1 ; c1ccc(-c2nc(-c3cccc(-c4ccc5ccccc5c4)c3)nc3oc4ccccc4c23)cc1 ; c1ccc(-c2nc(-c3cccc(-c4cccc(-c5ccc6ccccc6c5)c4)c3)nc3oc4ccccc4c23)cc1]

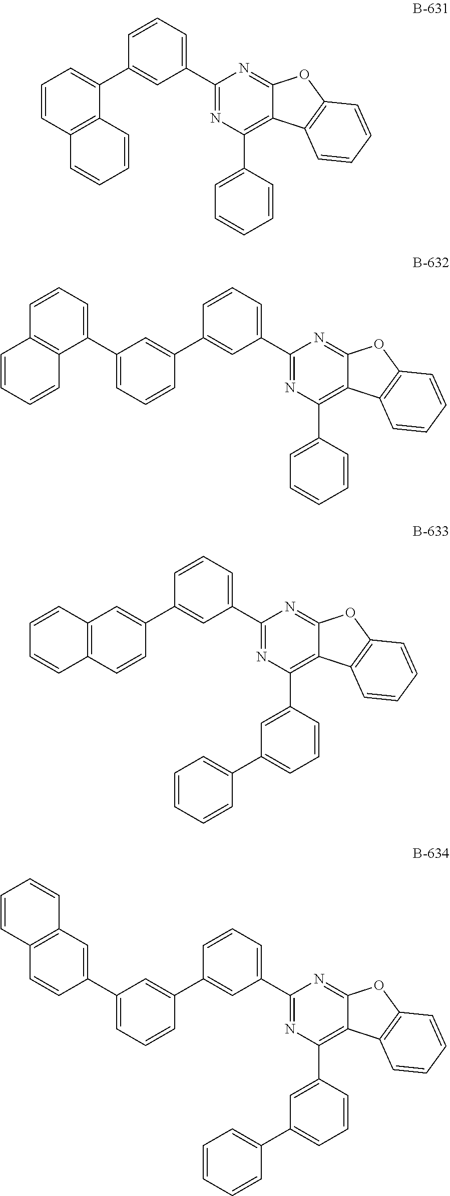 [SMILES: c1ccc(-c2nc(-c3cccc(-c4cccc5ccccc45)c3)nc3oc4ccccc4c23)cc1 ; c1ccc(-c2nc(-c3cccc(-c4cccc(-c5cccc6ccccc56)c4)c3)nc3oc4ccccc4c23)cc1 ; c1ccc(-c2cccc(-c3nc(-c4cccc(-c5ccc6ccccc6c5)c4)nc4oc5ccccc5c34)c2)cc1 ; c1ccc(-c2cccc(-c3nc(-c4cccc(-c5cccc(-c6ccc7ccccc7c6)c5)c4)nc4oc5ccccc5c34)c2)cc1]

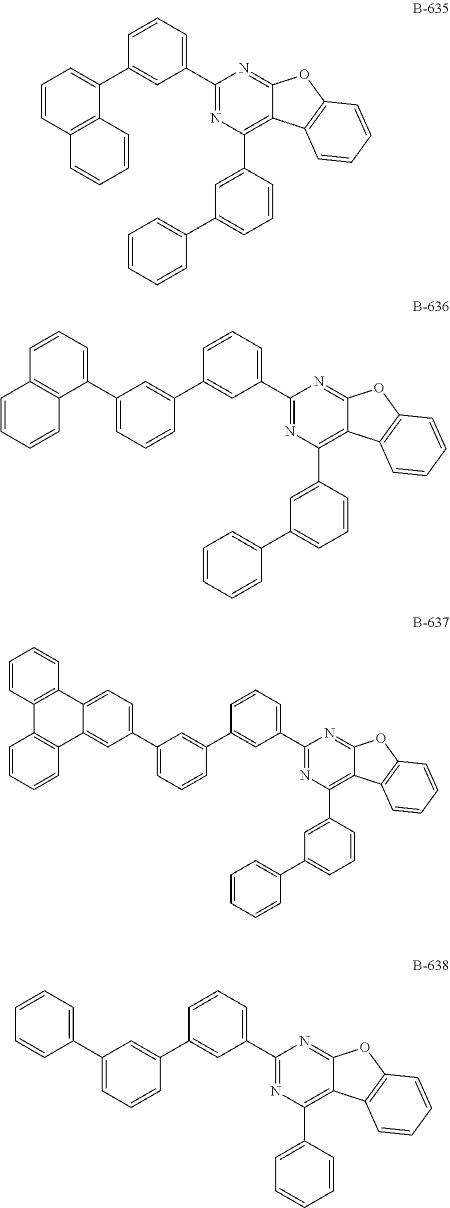 [SMILES: c1ccc(-c2cccc(-c3nc(-c4cccc(-c5cccc6ccccc56)c4)nc4oc5ccccc5c34)c2)cc1 ; c1ccc(-c2cccc(-c3nc(-c4cccc(-c5cccc(-c6cccc7ccccc67)c5)c4)nc4oc5ccccc5c34)c2)cc1 ; c1ccc(-c2cccc(-c3nc(-c4cccc(-c5cccc(-c6ccc7c8ccccc8c8ccccc8c7c6)c5)c4)nc4oc5ccccc5c34)c2)cc1 ; c1ccc(-c2cccc(-c3cccc(-c4nc(-c5ccccc5)c5c(n4)oc4ccccc45)c3)c2)cc1]

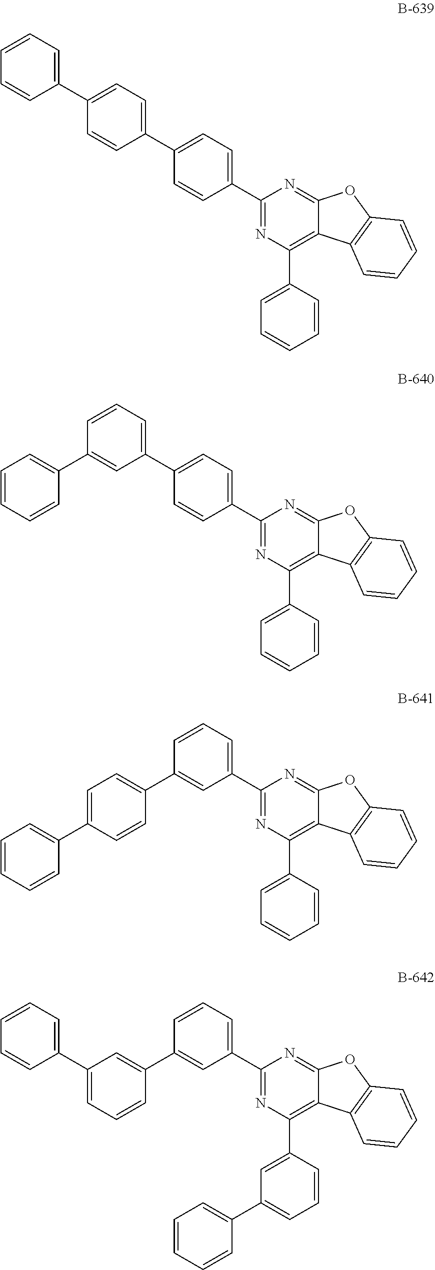 [SMILES: c1ccc(-c2ccc(-c3ccc(-c4nc(-c5ccccc5)c5c(n4)oc4ccccc45)cc3)cc2)cc1 ; c1ccc(-c2cccc(-c3ccc(-c4nc(-c5ccccc5)c5c(n4)oc4ccccc45)cc3)c2)cc1 ; c1ccc(-c2ccc(-c3cccc(-c4nc(-c5ccccc5)c5c(n4)oc4ccccc45)c3)cc2)cc1 ; c1ccc(-c2cccc(-c3cccc(-c4nc(-c5cccc(-c6ccccc6)c5)c5c(n4)oc4ccccc45)c3)c2)cc1]

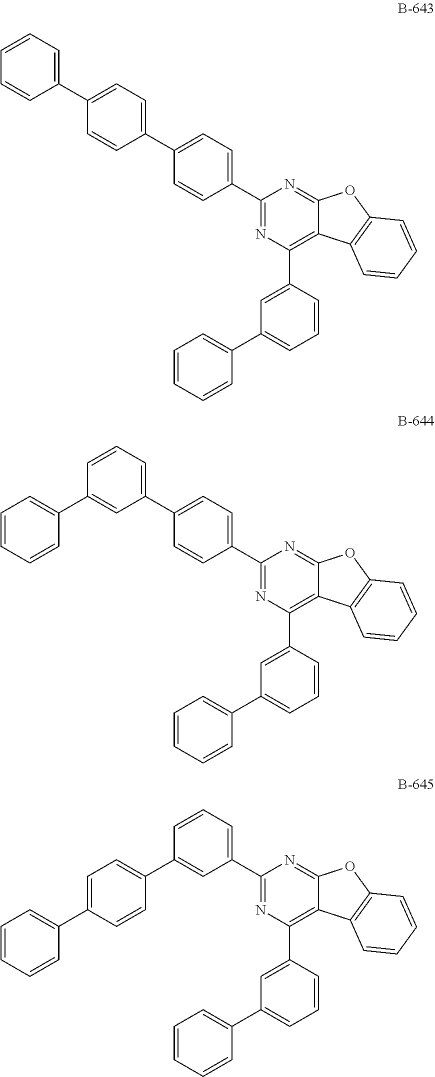 [SMILES: c1ccc(-c2ccc(-c3ccc(-c4nc(-c5cccc(-c6ccccc6)c5)c5c(n4)oc4ccccc45)cc3)cc2)cc1 ; c1ccc(-c2cccc(-c3ccc(-c4nc(-c5cccc(-c6ccccc6)c5)c5c(n4)oc4ccccc45)cc3)c2)cc1 ; c1ccc(-c2ccc(-c3cccc(-c4nc(-c5cccc(-c6ccccc6)c5)c5c(n4)oc4ccccc45)c3)cc2)cc1]

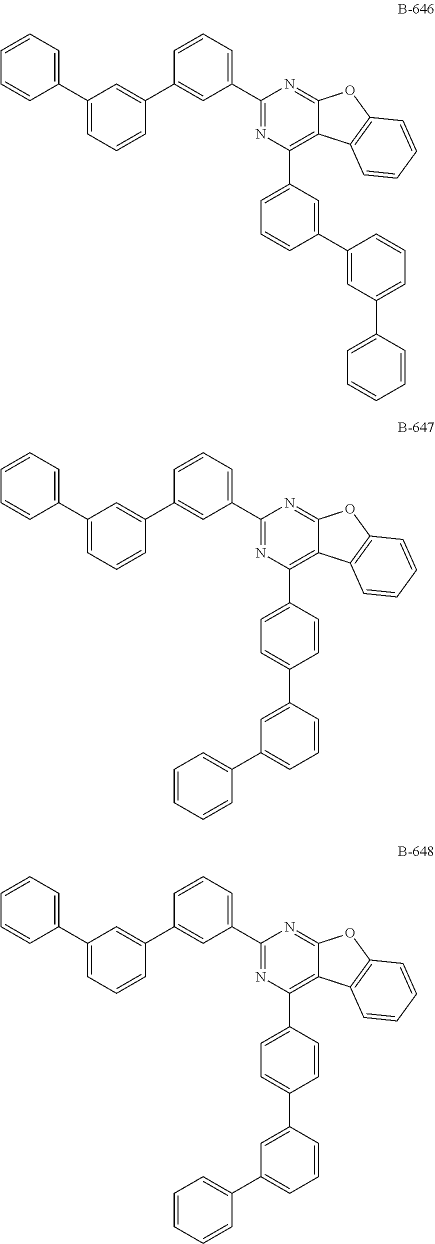 [SMILES: c1ccc(-c2cccc(-c3cccc(-c4nc(-c5cccc(-c6cccc(-c7ccccc7)c6)c5)c5c(n4)oc4ccccc45)c3)c2)cc1 ; c1ccc(-c2cccc(-c3ccc(-c4nc(-c5cccc(-c6cccc(-c7ccccc7)c6)c5)nc5oc6ccccc6c45)cc3)c2)cc1 ; c1ccc(-c2cccc(-c3ccc(-c4nc(-c5cccc(-c6cccc(-c7ccccc7)c6)c5)nc5oc6ccccc6c45)cc3)c2)cc1]

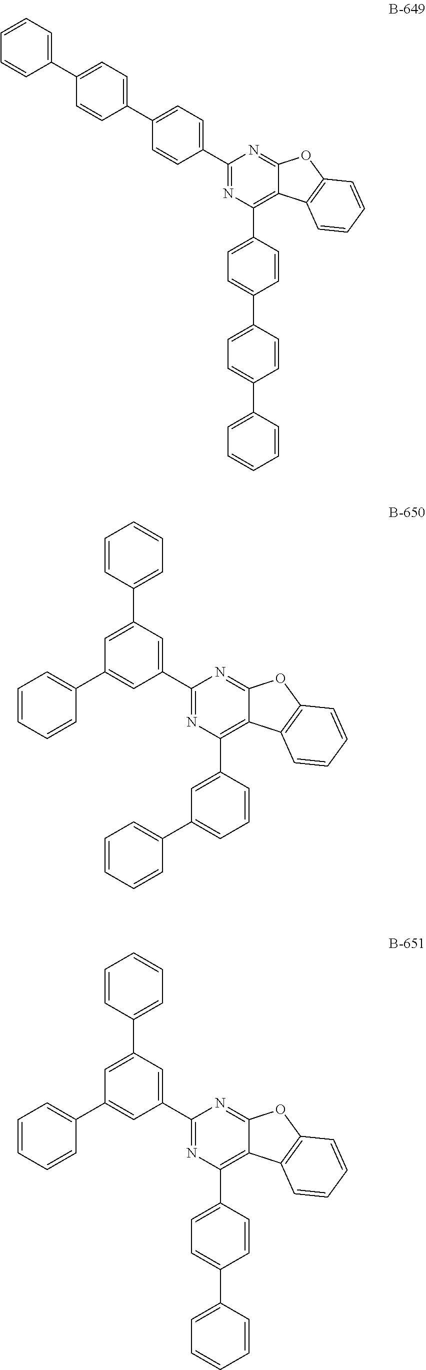 [SMILES: c1ccc(-c2ccc(-c3ccc(-c4nc(-c5ccc(-c6ccc(-c7ccccc7)cc6)cc5)c5c(n4)oc4ccccc45)cc3)cc2)cc1 ; c1ccc(-c2cc(-c3ccccc3)cc(-c3nc(-c4cccc(-c5ccccc5)c4)c4c(n3)oc3ccccc34)c2)cc1 ; c1ccc(-c2ccc(-c3nc(-c4cc(-c5ccccc5)cc(-c5ccccc5)c4)nc4oc5ccccc5c34)cc2)cc1]

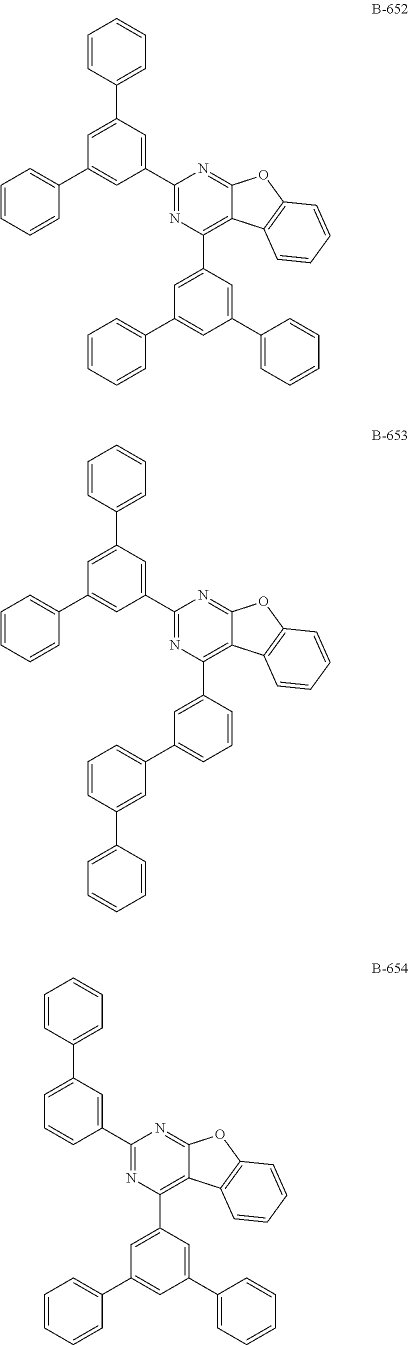 [SMILES: c1ccc(-c2cc(-c3ccccc3)cc(-c3nc(-c4cc(-c5ccccc5)cc(-c5ccccc5)c4)c4c(n3)oc3ccccc34)c2)cc1 ; c1ccc(-c2cccc(-c3cccc(-c4nc(-c5cc(-c6ccccc6)cc(-c6ccccc6)c5)nc5oc6ccccc6c45)c3)c2)cc1 ; c1ccc(-c2cccc(-c3nc(-c4cc(-c5ccccc5)cc(-c5ccccc5)c4)c4c(n3)oc3ccccc34)c2)cc1]

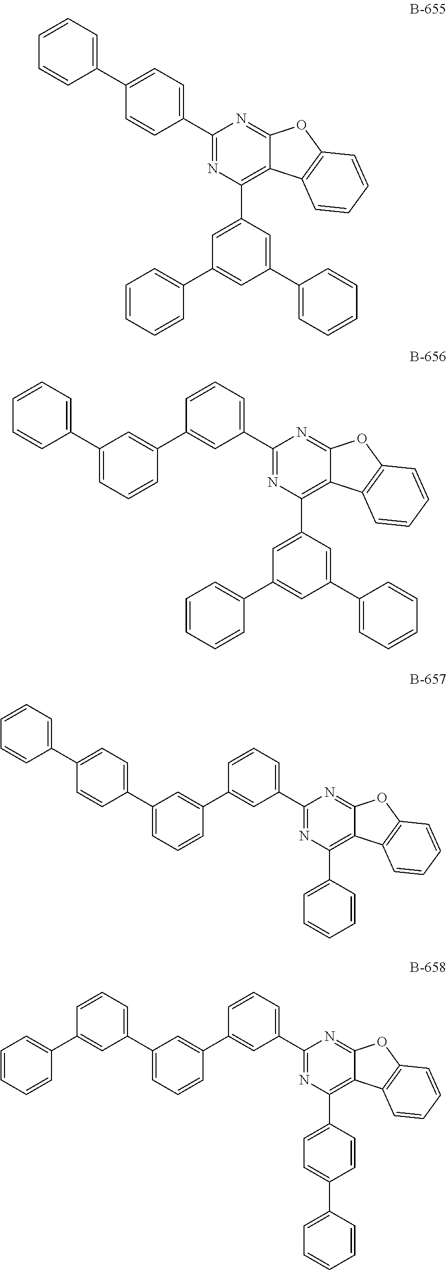 [SMILES: c1ccc(-c2ccc(-c3nc(-c4cc(-c5ccccc5)cc(-c5ccccc5)c4)c4c(n3)oc3ccccc34)cc2)cc1 ; c1ccc(-c2cccc(-c3cccc(-c4nc(-c5cc(-c6ccccc6)cc(-c6ccccc6)c5)c5c(n4)oc4ccccc45)c3)c2)cc1 ; c1ccc(-c2ccc(-c3cccc(-c4cccc(-c5nc(-c6ccccc6)c6c(n5)oc5ccccc56)c4)c3)cc2)cc1 ; c1ccc(-c2ccc(-c3nc(-c4cccc(-c5cccc(-c6cccc(-c7ccccc7)c6)c5)c4)nc4oc5ccccc5c34)cc2)cc1]

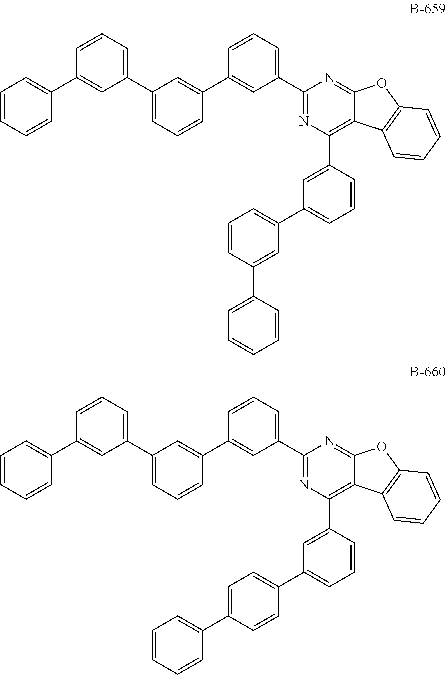 [SMILES: c1ccc(-c2cccc(-c3cccc(-c4cccc(-c5nc(-c6cccc(-c7cccc(-c8ccccc8)c7)c6)c6c(n5)oc5ccccc56)c4)c3)c2)cc1 ; c1ccc(-c2ccc(-c3cccc(-c4nc(-c5cccc(-c6cccc(-c7cccc(-c8ccccc8)c7)c6)c5)nc5oc6ccccc6c45)c3)cc2)cc1]

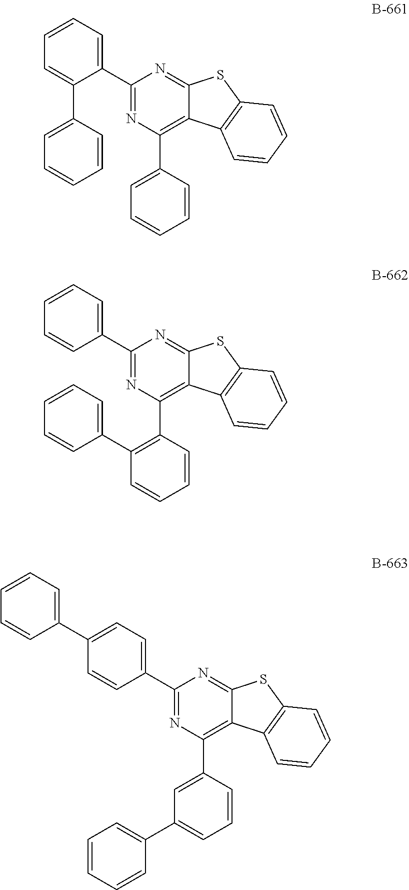 [SMILES: c1ccc(-c2ccccc2-c2nc(-c3ccccc3)c3c(n2)sc2ccccc23)cc1 ; c1ccc(-c2nc(-c3ccccc3-c3ccccc3)c3c(n2)sc2ccccc23)cc1 ; c1ccc(-c2ccc(-c3nc(-c4cccc(-c5ccccc5)c4)c4c(n3)sc3ccccc34)cc2)cc1]

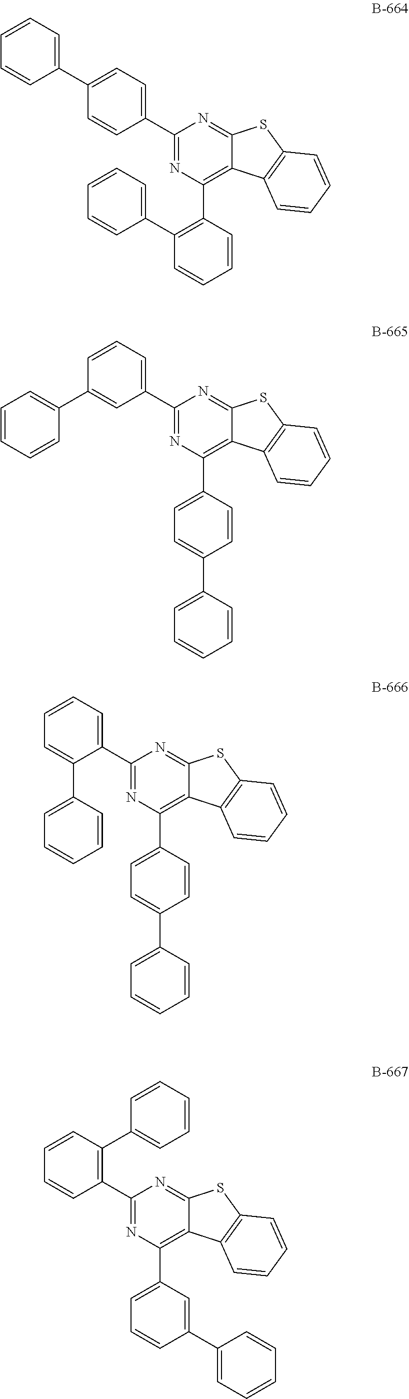 [SMILES: c1ccc(-c2ccc(-c3nc(-c4ccccc4-c4ccccc4)c4c(n3)sc3ccccc34)cc2)cc1 ; c1ccc(-c2ccc(-c3nc(-c4cccc(-c5ccccc5)c4)nc4sc5ccccc5c34)cc2)cc1 ; c1ccc(-c2ccc(-c3nc(-c4ccccc4-c4ccccc4)nc4sc5ccccc5c34)cc2)cc1 ; c1ccc(-c2cccc(-c3nc(-c4ccccc4-c4ccccc4)nc4sc5ccccc5c34)c2)cc1]

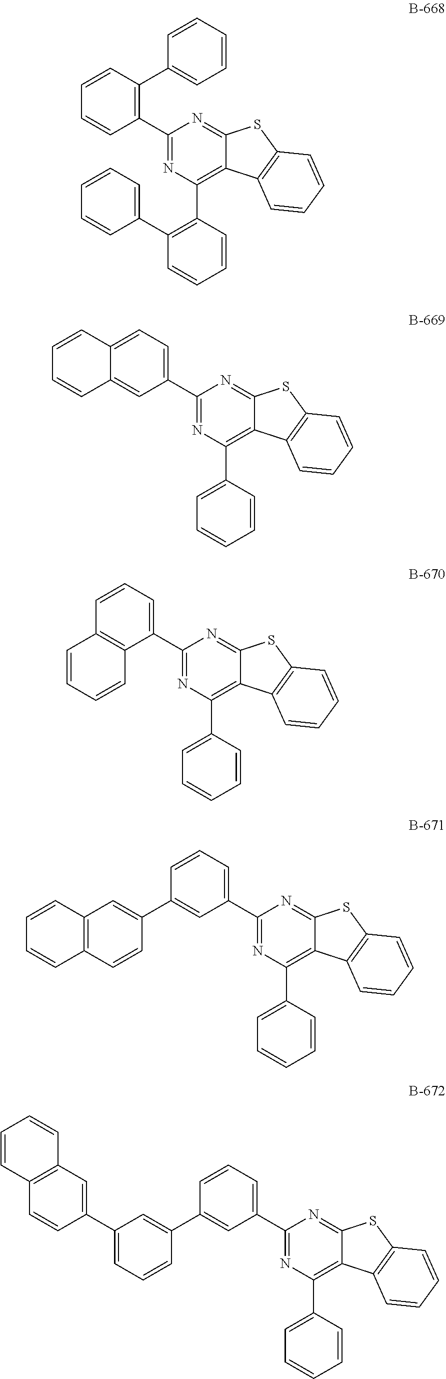 [SMILES: c1ccc(-c2ccccc2-c2nc(-c3ccccc3-c3ccccc3)c3c(n2)sc2ccccc23)cc1 ; c1ccc(-c2nc(-c3ccc4ccccc4c3)nc3sc4ccccc4c23)cc1 ; c1ccc(-c2nc(-c3cccc4ccccc34)nc3sc4ccccc4c23)cc1 ; c1ccc(-c2nc(-c3cccc(-c4ccc5ccccc5c4)c3)nc3sc4ccccc4c23)cc1 ; c1ccc(-c2nc(-c3cccc(-c4cccc(-c5ccc6ccccc6c5)c4)c3)nc3sc4ccccc4c23)cc1]

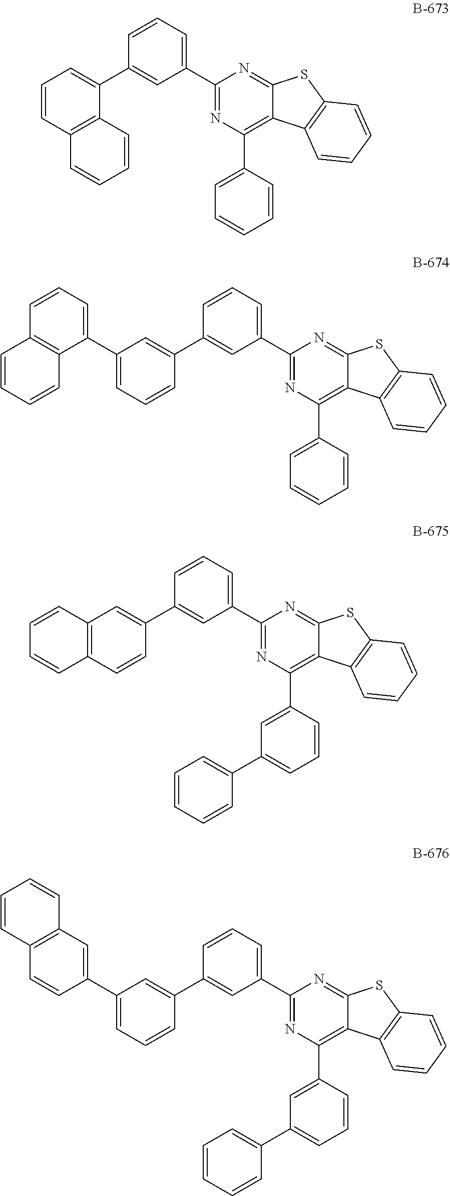 [SMILES: c1ccc(-c2nc(-c3cccc(-c4cccc5ccccc45)c3)nc3sc4ccccc4c23)cc1 ; c1ccc(-c2nc(-c3cccc(-c4cccc(-c5cccc6ccccc56)c4)c3)nc3sc4ccccc4c23)cc1 ; c1ccc(-c2cccc(-c3nc(-c4cccc(-c5ccc6ccccc6c5)c4)nc4sc5ccccc5c34)c2)cc1 ; c1ccc(-c2cccc(-c3nc(-c4cccc(-c5cccc(-c6ccc7ccccc7c6)c5)c4)nc4sc5ccccc5c34)c2)cc1]

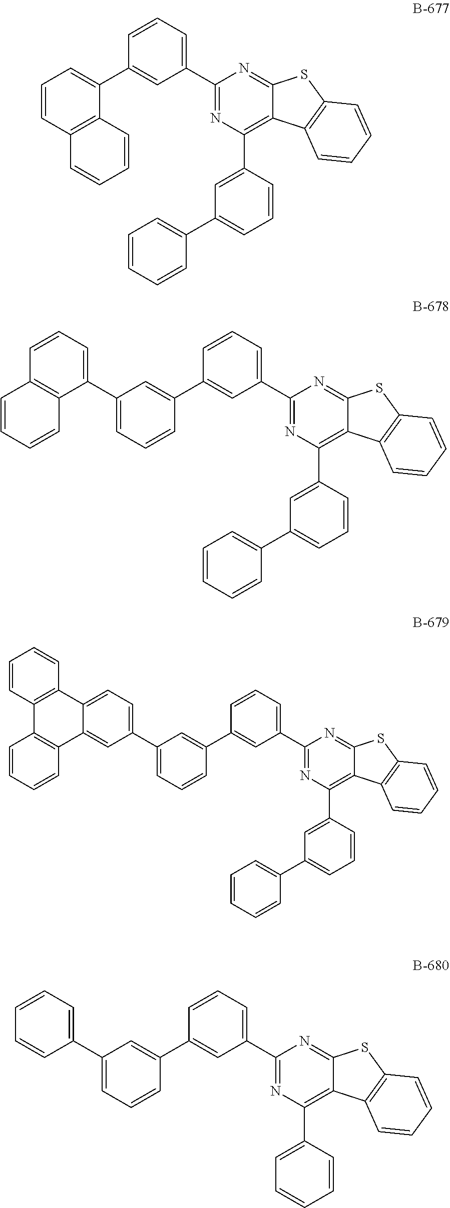 [SMILES: c1ccc(-c2cccc(-c3nc(-c4cccc(-c5cccc6ccccc56)c4)nc4sc5ccccc5c34)c2)cc1 ; c1ccc(-c2cccc(-c3nc(-c4cccc(-c5cccc(-c6cccc7ccccc67)c5)c4)nc4sc5ccccc5c34)c2)cc1 ; c1ccc(-c2cccc(-c3nc(-c4cccc(-c5cccc(-c6ccc7c8ccccc8c8ccccc8c7c6)c5)c4)nc4sc5ccccc5c34)c2)cc1 ; c1ccc(-c2cccc(-c3cccc(-c4nc(-c5ccccc5)c5c(n4)sc4ccccc45)c3)c2)cc1]

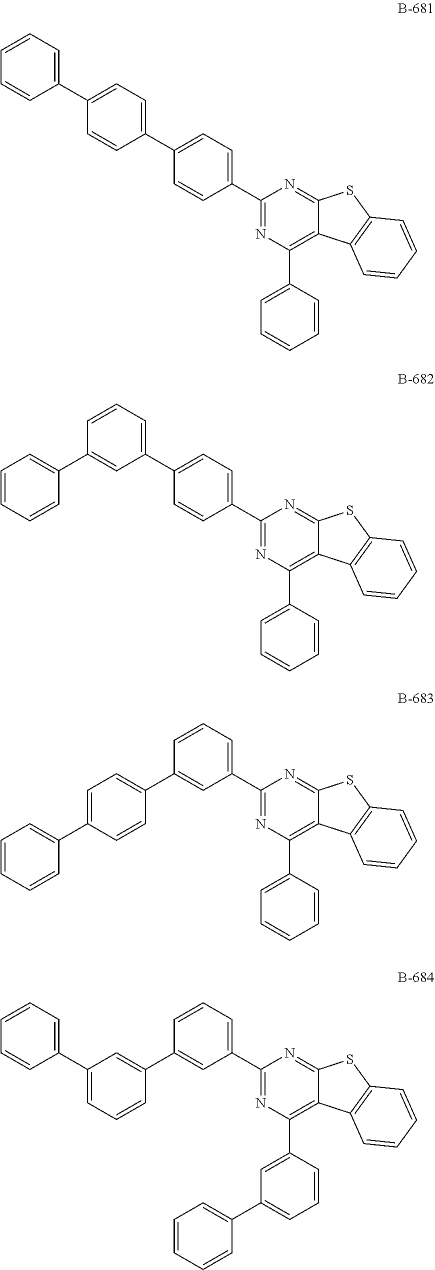 [SMILES: c1ccc(-c2ccc(-c3ccc(-c4nc(-c5ccccc5)c5c(n4)sc4ccccc45)cc3)cc2)cc1 ; c1ccc(-c2cccc(-c3ccc(-c4nc(-c5ccccc5)c5c(n4)sc4ccccc45)cc3)c2)cc1 ; c1ccc(-c2ccc(-c3cccc(-c4nc(-c5ccccc5)c5c(n4)sc4ccccc45)c3)cc2)cc1 ; c1ccc(-c2cccc(-c3cccc(-c4nc(-c5cccc(-c6ccccc6)c5)c5c(n4)sc4ccccc45)c3)c2)cc1]

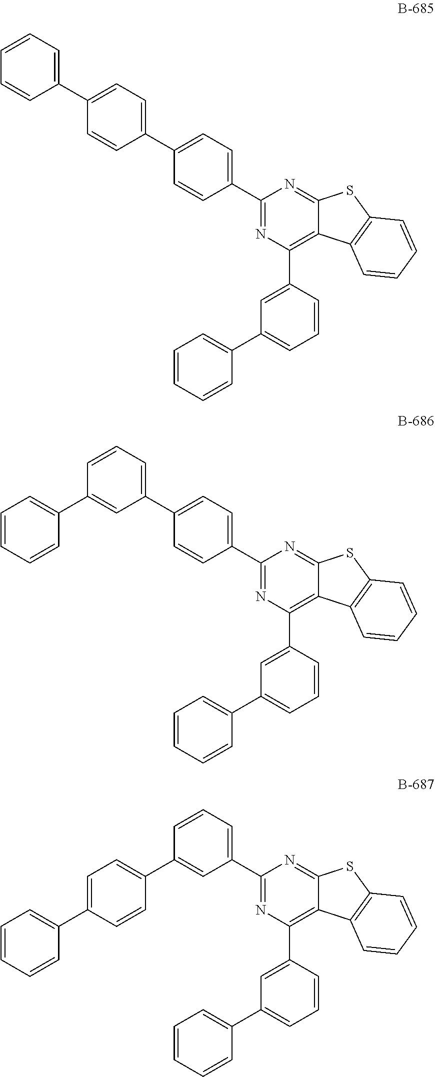 [SMILES: c1ccc(-c2ccc(-c3ccc(-c4nc(-c5cccc(-c6ccccc6)c5)c5c(n4)sc4ccccc45)cc3)cc2)cc1 ; c1ccc(-c2cccc(-c3ccc(-c4nc(-c5cccc(-c6ccccc6)c5)c5c(n4)sc4ccccc45)cc3)c2)cc1 ; c1ccc(-c2ccc(-c3cccc(-c4nc(-c5cccc(-c6ccccc6)c5)c5c(n4)sc4ccccc45)c3)cc2)cc1]

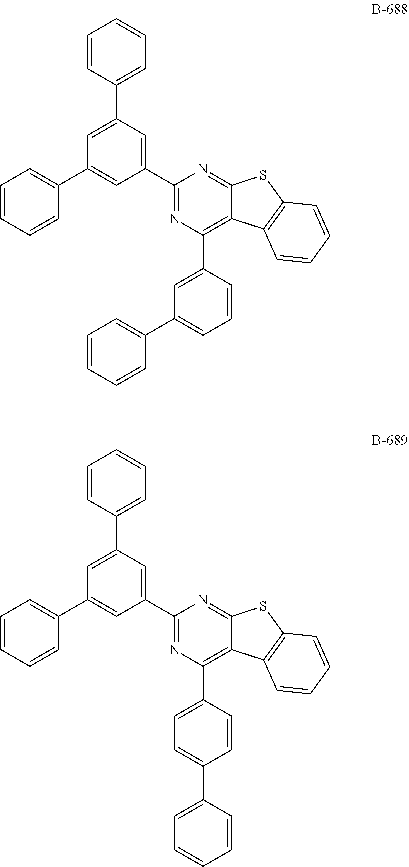 [SMILES: c1ccc(-c2cc(-c3ccccc3)cc(-c3nc(-c4cccc(-c5ccccc5)c4)c4c(n3)sc3ccccc34)c2)cc1 ; c1ccc(-c2ccc(-c3nc(-c4cc(-c5ccccc5)cc(-c5ccccc5)c4)nc4sc5ccccc5c34)cc2)cc1]

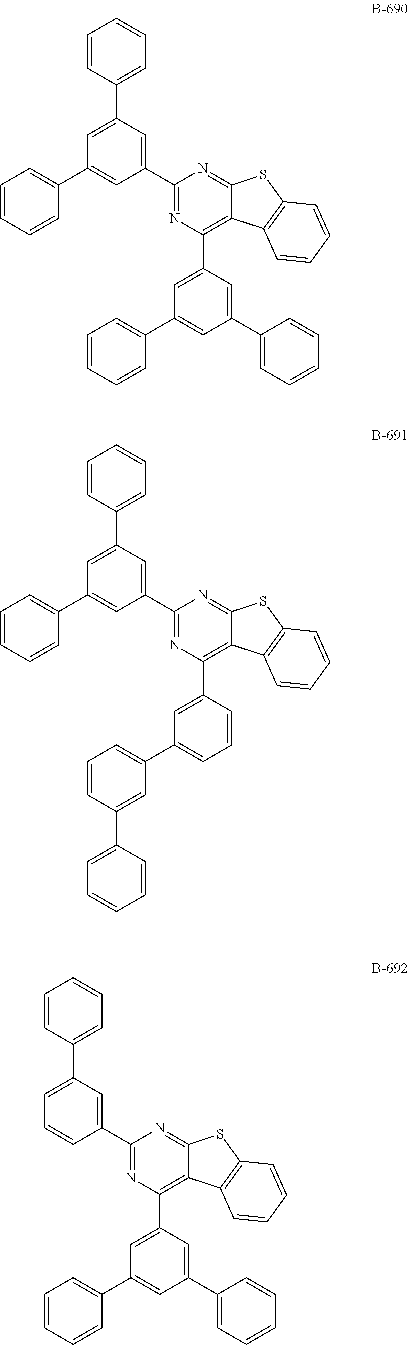 [SMILES: c1ccc(-c2cc(-c3ccccc3)cc(-c3nc(-c4cc(-c5ccccc5)cc(-c5ccccc5)c4)c4c(n3)sc3ccccc34)c2)cc1 ; c1ccc(-c2cccc(-c3cccc(-c4nc(-c5cc(-c6ccccc6)cc(-c6ccccc6)c5)nc5sc6ccccc6c45)c3)c2)cc1 ; c1ccc(-c2cccc(-c3nc(-c4cc(-c5ccccc5)cc(-c5ccccc5)c4)c4c(n3)sc3ccccc34)c2)cc1]

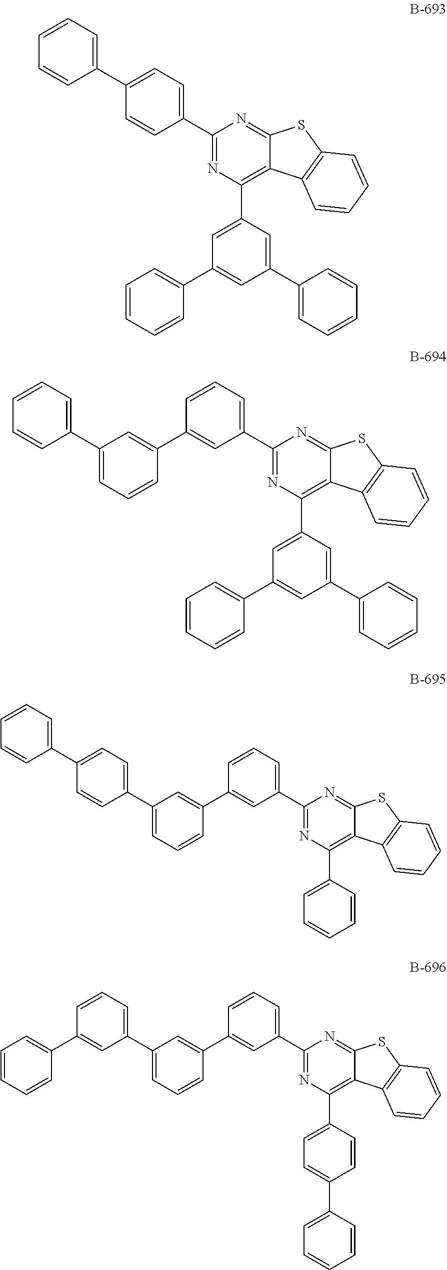 [SMILES: c1ccc(-c2ccc(-c3nc(-c4cc(-c5ccccc5)cc(-c5ccccc5)c4)c4c(n3)sc3ccccc34)cc2)cc1 ; c1ccc(-c2cccc(-c3cccc(-c4nc(-c5cc(-c6ccccc6)cc(-c6ccccc6)c5)c5c(n4)sc4ccccc45)c3)c2)cc1 ; c1ccc(-c2ccc(-c3cccc(-c4cccc(-c5nc(-c6ccccc6)c6c(n5)sc5ccccc56)c4)c3)cc2)cc1 ; c1ccc(-c2ccc(-c3nc(-c4cccc(-c5cccc(-c6cccc(-c7ccccc7)c6)c5)c4)nc4sc5ccccc5c34)cc2)cc1]

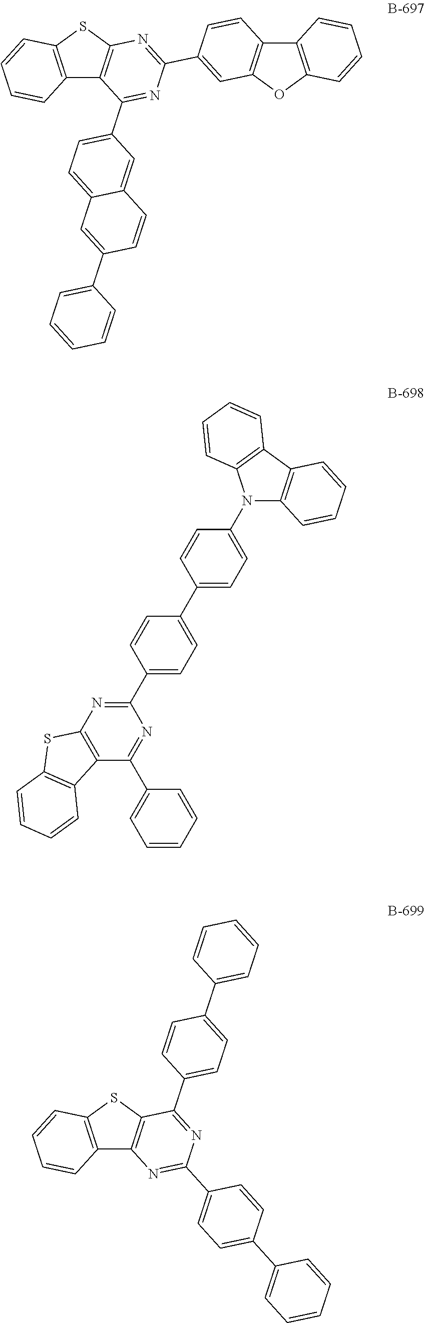 [SMILES: c1ccc(-c2ccc3cc(-c4nc(-c5ccc6c(c5)oc5ccccc56)nc5sc6ccccc6c45)ccc3c2)cc1 ; c1ccc(-c2nc(-c3ccc(-c4ccc(-n5c6ccccc6c6ccccc65)cc4)cc3)nc3sc4ccccc4c23)cc1 ; c1ccc(-c2ccc(-c3nc(-c4ccc(-c5ccccc5)cc4)c4sc5ccccc5c4n3)cc2)cc1]

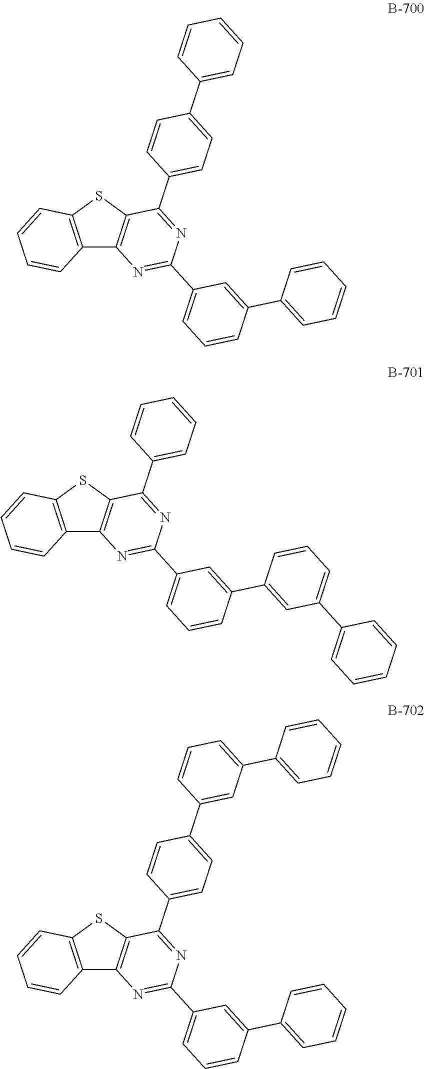 [SMILES: c1ccc(-c2ccc(-c3nc(-c4cccc(-c5ccccc5)c4)nc4c3sc3ccccc34)cc2)cc1 ; c1ccc(-c2cccc(-c3cccc(-c4nc(-c5ccccc5)c5sc6ccccc6c5n4)c3)c2)cc1 ; c1ccc(-c2cccc(-c3ccc(-c4nc(-c5cccc(-c6ccccc6)c5)nc5c4sc4ccccc45)cc3)c2)cc1]

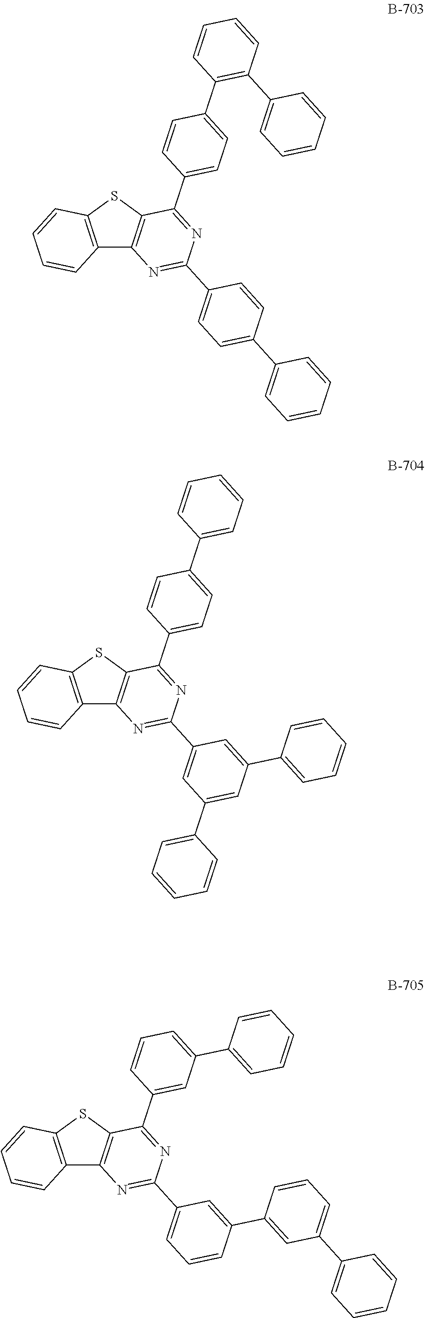 [SMILES: c1ccc(-c2ccc(-c3nc(-c4ccc(-c5ccccc5-c5ccccc5)cc4)c4sc5ccccc5c4n3)cc2)cc1 ; c1ccc(-c2ccc(-c3nc(-c4cc(-c5ccccc5)cc(-c5ccccc5)c4)nc4c3sc3ccccc34)cc2)cc1 ; c1ccc(-c2cccc(-c3cccc(-c4nc(-c5cccc(-c6ccccc6)c5)c5sc6ccccc6c5n4)c3)c2)cc1]

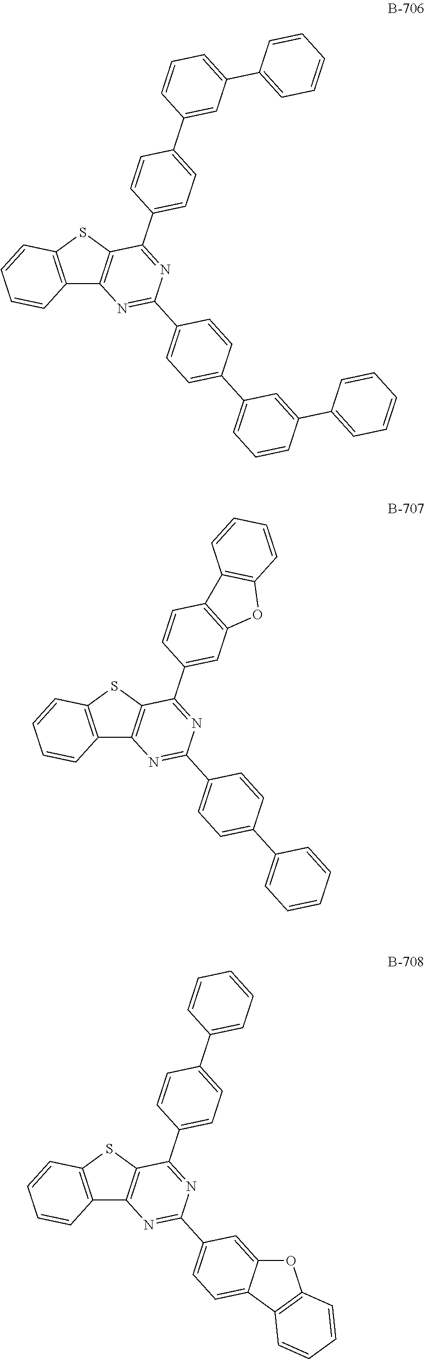 [SMILES: c1ccc(-c2cccc(-c3ccc(-c4nc(-c5ccc(-c6cccc(-c7ccccc7)c6)cc5)c5sc6ccccc6c5n4)cc3)c2)cc1 ; c1ccc(-c2ccc(-c3nc(-c4ccc5c(c4)oc4ccccc45)c4sc5ccccc5c4n3)cc2)cc1 ; c1ccc(-c2ccc(-c3nc(-c4ccc5c(c4)oc4ccccc45)nc4c3sc3ccccc34)cc2)cc1]

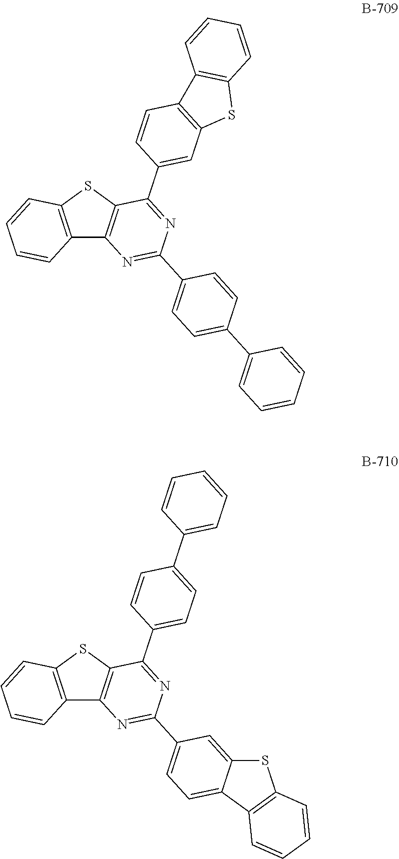 [SMILES: c1ccc(-c2ccc(-c3nc(-c4ccc5c(c4)sc4ccccc45)c4sc5ccccc5c4n3)cc2)cc1 ; c1ccc(-c2ccc(-c3nc(-c4ccc5c(c4)sc4ccccc45)nc4c3sc3ccccc34)cc2)cc1]

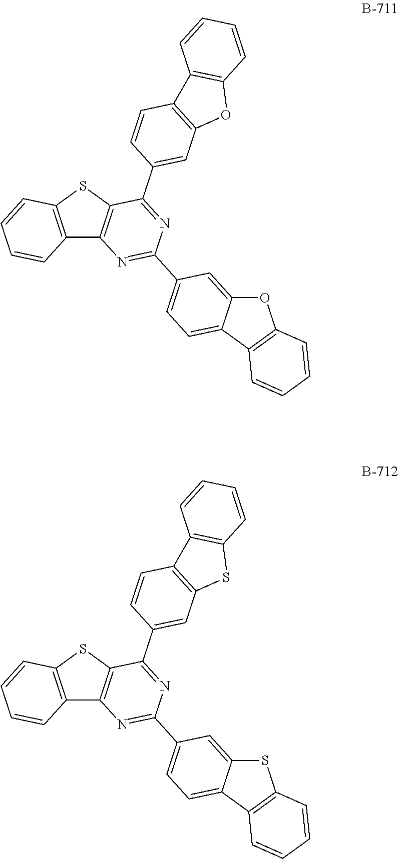 [SMILES: c1ccc2c(c1)oc1cc(-c3nc(-c4ccc5c(c4)oc4ccccc45)c4sc5ccccc5c4n3)ccc12 ; c1ccc2c(c1)sc1cc(-c3nc(-c4ccc5c(c4)sc4ccccc45)c4sc5ccccc5c4n3)ccc12]

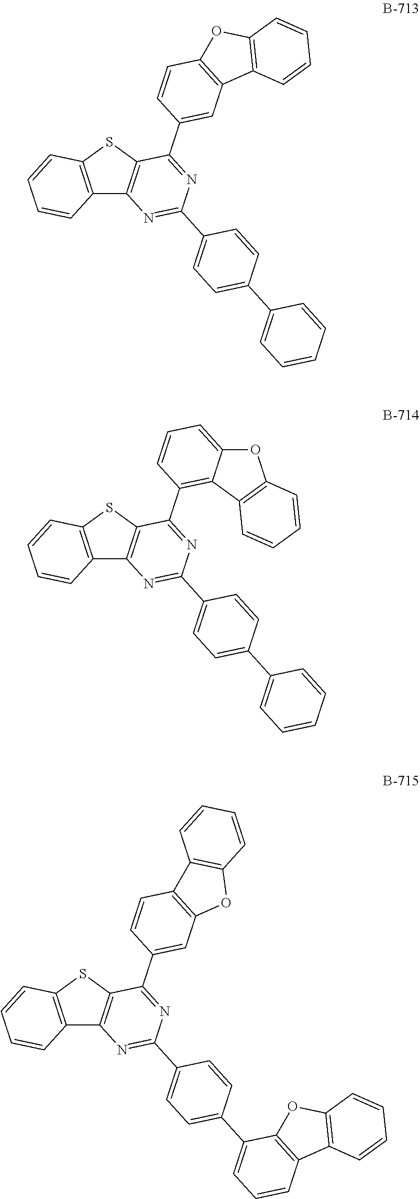 [SMILES: c1ccc(-c2ccc(-c3nc(-c4ccc5oc6ccccc6c5c4)c4sc5ccccc5c4n3)cc2)cc1 ; c1ccc(-c2ccc(-c3nc(-c4cccc5oc6ccccc6c45)c4sc5ccccc5c4n3)cc2)cc1 ; c1ccc2c(c1)oc1cc(-c3nc(-c4ccc(-c5cccc6c5oc5ccccc56)cc4)nc4c3sc3ccccc34)ccc12]

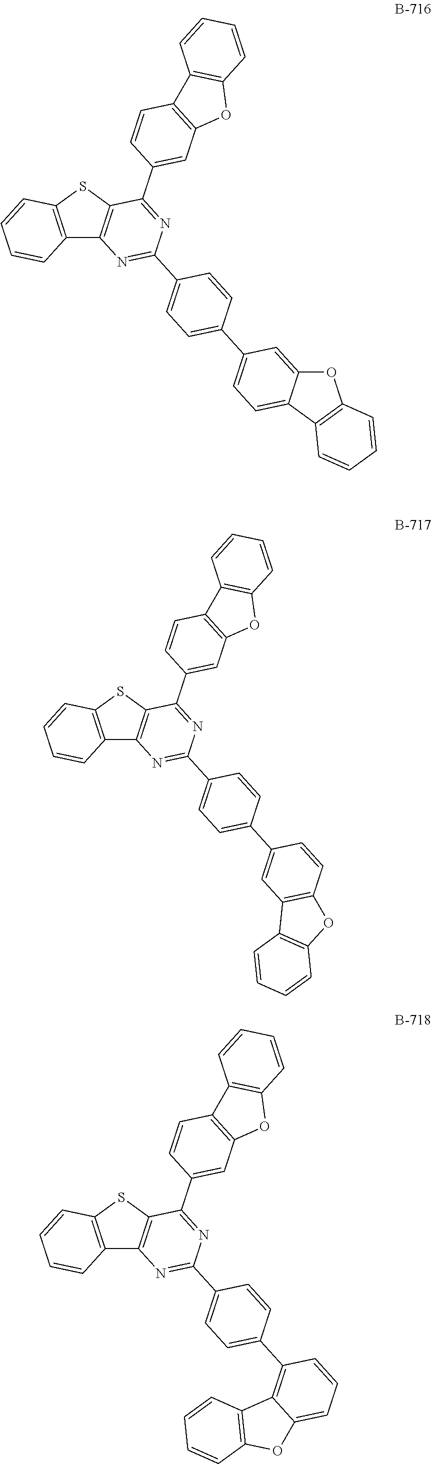

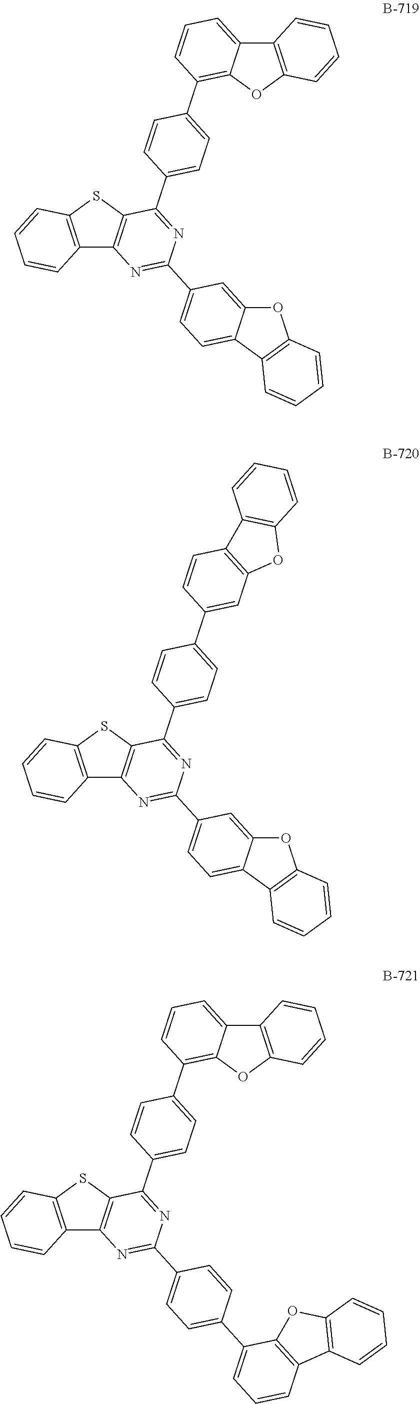 [SMILES: c1ccc2c(c1)oc1cc(-c3nc(-c4ccc(-c5cccc6c5oc5ccccc56)cc4)c4sc5ccccc5c4n3)ccc12 ; c1ccc2c(c1)oc1cc(-c3ccc(-c4nc(-c5ccc6c(c5)oc5ccccc56)nc5c4sc4ccccc45)cc3)ccc12 ; c1ccc2c(c1)oc1c(-c3ccc(-c4nc(-c5ccc(-c6cccc7c6oc6ccccc67)cc5)c5sc6ccccc6c5n4)cc3)cccc12]

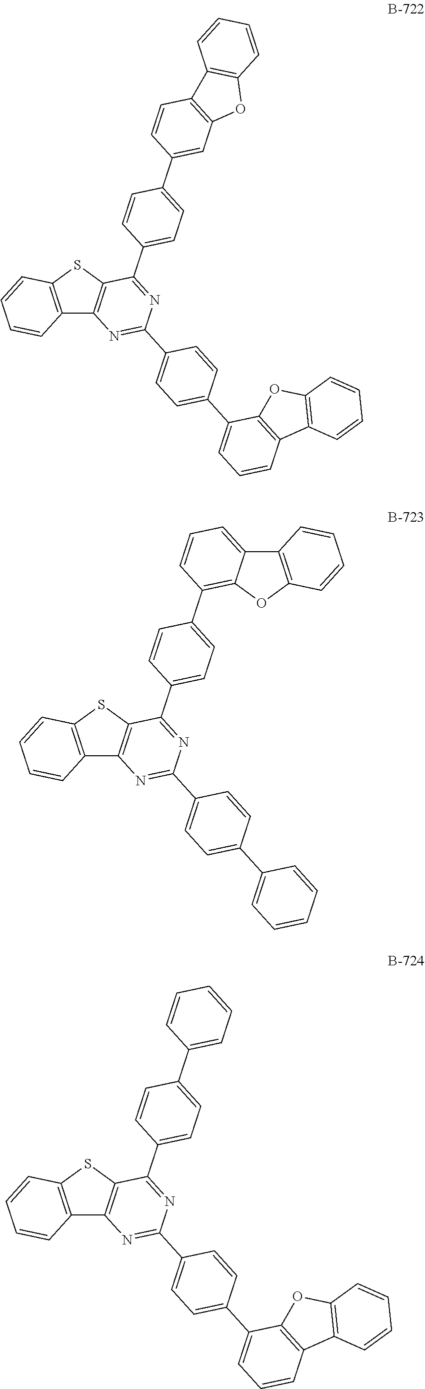 [SMILES: c1ccc2c(c1)oc1cc(-c3ccc(-c4nc(-c5ccc(-c6cccc7c6oc6ccccc67)cc5)nc5c4sc4ccccc45)cc3)ccc12 ; c1ccc(-c2ccc(-c3nc(-c4ccc(-c5cccc6c5oc5ccccc56)cc4)c4sc5ccccc5c4n3)cc2)cc1 ; c1ccc(-c2ccc(-c3nc(-c4ccc(-c5cccc6c5oc5ccccc56)cc4)nc4c3sc3ccccc34)cc2)cc1]

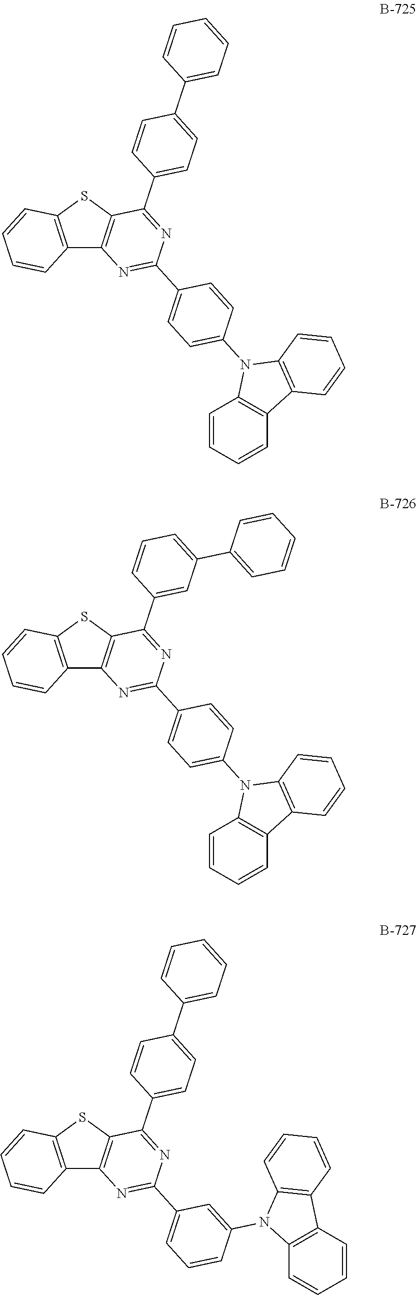 [SMILES: c1ccc(-c2ccc(-c3nc(-c4ccc(-n5c6ccccc6c6ccccc65)cc4)nc4c3sc3ccccc34)cc2)cc1 ; c1ccc(-c2cccc(-c3nc(-c4ccc(-n5c6ccccc6c6ccccc65)cc4)nc4c3sc3ccccc34)c2)cc1 ; c1ccc(-c2ccc(-c3nc(-c4cccc(-n5c6ccccc6c6ccccc65)c4)nc4c3sc3ccccc34)cc2)cc1]

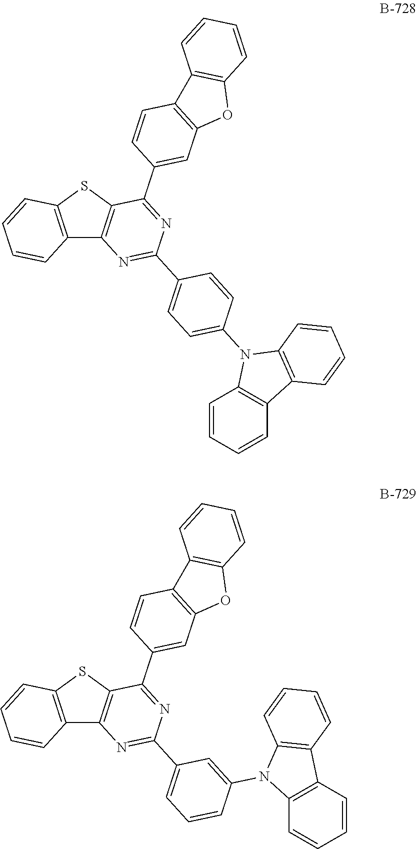 [SMILES: c1ccc2c(c1)oc1cc(-c3nc(-c4ccc(-n5c6ccccc6c6ccccc65)cc4)nc4c3sc3ccccc34)ccc12 ; c1cc(-c2nc(-c3ccc4c(c3)oc3ccccc34)c3sc4ccccc4c3n2)cc(-n2c3ccccc3c3ccccc32)c1]

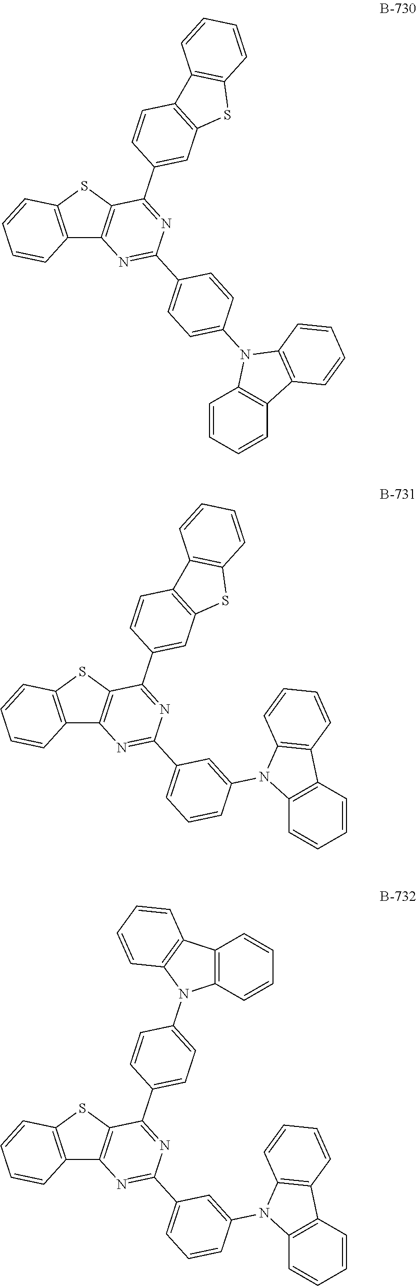 [SMILES: c1ccc2c(c1)sc1cc(-c3nc(-c4ccc(-n5c6ccccc6c6ccccc65)cc4)nc4c3sc3ccccc34)ccc12 ; c1cc(-c2nc(-c3ccc4c(c3)sc3ccccc34)c3sc4ccccc4c3n2)cc(-n2c3ccccc3c3ccccc32)c1 ; c1cc(-c2nc(-c3ccc(-n4c5ccccc5c5ccccc54)cc3)c3sc4ccccc4c3n2)cc(-n2c3ccccc3c3ccccc32)c1]

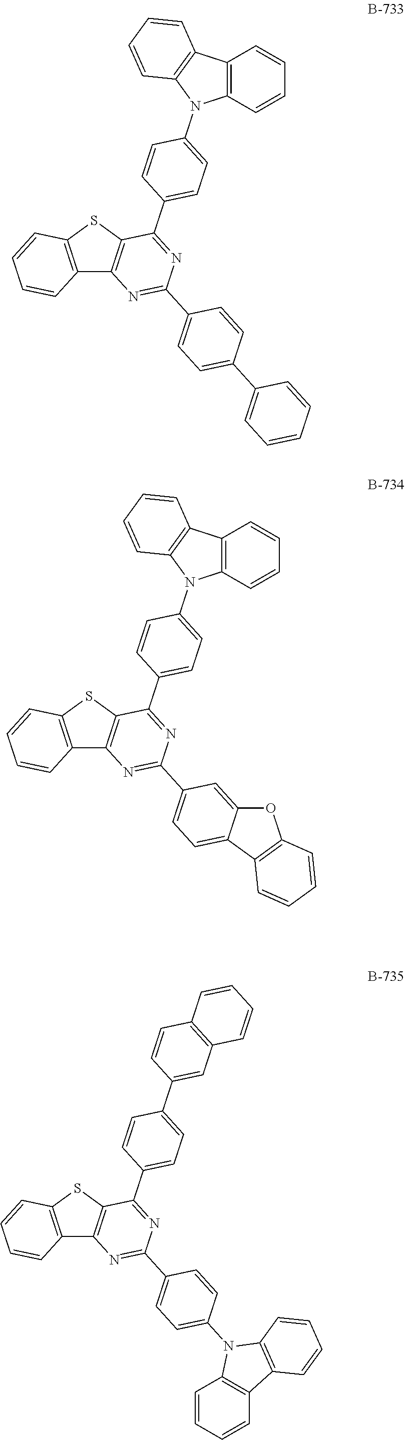 [SMILES: c1ccc(-c2ccc(-c3nc(-c4ccc(-n5c6ccccc6c6ccccc65)cc4)c4sc5ccccc5c4n3)cc2)cc1 ; c1ccc2c(c1)oc1cc(-c3nc(-c4ccc(-n5c6ccccc6c6ccccc65)cc4)c4sc5ccccc5c4n3)ccc12 ; c1ccc2cc(-c3ccc(-c4nc(-c5ccc(-n6c7ccccc7c7ccccc76)cc5)nc5c4sc4ccccc45)cc3)ccc2c1]

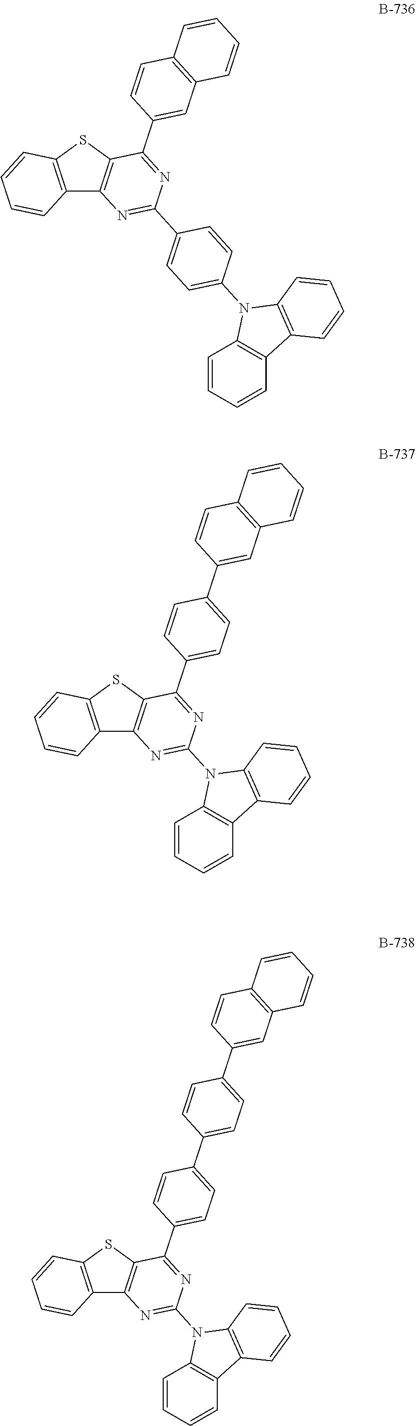 [SMILES: c1ccc2cc(-c3nc(-c4ccc(-n5c6ccccc6c6ccccc65)cc4)nc4c3sc3ccccc34)ccc2c1 ; c1ccc2cc(-c3ccc(-c4nc(-n5c6ccccc6c6ccccc65)nc5c4sc4ccccc45)cc3)ccc2c1 ; c1ccc2cc(-c3ccc(-c4ccc(-c5nc(-n6c7ccccc7c7ccccc76)nc6c5sc5ccccc56)cc4)cc3)ccc2c1]

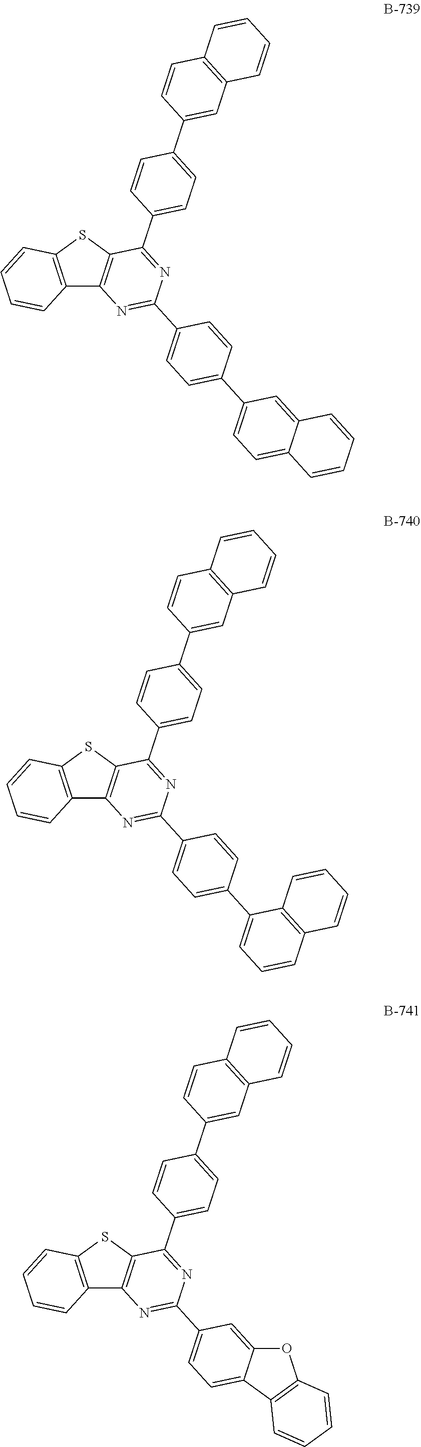 [SMILES: c1ccc2cc(-c3ccc(-c4nc(-c5ccc(-c6ccc7ccccc7c6)cc5)c5sc6ccccc6c5n4)cc3)ccc2c1 ; c1ccc2cc(-c3ccc(-c4nc(-c5ccc(-c6cccc7ccccc67)cc5)nc5c4sc4ccccc45)cc3)ccc2c1 ; c1ccc2cc(-c3ccc(-c4nc(-c5ccc6c(c5)oc5ccccc56)nc5c4sc4ccccc45)cc3)ccc2c1]

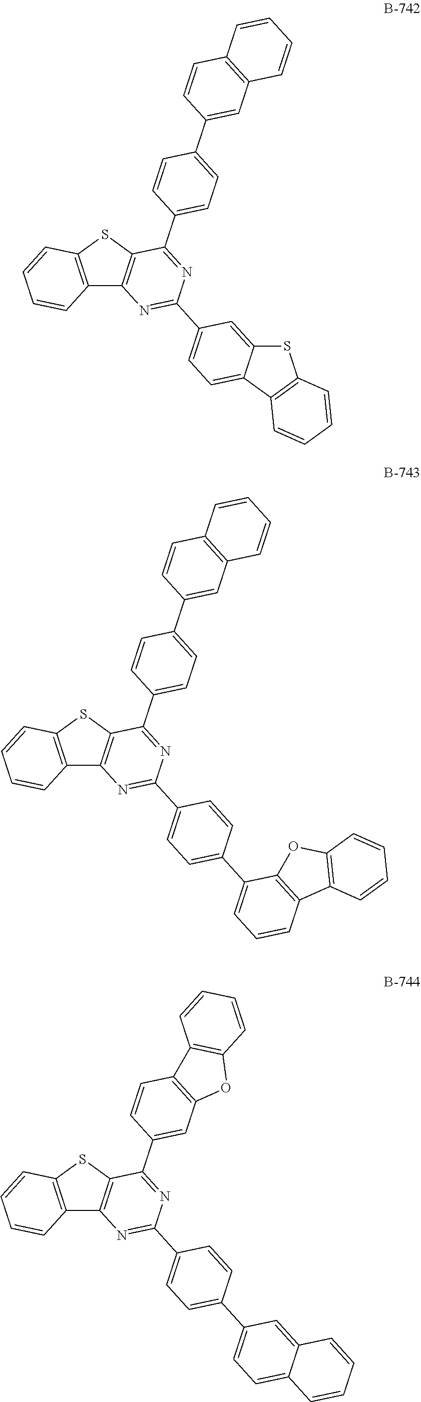 [SMILES: c1ccc2cc(-c3ccc(-c4nc(-c5ccc6c(c5)sc5ccccc56)nc5c4sc4ccccc45)cc3)ccc2c1 ; c1ccc2cc(-c3ccc(-c4nc(-c5ccc(-c6cccc7c6oc6ccccc67)cc5)nc5c4sc4ccccc45)cc3)ccc2c1 ; c1ccc2cc(-c3ccc(-c4nc(-c5ccc6c(c5)oc5ccccc56)c5sc6ccccc6c5n4)cc3)ccc2c1]

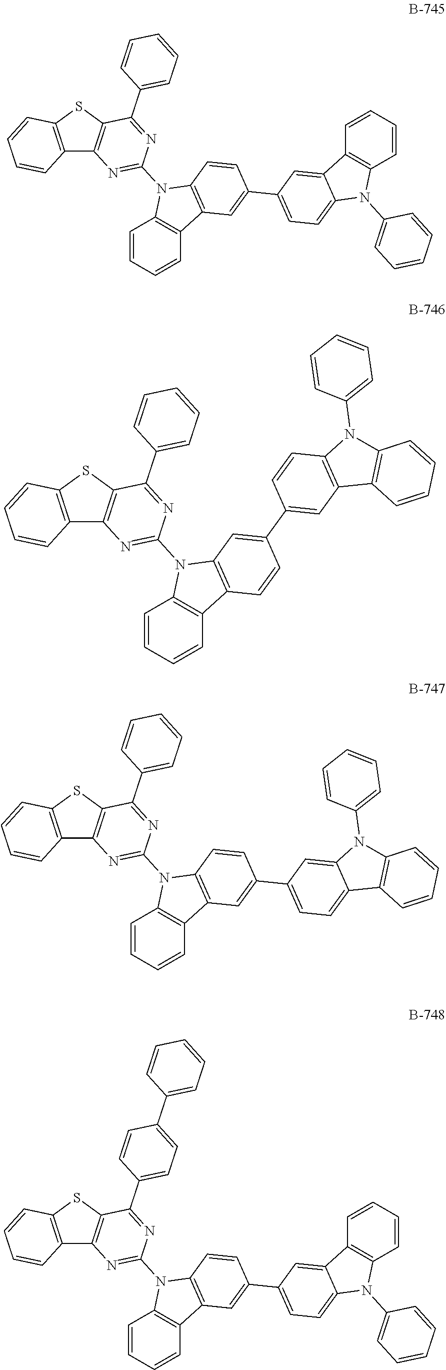 [SMILES: c1ccc(-c2nc(-n3c4ccccc4c4cc(-c5ccc6c(c5)c5ccccc5n6-c5ccccc5)ccc43)nc3c2sc2ccccc23)cc1 ; c1ccc(-c2nc(-n3c4ccccc4c4ccc(-c5ccc6c(c5)c5ccccc5n6-c5ccccc5)cc43)nc3c2sc2ccccc23)cc1 ; c1ccc(-c2nc(-n3c4ccccc4c4cc(-c5ccc6c7ccccc7n(-c7ccccc7)c6c5)ccc43)nc3c2sc2ccccc23)cc1 ; c1ccc(-c2ccc(-c3nc(-n4c5ccccc5c5cc(-c6ccc7c(c6)c6ccccc6n7-c6ccccc6)ccc54)nc4c3sc3ccccc34)cc2)cc1]

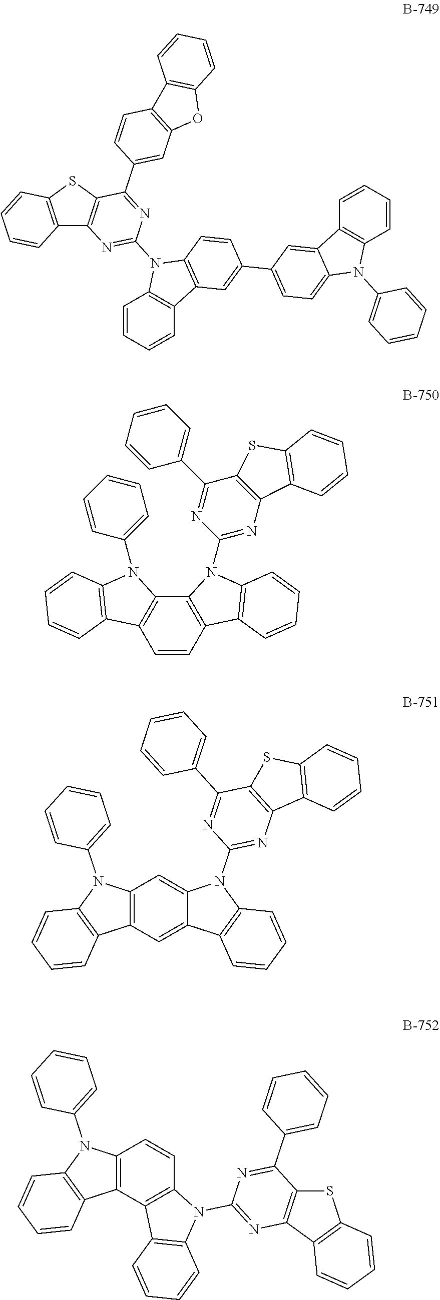 [SMILES: c1ccc(-n2c3ccccc3c3cc(-c4ccc5c(c4)c4ccccc4n5-c4nc(-c5ccc6c(c5)oc5ccccc56)c5sc6ccccc6c5n4)ccc32)cc1 ; c1ccc(-c2nc(-n3c4ccccc4c4ccc5c6ccccc6n(-c6ccccc6)c5c43)nc3c2sc2ccccc23)cc1 ; c1ccc(-c2nc(-n3c4ccccc4c4cc5c6ccccc6n(-c6ccccc6)c5cc43)nc3c2sc2ccccc23)cc1 ; c1ccc(-c2nc(-n3c4ccccc4c4c5c6ccccc6n(-c6ccccc6)c5ccc43)nc3c2sc2ccccc23)cc1]

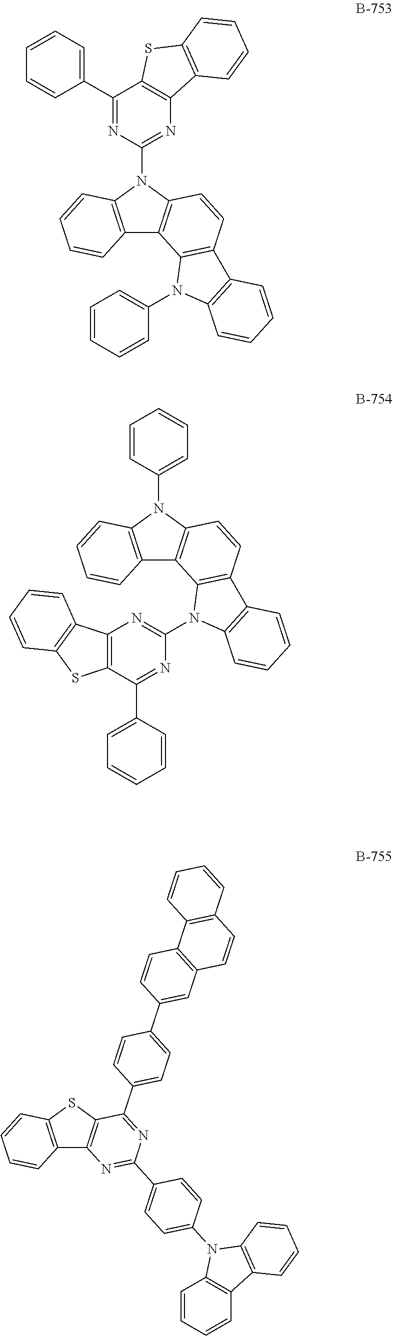 [SMILES: c1ccc(-c2nc(-n3c4ccccc4c4c3ccc3c5ccccc5n(-c5ccccc5)c34)nc3c2sc2ccccc23)cc1 ; c1ccc(-c2nc(-n3c4ccccc4c4ccc5c(c6ccccc6n5-c5ccccc5)c43)nc3c2sc2ccccc23)cc1 ; c1ccc2c(c1)ccc1cc(-c3ccc(-c4nc(-c5ccc(-n6c7ccccc7c7ccccc76)cc5)nc5c4sc4ccccc45)cc3)ccc12]

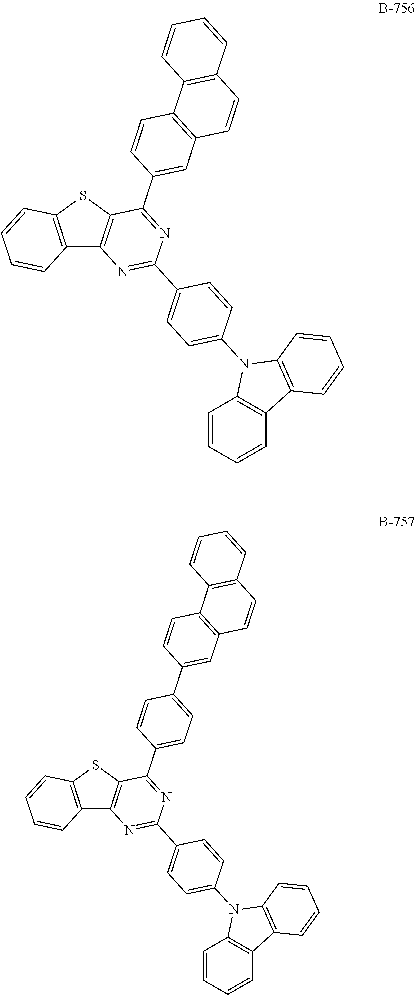 [SMILES: c1ccc2c(c1)ccc1cc(-c3nc(-c4ccc(-n5c6ccccc6c6ccccc65)cc4)nc4c3sc3ccccc34)ccc12 ; c1ccc2c(c1)ccc1cc(-c3ccc(-c4nc(-c5ccc(-n6c7ccccc7c7ccccc76)cc5)nc5c4sc4ccccc45)cc3)ccc12]

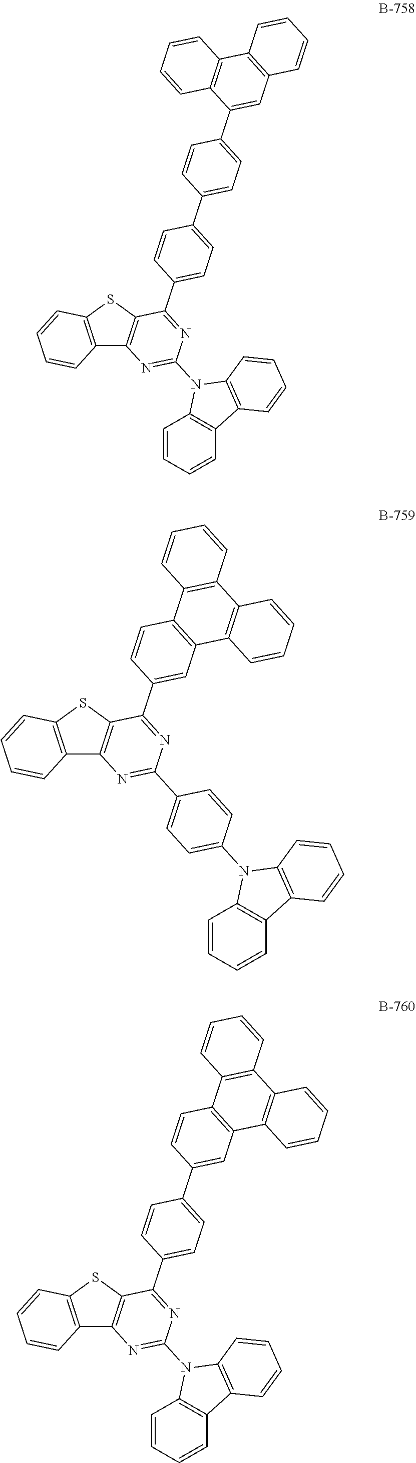 [SMILES: c1ccc2c(c1)cc(-c1ccc(-c3ccc(-c4nc(-n5c6ccccc6c6ccccc65)nc5c4sc4ccccc45)cc3)cc1)c1ccccc12 ; c1ccc2c(c1)sc1c(-c3ccc4c5ccccc5c5ccccc5c4c3)nc(-c3ccc(-n4c5ccccc5c5ccccc54)cc3)nc12 ; c1ccc2c(c1)sc1c(-c3ccc(-c4ccc5c6ccccc6c6ccccc6c5c4)cc3)nc(-n3c4ccccc4c4ccccc43)nc12]

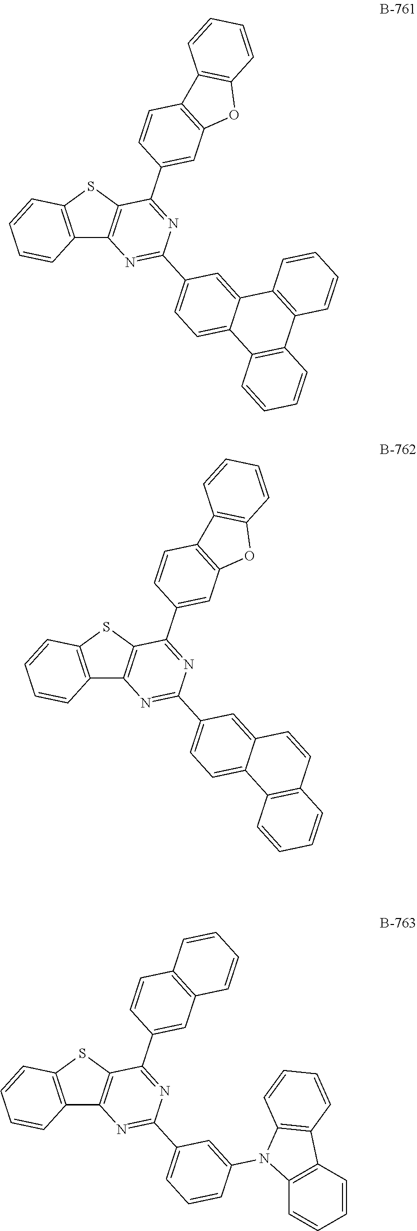 [SMILES: c1ccc2c(c1)oc1cc(-c3nc(-c4ccc5c6ccccc6c6ccccc6c5c4)nc4c3sc3ccccc34)ccc12 ; c1ccc2c(c1)ccc1cc(-c3nc(-c4ccc5c(c4)oc4ccccc45)c4sc5ccccc5c4n3)ccc12 ; c1cc(-c2nc(-c3ccc4ccccc4c3)c3sc4ccccc4c3n2)cc(-n2c3ccccc3c3ccccc32)c1]

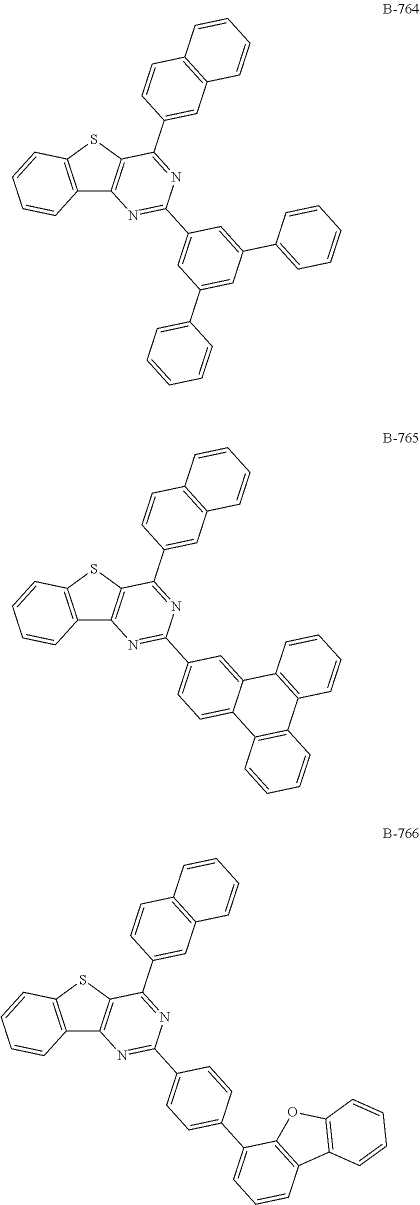 [SMILES: c1ccc(-c2cc(-c3ccccc3)cc(-c3nc(-c4ccc5ccccc5c4)c4sc5ccccc5c4n3)c2)cc1 ; c1ccc2cc(-c3nc(-c4ccc5c6ccccc6c6ccccc6c5c4)nc4c3sc3ccccc34)ccc2c1 ; c1ccc2cc(-c3nc(-c4ccc(-c5cccc6c5oc5ccccc56)cc4)nc4c3sc3ccccc34)ccc2c1]

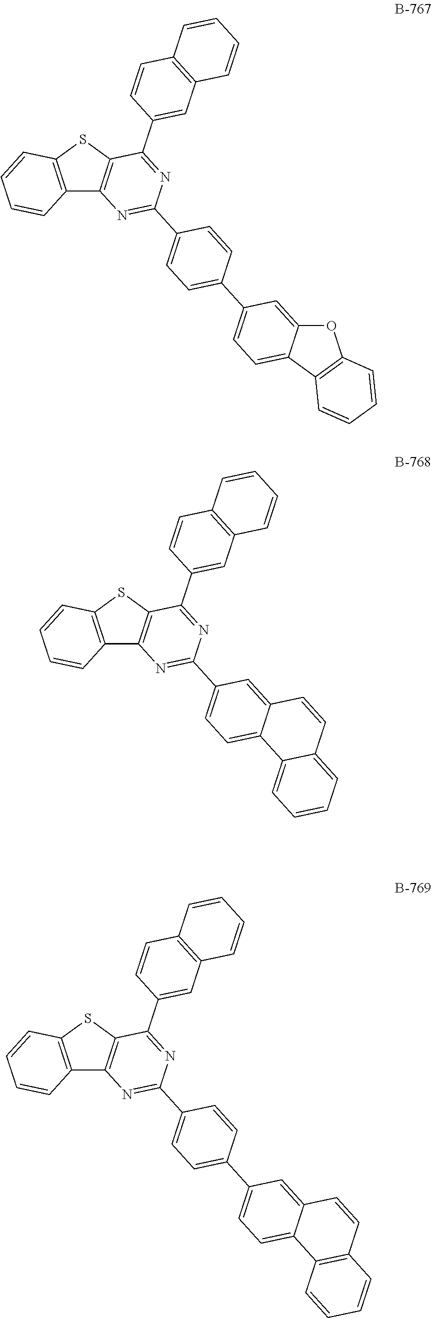 [SMILES: c1ccc2cc(-c3nc(-c4ccc(-c5ccc6c(c5)oc5ccccc56)cc4)nc4c3sc3ccccc34)ccc2c1 ; c1ccc2cc(-c3nc(-c4ccc5c(ccc6ccccc65)c4)nc4c3sc3ccccc34)ccc2c1 ; c1ccc2cc(-c3nc(-c4ccc(-c5ccc6c(ccc7ccccc76)c5)cc4)nc4c3sc3ccccc34)ccc2c1]

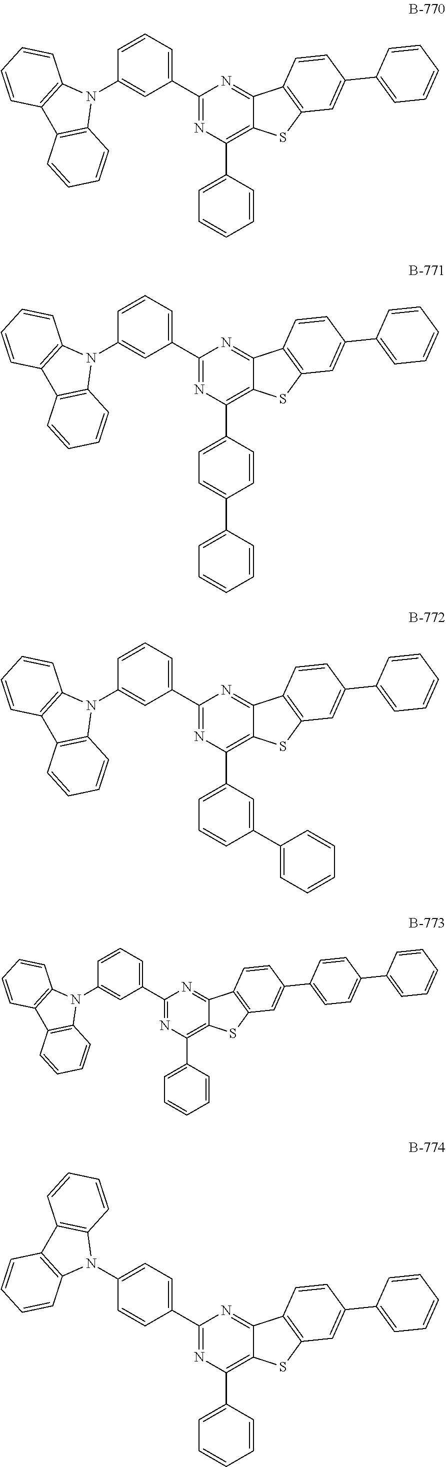 [SMILES: c1ccc(-c2ccc3c(c2)sc2c(-c4ccccc4)nc(-c4cccc(-n5c6ccccc6c6ccccc65)c4)nc23)cc1 ; c1ccc(-c2ccc(-c3nc(-c4cccc(-n5c6ccccc6c6ccccc65)c4)nc4c3sc3cc(-c5ccccc5)ccc34)cc2)cc1 ; c1ccc(-c2cccc(-c3nc(-c4cccc(-n5c6ccccc6c6ccccc65)c4)nc4c3sc3cc(-c5ccccc5)ccc34)c2)cc1 ; c1ccc(-c2ccc(-c3ccc4c(c3)sc3c(-c5ccccc5)nc(-c5cccc(-n6c7ccccc7c7ccccc76)c5)nc34)cc2)cc1 ; c1ccc(-c2ccc3c(c2)sc2c(-c4ccccc4)nc(-c4ccc(-n5c6ccccc6c6ccccc65)cc4)nc23)cc1]

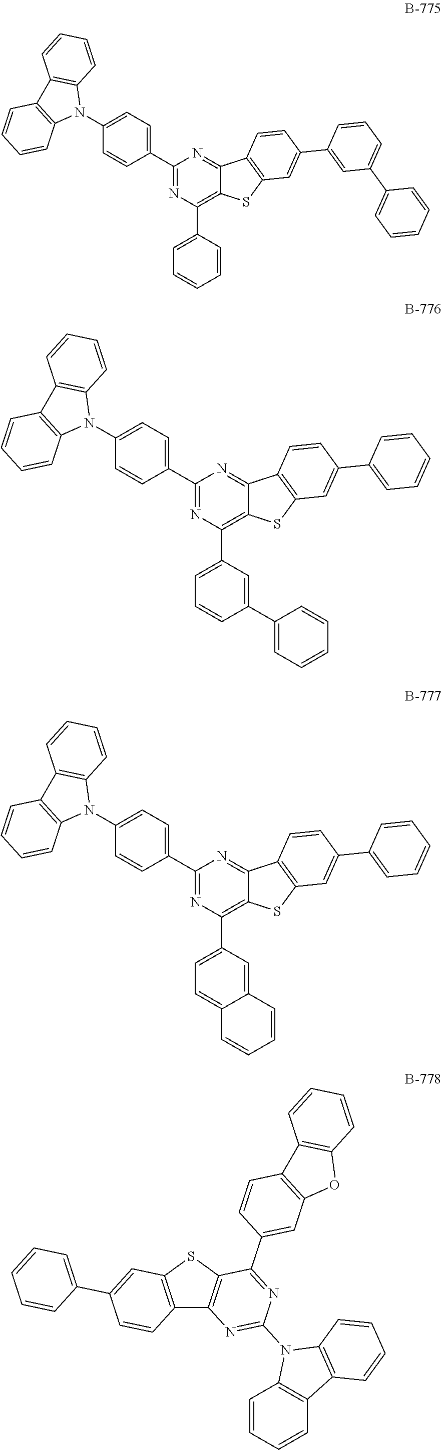 [SMILES: c1ccc(-c2cccc(-c3ccc4c(c3)sc3c(-c5ccccc5)nc(-c5ccc(-n6c7ccccc7c7ccccc76)cc5)nc34)c2)cc1 ; c1ccc(-c2cccc(-c3nc(-c4ccc(-n5c6ccccc6c6ccccc65)cc4)nc4c3sc3cc(-c5ccccc5)ccc34)c2)cc1 ; c1ccc(-c2ccc3c(c2)sc2c(-c4ccc5ccccc5c4)nc(-c4ccc(-n5c6ccccc6c6ccccc65)cc4)nc23)cc1 ; c1ccc(-c2ccc3c(c2)sc2c(-c4ccc5c(c4)oc4ccccc45)nc(-n4c5ccccc5c5ccccc54)nc23)cc1]

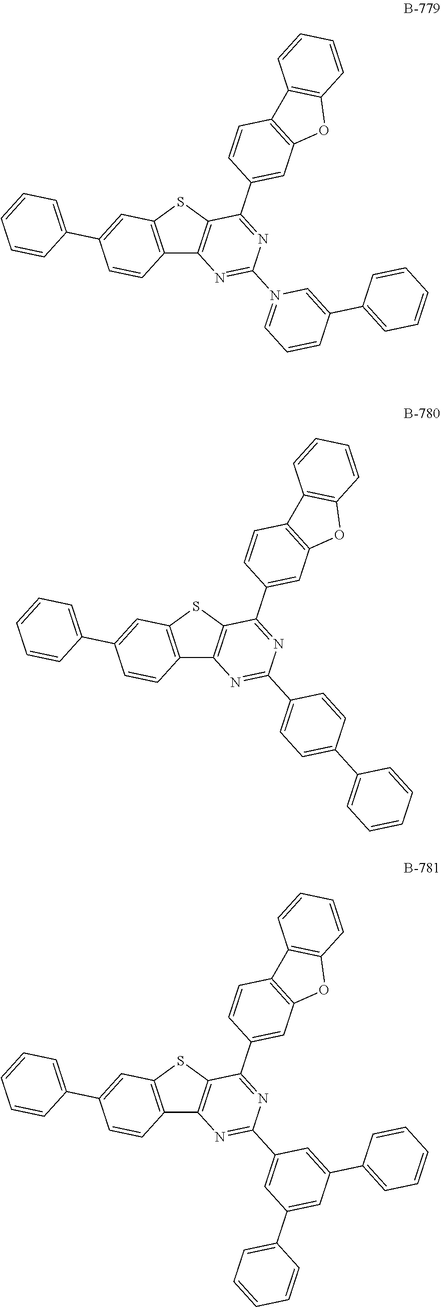

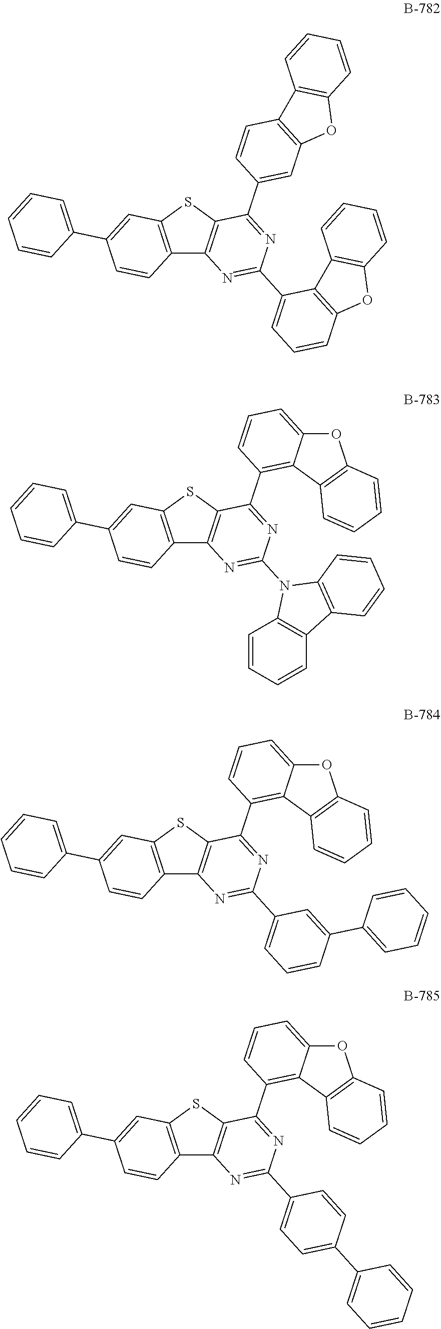 [SMILES: c1ccc(-c2ccc3c(c2)sc2c(-c4ccc5c(c4)oc4ccccc45)nc(-c4cccc5oc6ccccc6c45)nc23)cc1 ; c1ccc(-c2ccc3c(c2)sc2c(-c4cccc5oc6ccccc6c45)nc(-n4c5ccccc5c5ccccc54)nc23)cc1 ; c1ccc(-c2cccc(-c3nc(-c4cccc5oc6ccccc6c45)c4sc5cc(-c6ccccc6)ccc5c4n3)c2)cc1 ; c1ccc(-c2ccc(-c3nc(-c4cccc5oc6ccccc6c45)c4sc5cc(-c6ccccc6)ccc5c4n3)cc2)cc1]

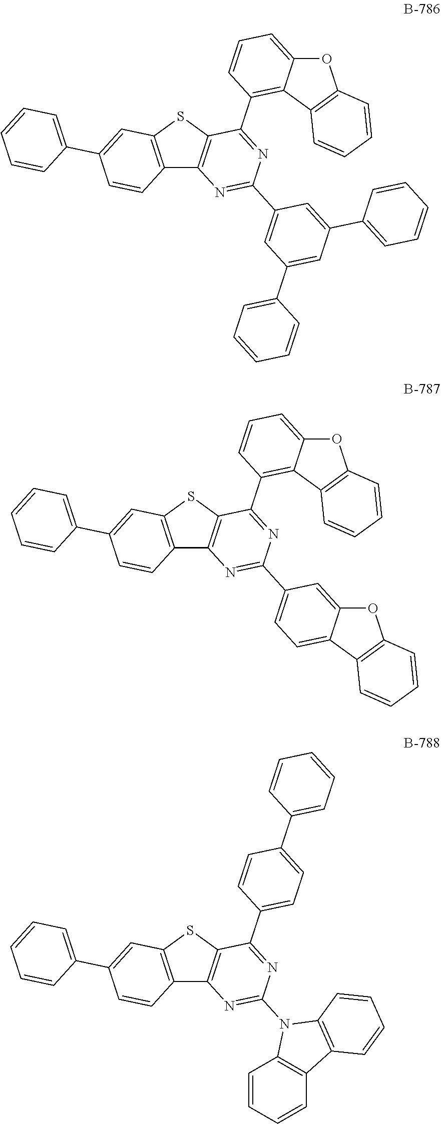 [SMILES: c1ccc(-c2cc(-c3ccccc3)cc(-c3nc(-c4cccc5oc6ccccc6c45)c4sc5cc(-c6ccccc6)ccc5c4n3)c2)cc1 ; c1ccc(-c2ccc3c(c2)sc2c(-c4cccc5oc6ccccc6c45)nc(-c4ccc5c(c4)oc4ccccc45)nc23)cc1 ; c1ccc(-c2ccc(-c3nc(-n4c5ccccc5c5ccccc54)nc4c3sc3cc(-c5ccccc5)ccc34)cc2)cc1]

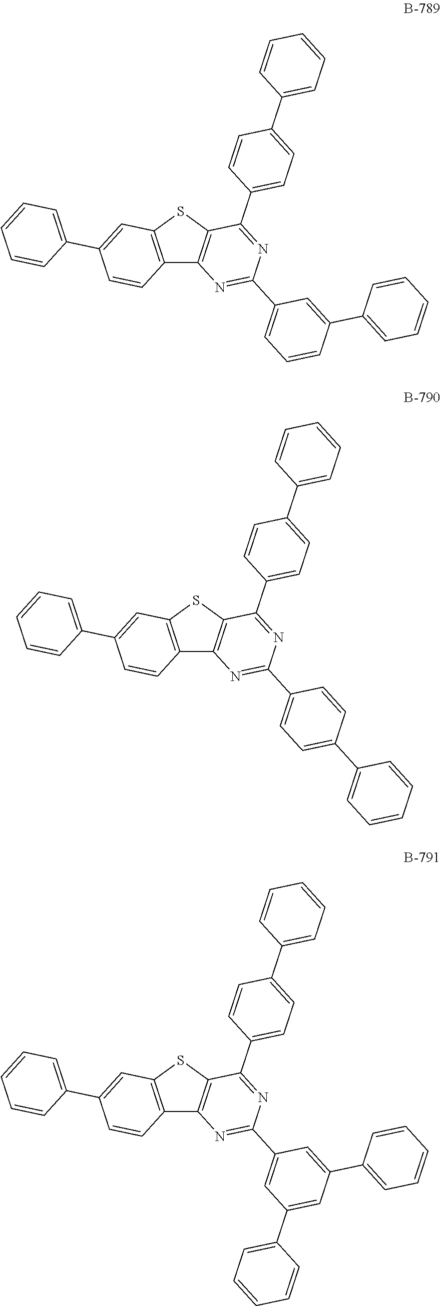 [SMILES: c1ccc(-c2ccc(-c3nc(-c4cccc(-c5ccccc5)c4)nc4c3sc3cc(-c5ccccc5)ccc34)cc2)cc1 ; c1ccc(-c2ccc(-c3nc(-c4ccc(-c5ccccc5)cc4)c4sc5cc(-c6ccccc6)ccc5c4n3)cc2)cc1 ; c1ccc(-c2ccc(-c3nc(-c4cc(-c5ccccc5)cc(-c5ccccc5)c4)nc4c3sc3cc(-c5ccccc5)ccc34)cc2)cc1]

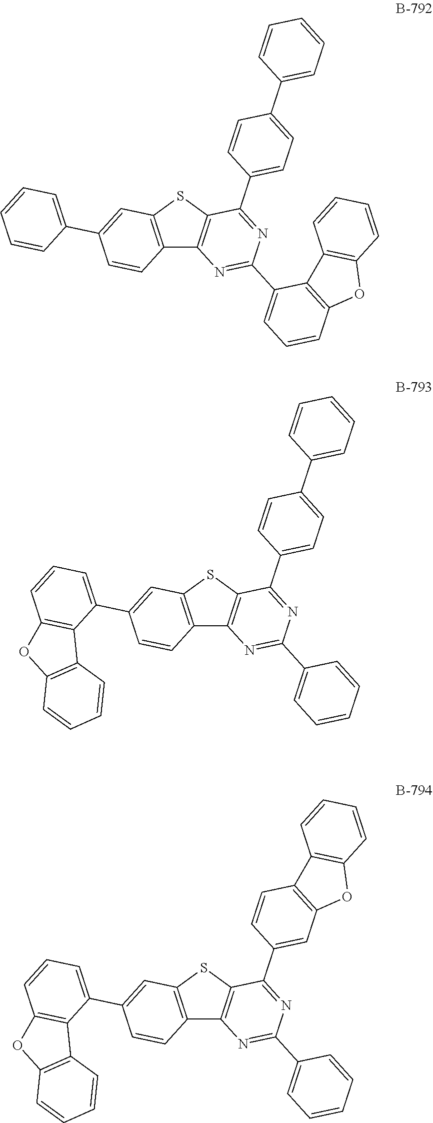 [SMILES: c1ccc(-c2ccc(-c3nc(-c4cccc5oc6ccccc6c45)nc4c3sc3cc(-c5ccccc5)ccc34)cc2)cc1 ; c1ccc(-c2ccc(-c3nc(-c4ccccc4)nc4c3sc3cc(-c5cccc6oc7ccccc7c56)ccc34)cc2)cc1 ; c1ccc(-c2nc(-c3ccc4c(c3)oc3ccccc34)c3sc4cc(-c5cccc6oc7ccccc7c56)ccc4c3n2)cc1]

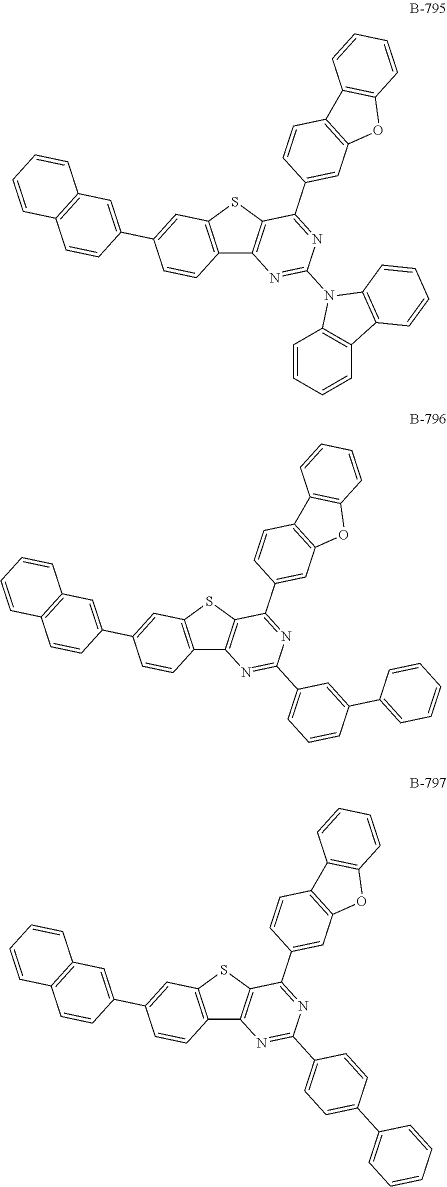 [SMILES: c1ccc2cc(-c3ccc4c(c3)sc3c(-c5ccc6c(c5)oc5ccccc56)nc(-n5c6ccccc6c6ccccc65)nc34)ccc2c1 ; c1ccc(-c2cccc(-c3nc(-c4ccc5c(c4)oc4ccccc45)c4sc5cc(-c6ccc7ccccc7c6)ccc5c4n3)c2)cc1 ; c1ccc(-c2ccc(-c3nc(-c4ccc5c(c4)oc4ccccc45)c4sc5cc(-c6ccc7ccccc7c6)ccc5c4n3)cc2)cc1]

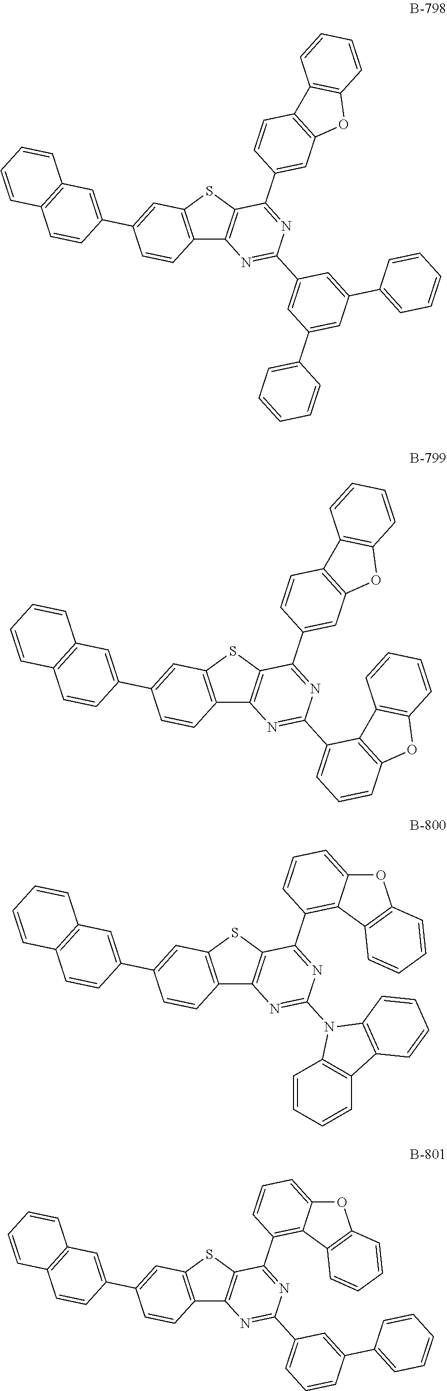 [SMILES: c1ccc(-c2cc(-c3ccccc3)cc(-c3nc(-c4ccc5c(c4)oc4ccccc45)c4sc5cc(-c6ccc7ccccc7c6)ccc5c4n3)c2)cc1 ; c1ccc2cc(-c3ccc4c(c3)sc3c(-c5ccc6c(c5)oc5ccccc56)nc(-c5cccc6oc7ccccc7c56)nc34)ccc2c1 ; c1ccc2cc(-c3ccc4c(c3)sc3c(-c5cccc6oc7ccccc7c56)nc(-n5c6ccccc6c6ccccc65)nc34)ccc2c1 ; c1ccc(-c2cccc(-c3nc(-c4cccc5oc6ccccc6c45)c4sc5cc(-c6ccc7ccccc7c6)ccc5c4n3)c2)cc1]

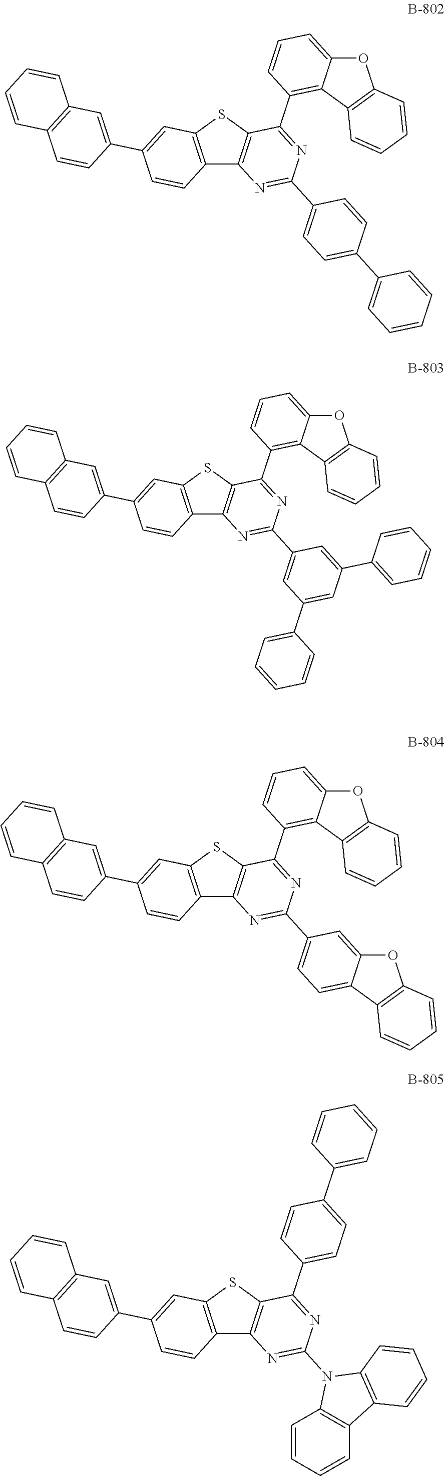 [SMILES: c1ccc(-c2ccc(-c3nc(-c4cccc5oc6ccccc6c45)c4sc5cc(-c6ccc7ccccc7c6)ccc5c4n3)cc2)cc1 ; c1ccc(-c2cc(-c3ccccc3)cc(-c3nc(-c4cccc5oc6ccccc6c45)c4sc5cc(-c6ccc7ccccc7c6)ccc5c4n3)c2)cc1 ; c1ccc2cc(-c3ccc4c(c3)sc3c(-c5cccc6oc7ccccc7c56)nc(-c5ccc6c(c5)oc5ccccc56)nc34)ccc2c1 ; c1ccc(-c2ccc(-c3nc(-n4c5ccccc5c5ccccc54)nc4c3sc3cc(-c5ccc6ccccc6c5)ccc34)cc2)cc1]

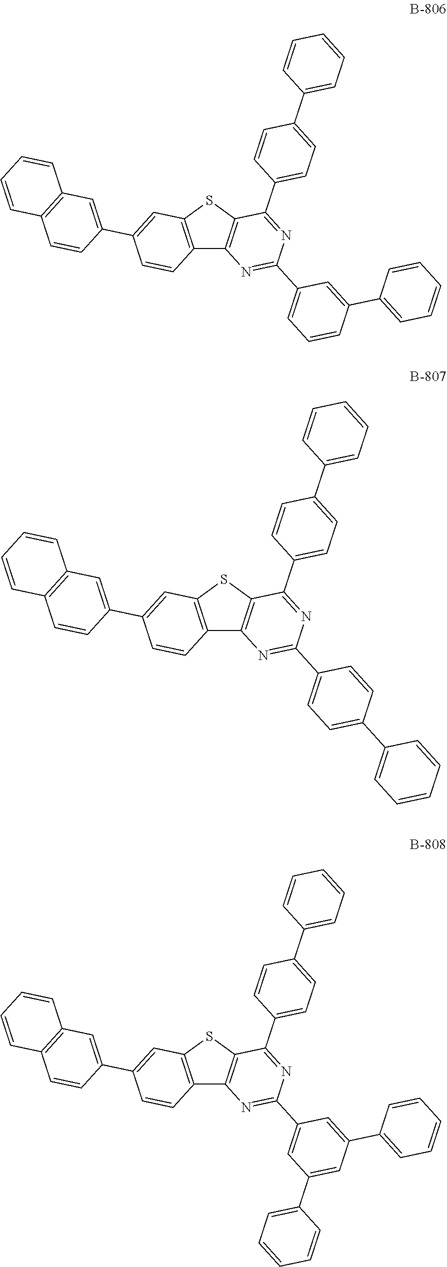 [SMILES: c1ccc(-c2ccc(-c3nc(-c4cccc(-c5ccccc5)c4)nc4c3sc3cc(-c5ccc6ccccc6c5)ccc34)cc2)cc1 ; c1ccc(-c2ccc(-c3nc(-c4ccc(-c5ccccc5)cc4)c4sc5cc(-c6ccc7ccccc7c6)ccc5c4n3)cc2)cc1 ; c1ccc(-c2ccc(-c3nc(-c4cc(-c5ccccc5)cc(-c5ccccc5)c4)nc4c3sc3cc(-c5ccc6ccccc6c5)ccc34)cc2)cc1]

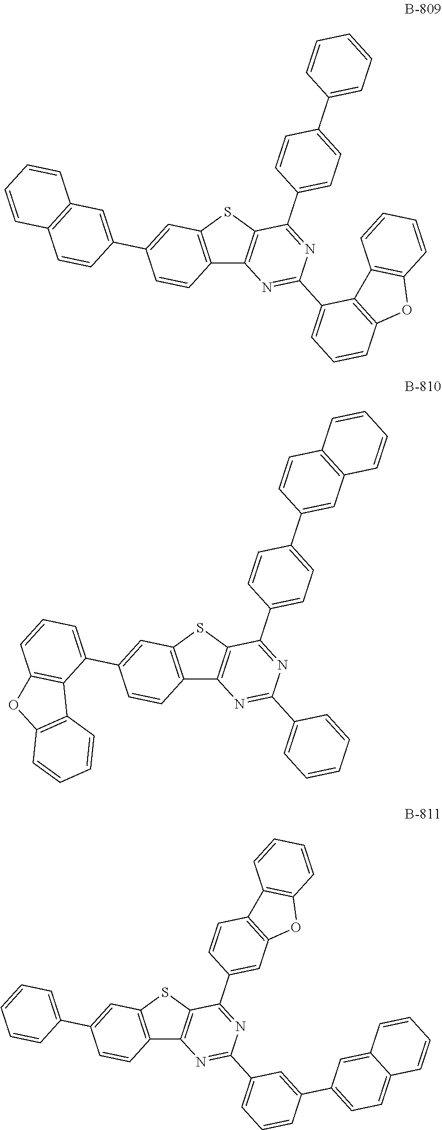 [SMILES: c1ccc(-c2ccc(-c3nc(-c4cccc5oc6ccccc6c45)nc4c3sc3cc(-c5ccc6ccccc6c5)ccc34)cc2)cc1 ; c1ccc(-c2nc(-c3ccc(-c4ccc5ccccc5c4)cc3)c3sc4cc(-c5cccc6oc7ccccc7c56)ccc4c3n2)cc1 ; c1ccc(-c2ccc3c(c2)sc2c(-c4ccc5c(c4)oc4ccccc45)nc(-c4cccc(-c5ccc6ccccc6c5)c4)nc23)cc1]

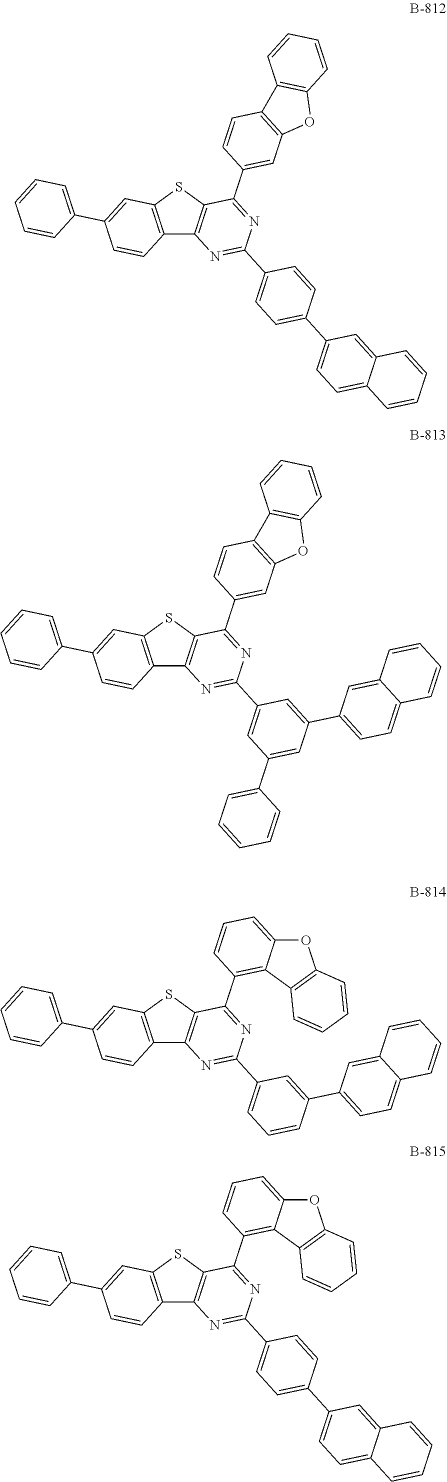

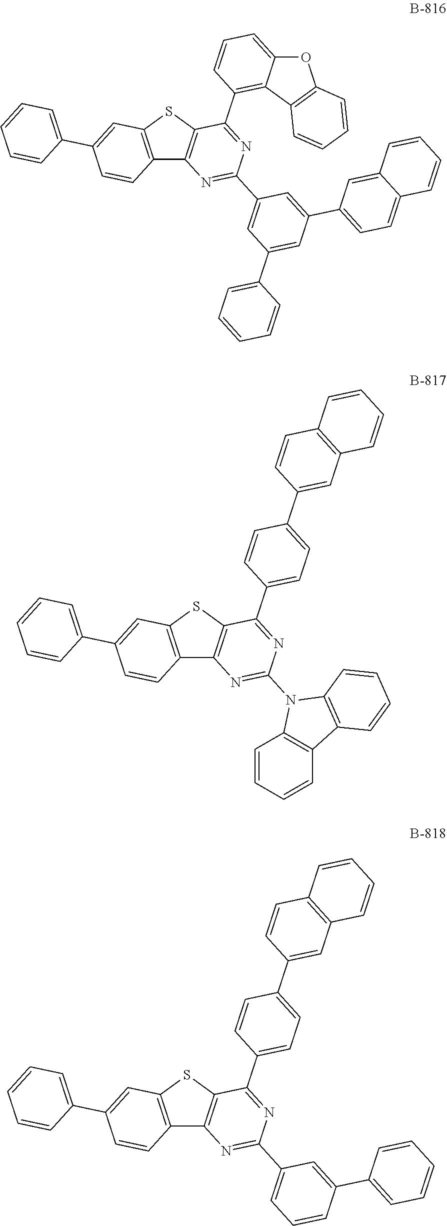 [SMILES: c1ccc(-c2cc(-c3ccc4ccccc4c3)cc(-c3nc(-c4cccc5oc6ccccc6c45)c4sc5cc(-c6ccccc6)ccc5c4n3)c2)cc1 ; c1ccc(-c2ccc3c(c2)sc2c(-c4ccc(-c5ccc6ccccc6c5)cc4)nc(-n4c5ccccc5c5ccccc54)nc23)cc1 ; c1ccc(-c2cccc(-c3nc(-c4ccc(-c5ccc6ccccc6c5)cc4)c4sc5cc(-c6ccccc6)ccc5c4n3)c2)cc1]

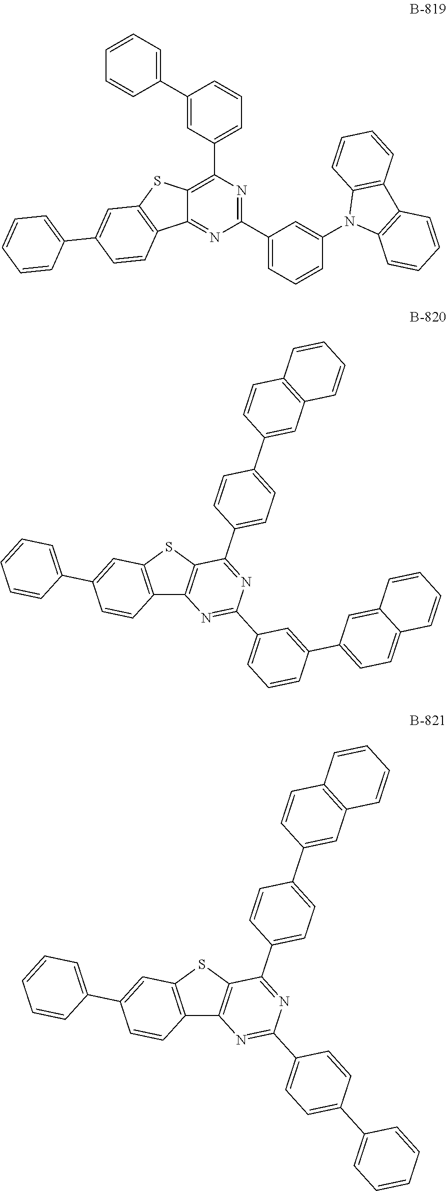 [SMILES: c1ccc(-c2cccc(-c3nc(-c4cccc(-n5c6ccccc6c6ccccc65)c4)nc4c3sc3cc(-c5ccccc5)ccc34)c2)cc1 ; c1ccc(-c2ccc3c(c2)sc2c(-c4ccc(-c5ccc6ccccc6c5)cc4)nc(-c4cccc(-c5ccc6ccccc6c5)c4)nc23)cc1 ; c1ccc(-c2ccc(-c3nc(-c4ccc(-c5ccc6ccccc6c5)cc4)c4sc5cc(-c6ccccc6)ccc5c4n3)cc2)cc1]

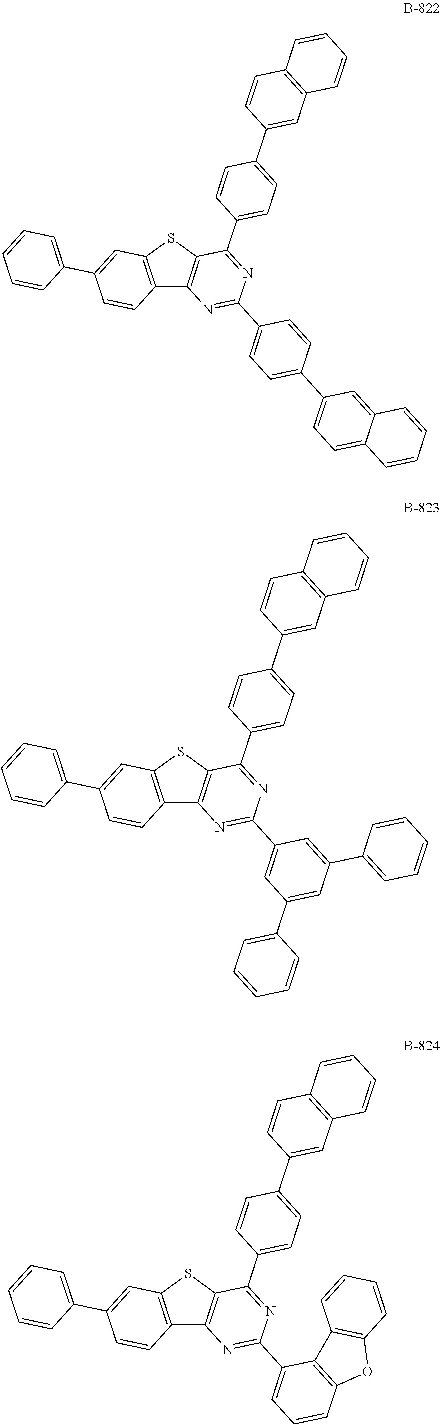 [SMILES: c1ccc(-c2ccc3c(c2)sc2c(-c4ccc(-c5ccc6ccccc6c5)cc4)nc(-c4ccc(-c5ccc6ccccc6c5)cc4)nc23)cc1 ; c1ccc(-c2cc(-c3ccccc3)cc(-c3nc(-c4ccc(-c5ccc6ccccc6c5)cc4)c4sc5cc(-c6ccccc6)ccc5c4n3)c2)cc1 ; c1ccc(-c2ccc3c(c2)sc2c(-c4ccc(-c5ccc6ccccc6c5)cc4)nc(-c4cccc5oc6ccccc6c45)nc23)cc1]

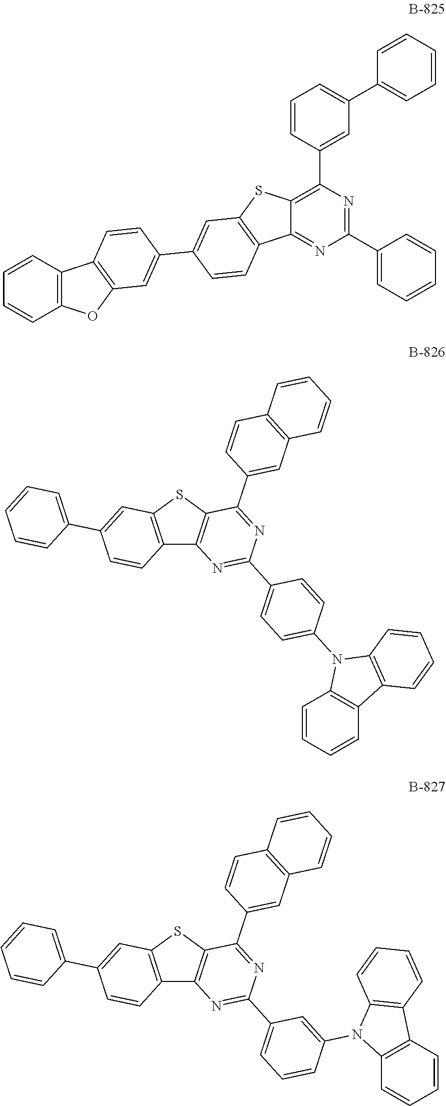 [SMILES: c1ccc(-c2cccc(-c3nc(-c4ccccc4)nc4c3sc3cc(-c5ccc6c(c5)oc5ccccc56)ccc34)c2)cc1 ; c1ccc(-c2ccc3c(c2)sc2c(-c4ccc5ccccc5c4)nc(-c4ccc(-n5c6ccccc6c6ccccc65)cc4)nc23)cc1 ; c1ccc(-c2ccc3c(c2)sc2c(-c4ccc5ccccc5c4)nc(-c4cccc(-n5c6ccccc6c6ccccc65)c4)nc23)cc1]

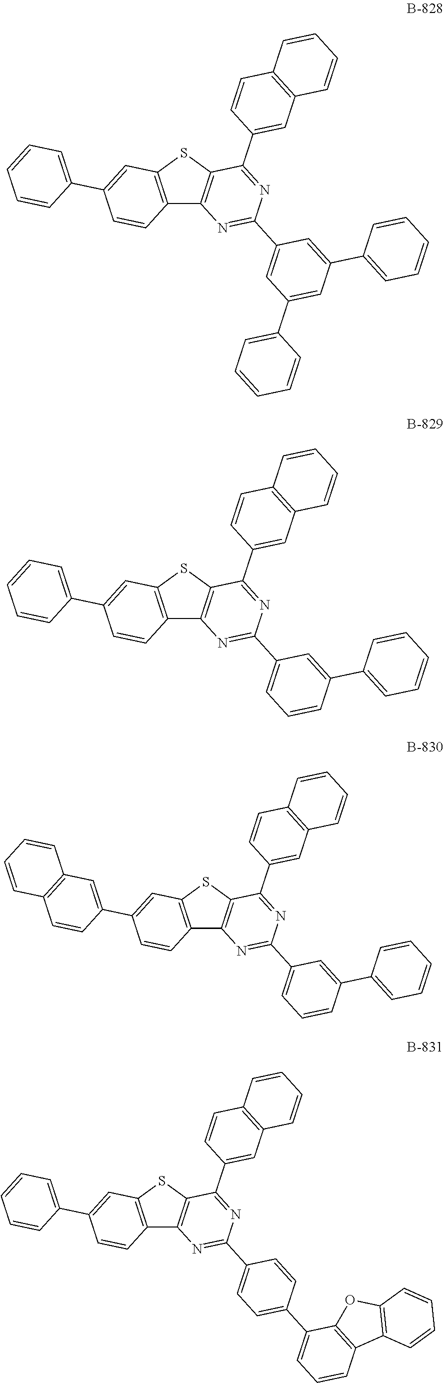 [SMILES: c1ccc(-c2cc(-c3ccccc3)cc(-c3nc(-c4ccc5ccccc5c4)c4sc5cc(-c6ccccc6)ccc5c4n3)c2)cc1 ; c1ccc(-c2cccc(-c3nc(-c4ccc5ccccc5c4)c4sc5cc(-c6ccccc6)ccc5c4n3)c2)cc1 ; c1ccc(-c2cccc(-c3nc(-c4ccc5ccccc5c4)c4sc5cc(-c6ccc7ccccc7c6)ccc5c4n3)c2)cc1 ; c1ccc(-c2ccc3c(c2)sc2c(-c4ccc5ccccc5c4)nc(-c4ccc(-c5cccc6c5oc5ccccc56)cc4)nc23)cc1]

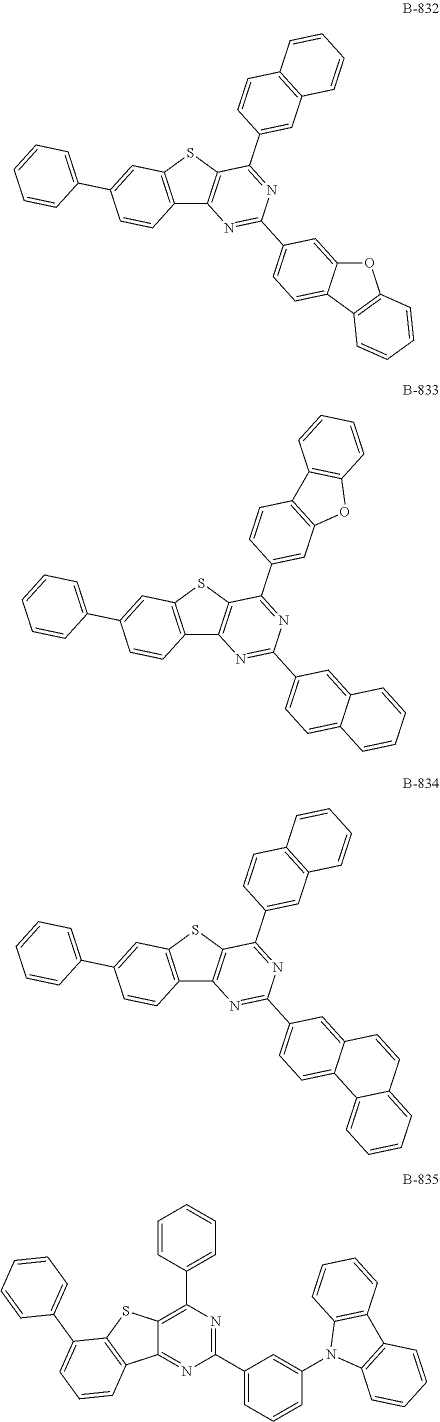 [SMILES: c1ccc(-c2ccc3c(c2)sc2c(-c4ccc5ccccc5c4)nc(-c4ccc5c(c4)oc4ccccc45)nc23)cc1 ; c1ccc(-c2ccc3c(c2)sc2c(-c4ccc5c(c4)oc4ccccc45)nc(-c4ccc5ccccc5c4)nc23)cc1 ; c1ccc(-c2ccc3c(c2)sc2c(-c4ccc5ccccc5c4)nc(-c4ccc5c(ccc6ccccc65)c4)nc23)cc1 ; c1ccc(-c2cccc3c2sc2c(-c4ccccc4)nc(-c4cccc(-n5c6ccccc6c6ccccc65)c4)nc23)cc1]

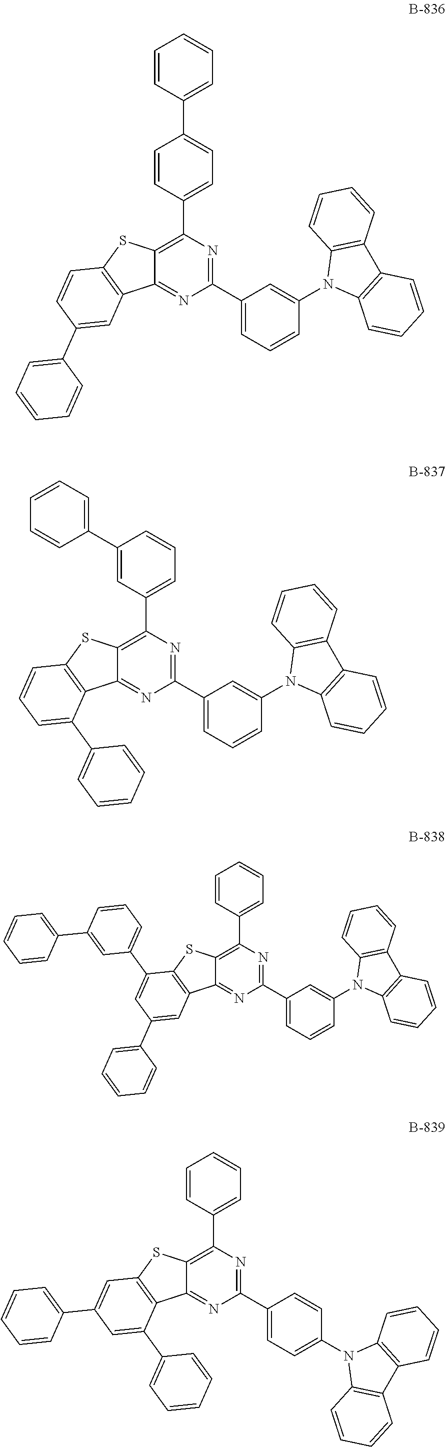 [SMILES: c1ccc(-c2ccc(-c3nc(-c4cccc(-n5c6ccccc6c6ccccc65)c4)nc4c3sc3ccc(-c5ccccc5)cc34)cc2)cc1 ; c1ccc(-c2cccc(-c3nc(-c4cccc(-n5c6ccccc6c6ccccc65)c4)nc4c3sc3cccc(-c5ccccc5)c34)c2)cc1 ; c1ccc(-c2cccc(-c3cc(-c4ccccc4)cc4c3sc3c(-c5ccccc5)nc(-c5cccc(-n6c7ccccc7c7ccccc76)c5)nc34)c2)cc1 ; c1ccc(-c2cc(-c3ccccc3)c3c(c2)sc2c(-c4ccccc4)nc(-c4ccc(-n5c6ccccc6c6ccccc65)cc4)nc23)cc1]

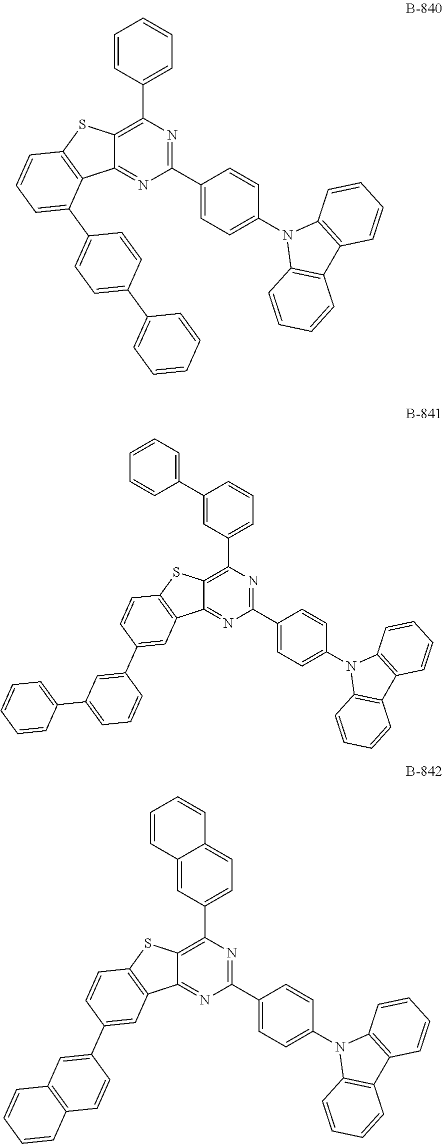 [SMILES: c1ccc(-c2ccc(-c3cccc4sc5c(-c6ccccc6)nc(-c6ccc(-n7c8ccccc8c8ccccc87)cc6)nc5c34)cc2)cc1 ; c1ccc(-c2cccc(-c3ccc4sc5c(-c6cccc(-c7ccccc7)c6)nc(-c6ccc(-n7c8ccccc8c8ccccc87)cc6)nc5c4c3)c2)cc1 ; c1ccc2cc(-c3ccc4sc5c(-c6ccc7ccccc7c6)nc(-c6ccc(-n7c8ccccc8c8ccccc87)cc6)nc5c4c3)ccc2c1]

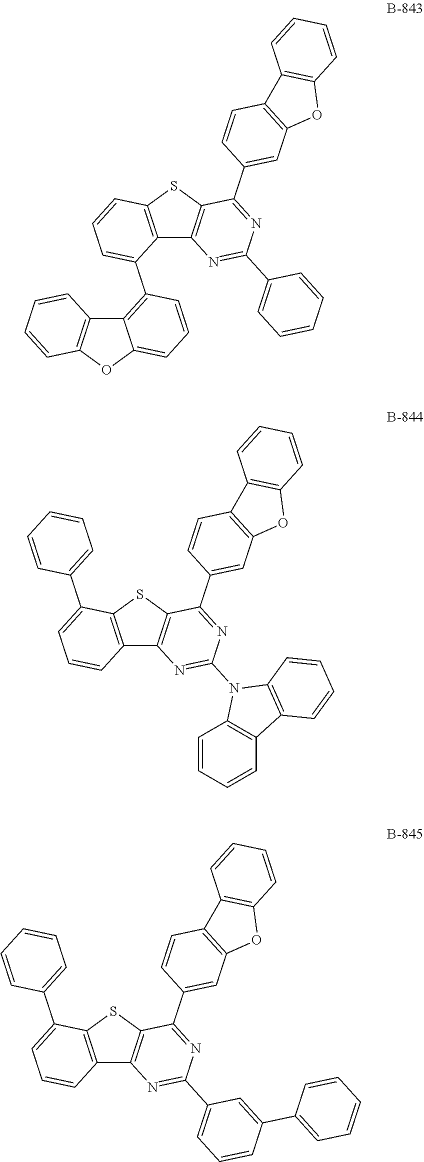 [SMILES: c1ccc(-c2nc(-c3ccc4c(c3)oc3ccccc34)c3sc4cccc(-c5cccc6oc7ccccc7c56)c4c3n2)cc1 ; c1ccc(-c2cccc3c2sc2c(-c4ccc5c(c4)oc4ccccc45)nc(-n4c5ccccc5c5ccccc54)nc23)cc1 ; c1ccc(-c2cccc(-c3nc(-c4ccc5c(c4)oc4ccccc45)c4sc5c(-c6ccccc6)cccc5c4n3)c2)cc1]

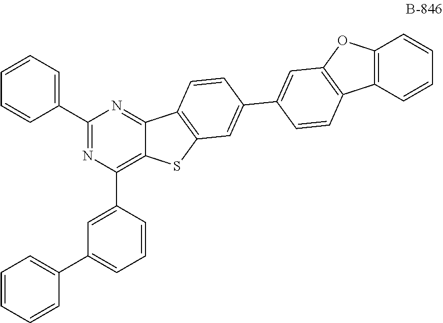 [SMILES: c1ccc(-c2cccc(-c3nc(-c4ccccc4)nc4c3sc3cc(-c5ccc6c(c5)oc5ccccc56)ccc34)c2)cc1]

The first compound for the organic optoelectronic element and the second compound for the organic optoelectronic element may be included in a weight ratio of 1:99 to 99:1. Within the range, a desirable weight ratio may be adjusted using a hole transport capability of the first compound for the organic optoelectronic element and an electron transport capability of the second compound for the organic optoelectronic element to realize bipolar characteristics and thus to improve efficiency and life-span. Within the range, they may be for example included in a weight ratio of about 10:90 to 90:10, about 20:80 to 80:20, about 30:70 to 70:30, about 40:60 to 60:40 or about 50:50. For example, they may be included in a weight ratio of 50:50 to 60:40, for example, 50:50 or 60:40.

For example, the composition for an organic optoelectronic element according to an embodiment of the present invention may include the compound represented by Chemical Formula 1A-1-b as the first compound for an organic optoelectronic element and the compound represented by Chemical Formula 2-Ias a second compound for an organic optoelectronic element.

For example, in Chemical Formula 1A-1-b, Ar may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted dibenzofuranyl group, a substituted or unsubstituted dibenzothiphenyl group, a substituted or unsubstituted carbazolyl group, or a combination thereof, L^{a}, L^{b}, L^{c}, and L¹ to L⁴ may independently be a single bond, a substituted or unsubstituted phenylene group, a substituted or unsubstituted biphenylene group, a substituted or unsubstituted terphenylene group, or a substituted or unsubstituted naphthylene group, R^{a}, R¹, R², and R⁴ may be independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof, and R^{b} and R^{c} may independently be a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, or a substituted or unsubstituted dibenzothiophenyl group,

in Chemical Formula 2-I, X may be O or S, L⁵ and L⁶ may independently be a single bond, a substituted or unsubstituted C6 to C20 arylene group, a substituted or unsubstituted C2 to C20 heterocyclic group, or a combination thereof, R⁵ and R⁶ are independently a substituted or unsubstituted phenyl group, a substituted or unsubstituted biphenyl group, a substituted or unsubstituted naphthyl group, a substituted or unsubstituted dibenzofuranyl group, a substituted or unsubstituted dibenzothiophenyl group, a substituted or unsubstituted carbazolyl group, or a combination thereof, and R⁷ to R¹⁰ may independently be hydrogen, deuterium, a substituted or unsubstituted C1 to C10 alkyl group, a substituted or unsubstituted C6 to C12 aryl group, or a substituted or unsubstituted C6 to C12 heterocyclic group.

The composition for an organic optoelectronic element may further include one or more compounds in addition to the first compound for an organic optoelectronic element and the second compound for an organic optoelectronic element.

The composition for an organic optoelectronic element may further include a dopant. The dopant may be, for example, a phosphorescent dopant, such as a red, green or blue phosphorescent dopant, and may be, for example, a red phosphorescent dopant.

The dopant is a material mixed with the first compound for an organic optoelectronic element and the second compound for an organic optoelectronic element in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L⁷MX^{a} [Chemical Formula Z]

In Chemical Formula Z, M is a metal, and L⁷ and X^{a} are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and the L⁷ and X^{a} may be, for example, a bidendate ligand.

The aforementioned composition may be formed by a dry film formation method such as chemical vapor deposition (CVD).

Hereinafter, an organic optoelectronic element including the aforementioned compound for an organic optoelectronic element or composition for an organic optoelectronic element is described.

The organic optoelectronic element may be any device to convert electrical energy into photoenergy and vice versa without particular limitation, and may be, for example an organic photoelectric device, an organic light emitting diode, an organic solar cell, and an organic photo-conductor drum.

Herein, an organic light emitting diode as one example of an organic optoelectronic element is described referring to drawings.

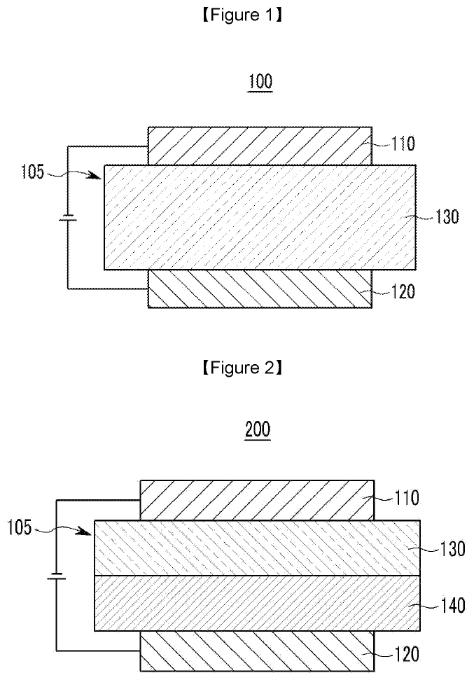

FIGS. 1 and 2 are cross-sectional views showing organic light emitting diodes according to embodiments.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 includes the light emitting layer 130 including the aforementioned composition.

The light emitting layer 130 may include, for example, the aforementioned composition.

The aforementioned composition may be, for example, a red light-emitting composition.

The light emitting layer 130 may include, for example, the first compound for an organic optoelectronic element and the second compound for an organic optoelectronic element, respectively, as a phosphorescent host.

Referring to FIG. 2, the organic light emitting diode 200 further includes a hole auxiliary layer 140 in addition to the light emitting layer 130. The hole auxiliary layer 140 may further increase hole injection and/or hole mobility and block electrons between the anode 120 and the light emitting layer 130. The hole auxiliary layer 140 may be, for example, a hole transport layer, a hole injection layer, and/or an electron blocking layer, and may include at least one layer.

The hole auxiliary layer 140 may include, for example, at least one of the compounds of Group E.

Specifically, the hole auxiliary layer 140 may include a hole transport layer between the anode 120 and the light emitting layer 130, and a hole transport auxiliary layer between the light emitting layer 130 and the hole transport layer and at least one of the compounds of Group E may be included in the hole transport auxiliary layer.

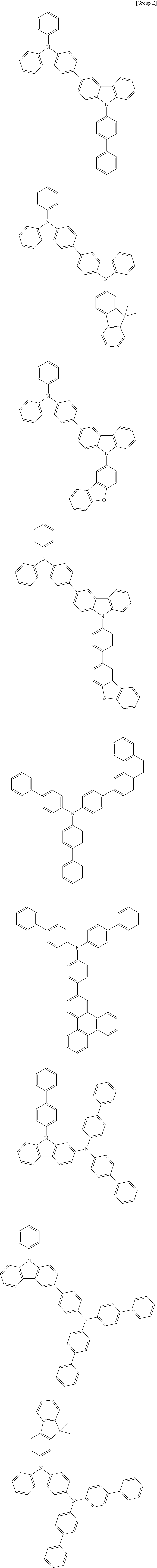

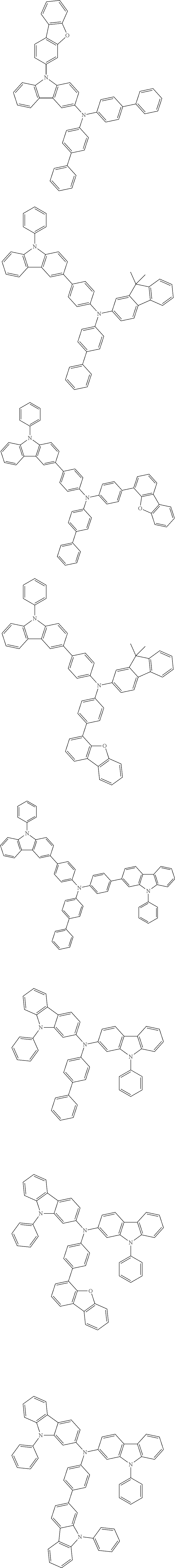

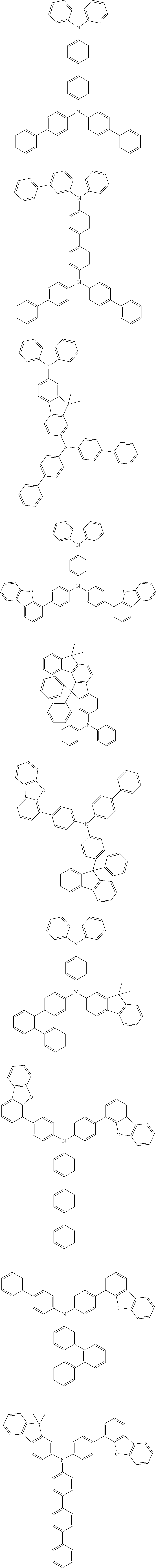

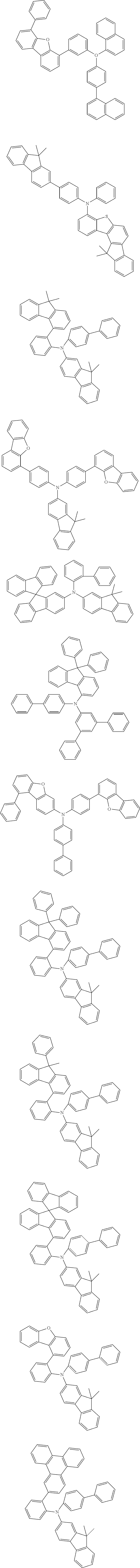

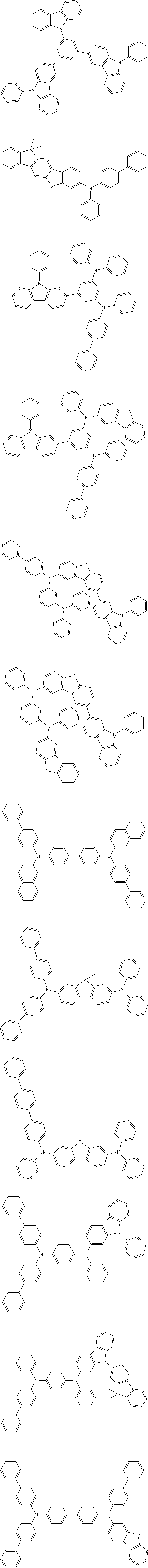

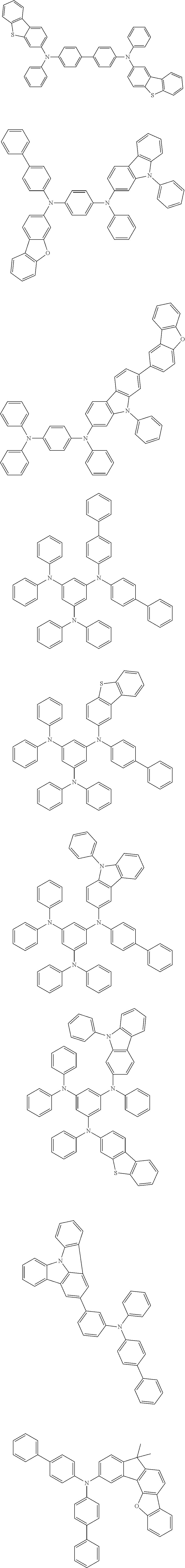

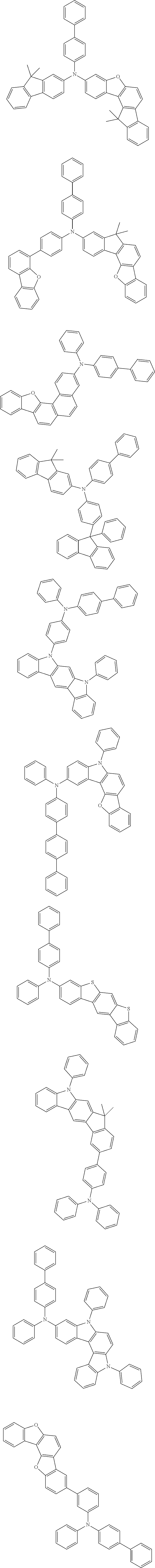

In addition to the aforementioned compounds, known compounds described in U.S. Pat. No. 5,061,569A, JP1993-009471A, WO1995-009147A1, JP1995-126615A, JP1998-095973A, and the like, and compounds having similar structures may be used for the hole transport auxiliary layer.

In addition, in an embodiment of the present invention, the organic light emitting diode may further include an electron transport layer, an electron injection layer, and a hole injection layer as the organic layer 105 in FIG. 1 or 2.

The organic light emitting diodes 100 and 200 may be manufactured by forming an anode or a cathode on a substrate, forming an organic layer by a dry film method such as evaporation, sputtering, plasma plating and ion plating, and forming a cathode or an anode thereon.

The organic light emitting diode may be applied to an organic light emitting display device.

### MODE FOR INVENTION

Hereinafter, the embodiments are illustrated in more detail with reference to examples. However, these examples are exemplary, and the present scope is not limited thereto.

### (Preparation of First Compound for Organic Optoelectronic Element)

### SYNTHESIS EXAMPLE 1

### Synthesis of Compound A-2

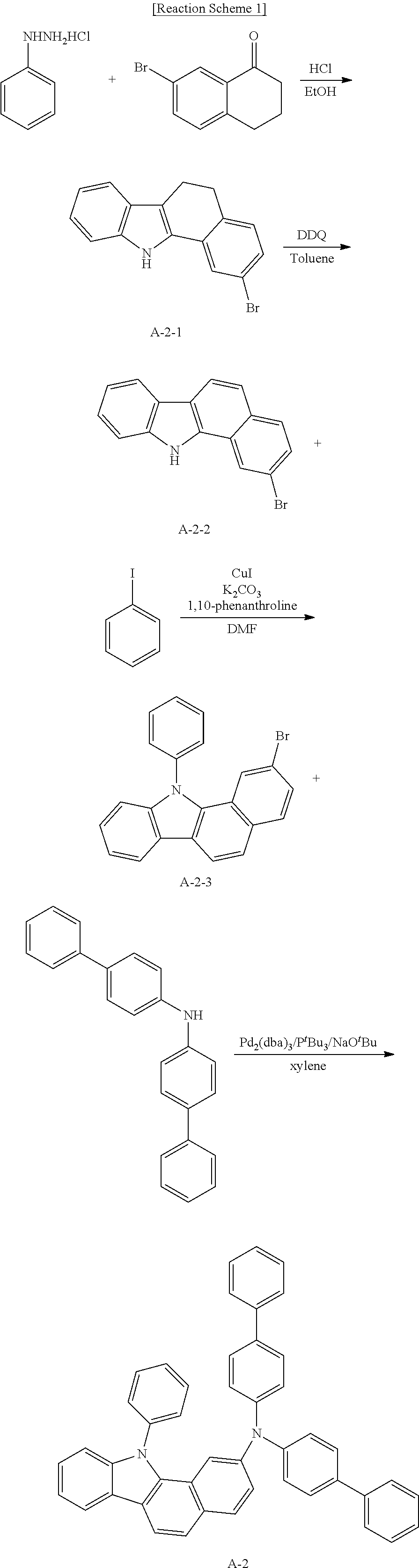

a) Synthesis of Intermediate_A-2-1

Phenylhydrazinehydrochloride (70.0 g, 484.1 mmol) and 7-bromo-3,4-dihydro-2H-naphthalen-1-one (108.9 g, 484.1 mmol) were put in a round bottom flask and dissolved in ethanol (1200 ml). At room temperature, 60 mL of hydrochloric acid was slowly added thereto in a dropwise fashion and then, stirred at 90° C. for 12 hours. When a reaction was complete, after removing the solvent under a reduced pressure, an excessive amount of EA was used for an extraction. After removing an organic solvent under an reduced pressure, the residue was stirred in a small amount of methanol to obtain 95.2 g (66%) of Intermediate A-2-1.

b) Synthesis of Intermediate A-2-2

Intermediate A-2-1 (95.2 g, 319.3 mmol) and 2,3-dichloro-5,6-dicyano-1,4-benzoquinone (108.7 g, 478.9 mmol) were put in a round bottom flask and then, dissolved in 600 ml of toluene. The solution was stirred at 80° C. for 12 hours. When a reaction was complete, after removing the reaction solvent, the residue was treated through column chromatography to obtain 41.3 g (44%) of Intermediate A-2-2.

c) Synthesis of Intermediate_A-2-3

Intermediate A-2-2 (41.3 g, 139.0 mmol), iodobenzene (199.2 g, 976.0 mmol), CuI (5.31 g, 28.0 mmol), K₂CO₃ (28.9 g, 209.0 mmol), and 1,10-phenanthroline (5.03 g, 28.0 mmol) were put in a round bottom flask and dissolved in 500 ml of DMF. The solution was stirred at 180° C. for 12 hours. When a reaction was complete, after removing the reaction solvent under a reduced pressure, the residue was dissolved in dichloromethane and then, silica gel-filtered. After concentrating the dichloromethane, hexane was used for a recrystallization to obtain 39.0 g (75%) of Intermediate A-2-3.

d) Synthesis of Compound A-2

Intermediate A-2-3 (23.2 g, 62.5 mmol), bis-biphenyl-4-yl-amine (21.1 g, 65.6 mmol), sodium t-butoxide (NaOtBu) (9.0 g, 93.8 mmol), Pd₂(dba)₃ (3.4 g, 3.7 mmol), and tri t-butylphosphine (P(tBu)₃) (4.5 g, 50% in toluene) were put in xylene (300 mL) and then, heated and refluxed under a nitrogen flow for 12 hours. After removing the xylene, 200 mL of methanol was added thereto, a solid crystallized therein was filtered, dissolved in toluene, filtered with silica gel/Celite, and then, an appropriate amount of the organic solvent was concentrated therefrom to obtain 29 g (76%) of Compound A-2.

LC/MS calculated for: C46H32N2 Exact Mass: 612.26 found for 612.32 [M+H]

### SYNTHESIS EXAMPLE 2

### Synthesis of Compound A-3

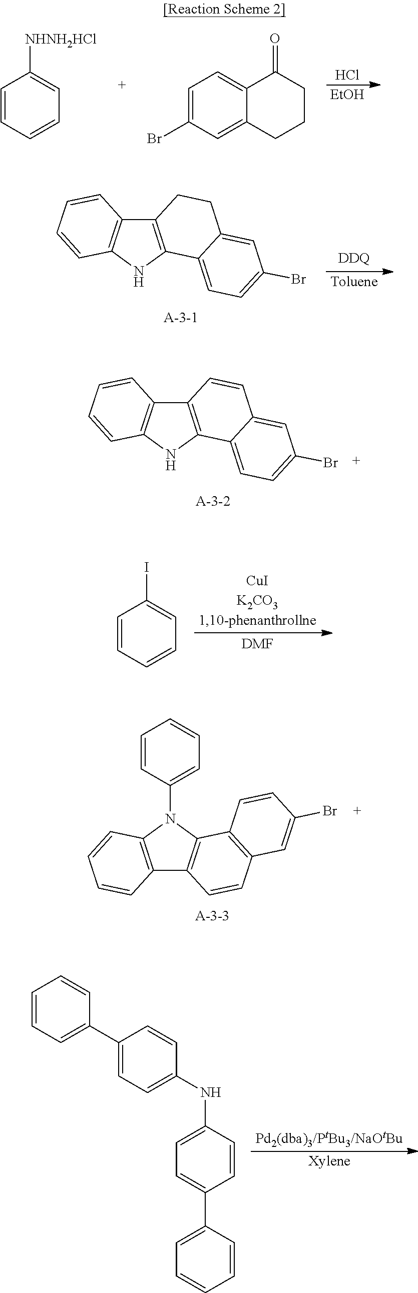

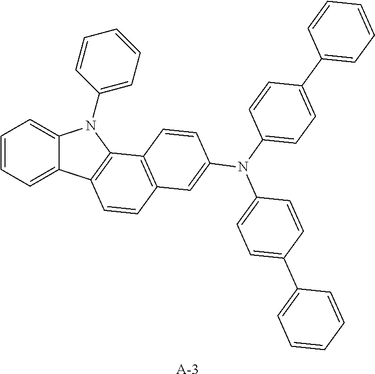 [SMILES: c1ccc(-c2ccc(N(c3ccc(-c4ccccc4)cc3)c3ccc4c(ccc5c6ccccc6n(-c6ccccc6)c45)c3)cc2)cc1 ; "A-3"]

a) Synthesis of Intermediate A-3-1

Intermediate A-3-1 was synthesized according to the same method as the a) of Synthesis Example 1 by using phenylhydrazinehydrochloride and 6-bromo-3,4-dihydro-2H-naphthalen-1-one respectively by 1.0 equivalent.

b) Synthesis of Intermediate A-3-2

Intermediate A-3-2 was synthesized according to the same method as the b) of Synthesis Example 1 by using Intermediate A-3-1 and 2,3-dichloro-5,6-dicyano-1,4-benzoquinone in an equivalent ratio of 1:1.5.

c) Synthesis of Intermediate A-3-3

Intermediate A-3-3 was synthesized according to the same method as the c) of Synthesis Example 1 by using Intermediate A-3-2 and iodobenzene in an equivalent ratio of 1:3.

d) Synthesis of Compound A-3

Compound A-3 was synthesized according to the same method as the d) of Synthesis Example 1 by using Intermediate A-3-3 and bis-biphenyl-4-yl-aminein an equivalent ratio of 1:1.

LC/MS calculated for: C46H32N2 Exact Mass: 612.26 found for 612.28 [M+H]

### SYNTHESIS EXAMPLE 3

### Synthesis of Compound A-5

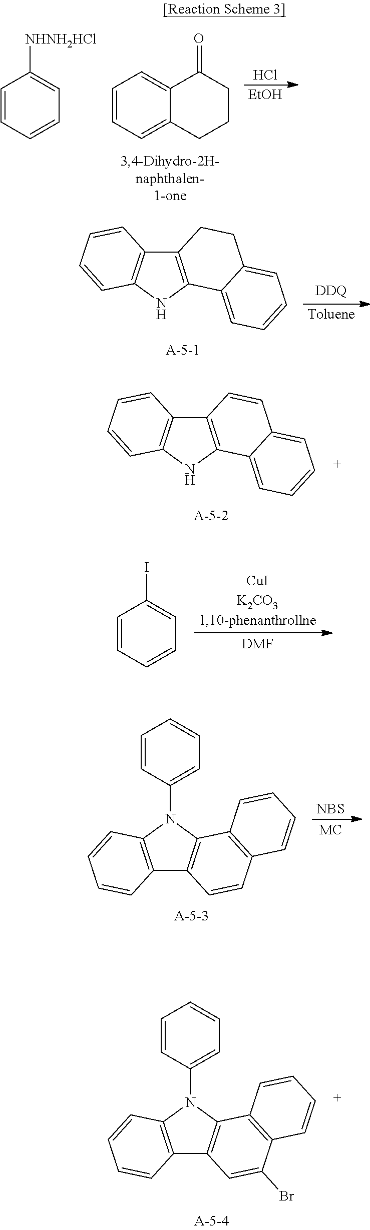

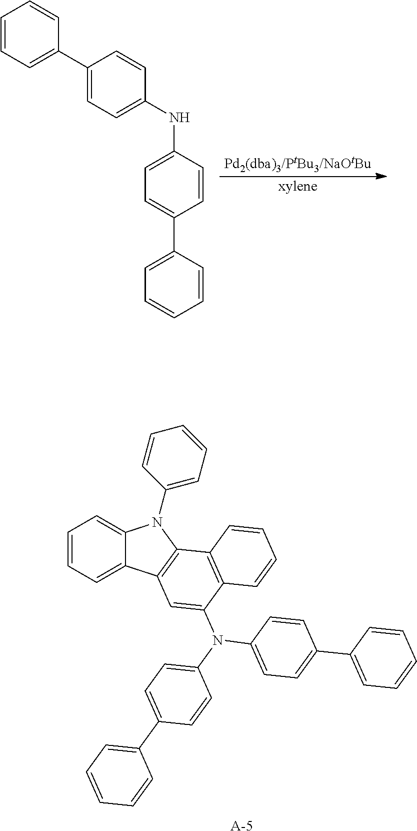 [SMILES: c1ccc(-c2ccc(Nc3ccc(-c4ccccc4)cc3)cc2)cc1 ; "xylene" ; "Pd2(dba)3/PtBu3/NaOtBu" ; c1ccc(-c2ccc(N(c3ccc(-c4ccccc4)cc3)c3cc4c5ccccc5n(-c5ccccc5)c4c4ccccc34)cc2)cc1 ; "A-5"]

a) Synthesis of Intermediate A-5-1

Intermediate A-5-1 was synthesized according to the same method as the a) of Synthesis Example 1 by using phenylhydrazinehydrochloride and 3,4-dihydro-2H-naphthalen-1-one in each amount of 1.0 equivalent.

b) Synthesis of Intermediate_A-5-2

Intermediate A-5-2 was synthesized according to the same method as the b) of Synthesis Example 1 by using intermediate A-5-1 and 2,3-dichloro-5,6-dicyano-1,4-benzoquinone in an equivalent ratio of 1:1.5.

c) Synthesis of Intermediate A-5-3

Intermediate A-5-3 was synthesized according to the same method as the c) of Synthesis Example 1 by using intermediate A-5-2 and iodobenzene in an equivalent ratio of 1:3.

d) Synthesis of Intermediate A-5-4

Intermediate A-5-3 (23.6 g, 80.6 mmol) was put in a round bottom flask and dissolved in 300 mL of dichloromethane. After dissolving N-Bromosuccinimide (NBS) (14.1 g, 79.0 mmol) in 100 mL of DMF, the solution was slowly added thereto in a dropwise fashion and then, stirred at room temperature for 2 hours. When a reaction was complete, after removing the reaction solvent, the residue was treated through column chromatography to obtain 25 g (83%) of Intermediate A-5-4.

e) Synthesis of Compound A-5

Compound A-5 was synthesized according to the same method as the d) of Synthesis Example 1 by using Intermediate A-5-4 and bis-biphenyl-4-yl-amine in an equivalent ratio of 1:1.

LC/MS calculated for: C46H32N2 Exact Mass: 612.26 found for 612.33 [M+H]

### SYNTHESIS EXAMPLE 4

### Synthesis of Compound A-7

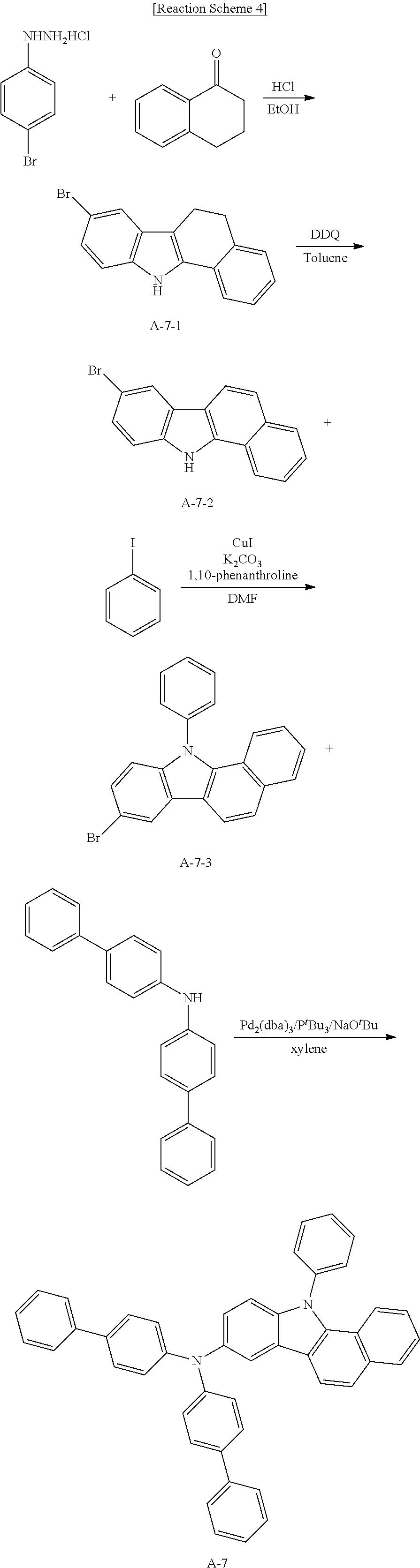

a) Synthesis of Intermediate A-7-1

Intermediate A-7-1 was synthesized according to the same method as the a) of Synthesis Example 1 by using 4-bromophenylhydrazine hydrochloride and 3,4-dihydro-2H-naphthalen-1-one respectively by 1.0 equivalent.

b) Synthesis of Intermediate_A-7-2

Intermediate A-7-2 was synthesized according to the same method as the b) of Synthesis Example 1 by using Intermediate A-7-1 and 2,3-dichloro-5,6-dicyano-1,4-benzoquinone in an equivalent ratio of 1:1.5.

c) Synthesis of Intermediate A-7-3

Intermediate A-7-3 was synthesized according to the same method as the c) of Synthesis Example 1 by using Intermediate A-7-2 and iodobenzene in an equivalent ratio of 1:3.

d) Synthesis of Compound A-7

Compound A-7 was synthesized according to the same method as the d) of Synthesis Example 1 by using Intermediate A-7-3 and bis-biphenyl-4-yl-amine in an equivalent ratio of 1:1.

LC/MS calculated for: C46H32N2 Exact Mass: 612.26 found for 612.30 [M+H]

### SYNTHESIS EXAMPLE 5

### Synthesis of Compound A-8

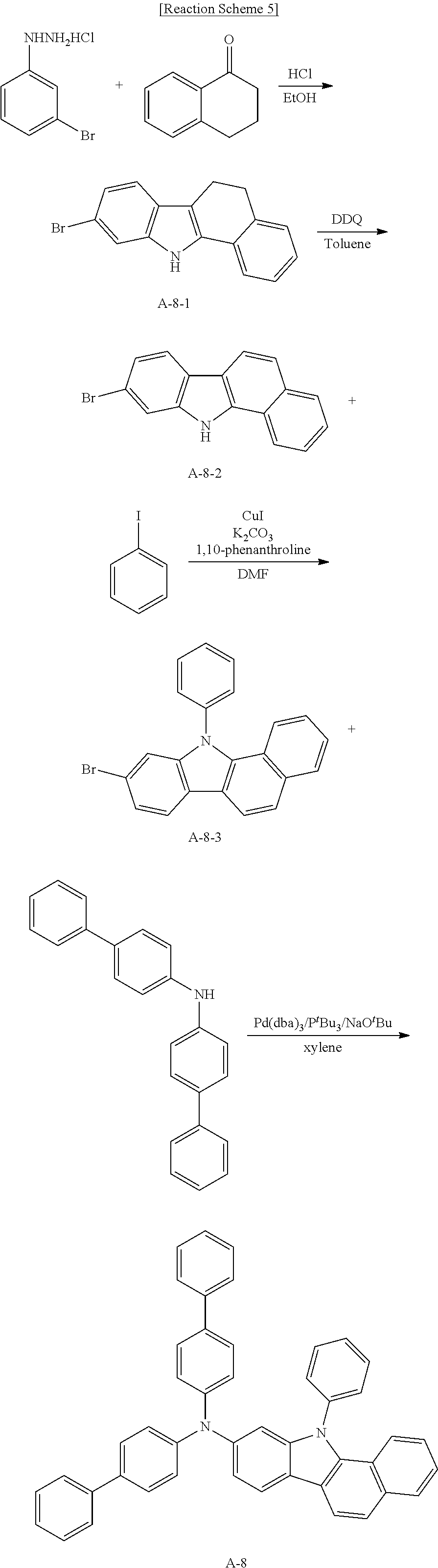

a) Synthesis of Intermediate A-8-1

Intermediate A-8-1 was synthesized according to the same method as the a) of Synthesis Example 1 by using 3-bromophenylhydrazinehydrochloride and 3,4-dihydro-2H-naphthalen-1-one respectively by 1.0 equivalent.

b) Synthesis of Intermediate A-8-2

Intermediate A-8-2 was synthesized according to the same method as the b) of Synthesis Example 1 by using Intermediate A-8-1 and 2,3-dichloro-5,6-dicyano-1,4-benzoquinone in an equivalent ratio of 1:1.5.

c) Synthesis of Intermediate A-8-3

Intermediate A-8-3 was synthesized according to the same method as the c) of Synthesis Example 1 by using Intermediate A-8-2 and iodobenzene in an equivalent ratio of 1:3.

d) Synthesis of Compound A-8

Compound A-8 was synthesized according to the same method as the d) of Synthesis Example 1 by using Intermediate A-8-3 and bis-biphenyl-4-yl-aminein an equivalent ratio of 1:1.

LC/MS calculated for: C46H32N2 Exact Mass: 612.26 found for 612.33 [M+H]

### SYNTHESIS EXAMPLE 6

### Synthesis of Compound A-11

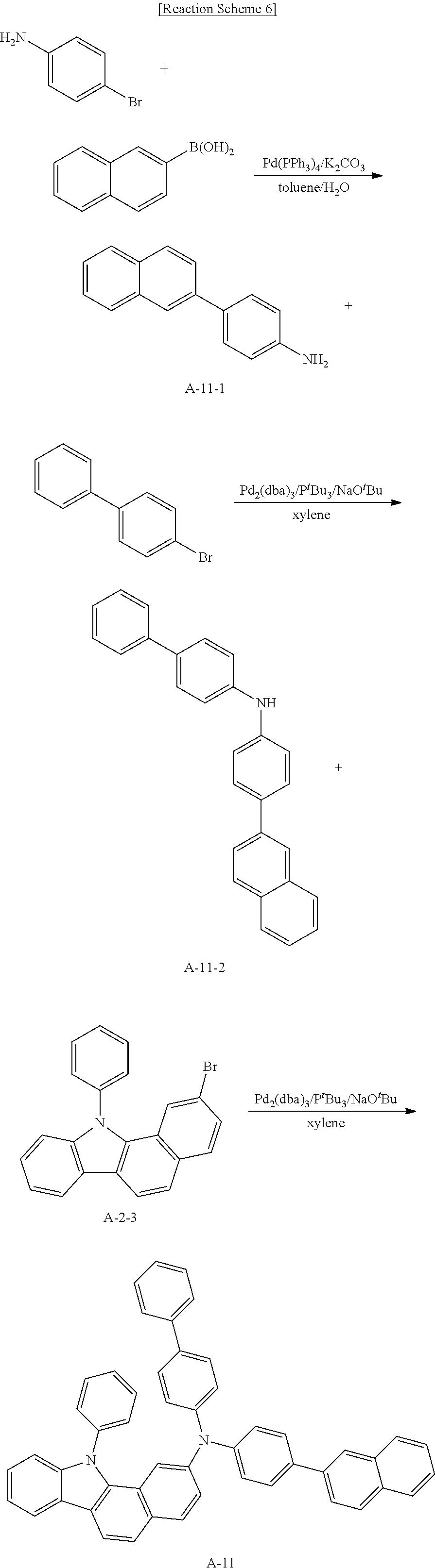

a) Synthesis of Intermediate A-11-1

4-bromo-phenylamine (50.0 g, 290.7 mmol), 2-naphthalene boronic acid (59.9 g, 171.9 mmol), K₂CO₃ (80.4 g, 581.3 mmol), and Pd(PPh₃)₄ (10.1 g, 8.7 mmol) were put in a round bottom flask and dissolved in 800 ml of toluene and 400 ml of distilled water and then, stirred at 80° C. for 12 hours. When a reaction was complete, after removing an aqueous layer therefrom, the residue was treated through column chromatography to obtain 40.0 g (63%) of Intermediate A-11-1.

b) Synthesis of Intermediate A-11-2

Intermediate A-11-1 (17.7 g, 80.8 mmol), 4-bromo-biphenyl (18.8 g, 80.8 mmol), sodium t-butoxide (NaOtBu) (11.6 g, 121.1 mmol), Pd₂(dba)₃ (4.4 g, 4.8 mmol), and tri t-butylphosphine (P(tBu)₃) (5.9 g, 50% in toluene) were added to xylene (400 mL) and then, heated and refluxed under a nitrogen flow for 12 hours. After removing the xylene, the residue was treated through column chromatography to obtain 20.0 g (67%) of Intermediate A-11-2.

c) Synthesis of Compound A-11

Compound A-11 was synthesized according to the same method as the d) of Synthesis Example 1 by using Intermediate A-11-2 and Intermediate A-2-3 in an equivalent ratio of 1:1.

LC/MS calculated for: C50H34N2 Exact Mass: 662.27 found for 662.31 [M+H]

### SYNTHESIS EXAMPLE 7

### Synthesis of Compound A-12

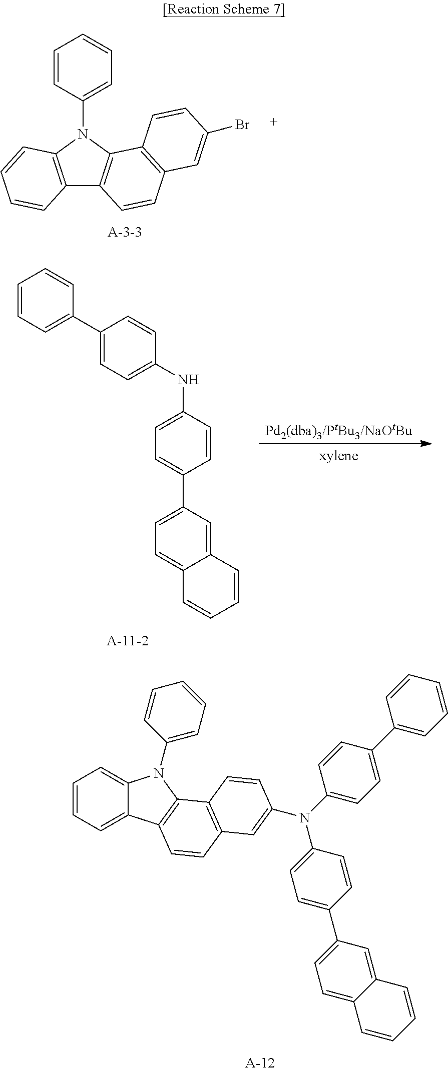 [SMILES: Brc1ccc2c(ccc3c4ccccc4n(-c4ccccc4)c23)c1 ; c1ccc(-c2ccc(Nc3ccc(-c4ccc5ccccc5c4)cc3)cc2)cc1 ; "Pd2(dba)3/PtBu3/NaOtBu" ; c1ccc(-c2ccc(N(c3ccc(-c4ccc5ccccc5c4)cc3)c3ccc4c(ccc5c6ccccc6n(-c6ccccc6)c45)c3)cc2)cc1 ; "A-12"]

Compound A-12 was synthesized according to the same method as the d) of Synthesis Example 1 by using Intermediate A-3-3 and Intermediate A-11-2 in an equivalent ratio of 1:1.

LC/MS calculated for: C50H34N2 Exact Mass: 662.27 found for 662.30 [M+H]

### SYNTHESIS EXAMPLE 8

### Synthesis of Compound A-29

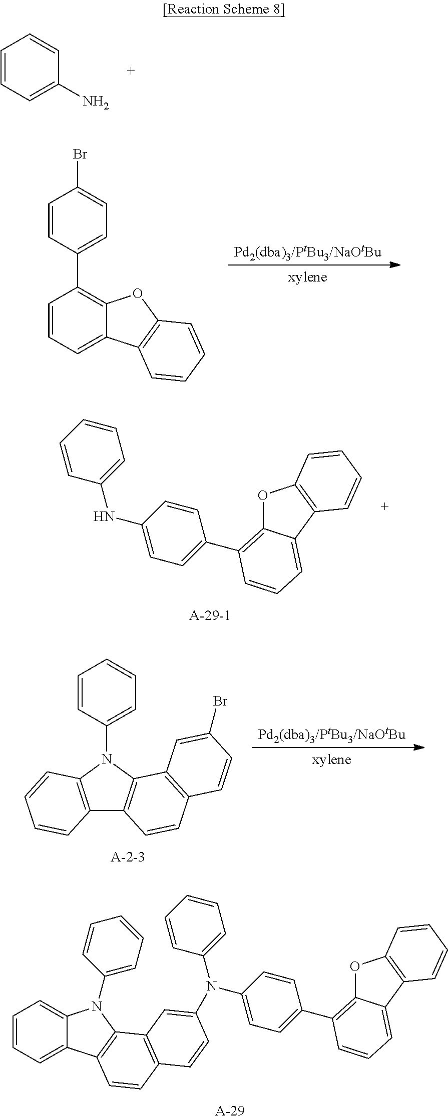

a) Synthesis of Intermediate A-29-1

Aniline (8.3 g, 89.5 mmol), 4-(4-bromo-phenyl)-dibenzofuran (23.1 g, 71.5 mmol), sodium t-butoxide (NaOtBu) (12.9 g, 134.2 mmol), Pd₂(dba)₃ (4.9 g, 5.4 mmol), and tri t-butylphosphine (P(tBu)₃) (6.5 g, 50% in toluene) were added to xylene (400 mL) and then, heated and refluxed under a nitrogen flow for 12 hours. After removing the xylene, the residue was treated through column chromatography to obtain 20.0 g (67%) of Intermediate A-29-1.

b) Synthesis of Compound A-29

Compound A-29 was synthesized according to the same method as the d) of Synthesis Example 1 by using Intermediate A-29-1 and Intermediate A-2-3 in an equivalent ratio of 1:1.

LC/MS calculated for: C46H30N20 Exact Mass: 626.24 found for 626.28 [M+H]

### SYNTHESIS EXAMPLE 9

### Synthesis of Compound A-38

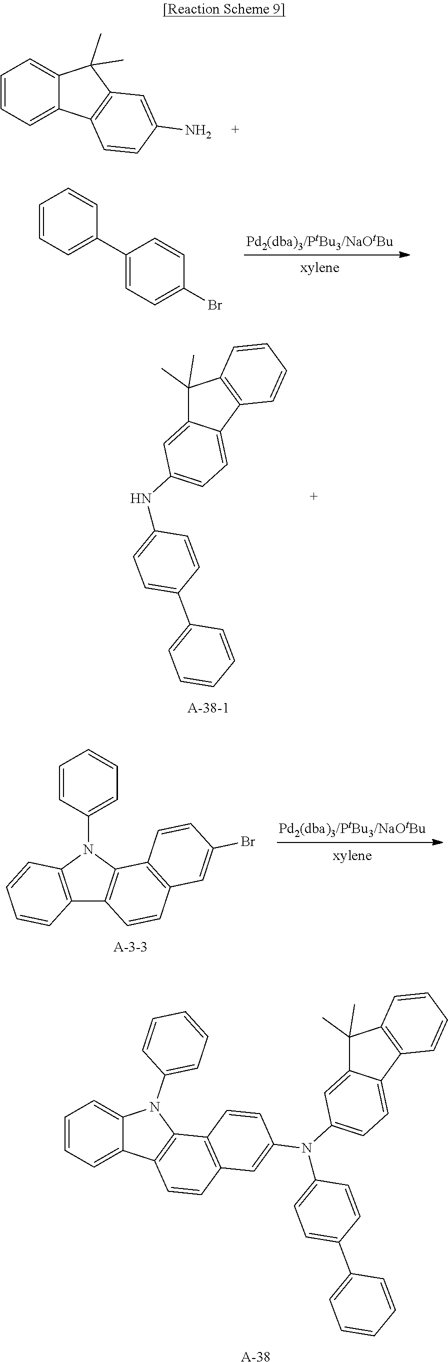 [SMILES: CC1(C)c2ccccc2-c2ccc(N)cc21 ; Brc1ccc(-c2ccccc2)cc1 ; CC1(C)c2ccccc2-c2ccc(Nc3ccc(-c4ccccc4)cc3)cc21 ; Brc1ccc2c(ccc3c4ccccc4n(-c4ccccc4)c23)c1 ; CC1(C)c2ccccc2-c2ccc(N(c3ccc(-c4ccccc4)cc3)c3ccc4c(ccc5c6ccccc6n(-c6ccccc6)c45)c3)cc21 ; "A-38"]

a) Synthesis of Intermediate A-38-1

9,9-dimethyl-9H-fluoren-2-ylamine (17.4 g, 83.0 mmol), 4-bromo-biphenyl (15.5 g, 66.4 mmol), sodium t-butoxide (NaOtBu) (12.0 g, 124.5 mmol), Pd₂(dba)₃ (4.6 g, 5.0 mmol), and tri t-butylphosphine (P(tBu)₃) (6.0 g, 50% in toluene) were added to xylene (400 mL) and then, heated and refluxed under a nitrogen flow for 12 hours. After removing the xylene, the residue was treated through column chromatography to obtain 18.0 g (60%) of Intermediate A-38-1.

b) Synthesis of Compound A-38

Compound A-38 was synthesized according to the same method as the d) of Synthesis Example 1 by using Intermediate A-38-1 and Intermediate A-3-3 in an equivalent ratio of 1:1.

LC/MS calculated for: C49H36N2 Exact Mass: 652.29 found for 652.33 [M+H]

### SYNTHESIS EXAMPLE 10

### Synthesis of Compound A-51

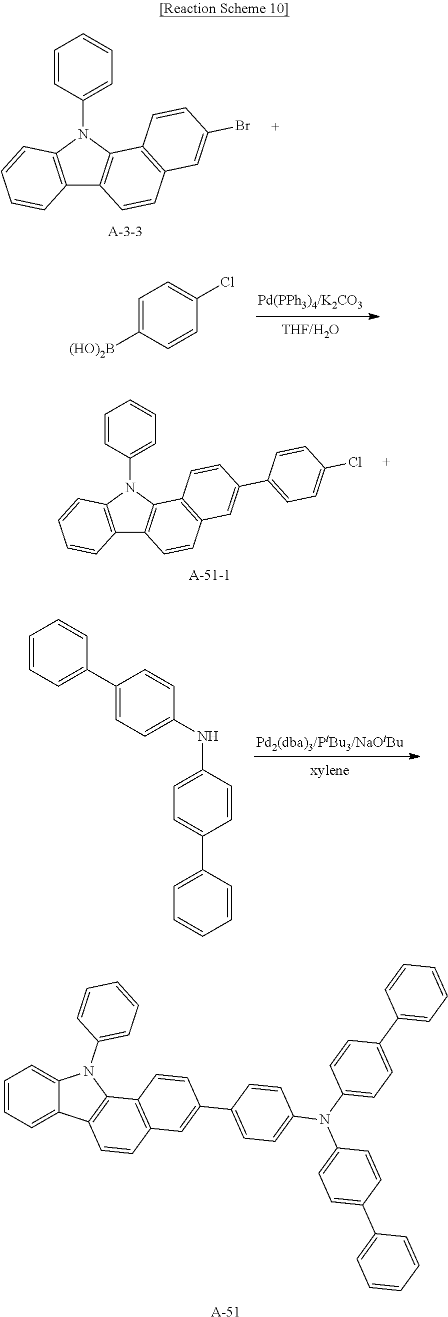 [SMILES: Brc1ccc2c(ccc3c4ccccc4n(-c4ccccc4)c23)c1 ; OB(O)c1ccc(Cl)cc1 ; Clc1ccc(-c2ccc3c(ccc4c5ccccc5n(-c5ccccc5)c34)c2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc5c(ccc6c7ccccc7n(-c7ccccc7)c56)c4)cc3)cc2)cc1 ; "A-51"]

a) Synthesis of Intermediate A-51-1

Intermediate A-3-3 (30.0 g, 80.6 mmol), 4-chlorophenyl boronic acid (15.1 g, 96.7 mmol), K₂CO₃ (22.3 g, 161.2 mmol), and Pd(PPh₃)₄ (2.8 g, 2.4 mmol) were put in a round bottom flask and dissolved in 200 ml of tetrahydrofuran and 100 ml of distilled water and then, stirred at 80° C. for 12 hours. When a reaction was complete, after removing an aqueous layer therefrom, the residue was treated through column chromatography to obtain 27.0 g (83%) of Intermediate A-51-1.

b) Synthesis of Compound A-51

Compound A-51 was synthesized according to the same method as the d) of Synthesis Example 1 by using Intermediate A-51-1 and bis-biphenyl-4-yl-amine in an equivalent ratio of 1:1.

LC/MS calculated for: C52H36N2 Exact Mass: 688.29 found for 688.34 [M+H]

### SYNTHESIS EXAMPLE 11

### Synthesis of Compound A-65

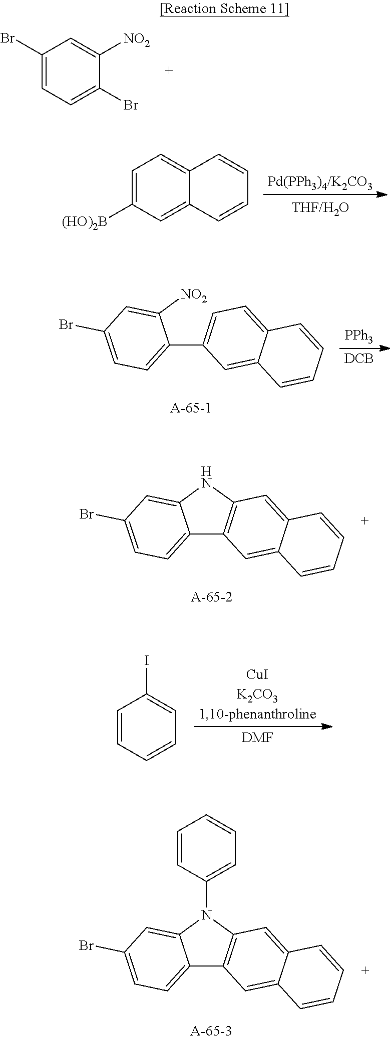

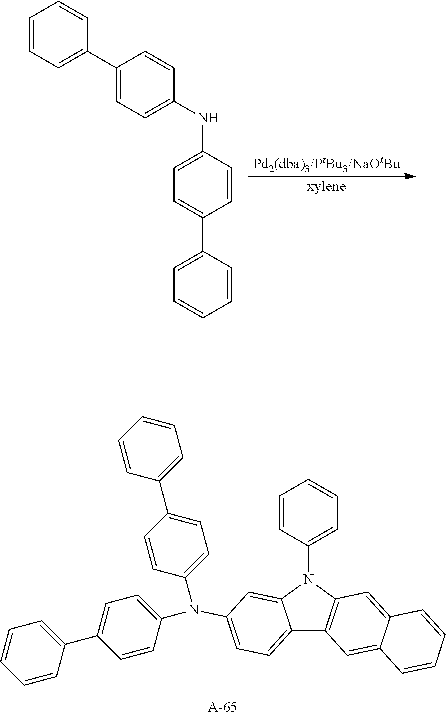 [SMILES: c1ccc(-c2ccc(Nc3ccc(-c4ccccc4)cc3)cc2)cc1 ; "xylene" ; "Pd2(dba)3/PtBu3/NaOtBu" ; c1ccc(-c2ccc(N(c3ccc(-c4ccccc4)cc3)c3ccc4c5cc6ccccc6cc5n(-c5ccccc5)c4c3)cc2)cc1 ; "A-65"]

a) Synthesis of Intermediate A-65-1

1,4-dibromo-2-nitro-benzene (30.0 g, 106.8 mmol), 2-naphthalene boronic acid (18.4 g, 106.8 mmol), K₂CO₃ (29.5 g, 213.6 mmol), and Pd(PPh₃)₄ (3.7 g, 3.2 mmol) were put in a round bottom flask, dissolved in 300 mL of tetrahydrofuran and 150 mL of distilled water and then, stirred at 80° C. for 12 hours. When a reaction was complete, after removing an aqueous layer therefrom, the residue was treated through column chromatography to obtain 27.0 g (77%) of Intermediate A-65-1.

b) Synthesis of Intermediate A-65-2

Intermediate A-65-1 (27.0 g, 82.3 mmol) and triphenylphosphine (86.3 g, 329.1 mmol) were put in a round bottom flask, dissolved in 300 mL of 1,2-dichlorobenzene and then, stirred at 180° C. for 12 hours. When a reaction was complete, after removing the solvent therefrom, the residue was treated through column chromatography to obtain 18.0 g (74%) of Intermediate A-65-2.

c) Synthesis of Intermediate A-65-3

Intermediate A-65-3 was synthesized according to the same method as the c) of Synthesis Example 1 by using Intermediate A-65-2 and iodobenzene in an equivalent ratio of 1:3.

d) Synthesis of Compound A-65

Compound A-65 was synthesized according to the same method as the d) of Synthesis Example 1 by using Intermediate A-65-3 and bis-biphenyl-4-yl-amine in an equivalent ratio of 1:1.

LC/MS calculated for: C46H32N2 Exact Mass: 612.26 found for 612.30 [M+H]

### SYNTHESIS EXAMPLE 12

### Synthesis of Compound A-72

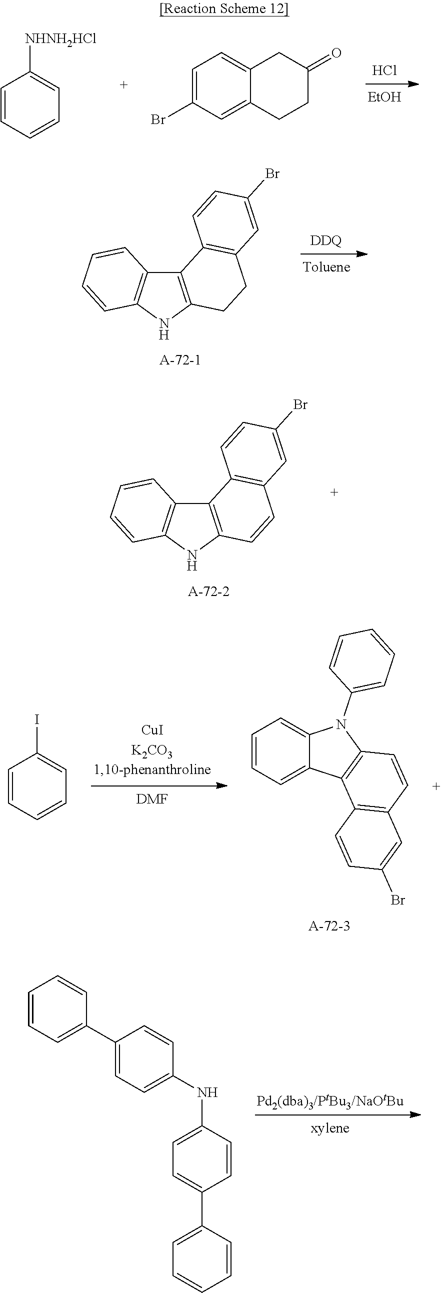

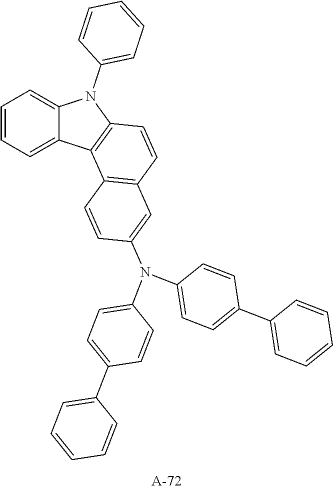 [SMILES: c1ccc(-c2ccc(N(c3ccc(-c4ccccc4)cc3)c3ccc4c(ccc5c4c4ccccc4n5-c4ccccc4)c3)cc2)cc1 ; "A-72"]

a) Synthesis of Intermediate A-72-1

Intermediate A-72-1 was synthesized according to the same method as the a) of Synthesis Example 1 by using phenylhydrazinehydrochloride and 6-bromo-3,4-dihydro-1H-naphthalen-2-one respectively by 1.0 equivalent.

b) Synthesis of Intermediate A-72-2

Intermediate A-72-2 was synthesized according to the same method as the b) of Synthesis Example 1 by using Intermediate A-72-1 and 2,3-dichloro-5,6-dicyano-1,4-benzoquinone in an equivalent ratio of 1:1.5.

c) Synthesis of Intermediate A-72-3

Intermediate A-72-3 was synthesized according to the same method as the c) of Synthesis Example 1 by using Intermediate A-72-2 and iodobenzene in an equivalent ratio of 1:3.

d) Synthesis of Compound A-72

Intermediate A-72 was synthesized according to the same method as the d) of Synthesis Example 1 by using Intermediate A-72-3 and bis-biphenyl-4-yl-amine in an equivalent ratio of 1:1.

LC/MS calculated for: C46H32N2 Exact Mass: 612.26 found for 612.31 [M+H]

### SYNTHESIS EXAMPLE 13

### Synthesis of Compound A-77

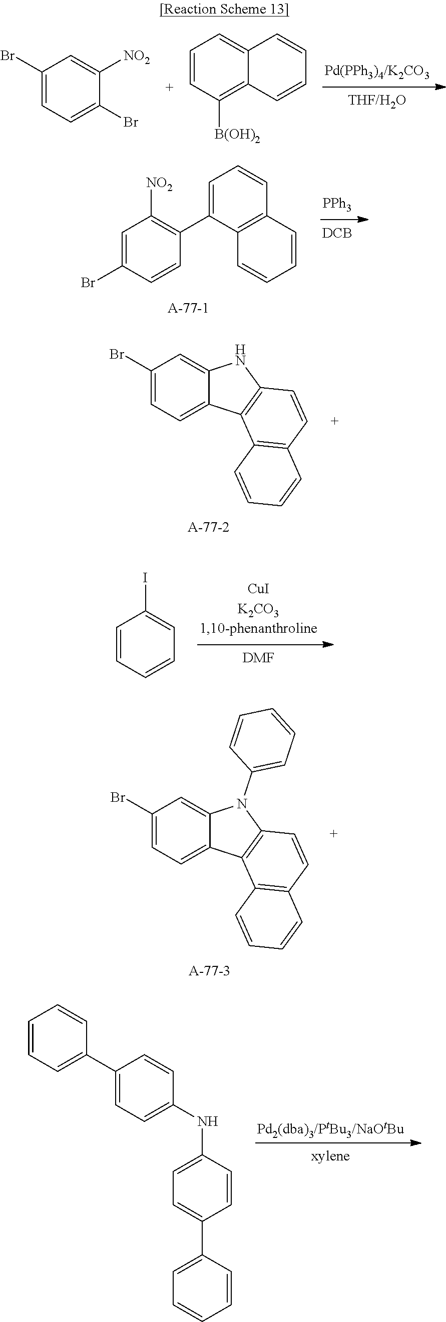

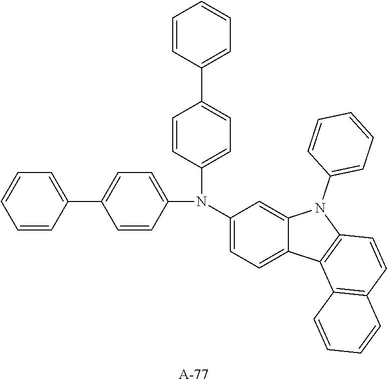 [SMILES: c1ccc(-c2ccc(N(c3ccc(-c4ccccc4)cc3)c3ccc4c5c6ccccc6ccc5n(-c5ccccc5)c4c3)cc2)cc1 ; "A-77"]

a) Synthesis of Intermediate A-77-1

Intermediate A-77-1 was synthesized according to the same method as the a) of Synthesis Example 11 by using 1,4-dibromo-2-nitro-benzene and 1-naphthalene boronic acid respectively by 1.0 equivalent.

b) Synthesis of Intermediate A-77-2

Intermediate A-77-2 was synthesized according to the same method as the b) of Synthesis Example 11 by using Intermediate A-77-1 and triphenylphosphine in an equivalent ratio of 1:4.

c) Synthesis of Intermediate A-77-3

Intermediate A-77-3 was synthesized according to the same method as the c) of Synthesis Example 1 by using Intermediate A-77-2 and iodobenzene in an equivalent ratio of 1:3.

d) Synthesis of Compound A-77

Compound A-77 was synthesized according to the same method as the d) of Synthesis Example 1 by using Intermediate A-77-3 and bis-biphenyl-4-yl-amine in an equivalent ratio of 1:1.

LC/MS calculated for: C46H32N2 Exact Mass: 612.26 found for 612.29 [M+H]

### COMPARATIVE SYNTHESIS EXAMPLE 1

### Synthesis of Comparative Compound V-1

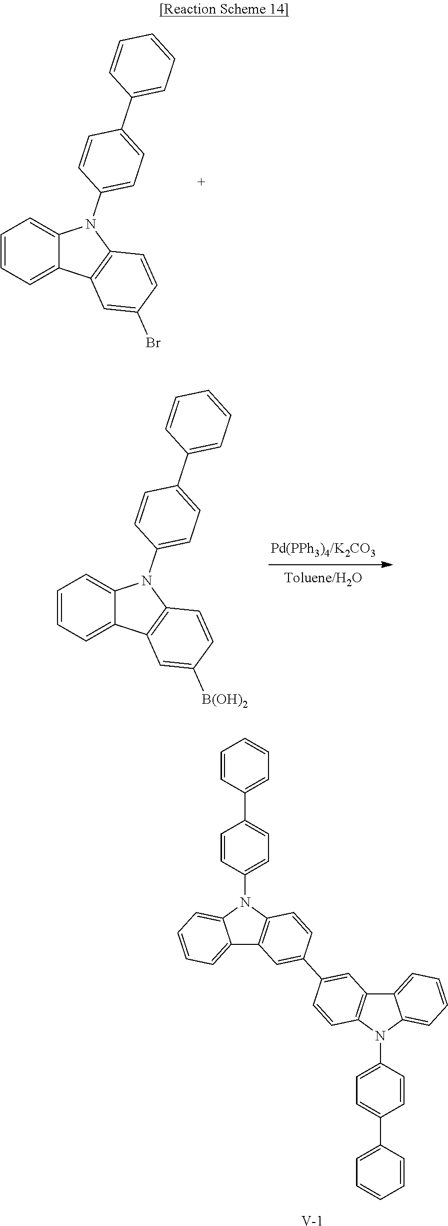

The compound of biphenylcarbazolyl bromide (12.33 g, 30.95 mmol) was dissolved in 200 mL of toluene under a nitrogen environment, and biphenylcarbazolylboronic acid (12.37 g, 34.05 mmol) and tetrakis(triphenylphosphine)palladium (1.07 g, 0.93 mmmol) were added thereto and then, stirred. Potassium carbonate (12.83 g, 92.86 mmol) saturated in water was added thereto and then, heated and refluxed at 90° C. for 12 hours. When a reaction was complete, water was added to the reaction solution, and the mixture was extracted with dichloromethane (DCM), treated with anhydrous MgSO₄ to remove moisture, filtered, and concentrated under a reduced pressure. The obtained residue was separated and purified through flash column chromatography to obtain Compound V-1 (18.7 g, 92%).

LC/MS calculated for: C48H32N2 Exact Mass: 636.26 found for 636.30 [M+H]

### COMPARATIVE SYNTHESIS EXAMPLE 2

### Synthesis of Comparative Compound V-2

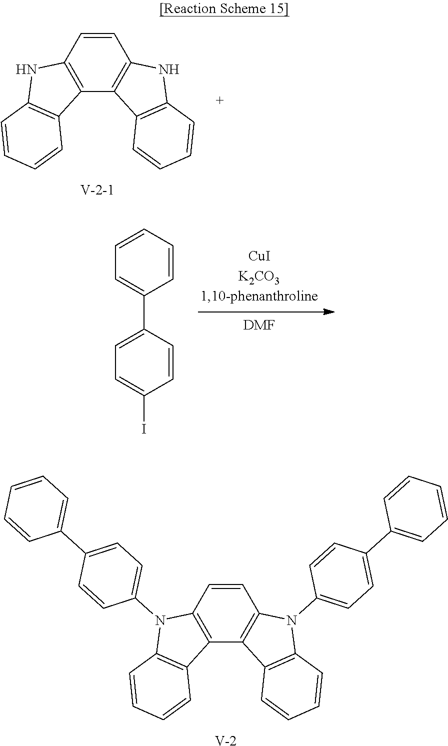

In a round-bottomed flask, 8 g (31.2 mmol) of Intermediate V-2-1 (5,8-dihydro-indolo[2,3-C]carbazole), 20.5 g (73.32 mmol) of 4-iodobiphenyl, 1.19 g (6.24 mmol) of CuI, 1.12 g (6.24 mmol) of 1,10-phenanthoroline, and 12.9 g (93.6 mmol) of K₂CO₃ were put, and 50 ml of DMF was added thereto and then, refluxed and stirred under a nitrogen atmosphere for 24 hours. When a reaction was complete, distilled water was added thereto, and crystals precipitated therein were filtered. The solids were dissolved in 250 ml of xylene, filtered through silica gel, and precipitated into a white solid, obtaining 16.2 g (Yield: 93%) of Compound V-2.

LC/MS calculated for: C42H28N2 Exact Mass: 560.23 found for 560.27 [M+H]

### (Preparation of Second Compound for Organic Optoelectronic Element)

### SYNTHESIS EXAMPLE 14

### Synthesis of Compound B-697

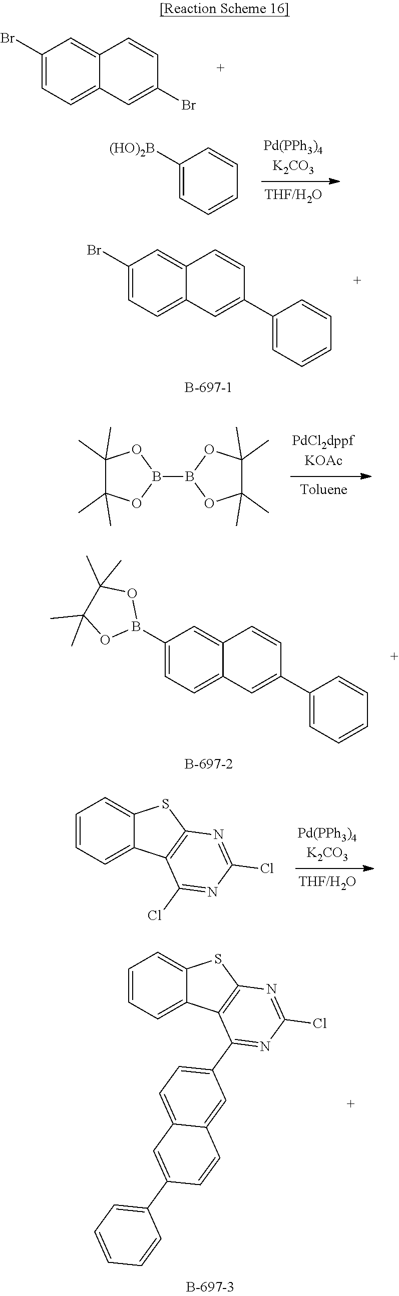

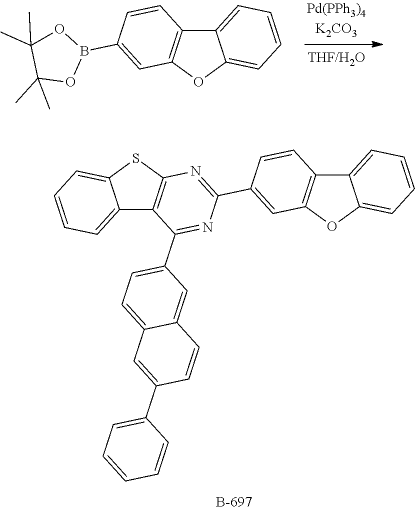 [SMILES: CC1(C)OB(c2ccc3c(c2)oc2ccccc23)OC1(C)C ; c1ccc(-c2ccc3cc(-c4nc(-c5ccc6c(c5)oc5ccccc56)nc5sc6ccccc6c45)ccc3c2)cc1]

a) Synthesis of Intermediate B-697-1

Intermediate B-697-1 was synthesized according to the same method as the a) of Synthesis Example 11 by using 2,6-dibromonaphthalene and phenylboronic acid respectively by 1.0 equivalent.

b) Synthesis of Intermediate B-697-2

Intermediate B-697-1 (50 g, 177 mmol), bis(pinacolato)diboron (67.26 g, 265 mmol), 1,1′-Bis(diphenylphosphino)ferrocene (PdCl₂dppf) (5.77 g, 7 mmol), and potassium acetate (51.99 g, 530 mmol) were put in a round bottom flask, and 800 mL of toluene was added thereto and then, refluxed and stirred at 130° C. for 12 hours. When a reaction was complete, after all evaporating the solvent therefrom under a reduced pressure, the residue was dissolved in dichloromethane and three times extracted with distilled water. A mixed solvent of dichloromethane and n-hexane was used for recrystallization to obtain 46.5 g (79.7%) of Intermediate B-697-2.

c) Synthesis of Intermediate B-697-3

Intermediate B-697-3 was synthesized according to the same method as the a) of Synthesis Example 11 by reacting Intermediate B-697-2 and 2,4-dichlorobenzo[4,5]thieno[2,3-d]pyrimidine in each amount of 1.0 equivalent and performing recrystallization with toluene.

d) Synthesis of Compound B-697

Compound B-697-3 was synthesized according to the same method as the a) of Synthesis Example 11 by reacting Intermediate B-697-3 and 2-(dibenzo[b,d]furan-3-yl)-4,4,5,5-tetramethyl-1,3,2-dioxaborolane in each amount 1.0 equivalent and performing recrystallization with chlorobenzene.

LC/MS calculated for: C38H22N2OS Exact Mass: 554.15 found for 555.26 [M+H]

### SYNTHESIS EXAMPLE 15

### Synthesis of Compound B-698

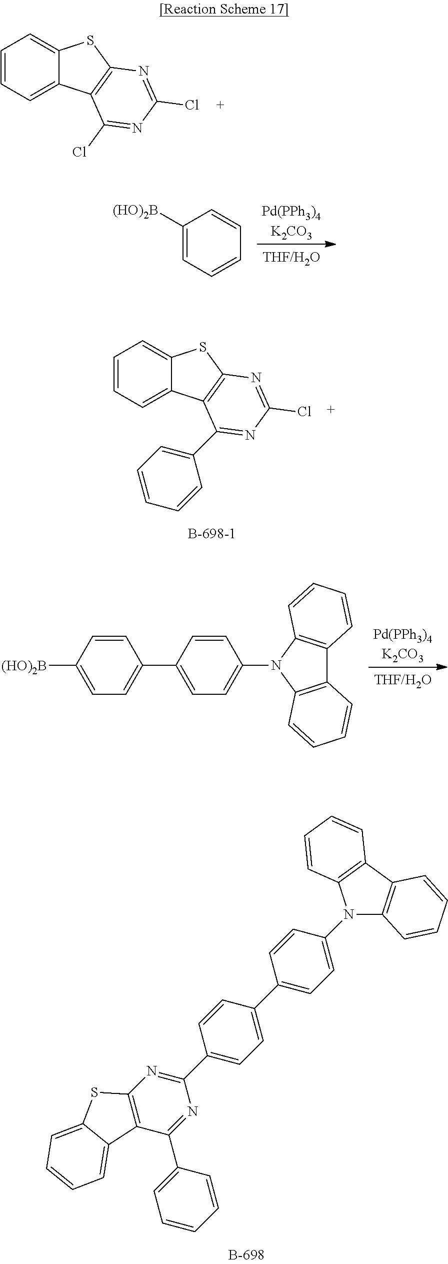

a) Synthesis of Intermediate B-698-1

Intermediate B-698-1 was synthesized according to the same method as the a) of Synthesis Example 11 by using 2,4-dichlorobenzo[4,5]thieno[2,3-d]pyrimidine and phenylboronic acid respectively by 1.0 equivalent.

b) Synthesis of Compound B-698

Compound B-698 was synthesized according to the same method as the a) of Synthesis Example 11 by reacting Intermediate B-698-1 and (4′-(9H-carbazol-9-yl)-[1,1′-biphenyl]-4-yl)boronic acid in each amount of 1.0 equivalent and performing recrystallization with chlorobenzene.

LC/MS calculated for: C40H25N3S Exact Mass: 579.18 found for 580.29 [M+H]

### SYNTHSIS EXAMPLE 16

### Synthesis of Compound B-716

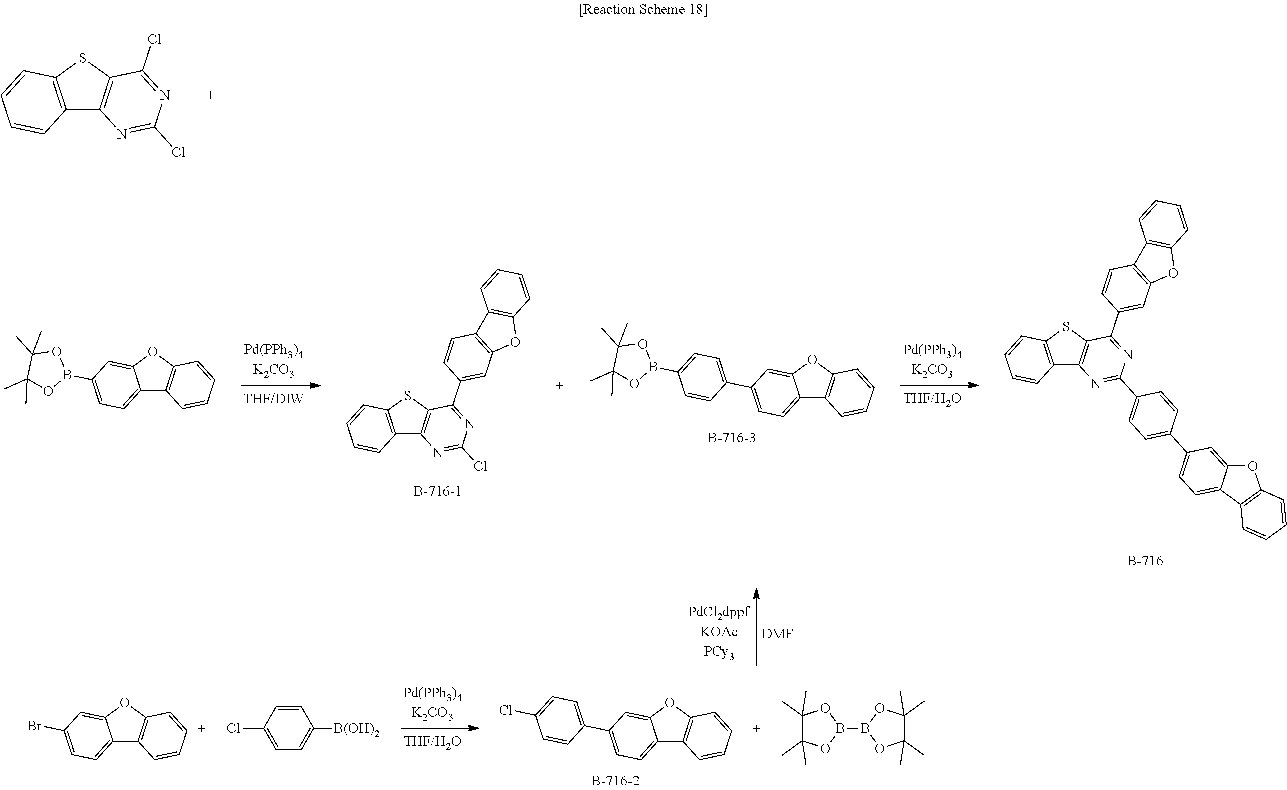

a) Synthesis of Intermediate B-716-1

Intermediate B-716-1 was synthesized according to the same method as the a) of Synthesis Example 11 by reacting 2,4-dichlorobenzo[4,5]thieno[3,2-d]pyrimidine and 2-(dibenzo[b,d]furan-3-yl)-4,4,5,5-tetramethyl-1,3,2-dioxaborolane in each amount of 1.0 equivalent and performing recrystallization with toluene.

b) Synthesis of Intermediate B-716-2

Intermediate B-716-2 was synthesized according to the same method as the a) of Synthesis Example 11 by reacting 3-bromodibenzofuran and 4-chlorophenylboronic acid in each amount of 1.0 equivalent and performing recrystallization with toluene.

c) Synthesis of Intermediate B-716-3

Intermediate B-716-2 (23 g, 83 mmol), bis(pinacolato)diboron (31.43 g, 124 mmol), 1,1′-bis(diphenylphosphino)ferrocene (PdCl₂dppf) (3.37 g, 4 mmol), tricyclohexylphosphine (5.55 g, 20 mmol), and potassium acetate (24.3 g, 248 mmol) were put in a round bottom flask, and 400 mL of N,N-dimethylformamide was added thereto and then, refluxed and stirred at 160° C. for 12 hours. When a reaction was complete, after all evaporating the solvent therefrom under a reduced pressure, the residue was dissolved in dichloromethane and three times extracted with distilled water. A mixed solvent of dichloromethane and n-hexane was used for recrystallization to obtain 24.9 g (81.5%) of Intermediate B-716-3.

d) Synthesis of Compound B-716

Intermediate B-716 was synthesized according to the same method as the a) of Synthesis Example 11 by using Intermediate B-716-1 and Intermediate B-716-3 respectively by 1.0 equivalent and performing recrystallization with dichlorobenzene.

LC/MS calculated for: C40H22N202S Exact Mass: 594.14 found for 595.28 [M+H]

### SYNTHESIS EXAMPLE 17

### Synthesis of Compound B-725

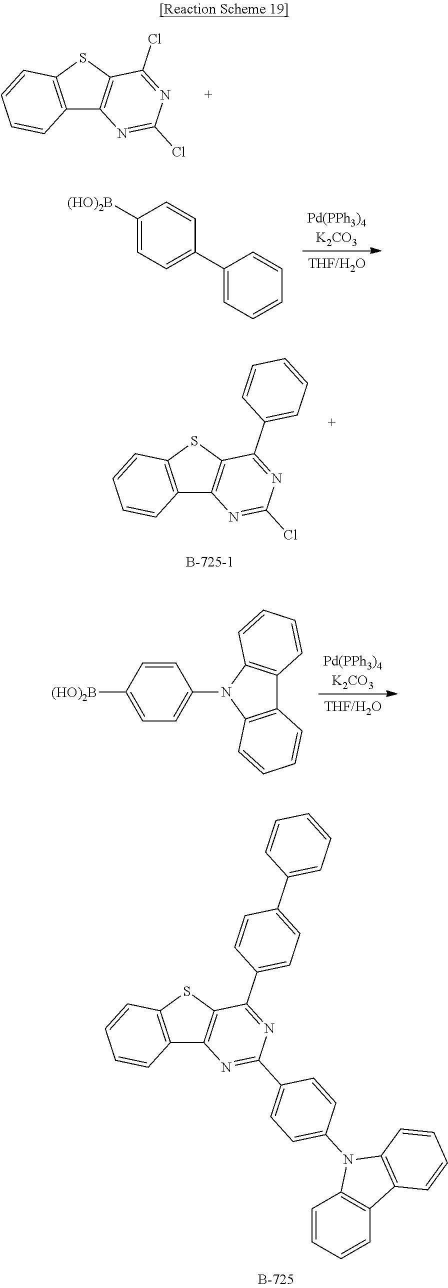

a) Synthesis of Intermediate B-725-1

Intermediate B-725-1 was synthesized according to the same method as the a) of Synthesis Example 11 by reacting 2,4-dichlorobenzo[4,5]thieno[3,2-d]pyrimidine and 4-biphenylboronic acid in each amount of 1.0 equivalent and performing recrystallization with toluene.

b) Synthesis of Compound B-725

Compound B-725 was synthesized according to the same method as the a) of Synthesis Example 11 by reacting Intermediate B-725-1 and (4-(9H-carbazol-9-yl)phenyl)boronic acid in each amount of 1.0 equivalent and performing recrystallization with dichlorobenzene.

LC/MS calculated for: C40H25N3S Exact Mass: 579.18 found for 580.22 [M+H]

### SYNTHESIS EXAMPLE 18

### Synthesis of Compound B-728

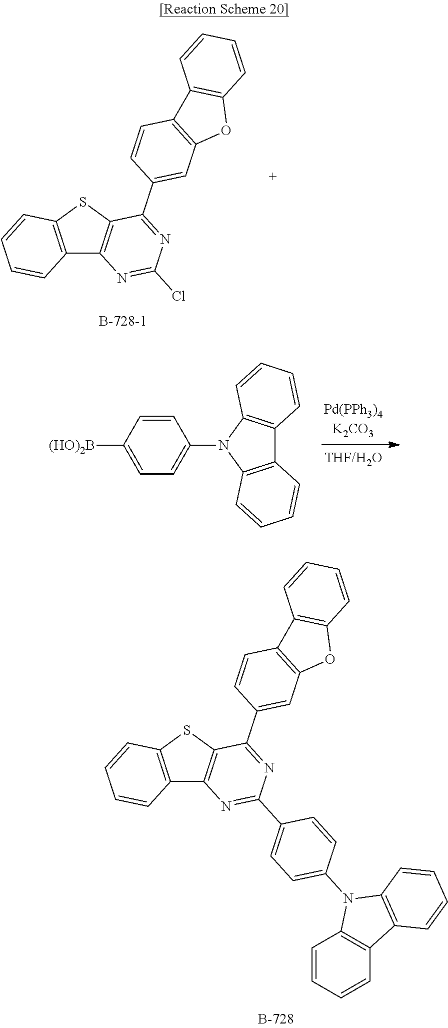 [SMILES: Clc1nc(-c2ccc3c(c2)oc2ccccc23)c2sc3ccccc3c2n1 ; OB(O)c1ccc(-n2c3ccccc3c3ccccc32)cc1 ; c1ccc2c(c1)oc1cc(-c3nc(-c4ccc(-n5c6ccccc6c6ccccc65)cc4)nc4c3sc3ccccc34)ccc12]

a) Synthesis of Compound B-728

Compound B-728 was synthesized according to the same method as the a) of Synthesis Example 11 by reacting Intermediate B-728-1 and (4-(9H-carbazol-9-yl)phenyl)boronic acid in each amount of 1.0 equivalent and performing recrystallization with dichlorobenzene.

LC/MS calculated for: C40H23N3OS Exact Mass: 593.16 found for 594.29 [M+H]

### SYNTHESIS EXAMPLE 19

### Synthesis of Compound B-729

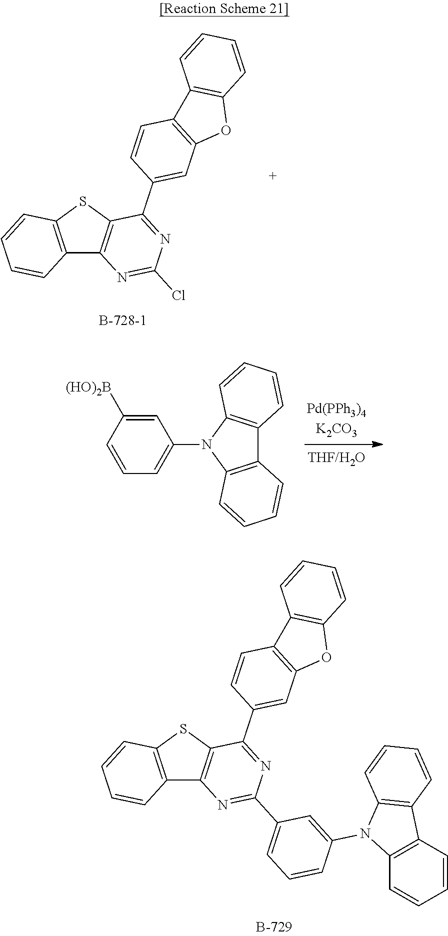 [SMILES: Clc1nc(-c2ccc3c(c2)oc2ccccc23)c2sc3ccccc3c2n1 ; OB(O)c1cccc(-n2c3ccccc3c3ccccc32)c1 ; c1cc(-c2nc(-c3ccc4c(c3)oc3ccccc34)c3sc4ccccc4c3n2)cc(-n2c3ccccc3c3ccccc32)c1]

a) Synthesis of Compound B-729

Compound B-729 was synthesized according to the same method as the a) of Synthesis Example 11 by reacting Intermediate B-728-1 and (3-(9H-carbazol-9-yl)phenyl)boronic acid in each amount of 1.0 equivalent and performing recrystallization with dichlorobenzene.

LC/MS calculated for: C40H23N3OS Exact Mass: 593.16 found for 594.27 [M+H]

### SYNTHESIS EXAMPLE 20

### Synthesis of Compound B-735

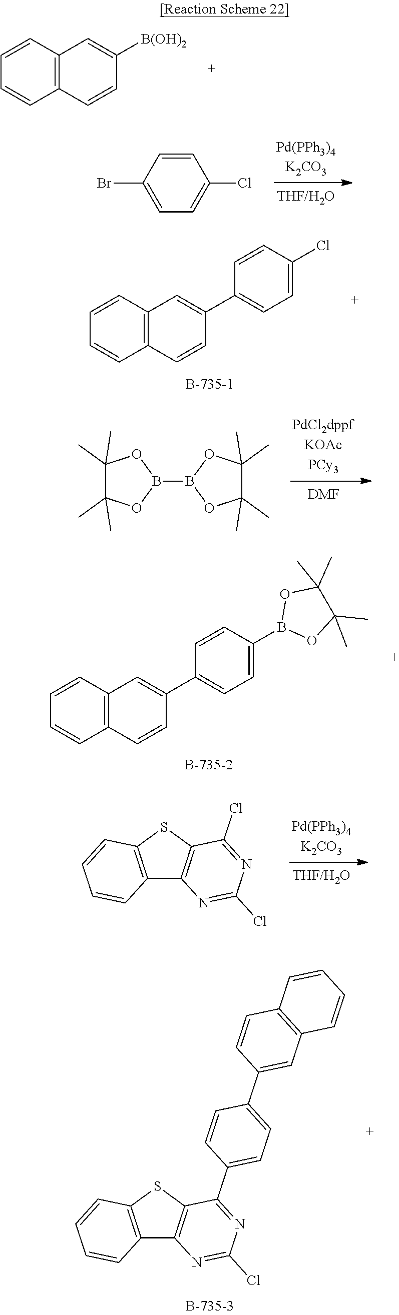

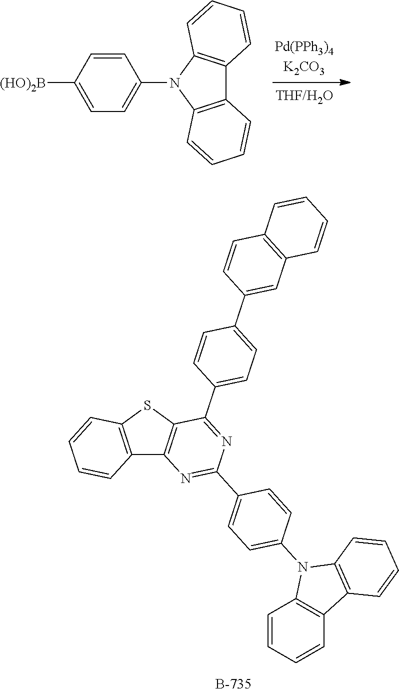 [SMILES: OB(O)c1ccc(-n2c3ccccc3c3ccccc32)cc1 ; c1ccc2cc(-c3ccc(-c4nc(-c5ccc(-n6c7ccccc7c7ccccc76)cc5)nc5c4sc4ccccc45)cc3)ccc2c1]

a) Synthesis of Intermediate B-735-1

Intermediate B-735-1 was synthesized according to the same method as the a) of Synthesis Example 11 by reacting ing 2-naphthaleneboronic acid and 1-bromo-4-chlorobenzene in each amount of 1.0 equivalent and performing recrystallization with toluene.

b) Synthesis of Intermediate B-735-2

Intermediate B-735-2 was synthesized according to the same method as the c) of Synthesis Example 11 by reacting Intermediate B-735-1 and performing recrystallization with a mixed solvent of dichloromethane and n-hexane.

c) Synthesis of Intermediate B-735-3

Intermediate B-735-3 was synthesized according to the same method as the a) of Synthesis Example 11 by reacting 2,4-dichlorobenzo[4,5]thieno[3,2-d]pyrimidine and Intermediate B-735-2 in each amount of 1.0 equivalent and performing recrystallization with chlorobenzene.

d) Synthesis of Compound B-735

Intermediate B-735-5 was synthesized according to the same method as the a) of Synthesis Example 11 by reacting Intermediate B-735-3 and (4-(9H-carbazol-9-yl)phenyl)boronic acid in each amount of 1.0 equivalent and performing recrystallization with dichlorobenzene.

LC/MS calculated for: C44H27N3S Exact Mass: 629.19 found for 630.34 [M+H]

### SYNTHESIS EXAMPLE 21

### Synthesis of Compound B-741

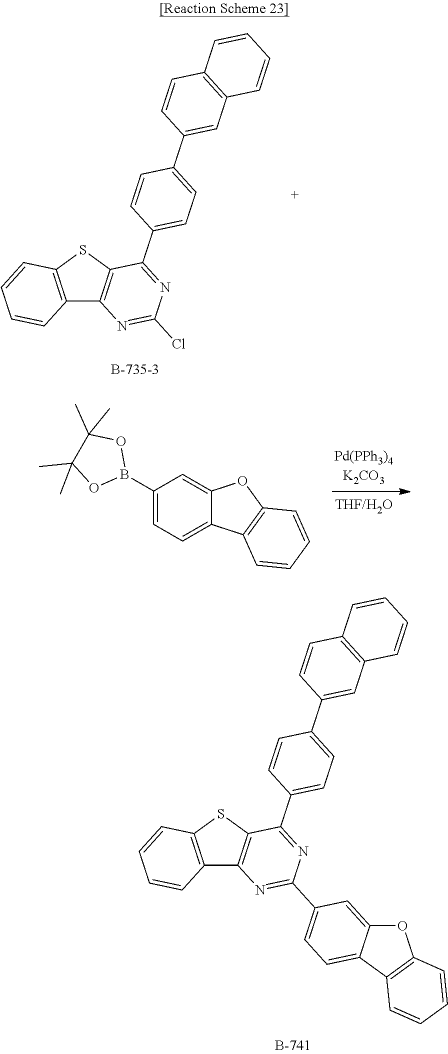 [SMILES: Clc1nc(-c2ccc(-c3ccc4ccccc4c3)cc2)c2sc3ccccc3c2n1 ; CC1(C)OB(c2ccc3c(c2)oc2ccccc23)OC1(C)C ; c1ccc2cc(-c3ccc(-c4nc(-c5ccc6c(c5)oc5ccccc56)nc5c4sc4ccccc45)cc3)ccc2c1]

a) Synthesis of Compound B-741

Compound B-741 was synthesized according to the same method as the a) of Synthesis Example 11 by reacting Intermediate B-735-3 and 2-(dibenzo[b,d]furan-3-yl)-4,4,5,5-tetramethyl-1,3,2-dioxaborolane in each amount of 1.0 equivalent and performing recrystallization with dichlorobenzene.

LC/MS calculated for: C38H22N2OS Exact Mass: 554.15 found for 555.27 [M+H]

### SYNTHESIS EXAMPLE 22

### Synthesis of Compound B-744

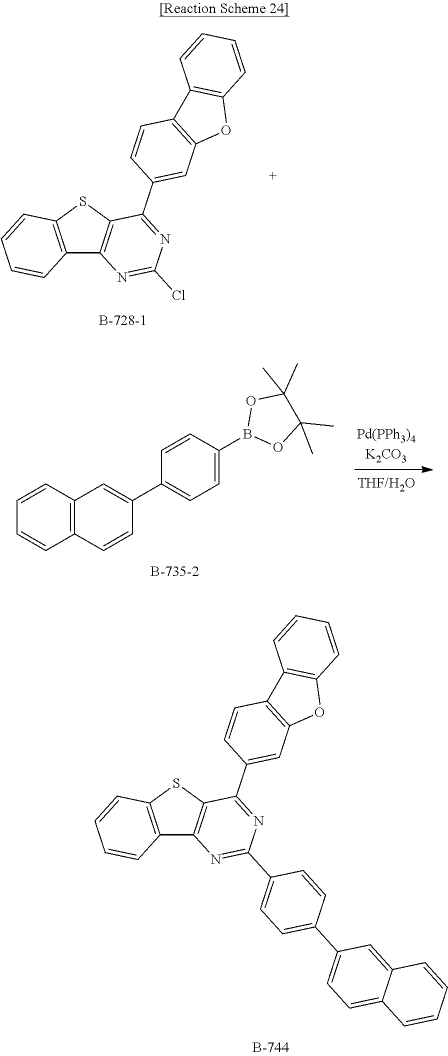 [SMILES: Clc1nc(-c2ccc3c(c2)oc2ccccc23)c2sc3ccccc3c2n1 ; CC1(C)OB(c2ccc(-c3ccc4ccccc4c3)cc2)OC1(C)C ; c1ccc2cc(-c3ccc(-c4nc(-c5ccc6c(c5)oc5ccccc56)c5sc6ccccc6c5n4)cc3)ccc2c1]

a) Synthesis of Compound B-744

Compound B-744 was synthesized according to the same method as the a) of Synthesis Example 11 by reacting Intermediate B-728-1 and Intermediate B-735-2 in each amount of 1.0 equivalent and performing recrystallization with dichlorobenzene.

LC/MS calculated for: C38H22N2OS Exact Mass: 554.15 found for 555.28 [M+H]

### SYNTHESIS EXAMPLE 23

### Synthesis of Compound B-772

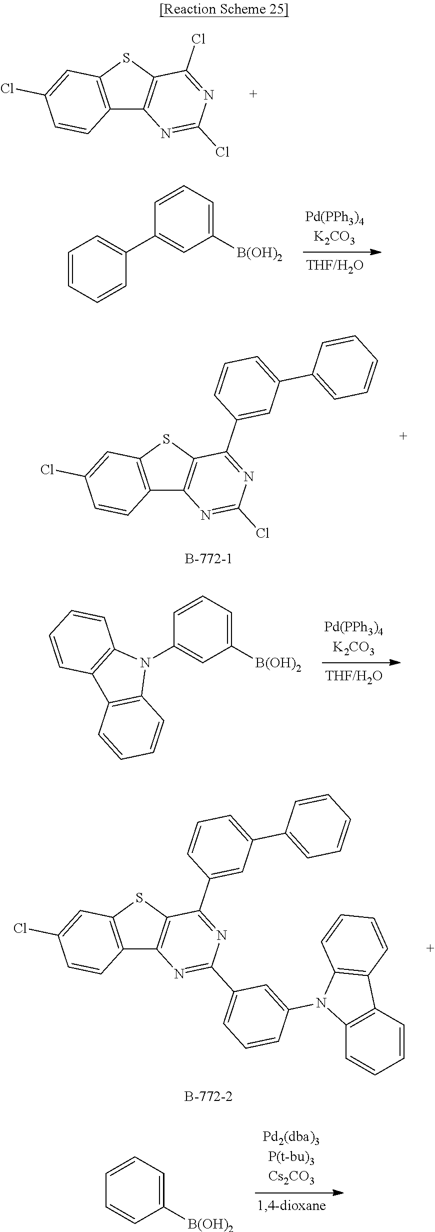 [SMILES: Clc1ccc2c(c1)sc1c(Cl)nc(Cl)nc12 ; OB(O)c1cccc(-c2ccccc2)c1 ; Clc1ccc2c(c1)sc1c(-c3cccc(-c4ccccc4)c3)nc(Cl)nc12 ; OB(O)c1cccc(-n2c3ccccc3c3ccccc32)c1 ; Clc1ccc2c(c1)sc1c(-c3cccc(-c4ccccc4)c3)nc(-c3cccc(-n4c5ccccc5c5ccccc54)c3)nc12 ; OB(O)c1ccccc1]

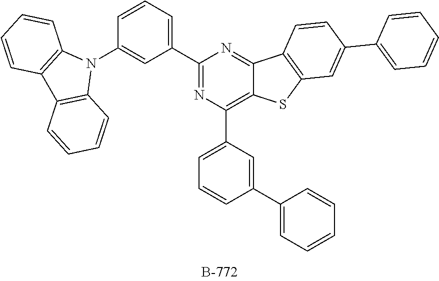 [SMILES: c1ccc(-c2cccc(-c3nc(-c4cccc(-n5c6ccccc6c6ccccc65)c4)nc4c3sc3cc(-c5ccccc5)ccc34)c2)cc1]

a) Synthesis of Intermediate B-772-1

Intermediate B-772-1 was synthesized according to the same method as the a) of Synthesis Example 11 by reacting 2,4,7-trichlorobenzo[4,5]thieno[3,2-d]pyrimidine and 3-biphenylboronic acid in each amount of 1.0 equivalent and performing recrystallization with chlorobenzene.

b) Synthesis of Intermediate B-772-2

Intermediate B-772-2 was synthesized according to the same method as the a) of Synthesis Example 11 by reacting Intermediate B-772-1 and (3-(9H-carbazol-9-yl)phenyl)boronic acid in each amount of 1.0 equivalent and performing recrystallization with chlorobenzene.

c) Synthesis of Compound B-772

Intermediate B-772-2 (15.0 g, 24 mmol), phenylboronic acid (3.57 g, 29 mmol), tris(dibenzylideneacetone)dipalladium(0) (Pd₂(dba)₃) (0.67 g, 0.7 mmol), and cesium carbonate (11.9 g, 37 mmol) were put in a round bottom flask, 100 mL of 1,4-dioxane was added thereto, and a 50% tri-tert-butylphosphine solution (1.4 mL, 3 mmol) was slowly added thereto in a dropwise fashion and then, refluxed and stirred at 100° C. for 12 hours. When a reaction was complete, the solvent was all evaporated therefrom under a reduced pressure. A product obtained therefrom was boiled and dissolved in dichlorobenzene and then, silica gel-filtered and recrystallized to obtain Compound B-772 (5.8 g, 68%).

LC/MS calculated for: C46H29N3S Exact Mass: 655.21 found for 656.35 [M+H]

### SYNTHESIS EXAMPLE 24

### Synthesis of Compound B-846

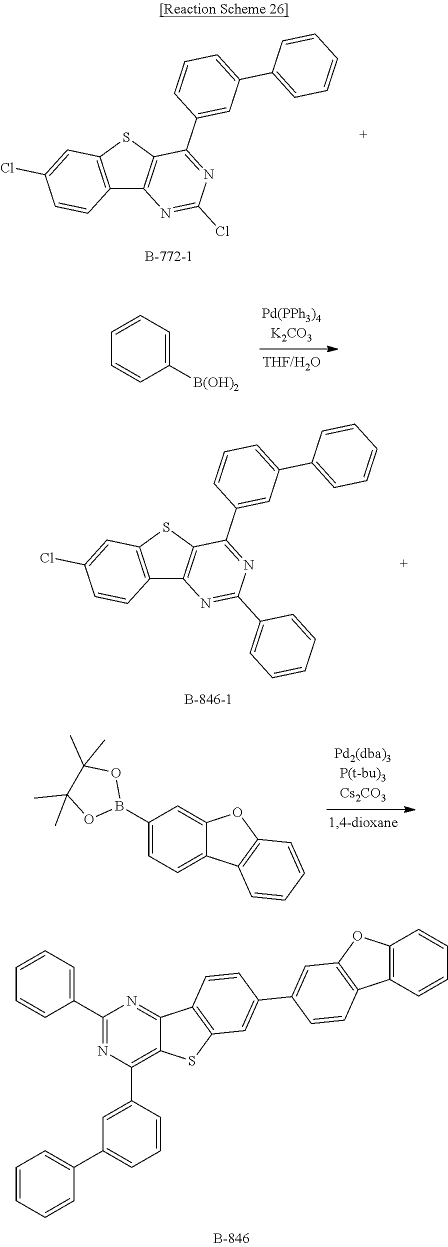 [SMILES: Clc1ccc2c(c1)sc1c(-c3cccc(-c4ccccc4)c3)nc(Cl)nc12 ; OB(O)c1ccccc1 ; Clc1ccc2c(c1)sc1c(-c3cccc(-c4ccccc4)c3)nc(-c3ccccc3)nc12 ; CC1(C)OB(c2ccc3c(c2)oc2ccccc23)OC1(C)C ; c1ccc(-c2cccc(-c3nc(-c4ccccc4)nc4c3sc3cc(-c5ccc6c(c5)oc5ccccc56)ccc34)c2)cc1]

a) Synthesis of Intermediate B-846-1

Intermediate B-846-1 was synthesized according to the same method as the a) of Synthesis Example 11 by reacting Intermediate B-772-1 and phenylboronic acid in each amount of 1.0 equivalent and performing recrystallization with chlorobenzene.

b) Synthesis of Compound B-846

Compound B-846 was synthesized according to the same method as the c) of Synthesis Example 23 by reacting Intermediate B-846-1 and 2-(dibenzo[b,d]furan-3-yl)-4,4,5,5-tetramethyl-1,3,2-dioxaborolane in each amount of 1.0 equivalent and performing recrystallization with dichlorobenzene.

LC/MS calculated for: C40H24N2OS Exact Mass: 580.16 found for 581.23 [M+H]

### (Manufacture of Organic Light Emitting Diode)

### EXAMPLE 1

The glass substrate coated with ITO (Indium tin oxide) at a thickness of 1500 Å was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, and Compound B was deposited to be 50 Å-thick on the injection layer, and then Compound C was deposited to be 700 Å-thick to form a hole transport layer. Compound C-1 was deposited to a thickness of 400 Å on the hole transport layer to form a hole transport auxiliary layer. Compounds A-2 and B-716 were simultaneously used as hosts on the hole transport auxiliary layer and doped with 2 wt % of [Ir(piq)₂acac] as a dopant to form a 400 Å-thick light emitting layer by vacuum deposition. Herein, Compound A-2 and Compound B-716 were used in a weight ratio of 5:5, and the ratio was separately described for the following examples. Subsequently, on the light emitting layer, a 300 Å-thick electron transport layer was formed by simultaneously vacuum-depositing Compound D and Liq in a weight ratio of 1:1, and on the electron transport layer, Liq and Al were sequentially vacuum-deposited to be 15 Å-thick and 1200 Å-thick, manufacturing an organic light emitting diode.

The organic light emitting diode had a five-layered organic thin layer, and specifically the following structure.

ITO/Compound A (700 Å)/Compound B (50 Å)/Compound C (700 Å)/EML [Compound A-2: B-716 [Ir(piq)₂acac] (2 wt %)] (400 Å)/Compound D:Liq (300 Å)/Liq (15 Å)/Al (1200 Å)

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN),

Compound C: N-(biphenyl-4-yl)-9,9-dimethyl-N-(4-(9-phenyl-9H-carbazol-3-yl)phenyl)-9H-fluoren-2-amine

Compound C-1: N,N-di([1,1′-biphenyl]-4-yl)-7,7-dimethyl-7H-fluoreno[4,3-b]benzofuran-10-amine

Compound D: 8-(4-(4,6-di(naphthalen-2-yl)-1,3,5-triazin-2-yl)phenyl)quinoline

### EXAMPLES 2 TO 14

Each organic light emitting diode was manufactured in the same manner as in Example 1, except that the composition was changed to the compositions shown in Table 1.

### COMPARATIVE EXAMPLES 1 AND 2

Each organic light emitting diode was manufactured in the same manner as in Example 1, except that the composition was changed to the compositions shown in Table 1.

### Evaluation

The power efficiency of the organic light emitting diodes according to Examples 1 to 14 and Comparative Examples 1 and 2 was evaluated.

Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000 A), while the voltage of the organic light emitting diodes was increased from 0 V to 10 V.

(3) Measurement of Electric Power Efficiency

Electric power efficiency (cd/A) at the same current density (10 mA/cm²) were calculated by using the current density and voltage from the items (1) and (2).

(4) Measurement of Life-Span

The luminance (cd/m²) was maintained at 9000 cd/m² and the time at which the current efficiency (cd/A) decreased to 97% was measured to obtain results.

(5) Measurement of Driving Voltage

A driving voltage of each diode was measured by using a current-voltage meter (Keithley 2400) at 15 mA/cm².

TABLE 1

| | | | First | | | | |
|---|---|---|---|---|---|---|---|
| | | | host: | | | | |
| | | | Second | | Electric | | Life- |
| | | | host | | power | Driving | span |
| | First | Second | Ratio | | Efficiency | voltage | (T97) |
| | host | host | (wt:wt) | Color | (cd/A) | (V) | (h) |
| Example 1 | A-2 | B-716 | 5:5 | red | 20.4 | 4.25 | 115 |
| Example 2 | A-2 | B-728 | 5:5 | red | 20.6 | 4.20 | 125 |
| Example 3 | A-2 | B-728 | 6:4 | red | 20.4 | 4.24 | 140 |
| Example 4 | A-2 | B-729 | 5:5 | red | 21.0 | 4.27 | 130 |
| Example 5 | A-2 | B-729 | 6:4 | red | 20.8 | 4.30 | 135 |
| Example 6 | A-2 | B-735 | 5:5 | red | 21.1 | 4.18 | 140 |
| Example 7 | A-2 | B-741 | 5:5 | red | 20.9 | 4.21 | 130 |
| Example 8 | A-2 | B-744 | 5:5 | red | 20.8 | 4.24 | 125 |
| Example 9 | A-2 | B-772 | 5:5 | red | 20.3 | 3.98 | 130 |
| Example 10 | A-2 | B-772 | 6:4 | red | 20.2 | 4.10 | 140 |
| Example 11 | A-2 | B-846 | 5:5 | red | 20.4 | 3.96 | 135 |
| Example 12 | A-2 | B-846 | 6:4 | red | 20.4 | 4.05 | 140 |
| Example 13 | A-11 | B-728 | 5:5 | red | 21.0 | 4.18 | 145 |
| Example 14 | A-29 | B-728 | 5:5 | red | 20.3 | 4.27 | 110 |
| Comparative | V-1 | B-728 | 5:5 | red | 16.2 | 4.88 | 5 |
| Example 1 | | | | | | | |
| Comparative | V-2 | B-728 | 5:5 | red | 19.5 | 4.55 | 30 |
| Example 2 | | | | | | | |

Referring to Table 1, the driving voltage, efficiency, and life-span of the organic light emitting diodes according to Examples 1 to 14 are significantly improved compared with the organic light emitting diodes according to Comparative Examples 1 and 2.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

1. A composition for an organic optoelectronic element, comprising:
a first compound for an organic optoelectronic element, represented by a combination of Chemical Formula 1 and Chemical Formula 2, and
a second compound for an organic optoelectronic element, represented by a combination of Chemical Formula 3 and Chemical Formula 4: 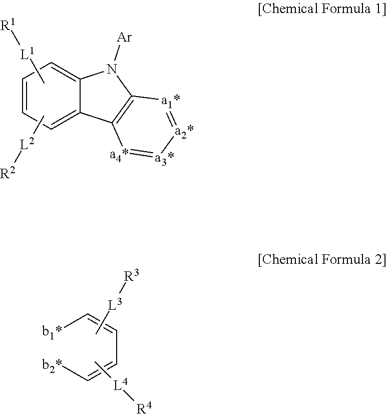
wherein, in Chemical Formula 1 and Chemical Formula 2,
Ar is a substituted or unsubstituted C6 to C30 aryl group, a substituted or unsubstituted C2 to C30 heterocyclic group, or a combination thereof,
adjacent two of a₁* to a₄* are C linked with b₁* and b₂*, respectively,
the rest of a₁* to a₄*, not linked with b₁* and b₂*, are independently C-L^{a}-R^{a},
L^{a} and L¹ to L⁴ are independently a single bond, a substituted or unsubstituted C6 to C20 arylene group, a substituted or unsubstituted C2 to C20 heterocyclic group, or a combination thereof,
R^{a} and R¹ to R⁴ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R^{a} and R¹ to R⁴ is a group represented by Chemical Formula a, 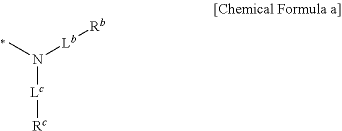
wherein, in Chemical Formula a,
L^{b} and L^{c} are independently a single bond, a substituted or unsubstituted C6 to C20 arylene group, a substituted or unsubstituted C2 to C20 heterocyclic group, or a combination thereof,
R^{b} and RC are independently a substituted or unsubstituted C6 to C30 aryl group, a substituted or unsubstituted C2 to C30 heterocyclic group, or a combination thereof, and
* is a linking point with L^{a} and L¹ to L⁴; 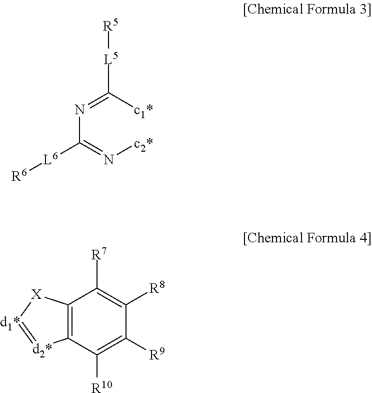
wherein, in Chemical Formula 3 and Chemical Formula 4,
X is O or S,
d₁* and d₂* are C linked with c₁* and c₂*, respectively, or d₁* and d₂* are C linked with c₂* and c₁*, respectively
L⁵ and L⁶ are independently a single bond, a substituted or unsubstituted C6 to C20 arylene group, a substituted or unsubstituted C2 to C20 heterocyclic group, or a combination thereof,
R⁵ to R¹⁰ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R⁵ and R⁶ is a substituted or unsubstituted C6 to C30 aryl group, a substituted or unsubstituted C2 to C30 heterocyclic group, or a combination thereof

2. The composition for an organic optoelectronic element of claim 1, wherein the first compound for an organic optoelectronic element is represented by one of Chemical Formula 1A to Chemical Formula 1C: 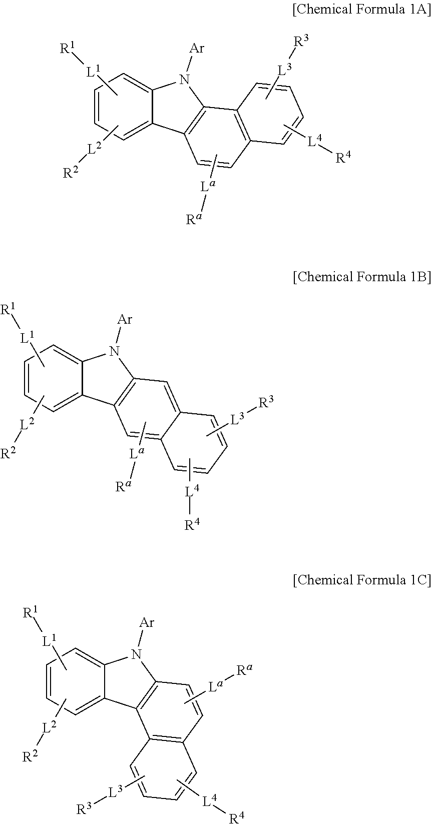
wherein, in Chemical Formula 1A to Chemical Formula 1C,
Ar is a substituted or unsubstituted C6 to C30 aryl group, a substituted or unsubstituted C2 to C30 heterocyclic group, or a combination thereof,
L^{a} and L¹ to L⁴ are independently a single bond, a substituted or unsubstituted C6 to C20 arylene group, a substituted or unsubstituted C2 to C20 heterocyclic group, or a combination thereof,
R^{a} and R¹ to R⁴ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R^{a} and R¹ to R⁴ is a group represented by Chemical Formula a, 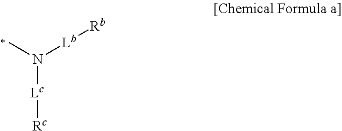
wherein, in Chemical Formula a,
L^{b} and L^{c} are independently a single bond, a substituted or unsubstituted C6 to C20 arylene group, a substituted or unsubstituted C2 to C20 heterocyclic group, or a combination thereof,
R^{b} and R^{c} are independently a substituted or unsubstituted C6 to C30 aryl group, a substituted or unsubstituted C2 to C30 heterocyclic group, or a combination thereof, and
* is a linking point with L^{a} and L¹ to L⁴.

3. The composition for an organic optoelectronic element of claim 1, wherein the first compound for an organic optoelectronic element is represented by one of Chemical Formula 1A-1 to Chemical Formula 1A-3, Chemical Formula 1B-1 to Chemical Formula 1B-3, and Chemical Formula 1C-1 to Chemical Formula 1C-3: 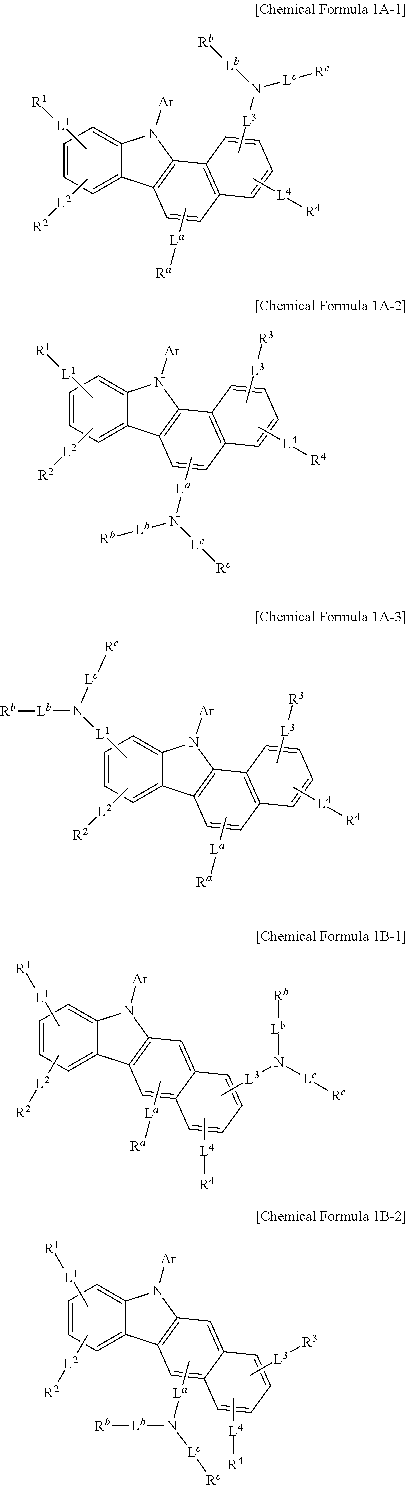 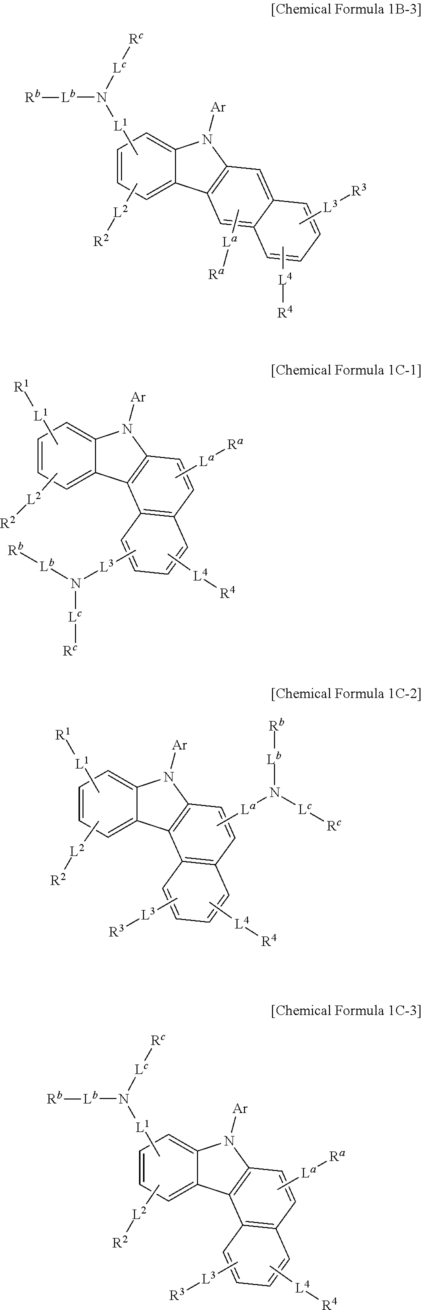
wherein, in Chemical Formula 1A-1 to Chemical Formula 1A-3, Chemical Formula 1B-1 to Chemical Formula 1B-3, and Chemical Formula 1C-1 to Chemical Formula 1C-3,
Ar is a substituted or unsubstituted C6 to C30 aryl group, a substituted or unsubstituted C2 to C30 heterocyclic group, or a combination thereof,
L^{a}, L^{b}, L^{c}, and L¹ to L⁴ are independently a single bond, a substituted or unsubstituted C6 to C20 arylene group, a substituted or unsubstituted C2 to C20 heterocyclic group, or a combination thereof,
R^{a} and R¹ to R⁴ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
R^{b} and R^{c} are independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group or a substituted or unsubstituted dibenzothiophenyl group.

4. The composition for an organic optoelectronic element of claim 1, wherein the first compound for an organic optoelectronic element is represented by Chemical Formula 1A-1-b: 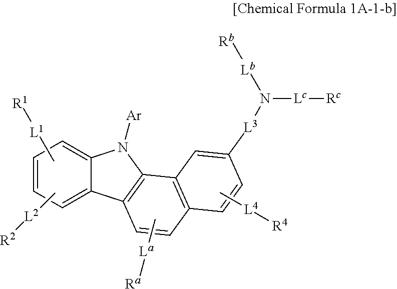
wherein, in Chemical Formula 1A-1-b,
Ar is a substituted or unsubstituted C6 to C30 aryl group, a substituted or unsubstituted C2 to C30 heterocyclic group, or a combination thereof,
L^{a}, L^{b}, L^{c}, and L¹ to L⁴ are independently a single bond, a substituted or unsubstituted C6 to C20 arylene group, a substituted or unsubstituted C2 to C20 heterocyclic group, or a combination thereof,
R^{a}, R², and R⁴ are independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof, and
R^{b} and R^{c} are independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group or a substituted or unsubstituted dibenzothiophenyl group.

5. The composition for an organic optoelectronic element of claim 1, wherein the second compound for an organic optoelectronic element is represented by Chemical Formula 2-I or Chemical Formula 2-II: 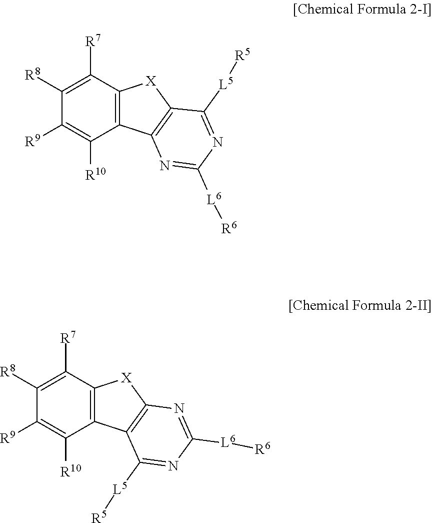
wherein, in Chemical Formula 2-I and 2-II,
X is O or S,
L⁵ and L⁶ are independently a single bond, a substituted or unsubstituted C6 to C20 arylene group, a substituted or unsubstituted C2 to C20 heterocyclic group, or a combination thereof,
R⁵ to R¹⁰ are independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R⁵ and R⁶ is a substituted or unsubstituted C6 to C30 aryl group, a substituted or unsubstituted C2 to C30 heterocyclic group, or a combination thereof

6. The composition for an organic optoelectronic element of claim 5, wherein R⁵ and R⁶ are independently a substituted or unsubstituted C6 to C30 aryl group, a substituted or unsubstituted C2 to C30 heterocyclic group, or a combination thereof.

7. The composition for an organic optoelectronic element of claim 6, wherein R⁵ and R⁶ are independently a substituted or unsubstituted phenyl group, a substituted or unsubstituted biphenyl group, a substituted or unsubstituted naphthyl group, a substituted or unsubstituted dibenzofuranyl group, a substituted or unsubstituted dibenzothiophenyl group, a substituted or unsubstituted carbazolyl group, or a combination thereof

8. The composition for an organic optoelectronic element of claim 1, wherein
the first compound for an organic optoelectronic element is represented by Chemical Formula 1A-1-b, and
the second compound for an organic optoelectronic element is represented by Chemical Formula 2-I: 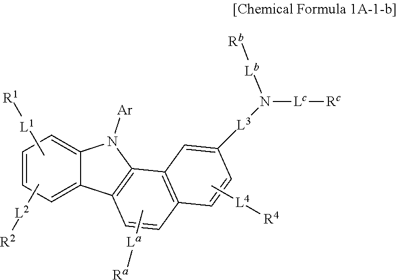
wherein, in Chemical Formula 1A-1-b,
Ar is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof,
L^{a}, L^{b}, L^{c}, and L¹ to L⁴ are independently a single bond, a substituted or unsubstituted phenylene group, a substituted or unsubstituted biphenylene group, a substituted or unsubstituted terphenylene group, or a substituted or unsubstituted naphthylene group,
R^{a}, R¹, R², and R⁴ are independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof, and
R^{b} and R^{c} are independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group or a substituted or unsubstituted dibenzothiophenyl group; 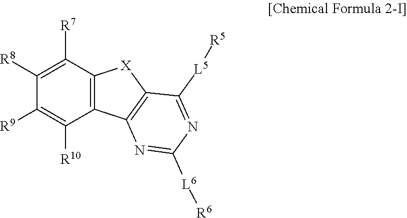
wherein, in Chemical Formula 2-I,
X is O or S,
L⁵ and L⁶ are independently a single bond, a substituted or unsubstituted C6 to C20 arylene group, a substituted or unsubstituted C2 to C20 heterocyclic group, or a combination thereof,
R⁵ and R⁶ are independently a substituted or unsubstituted phenyl group, a substituted or unsubstituted biphenyl group, a substituted or unsubstituted naphthyl group, a substituted or unsubstituted dibenzofuranyl group, a substituted or unsubstituted dibenzothiophenyl group, a substituted or unsubstituted carbazolyl group, or a combination thereof, and
R⁷ to R¹⁰ are independently hydrogen, deuterium, a substituted or unsubstituted C1 to C10 alkyl group, a substituted or unsubstituted C6 to C12 aryl group, or a combination thereof

9. The composition for an organic optoelectronic element of claim 1, further comprising a dopant.

10. An organic optoelectronic element, comprising:
an anode and a cathode facing each other,
an organic layer between the anode and the cathode,
wherein the organic layer includes the composition for an organic optoelectronic element of claim 1.

11. The organic optoelectronic element of claim 10, wherein
the organic layer includes a light emitting layer, and
the light emitting layer includes the composition for an organic optoelectronic element.

12. The organic optoelectronic element of claim 11, wherein the first compound for an organic optoelectronic element and the second compound for an organic optoelectronic element are each included as a phosphorescent host of the light emitting layer.

13. The organic optoelectronic element of claim 12, wherein the composition for an organic optoelectronic element is a red light emitting composition.

14. A display device comprising the organic optoelectronic element of claim 10.
